SEQUENCE LISTING (389 sequences)

SEQ ID NO: 1 - protein, 7 aa, Artificial Sequence
  Anti-Human Cleaved CDR L1
SVSSAVA

SEQ ID NO: 2 - protein, 6 aa, Artificial Sequence
  Anti-Human Cleaved CDR L2
SASSLY

SEQ ID NO: 3 - protein, 6 aa, Artificial Sequence
  Anti-Human Cleaved CDR L3 -- CL03 (Fab, IgG); CL03 IgG H1-NtoD; CL03 IgG H1-NtoD (H3-MtoA (“IgG101”), H3-MtoI, H3-MtoL, H3-MtoV); IgGA(01, 03-06); IgG(87, 89, 94, 97, 102)
SGQRPI

SEQ ID NO: 4 - protein, 6 aa, Artificial Sequence
  Anti-Human Cleaved CDR L3 -- CL07 (Fab, IgG, IgG H1-NtoD); IgG3H7L; IgGD09
SSSSLI

SEQ ID NO: 5 - protein, 6 aa, Artificial Sequence
  Anti-Human Cleaved CDR L3 -- IgGA08
SGERPI

SEQ ID NO: 6 - protein, 6 aa, Artificial Sequence
  Anti-Human Cleaved CDR L3 -- IgGA09
SGNVPA

SEQ ID NO: 7 - protein, 6 aa, Artificial Sequence
  Anti-Human Cleaved CDR L3 -- IgGA10
SGKRPM

SEQ ID NO: 8 - protein, 6 aa, Artificial Sequence
  Anti-Human Cleaved CDR L3 -- IgGA11
TGQRPM

SEQ ID NO: 9 - protein, 6 aa, Artificial Sequence
  Anti-Human Cleaved CDR L3 -- IgGB01
SMQWPK

SEQ ID NO: 10 - protein, 6 aa, Artificial Sequence
  Anti-Human Cleaved CDR L3 -- IgGB03
SLQRPM

SEQ ID NO: 11 - protein, 6 aa, Artificial Sequence
  Anti-Human Cleaved CDR L3 -- IgGB04
VELVPM

SEQ ID NO: 12 - protein, 6 aa, Artificial Sequence
  Anti-Human Cleaved CDR L3 -- IgGB07
AGKRPL

SEQ ID NO: 13 - protein, 6 aa, Artificial Sequence
  Anti-Human Cleaved CDR L3 -- IgGB08
SVDLTA

SEQ ID NO: 14 - protein, 6 aa, Artificial Sequence
  Anti-Human Cleaved CDR L3 -- IgGC01
LGVRAA

SEQ ID NO: 15 - protein, 6 aa, Artificial Sequence
  Anti-Human Cleaved CDR L3 -- IgGC03
SGRKPV

SEQ ID NO: 16 - protein, 6 aa, Artificial Sequence
  Anti-Human Cleaved CDR L3 -- IgGC04
TAQSPL

SEQ ID NO: 17 - protein, 6 aa, Artificial Sequence
  Anti-Human Cleaved CDR L3 -- IgGC05
SRQRPL

SEQ ID NO: 18 - protein, 6 aa, Artificial Sequence
  Anti-Human Cleaved CDR L3 -- IgGC06
SGLMPL

SEQ ID NO: 19 - protein, 6 aa, Artificial Sequence
  Anti-Human Cleaved CDR L3 -- IgGC09
SGHRPI

SEQ ID NO: 20 - protein, 16 aa, Artificial Sequence
  Anti-Human Cleaved CDR H3 -- IgGD02
KYHSYYSHTWWVSYGM

SEQ ID NO: 21 - protein, 6 aa, Artificial Sequence
  Anti-Human Cleaved CDR L3 -- IgGD02
SSQRPA

SEQ ID NO: 22 - protein, 6 aa, Artificial Sequence
  Anti-Human Cleaved CDR L3 -- IgGD03
SGRQPL

SEQ ID NO: 23 - protein, 6 aa, Artificial Sequence
  Anti-Human Cleaved CDR L3 -- IgGD04
FMRPAF

SEQ ID NO: 24 - protein, 6 aa, Artificial Sequence
  Anti-Human Cleaved CDR L3 -- IgGD07
SGQPSM

SEQ ID NO: 25 - protein, 6 aa, Artificial Sequence
  Anti-Human Cleaved CDR L3 -- IgGD08
SVLRPF

SEQ ID NO: 26 - protein, 6 aa, Artificial Sequence
  Anti-Human Cleaved CDR H1 -- CL03 (Fab, IgG); CL03 IgG H1-NtoD; CL03 IgG H1-NtoD (H3-MtoA (“IgG101”), H3-MtoI, H3-MtoL, H3-MtoV); IgG(94, 97, 102)
FSSYSI

SEQ ID NO: 27 - protein, 6 aa, Artificial Sequence
  Anti-Human Cleaved CDR H1 -- CL07 (Fab, IgG, IgG H1-NtoD)
FSSSSI

SEQ ID NO: 28 - protein, 7 aa, Artificial Sequence
  Anti-Human Cleaved CDR H1 -- IgG3H7L; IgGA(01, 03-06, 08-11); IgGB(01, 03, 04, 07, 08); IgGC(01, 03-06, 09); IgGD(02-04, 07-08)
  MISC_FEATURE (1)..(1): Wherein Xaa is either Asp or the absence of an amino acid
XFSSYSI

SEQ ID NO: 29 - protein, 7 aa, Artificial Sequence
  Anti-Human Cleaved CDR H1 -- IgGD09
  MISC_FEATURE (1)..(1): Wherein Xaa is either Asn or the absence of an amino acid
XFSSSSI

SEQ ID NO: 30 - protein, 10 aa, Artificial Sequence
  Anti-Human Cleaved CDR H2 -- CL03 (Fab, IgG); CL03 IgG H1-NtoD; CL03 IgG H1-NtoD (H3-MtoA (“IgG101”), H3-MtoI, H3-MtoL, H3-MtoV); IgG3H7L; IgGA(01, 03-06, 08-11); IgGB(01, 03, 04, 07, 08); IgGC(01, 03-06, 09); IgGD(02-04, 07-08); IgG(87, 89, 97)
SIYPYSGSTY

SEQ ID NO: 31 - protein, 10 aa, Artificial Sequence
  Anti-Human Cleaved CDR H2 -- CL07 (Fab, IgG, IgG H1-NtoD); IgGD09; IgG94
SIYPYSGSTS

SEQ ID NO: 32 - protein, 16 aa, Artificial Sequence
  Anti-Human Cleaved CDR H3 -- CL03 (Fab, IgG, IgG H1-NtoD); IgG3H7L; IgGA(08-10); IgGB01; IgGC(03, 05, 06, 09); IgGD(02, 08); IgG(87, 89, 94, 102)
QSHSYYSHTWWVSYGM

SEQ ID NO: 33 - protein, 16 aa, Artificial Sequence
  Anti-Human Cleaved CDR H3 -- CL03 IgG H1-NtoD H3-MtoA (“IgG101”)
QSHSYYSHTWWVSYGA

SEQ ID NO: 34 - protein, 16 aa, Artificial Sequence
  Anti-Human Cleaved CDR H3 -- CL03 IgG H1-NtoD H3-MtoI
QSHSYYSHTWWVSYGI

SEQ ID NO: 35 - protein, 16 aa, Artificial Sequence
  Anti-Human Cleaved CDR H3 -- CL03 IgG H1-NtoD H3-MtoL
QSHSYYSHTWWVSYGL

SEQ ID NO: 36 - protein, 16 aa, Artificial Sequence
  Anti-Human Cleaved CDR H3 -- CL03 IgG H1-NtoD H3-MtoV
QSHSYYSHTWWVSYGV

SEQ ID NO: 37 - protein, 18 aa, Artificial Sequence
  Anti-Human Cleaved CDR H3 -- CL07 (Fab, IgG, IgG H1-NtoD)
TYWVQFWYGHFSYYRPAL

SEQ ID NO: 38 - protein, 16 aa, Artificial Sequence
  Anti-Human Cleaved CDR H3 -- IgGA01
QNHSYYSHTWWVSYGM

SEQ ID NO: 39 - protein, 16 aa, Artificial Sequence
  Anti-Human Cleaved CDR H3 -- IgGA03
QTHSYYSHTWWVSYGM

SEQ ID NO: 40 - protein, 16 aa, Artificial Sequence
  Anti-Human Cleaved CDR H3 -- IgGA04
QGHSYYSHTWWVSYGM

SEQ ID NO: 41 - protein, 16 aa, Artificial Sequence
  Anti-Human Cleaved CDR H3 -- IgGA05
QAHSYYSHTWWVSYGM

SEQ ID NO: 42 - protein, 16 aa, Artificial Sequence
  Anti-Human Cleaved CDR H3 -- IgGA06
QGQSYYSHTWWVSYGM

SEQ ID NO: 43 - protein, 16 aa, Artificial Sequence
  Anti-Human Cleaved CDR H3 -- IgGA11; IgGB(04, 08); IgGC(01, 04); IgGD04
RSHSYYSHTWWVSYGM

SEQ ID NO: 44 - protein, 16 aa, Artificial Sequence
  Anti-Human Cleaved CDR H3 -- IgGB07, IgGD03
QDHSYYSHTWWVSYGM

SEQ ID NO: 45 - protein, 16 aa, Artificial Sequence
  Anti-Human Cleaved CDR H3 -- IgGB03
QSHSFYSHTWWVSYGM

SEQ ID NO: 46 - protein, 16 aa, Artificial Sequence
  Anti-Human Cleaved CDR H3 -- IgGD07
KSHSYYSHTWWVSYGM

SEQ ID NO: 47 - protein, 18 aa, Artificial Sequence
  Anti-Human Cleaved CDR H3 -- IgGD09
IYWVDFWYGHFSYYRPAL

SEQ ID NO: 48 - protein, 6 aa, Artificial Sequence
  Anti-Mouse Cleaved CDR L3 -- Fab 0114, IgG 0029 (Fab 0114)
SSWGLF

SEQ ID NO: 49 - protein, 6 aa, Artificial Sequence
  Anti-Mouse Cleaved CDR L3 -- Fab 0123, IgG 0030 (Fab 0123), IgG 0046, IgG 0050, IgG 0051, IgG 0055, IgG 0058, IgG 0059, IgG 0060, IgG 0062, IgG 0063, IgG 0065
TYSGPI

SEQ ID NO: 50 - protein, 6 aa, Artificial Sequence
  Anti-Mouse Cleaved CDR H1 -- Fab 0114, IgG 0029 (Fab 0114)
VSYSSI

SEQ ID NO: 51 - protein, 6 aa, Artificial Sequence
  Anti-Mouse Cleaved CDR H1 -- Fab 0123, IgG 0030 (Fab 0123), IgG 0046, IgG 0050, IgG 0051, IgG 0055, IgG 0058, IgG 0059, IgG 0060, IgG 0062, IgG 0063, IgG 0065
VYSYSI

SEQ ID NO: 52 - protein, 10 aa, Artificial Sequence
  Anti-Mouse Cleaved CDR H2 -- Fab 0114, IgG 0029 (Fab 0114), Fab 0123, IgG 0030 (Fab 0123), IgG 0046, IgG 0055
SISSSSGYTY

SEQ ID NO: 53 - protein, 10 aa, Artificial Sequence
  Anti-Mouse Cleaved CDR H2 -- IgG 0050, IgG 0059, IgG 0062
SISSSSGSTY

SEQ ID NO: 54 - protein, 10 aa, Artificial Sequence
  Anti-Mouse Cleaved CDR H2 -- IgG 0051, IgG 0060, IgG 0063
SISSSSGYTS

SEQ ID NO: 55 - protein, 10 aa, Artificial Sequence
  Anti-Mouse Cleaved CDR H2 -- IgG 0058, IgG 0065
SISSSSGSTS

SEQ ID NO: 56 - protein, 13 aa, Artificial Sequence
  Anti-Mouse Cleaved CDR H3 -- Fab 0114, IgG 0029 (Fab 0114)
QEGDWWVAYWPAL

SEQ ID NO: 57 - protein, 16 aa, Artificial Sequence
  Anti-Mouse Cleaved CDR H3 -- Fab 0123, IgG 0030 (Fab 0123)
YYWDPWQYWYYSYLAF

SEQ ID NO: 58 - protein, 16 aa, Artificial Sequence
  Anti-Mouse Cleaved CDR H3 -- IgG 0046, IgG 0050, IgG 0051
YYWDPWQYAYYSYLAF

SEQ ID NO: 59 - protein, 16 aa, Artificial Sequence
  Anti-Mouse Cleaved CDR H3 -- IgG 0055, IgG 0058, IgG 0059, IgG 0060
YYWDPWQYASYSYLAF

SEQ ID NO: 60 - protein, 16 aa, Artificial Sequence
  Anti-Mouse Cleaved CDR H3 -- IgG 0062, IgG 0063, IgG 0065
YYWDPWQYSSYSYLAF

SEQ ID NO: 61 - protein, 127 aa, Artificial Sequence
  Anti-Human Cleaved Variable Heavy Chain -- CL03 Fab, CL03 IgG
EVQLVESGGGLVQPGGSLRLSCAASGFNFSSYSIHWVRQAPGKGLEWVASIYPYSGSTYYADSVKGRFTISADTSKNTAYLQMNSLRAEDTAVYYCARQSHSYYSHTWWVSYGMDYWGQGTLVTVSS

SEQ ID NO: 62 - protein, 107 aa, Artificial Sequence
  Anti-Human Cleaved Variable Light Chain -- CL03 (Fab, IgG); CL03 IgG H1-NtoD; CL03 IgG H1-NtoD (H3-MtoA (“IgG101”), H3-MtoI, H3-MtoL, H3-MtoV); IgGA(01, 03-06), IgG(87, 89, 94, 97, 102)
DIQMTQSPSSLSASVGDRVTITCRASQSVSSAVAWYQQKPGKAPKLLIYSASSLYSGVPSRFSGSRSGTDFTLTISSLQPEDFATYYCQQSGQRPITFGQGTKVEIK

SEQ ID NO: 63 - protein, 336 aa, Artificial Sequence
  c-CDCP1 Cut 1 N-terminal fragment (NTF) of extracellular domain
FEIALPRESNITVLIKLGTPTLLAKPCYIVISKRHITMLSIKSGERIVFTFSCQSPENHFVIEIQKNIDCMSGPCPFGEVQLQPSTSLLPTLNRTFIWDVKAHKSIGLELQFSIPRLRQIGPGESCPDGVTHSISGRIDATVVRIGTFCSNGTVSRIKMQEGVKMALHLPWFHPRNVSGFSIANRSSIKRLCIIESVFEGEGSATLMSANYPEGFPEDELMTWQFVVPAHLRASVSFLNFNLSNCERKEERVEYYIPGSTTNPEVFKLEDKQPGNMAGNFNLSLQGCDQDAQSPGILRLQFQVLVQHPQNESNKIYVVDLSNERAMSLTIEPRPVK

SEQ ID NO: 64 - protein, 471 aa, Artificial Sequence
  c-CDCP1 Cut 1 C-terminal fragment (CTF) of extracellular domain, with transmembrane domain (TM) and intracellular domain (ICD)
QSRKFVPGCFVCLESRTCSSNLTLTSGSKHKISFLCDDLTRLWMNVEKTISCTDHRYCQRKSYSLQVPSDILHLPVELHDFSWKLLVPKDRLSLVLVPAQKLQQHTHEKPCNTSFSYLVASAIPSQDLYFGSFCPGGSIKQIQVKQNISVTLRTFAPSFQQEASRQGLTVSFIPYFKEEGVFTVTPDTKSKVYLRTPNWDRGLPSLTSVSWNISVPRDQVACLTFFKERSGVVCQTGRAFMIIQEQRTRAEEIFSLDEDVLPKPSFHHHSFWVNISNCSPTSGKQLDLLFSVTLTPRTVDLTVILIAAVGGGVLLLSALGLIICCVKKKKKKTNKGPAVGIYNDNINTEMPRQPKKFQKGRKDNDSHVYAVIEDTMVYGHLLQDSSGSFLQPEVDTYRPFQGTMGVCPPSPPTICSRAPTAKLATEEPPPRSPPESESEPYTFSHPNNGDVSSKDTDIPLLNTQEPMEPAE

SEQ ID NO: 65 - protein, 127 aa, Artificial Sequence
  Anti-Human Cleaved Variable Heavy Chain -- CL03 IgG H1-NtoD; IgG3H7L; IgGA(08-10); IgGB01; IgGC(03, 05, 06, 09); IgGD08
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQSHSYYSHTWWVSYGMDYWGQGTLVTVSS

SEQ ID NO: 66 - protein, 302 aa, Artificial Sequence
  c-CDCP1 Cut 1 C-terminal fragment (CTF) of extracellular domain, without transmembrane domain (TM) and intracellular domain (ICD) amino acid sequence
QSRKFVPGCFVCLESRTCSSNLTLTSGSKHKISFLCDDLTRLWMNVEKTISCTDHRYCQRKSYSLQVPSDILHLPVELHDFSWKLLVPKDRLSLVLVPAQKLQQHTHEKPCNTSFSYLVASAIPSQDLYFGSFCPGGSIKQIQVKQNISVTLRTFAPSFQQEASRQGLTVSFIPYFKEEGVFTVTPDTKSKVYLRTPNWDRGLPSLTSVSWNISVPRDQVACLTFFKERSGVVCQTGRAFMIIQEQRTRAEEIFSLDEDVLPKPSFHHHSFWVNISNCSPTSGKQLDLLFSVTLTPRTVDLT

SEQ ID NO: 67 - protein, 127 aa, Artificial Sequence
  Anti-Human Cleaved Variable Heavy Chain -- CL03 IgG H1-NtoD H3-MtoA (“IgG101”)
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQSHSYYSHTWWVSYGADYWGQGTLVTVSS

SEQ ID NO: 68 - protein, 339 aa, Artificial Sequence
  c-CDCP1 Cut 2 N-terminal fragment of extracellular domain
FEIALPRESNITVLIKLGTPTLLAKPCYIVISKRHITMLSIKSGERIVFTFSCQSPENHFVIEIQKNIDCMSGPCPFGEVQLQPSTSLLPTLNRTFIWDVKAHKSIGLELQFSIPRLRQIGPGESCPDGVTHSISGRIDATVVRIGTFCSNGTVSRIKMQEGVKMALHLPWFHPRNVSGFSIANRSSIKRLCIIESVFEGEGSATLMSANYPEGFPEDELMTWQFVVPAHLRASVSFLNFNLSNCERKEERVEYYIPGSTTNPEVFKLEDKQPGNMAGNFNLSLQGCDQDAQSPGILRLQFQVLVQHPQNESNKIYVVDLSNERAMSLTIEPRPVKQSR

SEQ ID NO: 69 - protein, 127 aa, Artificial Sequence
  Anti-Human Cleaved Variable Heavy Chain -- CL03 IgG H1-NtoD H3-MtoI
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQSHSYYSHTWWVSYGIDYWGQGTLVTVSS

SEQ ID NO: 70 - protein, 468 aa, Artificial Sequence
  c-CDCP1 Cut 2 C-terminal fragment of extracellular domain, with transmembrane domain (TM) and intracellular domain (ICD)
KFVPGCFVCLESRTCSSNLTLTSGSKHKISFLCDDLTRLWMNVEKTISCTDHRYCQRKSYSLQVPSDILHLPVELHDFSWKLLVPKDRLSLVLVPAQKLQQHTHEKPCNTSFSYLVASAIPSQDLYFGSFCPGGSIKQIQVKQNISVTLRTFAPSFQQEASRQGLTVSFIPYFKEEGVFTVTPDTKSKVYLRTPNWDRGLPSLTSVSWNISVPRDQVACLTFFKERSGVVCQTGRAFMIIQEQRTRAEEIFSLDEDVLPKPSFHHHSFWVNISNCSPTSGKQLDLLFSVTLTPRTVDLTVILIAAVGGGVLLLSALGLIICCVKKKKKKTNKGPAVGIYNDNINTEMPRQPKKFQKGRKDNDSHVYAVIEDTMVYGHLLQDSSGSFLQPEVDTYRPFQGTMGVCPPSPPTICSRAPTAKLATEEPPPRSPPESESEPYTFSHPNNGDVSSKDTDIPLLNTQEPMEPAE

SEQ ID NO: 71 - protein, 127 aa, Artificial Sequence
  Anti-Human Cleaved Variable Heavy Chain -- CL03 IgG H1-NtoD H3-MtoL
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQSHSYYSHTWWVSYGLDYWGQGTLVTVSS

SEQ ID NO: 72 - protein, 299 aa, Artificial Sequence
  c-CDCP1 Cut 2 C-terminal fragment of extracellular domain, without transmembrane domain (TM) and intracellular domain (ICD)
KFVPGCFVCLESRTCSSNLTLTSGSKHKISFLCDDLTRLWMNVEKTISCTDHRYCQRKSYSLQVPSDILHLPVELHDFSWKLLVPKDRLSLVLVPAQKLQQHTHEKPCNTSFSYLVASAIPSQDLYFGSFCPGGSIKQIQVKQNISVTLRTFAPSFQQEASRQGLTVSFIPYFKEEGVFTVTPDTKSKVYLRTPNWDRGLPSLTSVSWNISVPRDQVACLTFFKERSGVVCQTGRAFMIIQEQRTRAEEIFSLDEDVLPKPSFHHHSFWVNISNCSPTSGKQLDLLFSVTLTPRTVDLT

SEQ ID NO: 73 - protein, 127 aa, Artificial Sequence
  Anti-Human Cleaved Variable Heavy Chain -- CL03 IgG H1-NtoD H3-MtoV
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQSHSYYSHTWWVSYGVDYWGQGTLVTVSS

SEQ ID NO: 74 - protein, 340 aa, Artificial Sequence
  c-CDCP1 Cut 3 N-terminal fragment of extracellular domain
FEIALPRESNITVLIKLGTPTLLAKPCYIVISKRHITMLSIKSGERIVFTFSCQSPENHFVIEIQKNIDCMSGPCPFGEVQLQPSTSLLPTLNRTFIWDVKAHKSIGLELQFSIPRLRQIGPGESCPDGVTHSISGRIDATVVRIGTFCSNGTVSRIKMQEGVKMALHLPWFHPRNVSGFSIANRSSIKRLCIIESVFEGEGSATLMSANYPEGFPEDELMTWQFVVPAHLRASVSFLNFNLSNCERKEERVEYYIPGSTTNPEVFKLEDKQPGNMAGNFNLSLQGCDQDAQSPGILRLQFQVLVQHPQNESNKIYVVDLSNERAMSLTIEPRPVKQSRK

SEQ ID NO: 75 - protein, 129 aa, Artificial Sequence
  Anti-Human Cleaved Variable Heavy Chain -- CL07 Fab, CL07 IgG
EVQLVESGGGLVQPGGSLRLSCAASGFNFSSSSIHWVRQAPGKGLEWVASIYPYSGSTSYADSVKGRFTISADTSKNTAYLQMNSLRAEDTAVYYCARTYWVQFWYGHFSYYRPALDYWGQGTLVTVSS

SEQ ID NO: 76 - protein, 107 aa, Artificial Sequence
  Anti-Human Cleaved Variable Light Chain -- CL07 (Fab, IgG, IgG H1-NtoD); IgG3H7L; IgGD09
DIQMTQSPSSLSASVGDRVTITCRASQSVSSAVAWYQQKPGKAPKLLIYSASSLYSGVPSRFSGSRSGTDFTLTISSLQPEDFATYYCQQSSSSLITFGQGTKVEIK

SEQ ID NO: 77 - protein, 467 aa, Artificial Sequence
  c-CDCP1 Cut 3 C-terminal fragment of extracellular domain, with transmembrane domain (TM) and intracellular domain (ICD)
FVPGCFVCLESRTCSSNLTLTSGSKHKISFLCDDLTRLWMNVEKTISCTDHRYCQRKSYSLQVPSDILHLPVELHDFSWKLLVPKDRLSLVLVPAQKLQQHTHEKPCNTSFSYLVASAIPSQDLYFGSFCPGGSIKQIQVKQNISVTLRTFAPSFQQEASRQGLTVSFIPYFKEEGVFTVTPDTKSKVYLRTPNWDRGLPSLTSVSWNISVPRDQVACLTFFKERSGVVCQTGRAFMIIQEQRTRAEEIFSLDEDVLPKPSFHHHSFWVNISNCSPTSGKQLDLLFSVTLTPRTVDLTVILIAAVGGGVLLLSALGLIICCVKKKKKKTNKGPAVGIYNDNINTEMPRQPKKFQKGRKDNDSHVYAVIEDTMVYGHLLQDSSGSFLQPEVDTYRPFQGTMGVCPPSPPTICSRAPTAKLATEEPPPRSPPESESEPYTFSHPNNGDVSSKDTDIPLLNTQEPMEPAE

SEQ ID NO: 78 - protein, 298 aa, Artificial Sequence
  c-CDCP1 Cut 3 C-terminal fragment of extracellular domain, without transmembrane domain (TM) and intracellular domain (ICD)
FVPGCFVCLESRTCSSNLTLTSGSKHKISFLCDDLTRLWMNVEKTISCTDHRYCQRKSYSLQVPSDILHLPVELHDFSWKLLVPKDRLSLVLVPAQKLQQHTHEKPCNTSFSYLVASAIPSQDLYFGSFCPGGSIKQIQVKQNISVTLRTFAPSFQQEASRQGLTVSFIPYFKEEGVFTVTPDTKSKVYLRTPNWDRGLPSLTSVSWNISVPRDQVACLTFFKERSGVVCQTGRAFMIIQEQRTRAEEIFSLDEDVLPKPSFHHHSFWVNISNCSPTSGKQLDLLFSVTLTPRTVDLT

SEQ ID NO: 79 - protein, 129 aa, Artificial Sequence
  Anti-Human Cleaved Variable Heavy Chain -- CL07 IgG H1-NtoD
EVQLVESGGGLVQPGGSLRLSCAASGFDFSSSSIHWVRQAPGKGLEWVASIYPYSGSTSYADSVKGRFTISADTSKNTAYLQMNSLRAEDTAVYYCARTYWVQFWYGHFSYYRPALDYWGQGTLVTVSS

SEQ ID NO: 80 - protein, 127 aa, Artificial Sequence
  Anti-Human Cleaved Variable Heavy Chain -- IgG87
EVQLVESGGGLVQPGGSLRLSCAASGFDASSYSIHWVRQAPGKGLEWVASIYPYSGSTYYADSVKGRFTISADTSKNTAYLQMNSLRAEDTAVYYCARQSHSYYSHTWWVSYGMDYWGQGTLVTVSS

SEQ ID NO: 81 - protein, 127 aa, Artificial Sequence
  Anti-Human Cleaved Variable Heavy Chain -- IgG89
EVQLVESGGGLVQPGGSLRLSCAASGFDFSSYSAHWVRQAPGKGLEWVASIYPYSGSTYYADSVKGRFTISADTSKNTAYLQMNSLRAEDTAVYYCARQSHSYYSHTWWVSYGMDYWGQGTLVTVSS

SEQ ID NO: 82 - protein, 127 aa, Artificial Sequence
  Anti-Human Cleaved Variable Heavy Chain -- IgG94
EVQLVESGGGLVQPGGSLRLSCAASGFDFSSYSIHWVRQAPGKGLEWVASIYPYSGSTSYADSVKGRFTISADTSKNTAYLQMNSLRAEDTAVYYCARQSHSYYSHTWWVSYGMDYWGQGTLVTVSS

SEQ ID NO: 83 - protein, 127 aa, Artificial Sequence
  Anti-Human Cleaved Variable Heavy Chain -- IgGA01
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQNHSYYSHTWWVSYGMDYWGQGTLVTVSS

SEQ ID NO: 84 - protein, 127 aa, Artificial Sequence
  Anti-Human Cleaved Variable Heavy Chain -- IgG97
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQSHSYYSHTAWVSYGMDYWGQGTLVTVSS

SEQ ID NO: 85 - protein, 127 aa, Artificial Sequence
  Anti-Human Cleaved Variable Heavy Chain -- IgGA03
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQTHSYYSHTWWVSYGMDYWGQGTLVTVSS

SEQ ID NO: 86 - protein, 127 aa, Artificial Sequence
  Anti-Human Cleaved Variable Heavy Chain -- IgG102
EVQLVESGGGLVQPGGSLRLSCAASGFDFSSYSIHWVRQAPGKGLEWVASSYPYSGSTYYADSVKGRFTISADTSKNTAYLQMNSLRAEDTAVYYCARQSHSYYSHTWWVSYGMDYWGQGTLVTVSS

SEQ ID NO: 87 - protein, 127 aa, Artificial Sequence
  Anti-Human Cleaved Variable Heavy Chain -- IgGA04
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQGHSYYSHTWWVSYGMDYWGQGTLVTVSS

SEQ ID NO: 88 - protein, 381 aa, Artificial Sequence
  Anti-Human Cleaved Variable Heavy Chain -- IgG87
GAGGTTCAGCTGGTGGAGTCTGGCGGTGGCCTGGTGCAGCCAGGGGGCTCACTCCGTTTGTCCTGTGCAGCTTCTGGCTTCGACGCCTCTTCTTATTCTATACACTGGGTGCGTCAGGCCCCGGGTAAGGGCCTGGAATGGGTTGCATCTATTTATCCTTATTCTGGCTCTACTTATTATGCCGATAGCGTCAAGGGCCGTTTCACTATAAGCGCAGACACATCCAAAAACACAGCCTACCTACAAATGAACAGCTTAAGAGCTGAGGACACTGCCGTCTATTATTGTGCTCGCCAGTCTCATTCTTACTACTCTCATACTTGGTGGGTTTCTTACGGTATGGACTACTGGGGTCAAGGAACCCTGGTCACCGTCTCCTCG

SEQ ID NO: 89 - protein, 127 aa, Artificial Sequence
  Anti-Human Cleaved Variable Heavy Chain -- IgGA05
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQAHSYYSHTWWVSYGMDYWGQGTLVTVSS

SEQ ID NO: 90 - protein, 321 aa, Artificial Sequence
  Anti-Human Cleaved Variable Light Chain -- IgG87, IgG89, IgG94, IgG97, IgG102
GATATCCAGATGACCCAGTCCCCGAGCTCCCTGTCCGCCTCTGTGGGCGATAGGGTCACCATCACCTGCCGTGCCAGTCAGTCCGTGTCCAGCGCTGTAGCCTGGTATCAACAGAAACCAGGAAAAGCTCCGAAGCTTCTGATTTACTCGGCATCCAGCCTCTACTCTGGAGTCCCTTCTCGCTTCTCTGGTAGCCGTTCCGGGACGGATTTCACTCTGACCATCAGCAGTCTGCAGCCGGAAGACTTCGCAACTTATTACTGTCAGCAATCTGGTCAGCGTCCGATCACGTTCGGACAGGGTACCAAGGTGGAGATCAAA

SEQ ID NO: 91 - protein, 127 aa, Artificial Sequence
  Anti-Human Cleaved Variable Heavy Chain -- IgGA06
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQGQSYYSHTWWVSYGMDYWGQGTLVTVSS

SEQ ID NO: 92 - protein, 381 aa, Artificial Sequence
  Anti-Human Cleaved Variable Heavy Chain -- IgG89
GAGGTTCAGCTGGTGGAGTCTGGCGGTGGCCTGGTGCAGCCAGGGGGCTCACTCCGTTTGTCCTGTGCAGCTTCTGGCTTCGACTTCTCTTCTTATTCTGCGCACTGGGTGCGTCAGGCCCCGGGTAAGGGCCTGGAATGGGTTGCATCTATTTATCCTTATTCTGGCTCTACTTATTATGCCGATAGCGTCAAGGGCCGTTTCACTATAAGCGCAGACACATCCAAAAACACAGCCTACCTACAAATGAACAGCTTAAGAGCTGAGGACACTGCCGTCTATTATTGTGCTCGCCAGTCTCATTCTTACTACTCTCATACTTGGTGGGTTTCTTACGGTATGGACTACTGGGGTCAAGGAACCCTGGTCACCGTCTCCTCG

SEQ ID NO: 93 - protein, 381 aa, Artificial Sequence
  Anti-Human Cleaved Variable Heavy Chain -- IgG94
GAGGTTCAGCTGGTGGAGTCTGGCGGTGGCCTGGTGCAGCCAGGGGGCTCACTCCGTTTGTCCTGTGCAGCTTCTGGCTTCGACTTCTCTTCTTATTCTATACACTGGGTGCGTCAGGCCCCGGGTAAGGGCCTGGAATGGGTTGCATCTATTTATCCTTATTCTGGCTCTACTAGCTATGCCGATAGCGTCAAGGGCCGTTTCACTATAAGCGCAGACACATCCAAAAACACAGCCTACCTACAAATGAACAGCTTAAGAGCTGAGGACACTGCCGTCTATTATTGTGCTCGCCAGTCTCATTCTTACTACTCTCATACTTGGTGGGTTTCTTACGGTATGGACTACTGGGGTCAAGGAACCCTGGTCACCGTCTCCTCG

SEQ ID NO: 94 - protein, 107 aa, Artificial Sequence
  Anti-Human Cleaved Variable Light Chain -- IgGA08
DIQMTQSPSSLSASVGDRVTITCRASQSVSSAVAWYQQKPGKAPKLLIYSASSLYSGVPSRFSGSRSGTDFTLTISSLQPEDFATYYCQQSGERPITFGQGTKVEIK

SEQ ID NO: 95 - protein, 381 aa, Artificial Sequence
  Anti-Human Cleaved Variable Heavy Chain -- IgG97
GAGGTTCAGCTGGTGGAGTCTGGCGGTGGCCTGGTGCAGCCAGGGGGCTCACTCCGTTTGTCCTGTGCAGCTTCTGGCTTCGACTTCTCTTCTTATTCTATACACTGGGTGCGTCAGGCCCCGGGTAAGGGCCTGGAATGGGTTGCATCTATTTATCCTTATTCTGGCTCTACTTATTATGCCGATAGCGTCAAGGGCCGTTTCACTATAAGCGCAGACACATCCAAAAACACAGCCTACCTACAAATGAACAGCTTAAGAGCTGAGGACACTGCCGTCTATTATTGTGCTCGCCAGTCTCATTCTTACTACTCTCATACTTGGTGGGTTTCTTACGGTGCGGACTACTGGGGTCAAGGAACCCTGGTCACCGTCTCCTCG

SEQ ID NO: 96 - protein, 107 aa, Artificial Sequence
  Anti-Human Cleaved Variable Light Chain -- IgGA09
DIQMTQSPSSLSASVGDRVTITCRASQSVSSAVAWYQQKPGKAPKLLIYSASSLYSGVPSRFSGSRSGTDFTLTISSLQPEDFATYYCQQSGNVPATFGQGTKVEIK

SEQ ID NO: 97 - protein, 381 aa, Artificial Sequence
  Anti-Human Cleaved Variable Heavy Chain -- IgG102
GAGGTTCAGCTGGTGGAGTCTGGCGGTGGCCTGGTGCAGCCAGGGGGCTCACTCCGTTTGTCCTGTGCAGCTTCTGGCTTCGACTTCTCTTCTTATTCTATACACTGGGTGCGTCAGGCCCCGGGTAAGGGCCTGGAATGGGTTGCATCTAGCTATCCTTATTCTGGCTCTACTTATTATGCCGATAGCGTCAAGGGCCGTTTCACTATAAGCGCAGACACATCCAAAAACACAGCCTACCTACAAATGAACAGCTTAAGAGCTGAGGACACTGCCGTCTATTATTGTGCTCGCCAGTCTCATTCTTACTACTCTCATACTTGGTGGGTTTCTTACGGTATGGACTACTGGGGTCAAGGAACCCTGGTCACCGTCTCCTCG

SEQ ID NO: 98 - protein, 107 aa, Artificial Sequence
  Anti-Human Cleaved Variable Light Chain -- IgGA10
DIQMTQSPSSLSASVGDRVTITCRASQSVSSAVAWYQQKPGKAPKLLIYSASSLYSGVPSRFSGSRSGTDFTLTISSLQPEDFATYYCQQSGKRPMTFGQGTKVEIK

SEQ ID NO: 99 - protein, 127 aa, Artificial Sequence
  Anti-Human Cleaved Variable Heavy Chain -- IgGA11; IgGB(04, 08); IgGC(01, 04); IgGD(04, 07)
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARRSHSYYSHTWWVSYGMDYWGQGTLVTVSS

SEQ ID NO: 100 - protein, 107 aa, Artificial Sequence
  Anti-Human Cleaved Variable Light Chain -- IgGA11
DIQMTQSPSSLSASVGDRVTITCRASQSVSSAVAWYQQKPGKAPKLLIYSASSLYSGVPSRFSGSRSGTDFTLTISSLQPEDFATYYCQQTGQRPMTFGQGTKVEIK

SEQ ID NO: 101 - protein, 10 aa, Artificial Sequence
  Anti-Human Cleaved CDR H2 -- IgG102
SSYPYSGSTY

SEQ ID NO: 102 - protein, 107 aa, Artificial Sequence
  Anti-Human Cleaved Variable Light Chain -- IgGB01
DIQMTQSPSSLSASVGDRVTITCRASQSVSSAVAWYQQKPGKAPKLLIYSASSLYSGVPSRFSGSRSGTDFTLTISSLQPEDFATYYCQQSMQWPKTFGQGTKVEIK

SEQ ID NO: 103 - protein, 127 aa, Artificial Sequence
  Anti-Human Cleaved Variable Heavy Chain -- IgGB03
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQSHSFYSHTWWVSYGMDYWGQGTLVTVSS

SEQ ID NO: 104 - protein, 107 aa, Artificial Sequence
  Anti-Human Cleaved Variable Light Chain -- IgGB03
DIQMTQSPSSLSASVGDRVTITCRASQSVSSAVAWYQQKPGKAPKLLIYSASSLYSGVPSRFSGSRSGTDFTLTISSLQPEDFATYYCQQSLQRPMTFGQGTKVEIK

SEQ ID NO: 105 - protein, 16 aa, Artificial Sequence
  Anti-Human Cleaved CDR H3 -- IgG97
QSHSYYSHTAWVSYGM

SEQ ID NO: 106 - protein, 107 aa, Artificial Sequence
  Anti-Human Cleaved Variable Light Chain -- IgGB04
DIQMTQSPSSLSASVGDRVTITCRASQSVSSAVAWYQQKPGKAPKLLIYSASSLYSGVPSRFSGSRSGTDFTLTISSLQPEDFATYYCQQVELVPMTFGQGTKVEIK

SEQ ID NO: 107 - protein, 127 aa, Artificial Sequence
  Anti-Human Cleaved Variable Heavy Chain -- IgGB07, IgGD03
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQDHSYYSHTWWVSYGMDYWGQGTLVTVSS

SEQ ID NO: 108 - protein, 107 aa, Artificial Sequence
  Anti-Human Cleaved Variable Light Chain -- IgGB07
DIQMTQSPSSLSASVGDRVTITCRASQSVSSAVAWYQQKPGKAPKLLIYSASSLYSGVPSRFSGSRSGTDFTLTISSLQPEDFATYYCQQAGKRPLTFGQGTKVEIK

SEQ ID NO: 109 - protein, 6 aa, Artificial Sequence
  Anti-Human Cleaved CDR H1 -- IgG87
ASSYSI

SEQ ID NO: 110 - protein, 107 aa, Artificial Sequence
  Anti-Human Cleaved Variable Light Chain -- IgGB08
DIQMTQSPSSLSASVGDRVTITCRASQSVSSAVAWYQQKPGKAPKLLIYSASSLYSGVPSRFSGSRSGTDFTLTISSLQPEDFATYYCQQSVDLTATFGQGTKVEIK

SEQ ID NO: 111 - protein, 6 aa, Artificial Sequence
  Anti-Human Cleaved CDR H1 -- IgG89
FSSYSA

SEQ ID NO: 112 - protein, 107 aa, Artificial Sequence
  Anti-Human Cleaved Variable Light Chain -- IgGC01
DIQMTQSPSSLSASVGDRVTITCRASQSVSSAVAWYQQKPGKAPKLLIYSASSLYSGVPSRFSGSRSGTDFTLTISSLQPEDFATYYCQQLGVRAATFGQGTKVEIK

SEQ ID NO: 113 - skipped

SEQ ID NO: 114 - protein, 107 aa, Artificial Sequence
  Anti-Human Cleaved Variable Light Chain -- IgGC03
DIQMTQSPSSLSASVGDRVTITCRASQSVSSAVAWYQQKPGKAPKLLIYSASSLYSGVPSRFSGSRSGTDFTLTISSLQPEDFATYYCQQSGRKPVTFGQGTKVEIK

SEQ ID NO: 115 - skipped

SEQ ID NO: 116 - protein, 107 aa, Artificial Sequence
  Anti-Human Cleaved Variable Light Chain -- IgGC04
DIQMTQSPSSLSASVGDRVTITCRASQSVSSAVAWYQQKPGKAPKLLIYSASSLYSGVPSRFSGSRSGTDFTLTISSLQPEDFATYYCQQTAQSPLTFGQGTKVEIK

SEQ ID NO: 117 - skipped

SEQ ID NO: 118 - protein, 107 aa, Artificial Sequence
  Anti-Human Cleaved Variable Light Chain -- IgGC05
DIQMTQSPSSLSASVGDRVTITCRASQSVSSAVAWYQQKPGKAPKLLIYSASSLYSGVPSRFSGSRSGTDFTLTISSLQPEDFATYYCQQSRQRPLTFGQGTKVEIK

SEQ ID NO: 119 - skipped

SEQ ID NO: 120 - protein, 107 aa, Artificial Sequence
  Anti-Human Cleaved Variable Light Chain -- IgGC06
DIQMTQSPSSLSASVGDRVTITCRASQSVSSAVAWYQQKPGKAPKLLIYSASSLYSGVPSRFSGSRSGTDFTLTISSLQPEDFATYYCQQSGLMPLTFGQGTKVEIK

SEQ ID NO: 121 - skipped

SEQ ID NO: 122 - protein, 107 aa, Artificial Sequence
  Anti-Human Cleaved Variable Light Chain -- IgGC09
DIQMTQSPSSLSASVGDRVTITCRASQSVSSAVAWYQQKPGKAPKLLIYSASSLYSGVPSRFSGSRSGTDFTLTISSLQPEDFATYYCQQSGHRPITFGQGTKVEIK

SEQ ID NO: 123 - protein, 127 aa, Artificial Sequence
  Anti-Human Cleaved Variable Heavy Chain -- IgGD02
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARKYHSYYSHTWWVSYGMDYWGQGTLVTVSS

SEQ ID NO: 124 - protein, 107 aa, Artificial Sequence
  Anti-Human Cleaved Variable Light Chain -- IgGD02
DIQMTQSPSSLSASVGDRVTITCRASQSVSSAVAWYQQKPGKAPKLLIYSASSLYSGVPSRFSGSRSGTDFTLTISSLQPEDFATYYCQQSSQRPATFGQGTKVEIK

SEQ ID NO: 125 - skipped

SEQ ID NO: 126 - protein, 107 aa, Artificial Sequence
  Anti-Human Cleaved Variable Light Chain -- IgGD03
DIQMTQSPSSLSASVGDRVTITCRASQSVSSAVAWYQQKPGKAPKLLIYSASSLYSGVPSRFSGSRSGTDFTLTISSLQPEDFATYYCQQSGRQPLTFGQGTKVEIK

SEQ ID NO: 127 - skipped

SEQ ID NO: 128 - protein, 107 aa, Artificial Sequence
  Anti-Human Cleaved Variable Light Chain -- IgGD04
DIQMTQSPSSLSASVGDRVTITCRASQSVSSAVAWYQQKPGKAPKLLIYSASSLYSGVPSRFSGSRSGTDFTLTISSLQPEDFATYYCQQFMRPAFTFGQGTKVEIK

SEQ ID NO: 129 - skipped

SEQ ID NO: 130 - protein, 107 aa, Artificial Sequence
  Anti-Human Cleaved Variable Light Chain -- IgGD07
DIQMTQSPSSLSASVGDRVTITCRASQSVSSAVAWYQQKPGKAPKLLIYSASSLYSGVPSRFSGSRSGTDFTLTISSLQPEDFATYYCQQSGQPSMTFGQGTKVEIK

SEQ ID NO: 131 - skipped

SEQ ID NO: 132 - protein, 107 aa, Artificial Sequence
  Anti-Human Cleaved Variable Light Chain -- IgGD08
DIQMTQSPSSLSASVGDRVTITCRASQSVSSAVAWYQQKPGKAPKLLIYSASSLYSGVPSRFSGSRSGTDFTLTISSLQPEDFATYYCQQSVLRPFTFGQGTKVEIK

SEQ ID NO: 133 - protein, 129 aa, Artificial Sequence
  Anti-Human Cleaved Variable Heavy Chain -- IgGD09
EVQLVESGGGLVQPGGSLRLSCAASGFNFSSSSIHWVRQAPGKGLEWVASIYPYSGSTSYADSVKGRFTISADTSKNTAYLQMNSLRAEDTAVYYCARIYWVDFWYGHFSYYRPALDYWGQGTLVTVSS

SEQ ID NO: 134 - skipped

SEQ ID NO: 135 - protein, 124 aa, Artificial Sequence
  Anti-Mouse Cleaved Variable Heavy Chain -- Fab 0114, IgG 0029 (Fab 0114)
EVQLVESGGGLVQPGGSLRLSCAASGFNVSYSSIHWVRQAPGKGLEWVASISSSSGYTYYADSVKGRFTISADTSKNTAYLQMNSLRAEDTAVYYCARQEGDWWVAYWPALDYWGQGTLVTVSS

SEQ ID NO: 136 - protein, 107 aa, Artificial Sequence
  Anti-Mouse Cleaved Variable Light Chain -- Fab 0114, IgG 0029 (Fab 0114)
DIQMTQSPSSLSASVGDRVTITCRASQSVSSAVAWYQQKPGKAPKLLIYSASSLYSGVPSRFSGSRSGTDFTLTISSLQPEDFATYYCQQSSWGLFTFGQGTKVEIK

SEQ ID NO: 137-138 - skipped (2 entries)

SEQ ID NO: 139 - protein, 127 aa, Artificial Sequence
  Anti-Mouse Cleaved Variable Heavy Chain -- Fab 0123, IgG 0030 (Fab 0123)
EVQLVESGGGLVQPGGSLRLSCAASGFNVYSYSIHWVRQAPGKGLEWVASISSSSGYTYYADSVKGRFTISADTSKNTAYLQMNSLRAEDTAVYYCARYYWDPWQYWYYSYLAFDYWGQGTLVTVSS

SEQ ID NO: 140 - protein, 107 aa, Artificial Sequence
  Anti-Mouse Cleaved Variable Light Chain -- Fab 0123, IgG 0030 (Fab 0123), IgG 0046, IgG 0050, IgG 0051, IgG 0055, IgG 0058, IgG 0059, IgG 0060, IgG 0062, IgG 0063, IgG 0065
DIQMTQSPSSLSASVGDRVTITCRASQSVSSAVAWYQQKPGKAPKLLIYSASSLYSGVPSRFSGSRSGTDFTLTISSLQPEDFATYYCQQTYSGPITFGQGTKVEIK

SEQ ID NO: 141-142 - skipped (2 entries)

SEQ ID NO: 143 - protein, 127 aa, Artificial Sequence
  Anti-Mouse Cleaved Variable Heavy Chain -- IgG 0046
EVQLVESGGGLVQPGGSLRLSCAASGFNVYSYSIHWVRQAPGKGLEWVASISSSSGYTYYADSVKGRFTISADTSKNTAYLQMNSLRAEDTAVYYCARYYWDPWQYAYYSYLAFDYWGQGTLVTVSS

SEQ ID NO: 144 - skipped

SEQ ID NO: 145 - protein, 127 aa, Artificial Sequence
  Anti-Mouse Cleaved Variable Heavy Chain -- IgG 0050
EVQLVESGGGLVQPGGSLRLSCAASGFNVYSYSIHWVRQAPGKGLEWVASISSSSGSTYYADSVKGRFTISADTSKNTAYLQMNSLRAEDTAVYYCARYYWDPWQYAYYSYLAFDYWGQGTLVTVSS

SEQ ID NO: 146 - skipped

SEQ ID NO: 147 - protein, 127 aa, Artificial Sequence
  Anti-Mouse Cleaved Variable Heavy Chain -- IgG 0051
EVQLVESGGGLVQPGGSLRLSCAASGFNVYSYSIHWVRQAPGKGLEWVASISSSSGYTSYADSVKGRFTISADTSKNTAYLQMNSLRAEDTAVYYCARYYWDPWQYAYYSYLAFDYWGQGTLVTVSS

SEQ ID NO: 148 - skipped

SEQ ID NO: 149 - protein, 127 aa, Artificial Sequence
  Anti-Mouse Cleaved Variable Heavy Chain -- IgG 0055
EVQLVESGGGLVQPGGSLRLSCAASGFNVYSYSIHWVRQAPGKGLEWVASISSSSGYTYYADSVKGRFTISADTSKNTAYLQMNSLRAEDTAVYYCARYYWDPWQYASYSYLAFDYWGQGTLVTVSS

SEQ ID NO: 150 - skipped

SEQ ID NO: 151 - protein, 127 aa, Artificial Sequence
  Anti-Mouse Cleaved Variable Heavy Chain -- IgG 0058
EVQLVESGGGLVQPGGSLRLSCAASGFNVYSYSIHWVRQAPGKGLEWVASISSSSGSTSYADSVKGRFTISADTSKNTAYLQMNSLRAEDTAVYYCARYYWDPWQYASYSYLAFDYWGQGTLVTVSS

SEQ ID NO: 152 - skipped

SEQ ID NO: 153 - protein, 127 aa, Artificial Sequence
  Anti-Mouse Cleaved Variable Heavy Chain -- IgG 0059
EVQLVESGGGLVQPGGSLRLSCAASGFNVYSYSIHWVRQAPGKGLEWVASISSSSGSTYYADSVKGRFTISADTSKNTAYLQMNSLRAEDTAVYYCARYYWDPWQYASYSYLAFDYWGQGTLVTVSS

SEQ ID NO: 154 - skipped

SEQ ID NO: 155 - protein, 127 aa, Artificial Sequence
  Anti-Mouse Cleaved Variable Heavy Chain -- IgG 0060
EVQLVESGGGLVQPGGSLRLSCAASGFNVYSYSIHWVRQAPGKGLEWVASISSSSGYTSYADSVKGRFTISADTSKNTAYLQMNSLRAEDTAVYYCARYYWDPWQYASYSYLAFDYWGQGTLVTVSS

SEQ ID NO: 156 - skipped

SEQ ID NO: 157 - protein, 127 aa, Artificial Sequence
  Anti-Mouse Cleaved Variable Heavy Chain -- IgG 0062
EVQLVESGGGLVQPGGSLRLSCAASGFNVYSYSIHWVRQAPGKGLEWVASISSSSGSTYYADSVKGRFTISADTSKNTAYLQMNSLRAEDTAVYYCARYYWDPWQYSSYSYLAFDYWGQGTLVTVSS

SEQ ID NO: 158 - skipped

SEQ ID NO: 159 - protein, 127 aa, Artificial Sequence
  Anti-Mouse Cleaved Variable Heavy Chain -- IgG 0063
EVQLVESGGGLVQPGGSLRLSCAASGFNVYSYSIHWVRQAPGKGLEWVASISSSSGYTSYADSVKGRFTISADTSKNTAYLQMNSLRAEDTAVYYCARYYWDPWQYSSYSYLAFDYWGQGTLVTVSS

SEQ ID NO: 160 - skipped

SEQ ID NO: 161 - protein, 127 aa, Artificial Sequence
  Anti-Mouse Cleaved Variable Heavy Chain -- IgG 0065
EVQLVESGGGLVQPGGSLRLSCAASGFNVYSYSIHWVRQAPGKGLEWVASISSSSGSTSYADSVKGRFTISADTSKNTAYLQMNSLRAEDTAVYYCARYYWDPWQYSSYSYLAFDYWGQGTLVTVSS

SEQ ID NO: 162 - skipped

SEQ ID NO: 163 - DNA, 381 bp, Artificial Sequence
  Anti-Human Cleaved Variable Heavy Chain -- CL03 (Fab, IgG)
gaggttcagctggtggagtctggcggtggcctggtgcagccagggggctcactccgtttgtcctgtgcagcttctggcttcaacttctcttcttattctatacactgggtgcgtcaggccccgggtaagggcctggaatgggttgcatctatttatccttattctggctctacttattatgccgatagcgtcaagggccgtttcactataagcgcagacacatccaaaaacacagcctacctacaaatgaacagcttaagagctgaggacactgccgtctattattgtgctcgccagtctcattcttactactctcatacttggtgggtttcttacggtatggactactggggtcaaggaaccctggtcaccgtctcctcg

SEQ ID NO: 164 - DNA, 321 bp, Artificial Sequence
  Anti-Human Cleaved Variable Light Chain -- CL03 (Fab, IgG, IgG H1-NtoD); CL03 IgG H1-NtoD (H3-MtoA (“IgG101”), H3-MtoI, H3-MtoL, H3-MtoV); IgGA(01, 03-06)
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatctggtcagcgtccgatcacgttcggacagggtaccaaggtggagatcaaa

SEQ ID NO: 165-168 - skipped (4 entries)

SEQ ID NO: 169 - DNA, 381 bp, Artificial Sequence
  Anti-Human Cleaved Variable Heavy Chain -- CL03 IgG H1-NtoD; IgG3H7L; IgGA(08-10); IgGB01; IgGC(03, 05, 06, 09); IgGD08
gaggttcagctggtggagtctggcggtggcctggtgcagccagggggctcactccgtttgtcctgtgcagcttctggcttcgacttctcttcttattctatacactgggtgcgtcaggccccgggtaagggcctggaatgggttgcatctatttatccttattctggctctacttattatgccgatagcgtcaagggccgtttcactataagcgcagacacatccaaaaacacagcctacctacaaatgaacagcttaagagctgaggacactgccgtctattattgtgctcgccagtctcattcttactactctcatacttggtgggtttcttacggtatggactactggggtcaaggaaccctggtcaccgtctcctcg

SEQ ID NO: 170 - skipped

SEQ ID NO: 171 - DNA, 381 bp, Artificial Sequence
  Anti-Human Cleaved Variable Heavy Chain -- CL03 IgG H1-NtoD H3-MtoA (“IgG101”)
gaggttcagctggtggagtctggcggtggcctggtgcagccagggggctcactccgtttgtcctgtgcagcttctggcttcaacttctcttcttattctatacactgggtgcgtcaggccccgggtaagggcctggaatgggttgcatctatttatccttattctggctctacttattatgccgatagcgtcaagggccgtttcactataagcgcagacacatccaaaaacacagcctacctacaaatgaacagcttaagagctgaggacactgccgtctattattgtgctcgccagtctcattcttactactctcatacttggtgggtttcttacggtgcggactactggggtcaaggaaccctggtcaccgtctcctcg

SEQ ID NO: 172 - skipped

SEQ ID NO: 173 - DNA, 381 bp, Artificial Sequence
  Anti-Human Cleaved Variable Heavy Chain -- CL03 IgG H1-NtoD H3-MtoI
gaggttcagctggtggagtctggcggtggcctggtgcagccagggggctcactccgtttgtcctgtgcagcttctggcttcgacttctcttcttattctatacactgggtgcgtcaggccccgggtaagggcctggaatgggttgcatctatttatccttattctggctctacttattatgccgatagcgtcaagggccgtttcactataagcgcagacacatccaaaaacacagcctacctacaaatgaacagcttaagagctgaggacactgccgtctattattgtgctcgccagtctcattcttactactctcatacttggtgggtttcttacggtatcgactactggggtcaaggaaccctggtcaccgtctcctcg

SEQ ID NO: 174 - skipped

SEQ ID NO: 175 - DNA, 381 bp, Artificial Sequence
  Anti-Human Cleaved Variable Heavy Chain -- CL03 IgG H1-NtoD H3-MtoL
gaggttcagctggtggagtctggcggtggcctggtgcagccagggggctcactccgtttgtcctgtgcagcttctggcttcgacttctcttcttattctatacactgggtgcgtcaggccccgggtaagggcctggaatgggttgcatctatttatccttattctggctctacttattatgccgatagcgtcaagggccgtttcactataagcgcagacacatccaaaaacacagcctacctacaaatgaacagcttaagagctgaggacactgccgtctattattgtgctcgccagtctcattcttactactctcatacttggtgggtttcttacggtctggactactggggtcaaggaaccctggtcaccgtctcctcg

SEQ ID NO: 176 - skipped

SEQ ID NO: 177 - DNA, 381 bp, Artificial Sequence
  Anti-Human Cleaved Variable Heavy Chain -- CL03 IgG H1-NtoD H3-MtoV
gaggttcagctggtggagtctggcggtggcctggtgcagccagggggctcactccgtttgtcctgtgcagcttctggcttcgacttctcttcttattctatacactgggtgcgtcaggccccgggtaagggcctggaatgggttgcatctatttatccttattctggctctacttattatgccgatagcgtcaagggccgtttcactataagcgcagacacatccaaaaacacagcctacctacaaatgaacagcttaagagctgaggacactgccgtctattattgtgctcgccagtctcattcttactactctcatacttggtgggtttcttacggtgtggactactggggtcaaggaaccctggtcaccgtctcctcg

SEQ ID NO: 178 - skipped

SEQ ID NO: 179 - DNA, 387 bp, Artificial Sequence
  Anti-Human Cleaved Variable Heavy Chain -- CL07 Fab, CL07 IgG
gaggttcagctggtggagtctggcggtggcctggtgcagccagggggctcactccgtttgtcctgtgcagcttctggcttcaacttttcttcttcttctatacactgggtgcgtcaggccccgggtaagggcctggaatgggttgcatctatttatccttattctggctctacttcttatgccgatagcgtcaagggccgtttcactataagcgcagacacatccaaaaacacagcctacctacaaatgaacagcttaagagctgaggacactgccgtctattattgtgctcgcacttactgggttcagttctggtacggtcatttctcttactaccgtccggctttggactactggggtcaaggaaccctggtcaccgtctcctcg

SEQ ID NO: 180 - DNA, 321 bp, Artificial Sequence
  Anti-Human Cleaved Variable Light Chain -- CL07 Fab, CL07 IgG, CL07 IgG H1-NtoD, IgG3H7L
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatcttcttcttctctgatcacgttcggacagggtaccaaggtggagatcaaa

SEQ ID NO: 181-182 - skipped (2 entries)

SEQ ID NO: 183 - DNA, 387 bp, Artificial Sequence
  Anti-Human Cleaved Variable Heavy Chain -- CL07 IgG H1-NtoD
gaggttcagctggtggagtctggcggtggcctggtgcagccagggggctcactccgtttgtcctgtgcagcttctggcttcgacttttcttcttcttctatacactgggtgcgtcaggccccgggtaagggcctggaatgggttgcatctatttatccttattctggctctacttcttatgccgatagcgtcaagggccgtttcactataagcgcagacacatccaaaaacacagcctacctacaaatgaacagcttaagagctgaggacactgccgtctattattgtgctcgcacttactgggttcagttctggtacggtcatttctcttactaccgtccggctttggactactggggtcaaggaaccctggtcaccgtctcctcg

SEQ ID NO: 184-186 - skipped (3 entries)

SEQ ID NO: 187 - DNA, 381 bp, Artificial Sequence
  Anti-Human Cleaved Variable Heavy Chain -- IgGA01
gaggttcagctggtggagtctggcggtggcctggtgcagccagggggctcactccgtttgtcctgtgcagcttctggcttcgacttctcttcttattctatacactgggtgcgtcaggccccgggtaagggcctggaatgggttgcatctatttatccttattctggctctacttattatgccgatagcgtcaagggccgtttcactataagcgcagacacatccaaaaacacagcctacctacaaatgaacagcttaagagctgaggacactgccgtctattattgtgctcgccagaatcattcgtactactctcatacttggtgggtttcttacggtatggactactggggtcaaggaaccctggtcaccgtctcctcg

SEQ ID NO: 188 - skipped

SEQ ID NO: 189 - DNA, 381 bp, Artificial Sequence
  Anti-Human Cleaved Variable Heavy Chain -- IgGA03
gaggttcagctggtggagtctggcggtggcctggtgcagccagggggctcactccgtttgtcctgtgcagcttctggcttcgacttctcttcttattctatacactgggtgcgtcaggccccgggtaagggcctggaatgggttgcatctatttatccttattctggctctacttattatgccgatagcgtcaagggccgtttcactataagcgcagacacatccaaaaacacagcctacctacaaatgaacagcttaagagctgaggacactgccgtctattattgtgctcgccagacgcattcttactactctcatacttggtgggtttcttacggtatggactactggggtcaaggaaccctggtcaccgtctcctcg

SEQ ID NO: 190 - skipped

SEQ ID NO: 191 - DNA, 381 bp, Artificial Sequence
  Anti-Human Cleaved Variable Heavy Chain -- IgGA04
gaggttcagctggtggagtctggcggtggcctggtgcagccagggggctcactccgtttgtcctgtgcagcttctggcttcgacttctcttcttattctatacactgggtgcgtcaggccccgggtaagggcctggaatgggttgcatctatttatccttattctggctctacttattatgccgatagcgtcaagggccgtttcactataagcgcagacacatccaaaaacacagcctacctacaaatgaacagcttaagagctgaggacactgccgtctattattgtgctcgccaggggcattcttactactctcatacttggtgggtttcttacggtatggactactggggtcaaggaaccctggtcaccgtctcctcg

SEQ ID NO: 192 - skipped

SEQ ID NO: 193 - DNA, 381 bp, Artificial Sequence
  Anti-Human Cleaved Variable Heavy Chain -- IgGA05
gaggttcagctggtggagtctggcggtggcctggtgcagccagggggctcactccgtttgtcctgtgcagcttctggcttcgacttctcttcttattctatacactgggtgcgtcaggccccgggtaagggcctggaatgggttgcatctatttatccttattctggctctacttattatgccgatagcgtcaagggccgtttcactataagcgcagacacatccaaaaacacagcctacctacaaatgaacagcttaagagctgaggacactgccgtctattattgtgctcgccaggctcattcgtactactctcatacttggtgggtttcttacggtatggactactggggtcaaggaaccctggtcaccgtctcctcg

SEQ ID NO: 194 - skipped

SEQ ID NO: 195 - DNA, 381 bp, Artificial Sequence
  Anti-Human Cleaved Variable Heavy Chain -- IgGA06
gaggttcagctggtggagtctggcggtggcctggtgcagccagggggctcactccgtttgtcctgtgcagcttctggcttcgacttctcttcttattctatacactgggtgcgtcaggccccgggtaagggcctggaatgggttgcatctatttatccttattctggctctacttattatgccgatagcgtcaagggccgtttcactataagcgcagacacatccaaaaacacagcctacctacaaatgaacagcttaagagctgaggacactgccgtctattattgtgctcgccaggggcagtcttactactctcatacttggtgggtttcttacggtatggactactggggtcaaggaaccctggtcaccgtctcctcg

SEQ ID NO: 196-197 - skipped (2 entries)

SEQ ID NO: 198 - DNA, 321 bp, Artificial Sequence
  Anti-Human Cleaved Variable Light Chain -- IgGA08
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatcgggggagcggccgattacgttcggacagggtaccaaggtggagatcaaa

SEQ ID NO: 199 - skipped

SEQ ID NO: 200 - DNA, 321 bp, Artificial Sequence
  Anti-Human Cleaved Variable Light Chain -- IgGA09
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatctggtaatgtgccggcgacgttcggacagggtaccaaggtggagatcaaa

SEQ ID NO: 201 - skipped

SEQ ID NO: 202 - DNA, 321 bp, Artificial Sequence
  Anti-Human Cleaved Variable Light Chain -- IgGA10
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatcgggtaagcggccgatgacgttcggacagggtaccaaggtggagatcaaa

SEQ ID NO: 203 - DNA, 381 bp, Artificial Sequence
  Anti-Human Cleaved Variable Heavy Chain -- IgGA11, IgGB04, IgGB08, IgGC01, IgGC04, IgGD04, IgGD07
gaggttcagctggtggagtctggcggtggcctggtgcagccagggggctcactccgtttgtcctgtgcagcttctggcttcgacttctcttcttattctatacactgggtgcgtcaggccccgggtaagggcctggaatgggttgcatctatttatccttattctggctctacttattatgccgatagcgtcaagggccgtttcactataagcgcagacacatccaaaaacacagcctacctacaaatgaacagcttaagagctgaggacactgccgtctattattgtgctcgccggtctcattcgtactactcgcatacttggtgggtttcttacggtatggactactggggtcaaggaaccctggtcaccgtctcctcg

SEQ ID NO: 204 - DNA, 321 bp, Artificial Sequence
  Anti-Human Cleaved Variable Light Chain -- IgGA11
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaactggtcagcgtccgatgacgttcggacagggtaccaaggtggagatcaaa

SEQ ID NO: 205 - skipped

SEQ ID NO: 206 - DNA, 321 bp, Artificial Sequence
  Anti-Human Cleaved Variable Light Chain -- IgGB01
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatcgatgcagtggccgaagacgttcggacagggtaccaaggtggagatcaaa

SEQ ID NO: 207 - DNA, 381 bp, Artificial Sequence
  Anti-Human Cleaved Variable Heavy Chain -- IgGB03
gaggttcagctggtggagtctggcggtggcctggtgcagccagggggctcactccgtttgtcctgtgcagcttctggcttcgacttctcttcttattctatacactgggtgcgtcaggccccgggtaagggcctggaatgggttgcatctatttatccttattctggctctacttattatgccgatagcgtcaagggccgtttcactataagcgcagacacatccaaaaacacagcctacctacaaatgaacagcttaagagctgaggacactgccgtctattattgtgctcgccagtcgcattcgttctactctcatacttggtgggtttcttacggtatggactactggggtcaaggaaccctggtcaccgtctcctcg

SEQ ID NO: 208 - DNA, 321 bp, Artificial Sequence
  Anti-Human Cleaved Variable Light Chain -- IgGB03
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatctttgcagcggccgatgacgttcggacagggtaccaaggtggagatcaaa

SEQ ID NO: 209 - skipped

SEQ ID NO: 210 - DNA, 321 bp, Artificial Sequence
  Anti-Human Cleaved Variable Light Chain -- IgGB04
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaagtggagctggtgcctatgacgttcggacagggtaccaaggtggagatcaaa

SEQ ID NO: 211 - DNA, 381 bp, Artificial Sequence
  Anti-Human Cleaved Variable Heavy Chain -- IgGB07, IgGD03
gaggttcagctggtggagtctggcggtggcctggtgcagccagggggctcactccgtttgtcctgtgcagcttctggcttcgacttctcttcttattctatacactgggtgcgtcaggccccgggtaagggcctggaatgggttgcatctatttatccttattctggctctacttattatgccgatagcgtcaagggccgtttcactataagcgcagacacatccaaaaacacagcctacctacaaatgaacagcttaagagctgaggacactgccgtctattattgtgctcgccaggatcattcttactactctcatacttggtgggtttcttacggtatggactactggggtcaaggaaccctggtcaccgtctcctcg

SEQ ID NO: 212 - DNA, 321 bp, Artificial Sequence
  Anti-Human Cleaved Variable Light Chain -- IgGB07
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaagctgggaagcgtccgttgacgttcggacagggtaccaaggtggagatcaaa

SEQ ID NO: 213 - skipped

SEQ ID NO: 214 - DNA, 321 bp, Artificial Sequence
  Anti-Human Cleaved Variable Light Chain -- IgGB08
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatctgttgatttgactgcgacgttcggacagggtaccaaggtggagatcaaa

SEQ ID NO: 215 - skipped

SEQ ID NO: 216 - DNA, 321 bp, Artificial Sequence
  Anti-Human Cleaved Variable Light Chain -- IgGC01
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaattgggtgtgcgggcggcgacgttcggacagggtaccaaggtggagatcaaa

SEQ ID NO: 217 - skipped

SEQ ID NO: 218 - DNA, 321 bp, Artificial Sequence
  Anti-Human Cleaved Variable Light Chain -- IgGC03
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatctgggcggaagccggtgacgttcggacagggtaccaaggtggagatcaaa

SEQ ID NO: 219 - skipped

SEQ ID NO: 220 - DNA, 321 bp, Artificial Sequence
  Anti-Human Cleaved Variable Light Chain -- IgGC04
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaactgctcagagtccgctgacgttcggacagggtaccaaggtggagatcaaa

SEQ ID NO: 221 - skipped

SEQ ID NO: 222 - DNA, 321 bp, Artificial Sequence
  Anti-Human Cleaved Variable Light Chain -- IgGC05
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatctaggcagcggcctctgacgttcggacagggtaccaaggtggagatcaaa

SEQ ID NO: 223 - skipped

SEQ ID NO: 224 - DNA, 321 bp, Artificial Sequence
  Anti-Human Cleaved Variable Light Chain -- IgGC06
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatcggggctgatgccgttgacgttcggacagggtaccaaggtggagatcaaa

SEQ ID NO: 225 - skipped

SEQ ID NO: 226 - DNA, 321 bp, Artificial Sequence
  Anti-Human Cleaved Variable Light Chain -- IgGC09
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatcggggcatcggcctattacgttcggacagggtaccaaggtggagatcaaa

SEQ ID NO: 227 - DNA, 381 bp, Artificial Sequence
  Anti-Human Cleaved Variable Heavy Chain -- IgGD02
gaggttcagctggtggagtctggcggtggcctggtgcagccagggggctcactccgtttgtcctgtgcagcttctggcttcgacttctcttcttattctatacactgggtgcgtcaggccccgggtaagggcctggaatgggttgcatctatttatccttattctggctctacttattatgccgatagcgtcaagggccgtttcactataagcgcagacacatccaaaaacacagcctacctacaaatgaacagcttaagagctgaggacactgccgtctattattgtgctcgcaagtatcattcttactactctcatacttggtgggtttcttacggtatggactactggggtcaaggaaccctggtcaccgtctcctcg

SEQ ID NO: 228 - DNA, 321 bp, Artificial Sequence
  Anti-Human Cleaved Variable Light Chain -- IgGD02
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatcttctcagaggcctgcgacgttcggacagggtaccaaggtggagatcaaa

SEQ ID NO: 229 - skipped

SEQ ID NO: 230 - DNA, 321 bp, Artificial Sequence
  Anti-Human Cleaved Variable Light Chain -- IgGD03
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatcgggtcgtcagcctttgacgttcggacagggtaccaaggtggagatcaaa

SEQ ID NO: 231 - skipped

SEQ ID NO: 232 - DNA, 321 bp, Artificial Sequence
  Anti-Human Cleaved Variable Light Chain -- IgGD04
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatttatgcggccggcgtttacgttcggacagggtaccaaggtggagatcaaa

SEQ ID NO: 233 - skipped

SEQ ID NO: 234 - DNA, 321 bp, Artificial Sequence
  Anti-Human Cleaved Variable Light Chain -- IgGD07
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatcgggtcagccttcgatgacgttcggacagggtaccaaggtggagatcaaa

SEQ ID NO: 235 - skipped

SEQ ID NO: 236 - DNA, 321 bp, Artificial Sequence
  Anti-Human Cleaved Variable Light Chain -- IgGD08
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatcggtgttgcggccttttacgttcggacagggtaccaaggtggagatcaaa

SEQ ID NO: 237-238 - skipped (2 entries)

SEQ ID NO: 239 - DNA, 372 bp, Artificial Sequence
  Anti-Mouse Cleaved Variable Heavy Chain -- Fab 0114, IgG 0029 (Fab 0114)
gaggttcagctggtggagtctggcggtggcctggtgcagccagggggctcactccgtttgtcctgtgcagcttctggcttcaacgtctcttattcttctatacactgggtgcgtcaggccccgggtaagggcctggaatgggttgcatctatttcttcttcttctggctatacttattatgccgatagcgtcaagggccgtttcactataagcgcagacacatccaaaaacacagcctacctacaaatgaacagcttaagagctgaggacactgccgtctattattgtgctcgccaggaaggtgattggtgggttgcttactggccggctttggactactggggtcaaggaaccctggtcaccgtctcctcg

SEQ ID NO: 240 - DNA, 321 bp, Artificial Sequence
  Anti-Mouse Cleaved Variable Light Chain -- Fab 0114, IgG 0029 (Fab 0114)
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatcttcttggggtctgttcacgttcggacagggtaccaaggtggagatcaaa

SEQ ID NO: 241-242 - skipped (2 entries)

SEQ ID NO: 243 - DNA, 381 bp, Artificial Sequence
  Anti-Mouse Cleaved Variable Heavy Chain -- Fab 0123, IgG 0030 (Fab 0123)
gaggttcagctggtggagtctggcggtggcctggtgcagccagggggctcactccgtttgtcctgtgcagcttctggcttcaacgtctattcttattctatacactgggtgcgtcaggccccgggtaagggcctggaatgggttgcatctatttcttcttcttctggctatacttattatgccgatagcgtcaagggccgtttcactataagcgcagacacatccaaaaacacagcctacctacaaatgaacagcttaagagctgaggacactgccgtctattattgtgctcgctactactgggacccgtggcagtactggtactactcttacctggcttttgactactggggtcaaggaaccctggtcaccgtctcctcg

SEQ ID NO: 244 - DNA, 321 bp, Artificial Sequence
  Anti-Mouse Cleaved Variable Light Chain -- Fab 0123, IgG 0030 (Fab 0123), IgG 0046, IgG 0051, IgG 0055, IgG 0058, IgG 0059, IgG 0060, IgG 0062, IgG 0063, IgG 0065
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaacttactctggtccgatcacgttcggacagggtaccaaggtggagatcaaa

SEQ ID NO: 245-246 - skipped (2 entries)

SEQ ID NO: 247 - DNA, 381 bp, Artificial Sequence
  Anti-Mouse Cleaved Variable Heavy Chain -- IgG 0046
gaggttcagctggtggagtctggcggtggcctggtgcagccagggggctcactccgtttgtcctgtgcagcttctggcttcaacgtctattcttattctatacactgggtgcgtcaggccccgggtaagggcctggaatgggttgcatctatttcttcttcttctggctatacttattatgccgatagcgtcaagggccgtttcactataagcgcagacacatccaaaaacacagcctacctacaaatgaacagcttaagagctgaggacactgccgtctattattgtgctcgctactactgggacccgtggcagtacgcgtactactcttacctggcttttgactactggggtcaaggaaccctggtcaccgtctcctcg

SEQ ID NO: 248 - skipped

SEQ ID NO: 249 - DNA, 381 bp, Artificial Sequence
  Anti-Mouse Cleaved Variable Heavy Chain -- IgG 0050
gaggttcagctggtggagtctggcggtggcctggtgcagccagggggctcactccgtttgtcctgtgcagcttctggcttcaacgtctattcttattctatacactgggtgcgtcaggccccgggtaagggcctggaatgggttgcatctatttcttcttcttctggctctacttattatgccgatagcgtcaagggccgtttcactataagcgcagacacatccaaaaacacagcctacctacaaatgaacagcttaagagctgaggacactgccgtctattattgtgctcgctactactgggacccgtggcagtacgcgtactactcttacctggcttttgactactggggtcaaggaaccctggtcaccgtctcctcg

SEQ ID NO: 250 - DNA, 321 bp, Artificial Sequence; identical to SEQ ID NO: 244
  Anti-Mouse Cleaved Variable Light Chain -- IgG 0050

SEQ ID NO: 251 - DNA, 381 bp, Artificial Sequence
  Anti-Mouse Cleaved Variable Heavy Chain -- IgG 0051
gaggttcagctggtggagtctggcggtggcctggtgcagccagggggctcactccgtttgtcctgtgcagcttctggcttcaacgtctattcttattctatacactgggtgcgtcaggccccgggtaagggcctggaatgggttgcatctatttcttcttcttctggctatacttcttatgccgatagcgtcaagggccgtttcactataagcgcagacacatccaaaaacacagcctacctacaaatgaacagcttaagagctgaggacactgccgtctattattgtgctcgctactactgggacccgtggcagtacgcgtactactcttacctggcttttgactactggggtcaaggaaccctggtcaccgtctcctcg

SEQ ID NO: 252 - skipped

SEQ ID NO: 253 - DNA, 381 bp, Artificial Sequence
  Anti-Mouse Cleaved Variable Heavy Chain -- IgG 0055
gaggttcagctggtggagtctggcggtggcctggtgcagccagggggctcactccgtttgtcctgtgcagcttctggcttcaacgtctattcttattctatacactgggtgcgtcaggccccgggtaagggcctggaatgggttgcatctatttcttcttcttctggctatacttattatgccgatagcgtcaagggccgtttcactataagcgcagacacatccaaaaacacagcctacctacaaatgaacagcttaagagctgaggacactgccgtctattattgtgctcgctactactgggacccgtggcagtacgcgtcctactcttacctggcttttgactactggggtcaaggaaccctggtcaccgtctcctcg

SEQ ID NO: 254-255 - skipped (2 entries)

SEQ ID NO: 256 - DNA, 381 bp, Artificial Sequence
  Anti-Mouse Cleaved Variable Heavy Chain -- IgG 0058
gaggttcagctggtggagtctggcggtggcctggtgcagccagggggctcactccgtttgtcctgtgcagcttctggcttcaacgtctattcttattctatacactgggtgcgtcaggccccgggtaagggcctggaatgggttgcatctatttcttcttcttctggcagtactagttatgccgatagcgtcaagggccgtttcactataagcgcagacacatccaaaaacacagcctacctacaaatgaacagcttaagagctgaggacactgccgtctattattgtgctcgctactactgggacccgtggcagtacgcgtcctactcttacctggcttttgactactggggtcaaggaaccctggtcaccgtctcctcg

SEQ ID NO: 257 - skipped

SEQ ID NO: 258 - DNA, 381 bp, Artificial Sequence
  Anti-Mouse Cleaved Variable Heavy Chain -- IgG 0059
gaggttcagctggtggagtctggcggtggcctggtgcagccagggggctcactccgtttgtcctgtgcagcttctggcttcaacgtctattcttattctatacactgggtgcgtcaggccccgggtaagggcctggaatgggttgcatctatttcttcttcttctggcagtacttattatgccgatagcgtcaagggccgtttcactataagcgcagacacatccaaaaacacagcctacctacaaatgaacagcttaagagctgaggacactgccgtctattattgtgctcgctactactgggacccgtggcagtacgcgtcctactcttacctggcttttgactactggggtcaaggaaccctggtcaccgtctcctcg

SEQ ID NO: 259 - skipped

SEQ ID NO: 260 - DNA, 381 bp, Artificial Sequence
  Anti-Mouse Cleaved Variable Heavy Chain -- IgG 0060
gaggttcagctggtggagtctggcggtggcctggtgcagccagggggctcactccgtttgtcctgtgcagcttctggcttcaacgtctattcttattctatacactgggtgcgtcaggccccgggtaagggcctggaatgggttgcatctatttcttcttcttctggctatactagttatgccgatagcgtcaagggccgtttcactataagcgcagacacatccaaaaacacagcctacctacaaatgaacagcttaagagctgaggacactgccgtctattattgtgctcgctactactgggacccgtggcagtacgcgtcctactcttacctggcttttgactactggggtcaaggaaccctggtcaccgtctcctcg

SEQ ID NO: 261 - skipped

SEQ ID NO: 262 - DNA, 381 bp, Artificial Sequence
  Anti-Mouse Cleaved Variable Heavy Chain -- IgG 0062
gaggttcagctggtggagtctggcggtggcctggtgcagccagggggctcactccgtttgtcctgtgcagcttctggcttcaacgtctattcttattctatacactgggtgcgtcaggccccgggtaagggcctggaatgggttgcatctatttcttcttcttctggcagtacttattatgccgatagcgtcaagggccgtttcactataagcgcagacacatccaaaaacacagcctacctacaaatgaacagcttaagagctgaggacactgccgtctattattgtgctcgctactactgggacccgtggcagtacagttcctactcttacctggcttttgactactggggtcaaggaaccctggtcaccgtctcctcg

SEQ ID NO: 263 - skipped

SEQ ID NO: 264 - DNA, 381 bp, Artificial Sequence
  Anti-Mouse Cleaved Variable Heavy Chain -- IgG 0063
gaggttcagctggtggagtctggcggtggcctggtgcagccagggggctcactccgtttgtcctgtgcagcttctggcttcaacgtctattcttattctatacactgggtgcgtcaggccccgggtaagggcctggaatgggttgcatctatttcttcttcttctggctatactagttatgccgatagcgtcaagggccgtttcactataagcgcagacacatccaaaaacacagcctacctacaaatgaacagcttaagagctgaggacactgccgtctattattgtgctcgctactactgggacccgtggcagtacagttcctactcttacctggcttttgactactggggtcaaggaaccctggtcaccgtctcctcg

SEQ ID NO: 265 - skipped

SEQ ID NO: 266 - DNA, 381 bp, Artificial Sequence
  Anti-Mouse Cleaved Variable Heavy Chain -- IgG 0065
gaggttcagctggtggagtctggcggtggcctggtgcagccagggggctcactccgtttgtcctgtgcagcttctggcttcaacgtctattcttattctatacactgggtgcgtcaggccccgggtaagggcctggaatgggttgcatctatttcttcttcttctggcagtactagttatgccgatagcgtcaagggccgtttcactataagcgcagacacatccaaaaacacagcctacctacaaatgaacagcttaagagctgaggacactgccgtctattattgtgctcgctactactgggacccgtggcagtacagttcctactcttacctggcttttgactactggggtcaaggaaccctggtcaccgtctcctcg

SEQ ID NO: 267 - skipped

SEQ ID NO: 268 - protein, 6 aa, Artificial Sequence
  Synthetic sequence
  MISC_FEATURE (2)..(2): Wherein Xaa can be Glycine(G), Serine(S), Methionine(M), Leucine(L), Valine(V), or Arginine(R)
  MISC_FEATURE (3)..(3): Wherein Xaa can be Glutamine(Q), Serine(S), Glutamic acid (E), Asparagine(N), Lysine(K), Proline (P), Arginine(R), Leucine(L), or Histidine(H)
  MISC_FEATURE (4)..(4): Wherein Xaa can be Arginine(R), Serine(S), Valine(V), Tryptophan(W), Leucine(L), Lysine(K), Methionine (M), Glutamine(Q), or Proline(P)
  MISC_FEATURE (5)..(5): Wherein Xaa can be Proline (P), Leucine(L), Threonine(T), or Serine(S)
  MISC_FEATURE (6)..(6): Wherein Xaa can be Isoleucine(I), Alanine(A), Methionine(M), Lysine(K), Valine(V), Leucine(L), Phenylalanine(F)
SXXXXX

SEQ ID NO: 269 - protein, 7 aa, Artificial Sequence
  Synthetic sequence
  MISC_FEATURE (1)..(1): Wherein Xaa can be No Amino Acid, Aspartic acid(D), or Asparagine(N)
  MISC_FEATURE (5)..(5): Wherein Xaa can be Serine(S) or Tyrosine(Y)
XFSSXSI

SEQ ID NO: 270 - protein, 10 aa, Artificial Sequence
  Synthetic sequence
  MISC_FEATURE (10)..(10): Wherein Xaa can be Serine(S) or Tyrosine(Y)
SIYPYSGSTX

SEQ ID NO: 271 - protein, 16 aa, Artificial Sequence
  Synthetic sequence
  MISC_FEATURE (1)..(1): Wherein Xaa can be Glutamine(Q), Arginine(R), or Lysine(K)
  MISC_FEATURE (2)..(2): Wherein Xaa can be Serine(S), Asparagine(N), Threonine(T), Glycine(G), Alanine(A), or Aspartic acid(D)
  MISC_FEATURE (3)..(3): Wherein Xaa can be Tyrosine(Y) or Phenylalanine(F)
  MISC_FEATURE (5)..(5): Wherein Xaa can be Tyrosine(Y) or Phenylalanine(F)
  MISC_FEATURE (16)..(16): Wherein Xaa can be Methionine(M), Alanine(A), Isoleucine(I), Leucine(L), or Valine(V)
XXXSXYSHTWWVSYGX

SEQ ID NO: 272 - protein, 18 aa, Artificial Sequence
  Synthetic sequence
  MISC_FEATURE (1)..(1): Wherein Xaa can be Threonine(T) or Isoleucine(I)
  MISC_FEATURE (5)..(5): Wherein Xaa can be Glutamine(Q) or Aspartic acid(D)
XYWVXFWYGHFSYYRPAL

SEQ ID NO: 273 - protein, 836 aa, Artificial Sequence
  Human CDCP1
MAGLNCGVSIALLGVLLLGAARLPRGAEAFEIALPRESNITVLIKLGTPTLLAKPCYIVISKRHITMLSIKSGERIVFTFSCQSPENHFVIEIQKNIDCMSGPCPFGEVQLQPSTSLLPTLNRTFIWDVKAHKSIGLELQFSIPRLRQIGPGESCPDGVTHSISGRIDATVVRIGTFCSNGTVSRIKMQEGVKMALHLPWFHPRNVSGFSIANRSSIKRLCIIESVFEGEGSATLMSANYPEGFPEDELMTWQFVVPAHLRASVSFLNFNLSNCERKEERVEYYIPGSTTNPEVFKLEDKQPGNMAGNFNLSLQGCDQDAQSPGILRLQFQVLVQHPQNESNKIYVVDLSNERAMSLTIEPRPVKQSRKFVPGCFVCLESRTCSSNLTLTSGSKHKISFLCDDLTRLWMNVEKTISCTDHRYCQRKSYSLQVPSDILHLPVELHDFSWKLLVPKDRLSLVLVPAQKLQQHTHEKPCNTSFSYLVASAIPSQDLYFGSFCPGGSIKQIQVKQNISVTLRTFAPSFQQEASRQGLTVSFIPYFKEEGVFTVTPDTKSKVYLRTPNWDRGLPSLTSVSWNISVPRDQVACLTFFKERSGVVCQTGRAFMIIQEQRTRAEEIFSLDEDVLPKPSFHHHSFWVNISNCSPTSGKQLDLLFSVTLTPRTVDLTVILIAAVGGGVLLLSALGLIICCVKKKKKKTNKGPAVGIYNDNINTEMPRQPKKFQKGRKDNDSHVYAVIEDTMVYGHLLQDSSGSFLQPEVDTYRPFQGTMGVCPPSPPTICSRAPTAKLATEEPPPRSPPESESEPYTFSHPNNGDVSSKDTDIPLLNTQEPMEPAE

SEQ ID NO: 274 - protein, 649 aa, Artificial Sequence
  Human CDCP1 isoform 2
MQEGVKMALHLPWFHPRNVSGFSIANRSSIKRLCIIESVFEGEGSATLMSANYPEGFPEDELMTWQFVVPAHLRASVSFLNFNLSNCERKEERVEYYIPGSTTNPEVFKLEDKQPGNMAGNFNLSLQGCDQDAQSPGILRLQFQVLVQHPQNESNKIYVVDLSNERAMSLTIEPRPVKQSRKFVPGCFVCLESRTCSSNLTLTSGSKHKISFLCDDLTRLWMNVEKTISCTDHRYCQRKSYSLQVPSDILHLPVELHDFSWKLLVPKDRLSLVLVPAQKLQQHTHEKPCNTSFSYLVASAIPSQDLYFGSFCPGGSIKQIQVKQNISVTLRTFAPSFQQEASRQGLTVSFIPYFKEEGVFTVTPDTKSKVYLRTPNWDRGLPSLTSVSWNISVPRDQVACLTFFKERSGVVCQTGRAFMIIQEQRTRAEEIFSLDEDVLPKPSFHHHSFWVNISNCSPTSGKQLDLLFSVTLTPRTVDLTVILIAAVGGGVLLLSALGLIICCVKKKKKKTNKGPAVGIYNDNINTEMPRQPKKFQKGRKDNDSHVYAVIEDTMVYGHLLQDSSGSFLQPEVDTYRPFQGTMGVCPPSPPTICSRAPTAKLATEEPPPRSPPESESEPYTFSHPNNGDVSSKDTDIPLLNTQEPMEPAE

SEQ ID NO: 275 - protein, 343 aa, Artificial Sequence
  Human CDCP1 isoform 3
MAGLNCGVSIALLGVLLLGAARLPRGAEAFEIALPRESNITVLIKLGTPTLLAKPCYIVISKRHITMLSIKSGERIVFTFSCQSPENHFVIEIQKNIDCMSGPCPFGEVQLQPSTSLLPTLNRTFIWDVKAHKSIGLELQFSIPRLRQIGPGESCPDGVTHSISGRIDATVVRIGTFCSNGTVSRIKMQEGVKMALHLPWFHPRNVSGFSIANRSSIKRLCIIESVFEGEGSATLMSANYPEGFPEDELMTWQFVVPAHLRASVSFLNFNLSNCERKEERVEYYIPGSTTNPEVFKLEDKQPGNMAGNFNLSLQGCDQDAQSPGILRLQFQVLVQHPQNESSE

SEQ ID NO: 276 - protein, 807 aa, Artificial Sequence
  Extracellular domain of mature human CDCP1
FEIALPRESNITVLIKLGTPTLLAKPCYIVISKRHITMLSIKSGERIVFTFSCQSPENHFVIEIQKNIDCMSGPCPFGEVQLQPSTSLLPTLNRTFIWDVKAHKSIGLELQFSIPRLRQIGPGESCPDGVTHSISGRIDATVVRIGTFCSNGTVSRIKMQEGVKMALHLPWFHPRNVSGFSIANRSSIKRLCIIESVFEGEGSATLMSANYPEGFPEDELMTWQFVVPAHLRASVSFLNFNLSNCERKEERVEYYIPGSTTNPEVFKLEDKQPGNMAGNFNLSLQGCDQDAQSPGILRLQFQVLVQHPQNESNKIYVVDLSNERAMSLTIEPRPVKQSRKFVPGCFVCLESRTCSSNLTLTSGSKHKISFLCDDLTRLWMNVEKTISCTDHRYCQRKSYSLQVPSDILHLPVELHDFSWKLLVPKDRLSLVLVPAQKLQQHTHEKPCNTSFSYLVASAIPSQDLYFGSFCPGGSIKQIQVKQNISVTLRTFAPSFQQEASRQGLTVSFIPYFKEEGVFTVTPDTKSKVYLRTPNWDRGLPSLTSVSWNISVPRDQVACLTFFKERSGVVCQTGRAFMIIQEQRTRAEEIFSLDEDVLPKPSFHHHSFWVNISNCSPTSGKQLDLLFSVTLTPRTVDLTVILIAAVGGGVLLLSALGLIICCVKKKKKKTNKGPAVGIYNDNINTEMPRQPKKFQKGRKDNDSHVYAVIEDTMVYGHLLQDSSGSFLQPEVDTYRPFQGTMGVCPPSPPTICSRAPTAKLATEEPPPRSPPESESEPYTFSHPNNGDVSSKDTDIPLLNTQEPMEPAE

SEQ ID NO: 277 - protein, 833 aa, Artificial Sequence
  Mouse CDCP1
MAHSACGFSVALLGALLLGTARLLRGTEASEIALPQRSGVTVSIKLGNPALPVKICYIVMSRQHITELIIRPGERKSFTFSCSNPEKHFVLKIEKNIDCMSGPCPFGEVHLQPSTSELPILNRTFIWDVRAHKSIGLELQFATPRLRQIGPGESCADGVTHSISGHIDATEVRIGTFCSNGTVSRIKMQEGVKMALHLPWFHRRNVSGFSIANRSSIKRLCIIESVFEGEGSATLMSANYPGGFPEDELMTWQFVVPAHLRASVSFLNFNVSNCERKEERVEYYIPGSTTNPEVFRLEDKQPGNMAGNFNLSLQGCDQDAQSPGILRLQFQVLVQRPQDESNKTYMVDLSRERTMSLTIEPRPVKHGRRFVPGCFVCLESRTCSTNVTLTAGSIHKISFLCDDLTRLWVNVEKTLSCLDHRYCYRQSFKLQVPDYILQLPVQLHDFSWKLLVPKDKLSLMLVPGQKLQQHTQERPCNTSFGYHVTSTTPGQDLYFGSFCSGGSIEKIQVKQNSSVTLRAYAPSFQQEVSKQGLIVSYTPYFKEEGIFTVTPDTKNKVYLRSPNWDRGLPALSSVSWNISVPSNQVACLTVLKERSGLACQSGRAFMIIQEQQSRAEEIFSLEEEVLPKPSFHHHSFWVNISNCSPMNGKQLDLLFWVTLTPRTVDLAVVIGAAGGGALLLFALVLIICFVKKKKKVDKGPAVGIYNGNVNTQMPQTQKFPKGRKDNDSHVYAVIEDTMVYGHLLQDSGGSFIQPEVDTYRPFQGPMGDCPPTPPPLFSRTPTAKFTAEELAPSSPPESESEPYTFSHPNKGEIGVRETDIPLLHTQGPVETEE

SEQ ID NO: 278 - protein, 836 aa, Artificial Sequence
  Cynomolgus CDCP1
MAGLNCGVTIALLGVLLLGAARLPRAAEAFEIALPRESNITVLIKLGTPTLLAKPCYIVISKRHTTMLSIKPGERILFTFSCQSPENHFVIEIQKNIDCMSGPCPFGEVQLQPSTSLLPTLNRTFIWDVKAHKSIGLELQFSIPLLRQIGPGESCPGGVTYSISGRIDATVVRIGTFCSNGTVSRIKMQEGVKMALHLPWFHPRNVSGFSIANRSSIKRLCIIESVFEGEGSATLMSANYPEGFPEDELMTWQFVIPAHLRASVSFLNFNLSNCERKEERVEYYIPGSTTNPEVFKLEDKQPGNMAGNFNLSLQGCDQDAQNPGILRLQFQVLVQHPQNESNKIYVVDLSNERATSLTIEPRPVKQSRKFVPGCFVCLESRTCSTNLTLTSGSKHKISFLCDNLTRLWMNVEKNISCTDHRYCQRKSYSLQVPSDILHLPVELHDFSWKLLVPKDRLSLVLVPAQKLQQHTHEKPCNTSFSYLVASAIPSQDLYFGSFCPGGSIEQIQVKQNISVTLRTFAPSFRQEASRQGLTVSFIPYFKEEGVFTVTPDTKSKVYLRTPNWDRGLPSLTSVSWNISVPRDQVACLTFFKERTGVVCQTGRAFMIIQEQRTRAEEIFSLDEDALPKPRFHHHSFWVNISNCSPASGKQLDLLFWVTLTPRTVDLTVILITVVGGGAVLLSALGLIICCVKKKKKKTNKGPAVGVYNGNINTEMPRQPKKFQKGRKDNDSHVYAVIEDTMVYGHLLQDSGGSFLQPEVDTYRPFQGTMGVCPPSPPTICSRAPTAKLAAEELPPCSPPESESEPYTFSHPNNGDINSKETDIPLLNTQEPVEPAE

SEQ ID NO: 279 - protein, 233 aa, Artificial Sequence
  Anti-Human Cleaved Heavy Chain -- CL03 Fab
EISEVQLVESGGGLVQPGGSLRLSCAASGFNFSSYSIHWVRQAPGKGLEWVASIYPYSGSTYYADSVKGRFTISADTSKNTAYLQMNSLRAEDTAVYYCARQSHSYYSHTWWVSYGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 280 - protein, 457 aa, Artificial Sequence
  Anti-Human Cleaved Heavy Chain -- CL03 IgG
EVQLVESGGGLVQPGGSLRLSCAASGFNFSSYSIHWVRQAPGKGLEWVASIYPYSGSTYYADSVKGRFTISADTSKNTAYLQMNSLRAEDTAVYYCARQSHSYYSHTWWVSYGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 281 - protein, 457 aa, Artificial Sequence
  Anti-Human Cleaved Heavy Chain -- CL03 IgG H1-NtoD; IgG3H7L; IgGA(08-10); IgGB01; IgGC(03, 05, 06, 09); IgGD08
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQSHSYYSHTWWVSYGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 282 - protein, 457 aa, Artificial Sequence
  Anti-Human Cleaved Heavy Chain -- CL03 IgG H1-NtoD H3-MtoA (“IgG101”)
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQSHSYYSHTWWVSYGADYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 283 - protein, 457 aa, Artificial Sequence
  Anti-Human Cleaved Heavy Chain -- CL03 IgG H1-NtoD H3-MtoI
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQSHSYYSHTWWVSYGIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 284 - protein, 457 aa, Artificial Sequence
  Anti-Human Cleaved Heavy Chain -- CL03 IgG H1-NtoD H3-MtoL
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQSHSYYSHTWWVSYGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 285 - protein, 457 aa, Artificial Sequence
  Anti-Human Cleaved Heavy Chain -- CL03 IgG H1-NtoD H3-MtoV
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQSHSYYSHTWWVSYGVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 286 - protein, 235 aa, Artificial Sequence
  Anti-Human Cleaved Heavy Chain -- CL07 Fab
EISEVQLVESGGGLVQPGGSLRLSCAASGFNFSSSSIHWVRQAPGKGLEWVASIYPYSGSTSYADSVKGRFTISADTSKNTAYLQMNSLRAEDTAVYYCARTYWVQFWYGHFSYYRPALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 287 - protein, 459 aa, Artificial Sequence
  Anti-Human Cleaved Heavy Chain -- CL07 IgG
EVQLVESGGGLVQPGGSLRLSCAASGFNFSSSSIHWVRQAPGKGLEWVASIYPYSGSTSYADSVKGRFTISADTSKNTAYLQMNSLRAEDTAVYYCARTYWVQFWYGHFSYYRPALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 288 - protein, 459 aa, Artificial Sequence
  Anti-Human Cleaved Heavy Chain -- CL07 IgG H1-NtoD
EVQLVESGGGLVQPGGSLRLSCAASGFDFSSSSIHWVRQAPGKGLEWVASIYPYSGSTSYADSVKGRFTISADTSKNTAYLQMNSLRAEDTAVYYCARTYWVQFWYGHFSYYRPALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 289 - skipped

SEQ ID NO: 290 - protein, 457 aa, Artificial Sequence
  Anti-Human Cleaved Heavy Chain -- IgGA01
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQNHSYYSHTWWVSYGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 291 - protein, 457 aa, Artificial Sequence
  Anti-Human Cleaved Heavy Chain -- IgGA03
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQTHSYYSHTWWVSYGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 292 - protein, 457 aa, Artificial Sequence
  Anti-Human Cleaved Heavy Chain -- IgGA04
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQGHSYYSHTWWVSYGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 293 - protein, 457 aa, Artificial Sequence
  Anti-Human Cleaved Heavy Chain -- IgGA05
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQAHSYYSHTWWVSYGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 294 - protein, 457 aa, Artificial Sequence
  Anti-Human Cleaved Heavy Chain -- IgGA06
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQGQSYYSHTWWVSYGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 295-297 - skipped (3 entries)

SEQ ID NO: 298 - protein, 457 aa, Artificial Sequence
  Anti-Human Cleaved Heavy Chain -- IgGA11, IgGB04, IgGB08, IgGC01, IgGC04, IgGD04, IgGD07
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARRSHSYYSHTWWVSYGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 299 - skipped

SEQ ID NO: 300 - protein, 457 aa, Artificial Sequence
  Anti-Human Cleaved Heavy Chain -- IgGB03
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQSHSFYSHTWWVSYGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 301 - skipped

SEQ ID NO: 302 - protein, 457 aa, Artificial Sequence
  Anti-Human Cleaved Heavy Chain -- IgGB07, IgGD03
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQDHSYYSHTWWVSYGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 303-309 - skipped (7 entries)

SEQ ID NO: 310 - protein, 457 aa, Artificial Sequence
  Anti-Human Cleaved Heavy Chain -- IgGD02
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARKYHSYYSHTWWVSYGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 311-314 - skipped (4 entries)

SEQ ID NO: 315 - protein, 459 aa, Artificial Sequence
  Anti-Human Cleaved Heavy Chain -- IgGD09
EVQLVESGGGLVQPGGSLRLSCAASGFNFSSSSIHWVRQAPGKGLEWVASIYPYSGSTSYADSVKGRFTISADTSKNTAYLQMNSLRAEDTAVYYCARIYWVDFWYGHFSYYRPALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 316 - protein, 217 aa, Artificial Sequence
  Anti-Human Cleaved Light Chain -- CL03 Fab
MASDIQMTQSPSSLSASVGDRVTITCRASQSVSSAVAWYQQKPGKAPKLLIYSASSLYSGVPSRFSGSRSGTDFTLTISSLQPEDFATYYCQQSGQRPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 317 - protein, 214 aa, Artificial Sequence
  Anti-Human Cleaved Light Chain -- CL03 IgG; CL03 IgG H1-NtoD; CL03 IgG H1-NtoD (H3-MtoA (“IgG101”), H3-MtoI, H3-MtoL, H3-MtoV)
DIQMTQSPSSLSASVGDRVTITCRASQSVSSAVAWYQQKPGKAPKLLIYSASSLYSGVPSRFSGSRSGTDFTLTISSLQPEDFATYYCQQSGQRPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 318-322 - skipped (5 entries)

SEQ ID NO: 323 - protein, 217 aa, Artificial Sequence
  Anti-Human Cleaved Light Chain -- CL07 Fab
MASDIQMTQSPSSLSASVGDRVTITCRASQSVSSAVAWYQQKPGKAPKLLIYSASSLYSGVPSRFSGSRSGTDFTLTISSLQPEDFATYYCQQSSSSLITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 324 - protein, 214 aa, Artificial Sequence
  Anti-Human Cleaved Light Chain -- CL07 IgG, CL07 IgG H1-NtoD
DIQMTQSPSSLSASVGDRVTITCRASQSVSSAVAWYQQKPGKAPKLLIYSASSLYSGVPSRFSGSRSGTDFTLTISSLQPEDFATYYCQQSSSSLITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 325 - skipped

SEQ ID NO: 326 - protein, 211 aa, Artificial Sequence
  Anti-Human Cleaved Light Chain -- IgG3H7L
MTQSPSSLSASVGDRVTITCRASQSVSSAVAWYQQKPGKAPKLLIYSASSLYSGVPSRFSGSRSGTDFTLTISSLQPEDFATYYCQQSSSSLITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 327 - protein, 211 aa, Artificial Sequence
  Anti-Human Cleaved Light Chain -- IgGA01, IgGA03, IgGA04, IgGA05, IgGA06
MTQSPSSLSASVGDRVTITCRASQSVSSAVAWYQQKPGKAPKLLIYSASSLYSGVPSRFSGSRSGTDFTLTISSLQPEDFATYYCQQSGQRPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 328-331 - skipped (4 entries)

SEQ ID NO: 332 - protein, 211 aa, Artificial Sequence
  Anti-Human Cleaved Light Chain -- IgGA08
MTQSPSSLSASVGDRVTITCRASQSVSSAVAWYQQKPGKAPKLLIYSASSLYSGVPSRFSGSRSGTDFTLTISSLQPEDFATYYCQQSGERPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 333 - protein, 211 aa, Artificial Sequence
  Anti-Human Cleaved Light Chain -- IgGA09
MTQSPSSLSASVGDRVTITCRASQSVSSAVAWYQQKPGKAPKLLIYSASSLYSGVPSRFSGSRSGTDFTLTISSLQPEDFATYYCQQSGNVPATFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 334 - protein, 211 aa, Artificial Sequence
  Anti-Human Cleaved Light Chain -- IgGA10
MTQSPSSLSASVGDRVTITCRASQSVSSAVAWYQQKPGKAPKLLIYSASSLYSGVPSRFSGSRSGTDFTLTISSLQPEDFATYYCQQSGKRPMTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 335 - protein, 211 aa, Artificial Sequence
  Anti-Human Cleaved Light Chain -- IgGA11
MTQSPSSLSASVGDRVTITCRASQSVSSAVAWYQQKPGKAPKLLIYSASSLYSGVPSRFSGSRSGTDFTLTISSLQPEDFATYYCQQTGQRPMTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 336 - protein, 211 aa, Artificial Sequence
  Anti-Human Cleaved Light Chain -- IgGB01
MTQSPSSLSASVGDRVTITCRASQSVSSAVAWYQQKPGKAPKLLIYSASSLYSGVPSRFSGSRSGTDFTLTISSLQPEDFATYYCQQSMQWPKTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 337 - protein, 211 aa, Artificial Sequence
  Anti-Human Cleaved Light Chain -- IgGB03
MTQSPSSLSASVGDRVTITCRASQSVSSAVAWYQQKPGKAPKLLIYSASSLYSGVPSRFSGSRSGTDFTLTISSLQPEDFATYYCQQSLQRPMTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 338 - protein, 211 aa, Artificial Sequence
  Anti-Human Cleaved Light Chain -- IgGB04
MTQSPSSLSASVGDRVTITCRASQSVSSAVAWYQQKPGKAPKLLIYSASSLYSGVPSRFSGSRSGTDFTLTISSLQPEDFATYYCQQVELVPMTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 339 - protein, 211 aa, Artificial Sequence
  Anti-Human Cleaved Light Chain -- IgGB07
MTQSPSSLSASVGDRVTITCRASQSVSSAVAWYQQKPGKAPKLLIYSASSLYSGVPSRFSGSRSGTDFTLTISSLQPEDFATYYCQQAGKRPLTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 340 - protein, 211 aa, Artificial Sequence
  Anti-Human Cleaved Light Chain -- IgGB08
MTQSPSSLSASVGDRVTITCRASQSVSSAVAWYQQKPGKAPKLLIYSASSLYSGVPSRFSGSRSGTDFTLTISSLQPEDFATYYCQQSVDLTATFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 341 - protein, 211 aa, Artificial Sequence
  Anti-Human Cleaved Light Chain -- IgGC01
MTQSPSSLSASVGDRVTITCRASQSVSSAVAWYQQKPGKAPKLLIYSASSLYSGVPSRFSGSRSGTDFTLTISSLQPEDFATYYCQQLGVRAATFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 342 - protein, 211 aa, Artificial Sequence
  Anti-Human Cleaved Light Chain -- IgGC03
MTQSPSSLSASVGDRVTITCRASQSVSSAVAWYQQKPGKAPKLLIYSASSLYSGVPSRFSGSRSGTDFTLTISSLQPEDFATYYCQQSGRKPVTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 343 - protein, 211 aa, Artificial Sequence
  Anti-Human Cleaved Light Chain -- IgGC04
MTQSPSSLSASVGDRVTITCRASQSVSSAVAWYQQKPGKAPKLLIYSASSLYSGVPSRFSGSRSGTDFTLTISSLQPEDFATYYCQQTAQSPLTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 344 - protein, 211 aa, Artificial Sequence
  Anti-Human Cleaved Light Chain -- IgGC05
MTQSPSSLSASVGDRVTITCRASQSVSSAVAWYQQKPGKAPKLLIYSASSLYSGVPSRFSGSRSGTDFTLTISSLQPEDFATYYCQQSRQRPLTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 345 - protein, 211 aa, Artificial Sequence
  Anti-Human Cleaved Light Chain -- IgGC06
MTQSPSSLSASVGDRVTITCRASQSVSSAVAWYQQKPGKAPKLLIYSASSLYSGVPSRFSGSRSGTDFTLTISSLQPEDFATYYCQQSGLMPLTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 346 - protein, 211 aa, Artificial Sequence
  Anti-Human Cleaved Light Chain -- IgGC09
MTQSPSSLSASVGDRVTITCRASQSVSSAVAWYQQKPGKAPKLLIYSASSLYSGVPSRFSGSRSGTDFTLTISSLQPEDFATYYCQQSGHRPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 347 - protein, 211 aa, Artificial Sequence
  Anti-Human Cleaved Light Chain -- IgGD02
MTQSPSSLSASVGDRVTITCRASQSVSSAVAWYQQKPGKAPKLLIYSASSLYSGVPSRFSGSRSGTDFTLTISSLQPEDFATYYCQQSSQRPATFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 348 - protein, 211 aa, Artificial Sequence
  Anti-Human Cleaved Light Chain -- IgGD03
MTQSPSSLSASVGDRVTITCRASQSVSSAVAWYQQKPGKAPKLLIYSASSLYSGVPSRFSGSRSGTDFTLTISSLQPEDFATYYCQQSGRQPLTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 349 - protein, 211 aa, Artificial Sequence
  Anti-Human Cleaved Light Chain -- IgGD04
MTQSPSSLSASVGDRVTITCRASQSVSSAVAWYQQKPGKAPKLLIYSASSLYSGVPSRFSGSRSGTDFTLTISSLQPEDFATYYCQQFMRPAFTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 350 - protein, 211 aa, Artificial Sequence
  Anti-Human Cleaved Light Chain -- IgGD07
MTQSPSSLSASVGDRVTITCRASQSVSSAVAWYQQKPGKAPKLLIYSASSLYSGVPSRFSGSRSGTDFTLTISSLQPEDFATYYCQQSGQPSMTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 351 - protein, 211 aa, Artificial Sequence
  Anti-Human Cleaved Light Chain -- IgGD08
MTQSPSSLSASVGDRVTITCRASQSVSSAVAWYQQKPGKAPKLLIYSASSLYSGVPSRFSGSRSGTDFTLTISSLQPEDFATYYCQQSVLRPFTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 352 - protein, 210 aa, Artificial Sequence
  Anti-Human Cleaved Light Chain -- IgGD09
MTQSPSSLSASVGDRVTITCRASQSVSSAVAWYQQKPGKAPKLLIYSASSLYSGVPSRFSGSRSGTDFTLTISSLQPEDFATYYCQQSSSSLITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 353 - protein, 257 aa, Artificial Sequence
  Anti-Mouse Cleaved Heavy Chain -- Fab 0114
EISEVQLVESGGGLVQPGGSLRLSCAASGFNVSYSSIHWVRQAPGKGLEWVASISSSSGYTYYADSVKGRFTISADTSKNTAYLQMNSLRAEDTAVYYCARQEGDWWVAYWPALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGSGSAGGLNDIFEAQKIEWHE

SEQ ID NO: 354 - protein, 475 aa, Artificial Sequence
  Anti-Mouse Cleaved Heavy Chain -- IgG 0029 (Fab 0114)
EVQLVESGGGLVQPGGSLRLSCAASGFNVSYSSIHWVRQAPGKGLEWVASISSSSGYTYYADSVKGRFTISADTSKNTAYLQMNSLRAEDTAVYYCARQEGDWWVAYWPALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGLNDIFEAQKIEWHEG

SEQ ID NO: 355 - protein, 260 aa, Artificial Sequence
  Anti-Mouse Cleaved Heavy Chain -- Fab 0123
EISEVQLVESGGGLVQPGGSLRLSCAASGFNVYSYSIHWVRQAPGKGLEWVASISSSSGYTYYADSVKGRFTISADTSKNTAYLQMNSLRAEDTAVYYCARYYWDPWQYWYYSYLAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGSGSAGGLNDIFEAQKIEWHE

SEQ ID NO: 356 - protein, 478 aa, Artificial Sequence
  Anti-Mouse Cleaved Heavy Chain -- IgG 0030 (Fab 0123)
EVQLVESGGGLVQPGGSLRLSCAASGFNVYSYSIHWVRQAPGKGLEWVASISSSSGYTYYADSVKGRFTISADTSKNTAYLQMNSLRAEDTAVYYCARYYWDPWQYWYYSYLAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGLNDIFEAQKIEWHEG

SEQ ID NO: 357 - protein, 457 aa, Artificial Sequence
  Anti-Mouse Cleaved Heavy Chain -- IgG 0046
EVQLVESGGGLVQPGGSLRLSCAASGFNVYSYSIHWVRQAPGKGLEWVASISSSSGYTYYADSVKGRFTISADTSKNTAYLQMNSLRAEDTAVYYCARYYWDPWQYAYYSYLAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 358 - protein, 457 aa, Artificial Sequence
  Anti-Mouse Cleaved Heavy Chain -- IgG 0050
EVQLVESGGGLVQPGGSLRLSCAASGFNVYSYSIHWVRQAPGKGLEWVASISSSSGSTYYADSVKGRFTISADTSKNTAYLQMNSLRAEDTAVYYCARYYWDPWQYAYYSYLAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 359 - protein, 457 aa, Artificial Sequence
  Anti-Mouse Cleaved Heavy Chain -- IgG 0051
EVQLVESGGGLVQPGGSLRLSCAASGFNVYSYSIHWVRQAPGKGLEWVASISSSSGYTSYADSVKGRFTISADTSKNTAYLQMNSLRAEDTAVYYCARYYWDPWQYAYYSYLAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 360 - protein, 457 aa, Artificial Sequence
  Anti-Mouse Cleaved Heavy Chain -- IgG 0055
EVQLVESGGGLVQPGGSLRLSCAASGFNVYSYSIHWVRQAPGKGLEWVASISSSSGYTYYADSVKGRFTISADTSKNTAYLQMNSLRAEDTAVYYCARYYWDPWQYASYSYLAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 361 - protein, 457 aa, Artificial Sequence
  Anti-Mouse Cleaved Heavy Chain -- IgG 0058
EVQLVESGGGLVQPGGSLRLSCAASGFNVYSYSIHWVRQAPGKGLEWVASISSSSGSTSYADSVKGRFTISADTSKNTAYLQMNSLRAEDTAVYYCARYYWDPWQYASYSYLAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 362 - protein, 457 aa, Artificial Sequence
  Anti-Mouse Cleaved Heavy Chain -- IgG 0059
EVQLVESGGGLVQPGGSLRLSCAASGFNVYSYSIHWVRQAPGKGLEWVASISSSSGSTYYADSVKGRFTISADTSKNTAYLQMNSLRAEDTAVYYCARYYWDPWQYASYSYLAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 363 - protein, 457 aa, Artificial Sequence
  Anti-Mouse Cleaved Heavy Chain -- IgG 0060
EVQLVESGGGLVQPGGSLRLSCAASGFNVYSYSIHWVRQAPGKGLEWVASISSSSGYTSYADSVKGRFTISADTSKNTAYLQMNSLRAEDTAVYYCARYYWDPWQYASYSYLAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 364 - protein, 457 aa, Artificial Sequence
  Anti-Mouse Cleaved Heavy Chain -- IgG 0062
EVQLVESGGGLVQPGGSLRLSCAASGFNVYSYSIHWVRQAPGKGLEWVASISSSSGSTYYADSVKGRFTISADTSKNTAYLQMNSLRAEDTAVYYCARYYWDPWQYSSYSYLAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 365 - protein, 457 aa, Artificial Sequence
  Anti-Mouse Cleaved Heavy Chain -- IgG 0063
EVQLVESGGGLVQPGGSLRLSCAASGFNVYSYSIHWVRQAPGKGLEWVASISSSSGYTSYADSVKGRFTISADTSKNTAYLQMNSLRAEDTAVYYCARYYWDPWQYSSYSYLAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 366 - protein, 457 aa, Artificial Sequence
  Anti-Mouse Cleaved Heavy Chain -- IgG 0065
EVQLVESGGGLVQPGGSLRLSCAASGFNVYSYSIHWVRQAPGKGLEWVASISSSSGSTSYADSVKGRFTISADTSKNTAYLQMNSLRAEDTAVYYCARYYWDPWQYSSYSYLAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 367 - protein, 217 aa, Artificial Sequence
  Anti-Mouse Cleaved Light Chain -- Fab 0114
MASDIQMTQSPSSLSASVGDRVTITCRASQSVSSAVAWYQQKPGKAPKLLIYSASSLYSGVPSRFSGSRSGTDFTLTISSLQPEDFATYYCQQSSWGLFTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 368 - protein, 215 aa, Artificial Sequence
  Anti-Mouse Cleaved Light Chain -- IgG 0029 (Fab 0114)
SDIQMTQSPSSLSASVGDRVTITCRASQSVSSAVAWYQQKPGKAPKLLIYSASSLYSGVPSRFSGSRSGTDFTLTISSLQPEDFATYYCQQSSWGLFTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 369 - protein, 217 aa, Artificial Sequence
  Anti-Mouse Cleaved Light Chain -- Fab 0123
MASDIQMTQSPSSLSASVGDRVTITCRASQSVSSAVAWYQQKPGKAPKLLIYSASSLYSGVPSRFSGSRSGTDFTLTISSLQPEDFATYYCQQTYSGPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 370 - protein, 215 aa, Artificial Sequence
  Anti-Mouse Cleaved Light Chain -- IgG 0030 (Fab 0123)
SDIQMTQSPSSLSASVGDRVTITCRASQSVSSAVAWYQQKPGKAPKLLIYSASSLYSGVPSRFSGSRSGTDFTLTISSLQPEDFATYYCQQTYSGPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 371 - protein, 211 aa, Artificial Sequence
  Anti-Mouse Cleaved Light Chain -- IgG 0046, IgG 0051, IgG 0055, IgG 0058, IgG 0059, IgG 0060, IgG 0062, IgG 0063, IgG 0065
MTQSPSSLSASVGDRVTITCRASQSVSSAVAWYQQKPGKAPKLLIYSASSLYSGVPSRFSGSRSGTDFTLTISSLQPEDFATYYCQQTYSGPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 372 - protein, 214 aa, Artificial Sequence
  Anti-Mouse Cleaved Light Chain -- IgG 0050
DIQMTQSPSSLSASVGDRVTITCRASQSVSSAVAWYQQKPGKAPKLLIYSASSLYSGVPSRFSGSRSGTDFTLTISSLQPEDFATYYCQQTYSGPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 373-380 - skipped (8 entries)

SEQ ID NO: 381 - protein, 330 aa, Artificial Sequence [sequence omitted]

SEQ ID NO: 382 - protein, 330 aa, Artificial Sequence
  Allotypic variant of the wild-type human IgG1 constant domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 383 - protein, 330 aa, Artificial Sequence
  Effectorless human IgG1 constant domain - “IgG1.1f”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 384 - protein, 330 aa, Artificial Sequence
  Effectorless human IgG1 constant domain - “IgG1.3f”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 385 - protein, 327 aa, Artificial Sequence
  Human IgG4
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 386 - protein, 107 aa, Artificial Sequence [sequence omitted]

SEQ ID NO: 387 - protein, 5 aa, Artificial Sequence
  C-terminal sequence of heavy chain constant region
LSPGK

SEQ ID NO: 388 - protein, 4 aa, Artificial Sequence
  C-terminal sequence of heavy chain constant region
LSPG

SEQ ID NO: 389 - protein, 3 aa, Artificial Sequence
  C-terminal sequence of heavy chain constant region
LSP

(19) United States
(12) Patent Application Publication
(10) Pub. No.: US 2024/0376224 A1
(43) Pub. Date: Nov. 14, 2024

(54) ANTIBODIES AGAINST CLEAVED CDCP1 AND USES THEREOF

(71) Applicant: The Regents of the University of California, Oakland, CA (US)

(72) Inventors: James WELLS, San Francisco, CA (US); Shion A. LIM, San Francisco, CA (US); Jie ZHOU, San Francisco, CA (US); Alexander MARTINKO, San Francisco, CA (US); Kevin K. LEUNG, San Francisco, CA (US)

(21) Appl. No.: 18/553,143

(22) PCT Filed: Apr. 1, 2022
(86) PCT No.: PCT/US2022/023106

§ 371 Date: Sep. 28, 2023

Related U.S. Application Data
(60) Provisional application No. 63/170,338, filed on Apr. 2, 2021.

(51) Int. Cl. C07K 16/28; A61K 35/17; A61K 39/00; A61K 45/06; A61K 47/68; A61P 35/00; G01N 33/68

(52) U.S. Cl. CPC C07K 16/2896; A61K 35/17; A61K 39/4611; A61K 39/4631; A61K 45/06; A61K 47/68031; A61K 47/6849; A61P 35/00; G01N 33/6893; C07K 2317/14; C07K 2317/31; C07K 2317/41; C07K 2317/565; C07K 2317/73; C07K 2317/77; C07K 2317/92

(57) ABSTRACT

The disclosure provides antibodies that specifically bind to a cleaved CDCP1 and antigen binding fragments thereof and methods of use thereof. In some aspects, the disclosure is directed to methods of treating a cancer in a subject, comprising administering to the subject an antibody or antigen-binding fragment thereof that specifically binds to a cleaved human CDCP1.

### CROSS REFERENCE TO RELATED APPLICATIONS

This application claims the benefit of the filing date of U.S. Provisional Patent Application No. 63/170,338, filed Apr. 2, 2021, the contents of which are incorporated herein by reference in their entirety.

### REFERENCE TO SEQUENCE LISTING SUBMITTED ELECTRONICALLY VIA EFS-WEB

The content of the electronically submitted sequence listing in ASCII text file (Name: 3338_242PC01_Seqlisting_ST25.txt; Size: 426,827 bytes; and Date of Creation: Mar. 31, 2022) filed with the application is herein incorporated by reference in its entirety.

### FIELD OF THE DISCLOSURE

The present disclosure provides antibodies that specifically bind to a cleaved CDCP1 (e.g., human or mouse CDCP1), and antigen-binding fragments thereof, compositions comprising such antibodies, and methods of using such antibodies for preventing or treating diseases or conditions which comprise a tumor (e.g., cancers that have cleaved CDCP1 present on the cancer cell surface) in a subject.

### BACKGROUND OF THE DISCLOSURE

CUB domain-containing protein 1 (CDCP1) CDCP1 is a 135-kDa, heavily glycosylated, single-pass membrane protein with largely unknown function. See e.g., Stephen, A. G. et al., Cancer Cell, 25 (3): 272-281 (2014); Papke, B. et al., Science, 355 (6330): 1158-1163 (2017). Overexpression of CDCP1 correlates with increased malignancy and poor prognosis in pancreatic, lung, colon, kidney, breast, and prostate cancer. See e.g., Martinko, A. J. et al., eLife, 7: e31098 (2018); Uekita, T. et al., Cancer Science, 102 (11): 1943-1948 (2011); Scherl-Mostageer, M. et al., Oncogene, 20 (32): 4402-4408 (2001). CDCP1 was also found upregulated and critical for growth in K-Ras driven cancer cells. See e.g., Uekita, T. et al., Cancer Science, 102 (11): 1943-1948 (2011).

CDCP1 is proteolytically processed between the first and second CUB domains presumably by serine proteases. See e.g., Casar, B. et al., Oncogene 33:255-268 (2014). Proteolysis, along with overexpression, has been associated with CDCP1 activation. See e.g., He, Y. et al., Oncogene 35:468-478 (2016); Brown, T. A. et al., J. Biol. Chem. 279:14772-14783 (2004). There is strong evidence that both overexpression and proteolytic activation of CDCP1 contributes to loss of cell adhesion, increased migration, and poor prognosis/survival in cancer patients. See e.g., Uekita, T. et al., Cancer Science, 102 (11): 1943-1948 (2011); Casar, B. et al., Oncogene 33:255-268 (2014).

Furthermore, it has been shown that cleaved CDCP1 is more prevalent in aggressive cancers, see e.g., Wright, H. J. et al. Oncogene, 35:4762-4772 (2016); Adams, M. N. et al., Oncogene 34:1375-1383 (2015); He, Y. et al., J. Biol. Chem., 285:26162-26173 (2010), while CDCP1 on normal tissue has been found predominantly in the full-length form. See e.g., Alvares, S. M., et al., Biochim. Biophys. Acta-Gen. Subj., 1780:486-496 (2008); McGovern, J. A. et al., Br. J. Dermatol., 168:496-503 (2013); Wong, C. H. et al., Clin. Cancer Res., 15:2311-2322 (2009).

Thus, an antibody that specifically binds to a cleaved human CDCP1 can be used for the diagnosis and prevention or treatment of diseases in which cleaved CDCP1 is overexpressed (e.g., K-Ras-driven tumors). Accordingly, there is a need to develop antibodies that specifically bind to the cleaved CDCP1 and that are capable of modulating the cleaved CDCP1 activity.

### SUMMARY OF THE DISCLOSURE

One aspect of the present disclosure is directed to an isolated antibody or antigen-binding fragment thereof that specifically binds to a cleaved human complement C1r/C1s, Uegf, Bmp1 (CUB)-domain containing protein 1 (CDCP1), wherein the antibody or antigen-binding fragment thereof preferentially binds to the cleaved CDCP1.

In some aspects, the antibody or antigen-binding fragment thereof of the present disclosure does not bind to a full-length human CDCP1 at a detectable level.

In some aspects, the binding between the antibody or antigen-binding fragment thereof and the cleaved CDCP1 or the full-length CDCP1 is measured by biolayer interferometry analysis using an Octet instrument (ForteBio).

In some aspects, the cleaved CDCP1 comprises a first cleaved domain and second cleaved domain, wherein the first cleaved domain and the second cleaved domain are not linked.

In some aspects, the cleaved CDCP1 comprises a membrane-bound complex.

In some aspects, the first cleaved domain consists of the amino acid sequence as set forth in SEQ ID NO: 63, 68, or 74.

In some aspects, the second cleaved domain consists of the amino acid sequence as set forth in SEQ ID NO: 64, 70, or 77.

In some aspects, the cleaved CDCP1 is generated by being cleaved at residue K365, R368, and/or K369 of SEQ ID NO: 273.

In some aspects, the cleaved CDCP1 is post translationally modified, wherein the post translational modification comprises phosphorylation and N-linked glycosylation.

Some aspects of the present disclosure are directed to an isolated antibody or antigen-binding fragment thereof that specifically binds to a cleaved human CDCP1 and comprises a light chain variable region (VL) and a heavy chain variable region (VH); wherein the VL comprises a VL complementarity determining region (CDR) 1, a VL-CDR2, and a VL-CDR3, and the VH comprises a VH-CDR1, a VH-CDR2, and a VH-CDR3 sequences of SEQ ID NOs: 1 (SVSSAVA), 2 (SASSLY), 268 (SX₁X₂X₃X₄X₅), 269 (X₆FSSX₇SI), 270 (SIYPYSGSTX₈), and 271 (X₉X₁₀X₁₂SX₁₂YSHTWWVSYGX₁₃) or 272 (X₁₄YWVX₁₅FWYGHFSYYRPAL), respectively, wherein:
X₁=Glycine (G), Serine(S), Methionine (M), Leucine (L), Valine (V), or Arginine (R);
X₂=Glutamine (Q), Serine(S), Glutamic acid (E), Asparagine (N), Lysine (K), Proline (P), Arginine (R), Leucine (L), or Histidine (H);
X₃=Arginine (R), Serine(S), Valine (V), Tryptophan (W), Leucine (L), Lysine (K), Methionine (M), Glutamine (Q), or Proline (P);
X₄=Proline (P), Leucine (L), Threonine (T), or Serine(S);
X₅=Isoleucine (I), Alanine (A), Methionine (M), Lysine (K), Valine (V), Leucine (L), Phenylalanine (F);
X₆=No Amino Acid, Aspartic acid (D), or Asparagine (N);
X₇=Serine(S) or Tyrosine (Y);
X₈=Serine(S) or Tyrosine (Y);
X₉=Glutamine (Q), Arginine (R), or Lysine (K);
X₁₀=Serine(S), Asparagine (N), Threonine (T), Glycine (G), Alanine (A), or Aspartic acid (D);
X₁₁=Glutamine (Q) or Histidine (H);
X₁₂=Tyrosine (Y) or Phenylalanine (F);
X₁₃=Methionine (M), Alanine (A), Isoleucine (I), Leucine (L), or Valine (V);
X₁₄=Threonine (T) or Isoleucine (I); and
X₁₅=Glutamine (Q) or Aspartic acid (D).

Some aspects of the present disclosure are directed to an isolated antibody or antigen-binding fragment thereof that specifically binds to a cleaved human CDCP1 and comprises a light chain variable region (VL) and a heavy chain variable region (VH); wherein the VL comprises a VL complementarity determining region (CDR) 1 sequence of SEQ ID NO: 1 (SVSSAVA), a VL-CDR2 sequence of SEQ ID NO: 2 (SASSLY), and a VL-CDR3 sequence of SEQ ID NOs: 8 (TGQRPM), 23 (FMRPAF), 16 (TAQSPL), 11 (VELVPM), 12 (AGKRPL), or 14 (LGVRAA), and the VH comprises a VH-CDR1 sequence of SEQ ID NO: 269 (X₁FSSX₂SI), a VH-CDR2 sequence of SEQ ID NO: 270 (SIYPYSGSTX₃), and a VH-CDR3 sequence of SEQ ID NO: 271 (X₄X₅X₆SX₇YSHTWWVSYGX₈) or SEQ ID NO: 272 (X₉YWVX₁₀FWYGHFSYYRPAL), respectively, wherein:
X₁=No Amino Acid, Aspartic acid (D), or Asparagine (N);
X₂=Serine(S) or Tyrosine (Y);
X₃=Serine(S) or Tyrosine (Y);
X₄=Glutamine (Q), Arginine (R), or Lysine (K);
X₅=Serine(S), Asparagine (N), Threonine (T), Glycine (G), Alanine (A), or Aspartic acid (D);
X₆=Glutamine (Q) or Histidine (H);
X₇=Tyrosine (Y) or Phenylalanine (F);
X₈=Methionine (M), Alanine (A), Isoleucine (I), Leucine (L), or Valine (V);
X₉=Threonine (T) or Isoleucine (I); and
X₁₀=Glutamine (Q) or Aspartic acid (D).

In some aspects, an isolated antibody or antigen-binding fragment of the present disclosure comprises a light chain variable region (VL) and a heavy chain variable region (VH); wherein the VL comprises a VL complementarity determining region (CDR) 1, a VL-CDR2, and a VL-CDR3 and the VH comprises a VH-CDR1, a VH-CDR2, and a VH-CDR3; wherein the VL-CDR3 comprises an amino acid sequence selected from the group consisting of SEQ ID NOs: 3-25. In some aspects, the VL-CDR2 comprises an amino acid of SEQ ID NO: 2. In some aspects, the VL-CDR1 comprises an amino acid sequence of SEQ ID NO: 1. In some aspects, the VH-CDR1 comprises an amino acid sequence selected from the group consisting of SEQ ID NOs: 26-29, 109, and 111. In some aspects, the VH-CDR2 comprises an amino acid sequence of SEQ ID NO: 30 or 31. In some aspects, the VH-CDR3 comprises an amino acid sequence selected from the group consisting of SEQ ID NOs: 20, 32-47, and 105.

In some aspects, the VL-CDR1 comprises the amino acid sequence set forth in SEQ ID NO: 1, the VL-CDR2 comprises the amino acid sequence set forth in SEQ ID NO: 2, the VL-CDR3 comprises the amino acid sequence set forth in SEQ ID NO: 3, the VH-CDR1 comprises the amino acid sequence set forth in SEQ ID NO: 26, the VH-CDR2 comprises the amino acid sequence set forth in SEQ ID NO: 30, and the VH-CDR3 comprises the amino acid sequence set forth in SEQ ID NO: 32;

In some aspects, the VL-CDR1 comprises the amino acid sequence set forth in SEQ ID NO: 1, the VL-CDR2 comprises the amino acid sequence set forth in SEQ ID NO: 2, the VL-CDR3 comprises the amino acid sequence set forth in SEQ ID NO: 3, the VH-CDR1 comprises the amino acid sequence set forth in SEQ ID NO: 26, the VH-CDR2 comprises the amino acid sequence set forth in SEQ ID NO: 30, and the VH-CDR3 comprises the amino acid sequence set forth in SEQ ID NO: 33;

In some aspects, the VL-CDR1 comprises the amino acid sequence set forth in SEQ ID NO: 1, the VL-CDR2 comprises the amino acid sequence set forth in SEQ ID NO: 2, the VL-CDR3 comprises the amino acid sequence set forth in SEQ ID NO: 3, the VH-CDR1 comprises the amino acid sequence set forth in SEQ ID NO: 26, the VH-CDR2 comprises the amino acid sequence set forth in SEQ ID NO: 30, and the VH-CDR3 comprises the amino acid sequence set forth in SEQ ID NO: 34;

In some aspects, the VL-CDR1 comprises the amino acid sequence set forth in SEQ ID NO: 1, the VL-CDR2 comprises the amino acid sequence set forth in SEQ ID NO: 2, the VL-CDR3 comprises the amino acid sequence set forth in SEQ ID NO: 3, the VH-CDR1 comprises the amino acid sequence set forth in SEQ ID NO: 26, the VH-CDR2 comprises the amino acid sequence set forth in SEQ ID NO: 30, and the VH-CDR3 comprises the amino acid sequence set forth in SEQ ID NO: 35; or

In some aspects, the VL-CDR1 comprises the amino acid sequence set forth in SEQ ID NO: 1, the VL-CDR2 comprises the amino acid sequence set forth in SEQ ID NO: 2, the VL-CDR3 comprises the amino acid sequence set forth in SEQ ID NO: 3, the VH-CDR1 comprises the amino acid sequence set forth in SEQ ID NO: 26, the VH-CDR2 comprises the amino acid sequence set forth in SEQ ID NO: 30, and the VH-CDR3 comprises the amino acid sequence set forth in SEQ ID NO: 36.

In some aspects, the antibody or antigen-binding fragment thereof of the present disclosure comprises the VH comprising an amino acid sequence at least about 70%, at least about 80%, at least about 90%, at least about 95%, at least about 96%, at least about 97%, at least about 98%, or at least about 99% sequence identity to an amino acid sequence selected from the group consisting of SEQ ID NOs: 61, 65, 67, 69, 71, 73, 75, 79, 80, 81, 82, 83, 84, 85, 86, 87, 89, 91, 99, 103, 107, 123, and 133.

In some aspects, the antibody or antigen-binding fragment thereof of the present disclosure comprises the VH comprising an amino acid sequence selected from the group consisting of SEQ ID NOs: 61, 65, 67, 69, 71, 73, 75, 79, 80, 81, 82, 83, 84, 85, 86, 87, 89, 91, 99, 103, 107, 123, and 133.

In some aspects, the antibody or antigen-binding fragment thereof of the present disclosure comprises the VL comprising an amino acid sequence at least about 70%, at least about 80%, at least about 90%, at least about 95%, at least about 96%, at least about 97%, at least about 98%, or at least about 99% sequence identity to an amino acid sequence selected from the group consisting of SEQ ID NOs: 62, 76, 94, 96, 98, 100, 102, 104, 106, 108, 110, 112, 114, 116, 118, 120, 122, 124, 126, 128, 130, and 132.

In some aspects, the antibody or antigen-binding fragment thereof of the present disclosure comprises the VL comprising an amino acid sequence selected from the group consisting of SEQ ID NOs: 62, 76, 94, 96, 98, 100, 102, 104, 106, 108, 110, 112, 114, 116, 118, 120, 122, 124, 126, 128, 130, and 132.

In some aspects, the antibody or antigen-binding fragment thereof of the present disclosure comprises the VH and the VL, wherein:
(a) the VH of the reference antibody comprises the amino acid sequence set forth in SEQ ID NO: 61 and the VL of the reference antibody comprises the amino acid sequence set forth in SEQ ID NO: 62;
(b) the VH of the reference antibody comprises the amino acid sequence set forth in SEQ ID NO: 65 and the VL of the reference antibody comprises the amino acid sequence set forth in SEQ ID NO: 62;
(c) the VH of the reference antibody comprises the amino acid sequence set forth in SEQ ID NO 67 and the VL of the reference antibody comprises the amino acid sequence set forth in SEQ ID NO: 62;
(d) the VH of the reference antibody comprises the amino acid sequence set forth in SEQ ID NO: 69 and the VL of the reference antibody comprises the amino acid sequence set forth in SEQ ID NO: 62;
(e) the VH of the reference antibody comprises the amino acid sequence set forth in SEQ ID NO 71; and the VL of the reference antibody comprises the amino acid sequence set forth in SEQ ID NO: 62;
(f) the VH of the reference antibody comprises the amino acid sequence set forth in SEQ ID NO: 73 and the VL of the reference antibody comprises the amino acid sequence set forth in SEQ ID NO: 62;
(g) the VH of the reference antibody comprises the amino acid sequence set forth in SEQ ID NO: 75 and the VL of the reference antibody comprises the amino acid sequence set forth in SEQ ID NO: 76;
(h) the VH of the reference antibody comprises the amino acid sequence set forth in SEQ ID NO: 79 and the VL of the reference antibody comprises the amino acid sequence set forth in SEQ ID NO: 76;
(i) the VH of the reference antibody comprises the amino acid sequence set forth in SEQ ID NO: 65 and the VL of the reference antibody comprises the amino acid sequence set forth in SEQ ID NO: 76;
(j) the VH of the reference antibody comprises the amino acid sequence set forth in SEQ ID NO: 83 and the VL of the reference antibody comprises the amino acid sequence set forth in SEQ ID NO: 62;
(k) the VH of the reference antibody comprises the amino acid sequence set forth in SEQ ID NO: 85 and the VL of the reference antibody comprises the amino acid sequence set forth in SEQ ID NO: 62;
(l) the VH of the reference antibody comprises the amino acid sequence set forth in SEQ ID NO: 87 and the VL of the reference antibody comprises the amino acid sequence set forth in SEQ ID NO: 62;
(m) the VH of the reference antibody comprises the amino acid sequence set forth in SEQ ID NO: 89 and the VL of the reference antibody comprises the amino acid sequence set forth in SEQ ID NO: 62;
(n) the VH of the reference antibody comprises the amino acid sequence set forth in SEQ ID NO: 91 and the VL of the reference antibody comprises the amino acid sequence set forth in SEQ ID NO: 62;
(o) the VH of the reference antibody comprises the amino acid sequence set forth in SEQ ID NO: 65 and the VL of the reference antibody comprises the amino acid sequence set forth in SEQ ID NO: 94;
(p) the VH of the reference antibody comprises the amino acid sequence set forth in SEQ ID NO: 65 and the VL of the reference antibody comprises the amino acid sequence set forth in SEQ ID NO: 96;
(q) the VH of the reference antibody comprises the amino acid sequence set forth in SEQ ID NO: 65 and the VL of the reference antibody comprises the amino acid sequence set forth in SEQ ID NO: 98;
(r) the VH of the reference antibody comprises the amino acid sequence set forth in SEQ ID NO: 99 and the VL of the reference antibody comprises the amino acid sequence set forth in SEQ ID NO: 100;
(s) the VH of the reference antibody comprises the amino acid sequence set forth in SEQ ID NO: 65 and the VL of the reference antibody comprises the amino acid sequence set forth in SEQ ID NO: 102;
(t) the VH of the reference antibody comprises the amino acid sequence set forth in SEQ ID NO: 103 and the VL of the reference antibody comprises the amino acid sequence set forth in SEQ ID NO: 104;
(u) the VH of the reference antibody comprises the amino acid sequence set forth in SEQ ID NO: 99 and the VL of the reference antibody comprises the amino acid sequence set forth in SEQ ID NO: 106;
(v) the VH of the reference antibody comprises the amino acid sequence set forth in SEQ ID NO: 107 and the VL of the reference antibody comprises the amino acid sequence set forth in SEQ ID NO: 108;
(w) the VH of the reference antibody comprises the amino acid sequence set forth in SEQ ID NO: 99 and the VL of the reference antibody comprises the amino acid sequence set forth in SEQ ID NO: 110;
(x) the VH of the reference antibody comprises the amino acid sequence set forth in SEQ ID NO: 99 and the VL of the reference antibody comprises the amino acid sequence set forth in SEQ ID NO: 112;
(y) the VH of the reference antibody comprises the amino acid sequence set forth in SEQ ID NO: 65 and the VL of the reference antibody comprises the amino acid sequence set forth in SEQ ID NO: 114;
(aa) the VH of the reference antibody comprises the amino acid sequence set forth in SEQ ID NO: 99 and the VL of the reference antibody comprises the amino acid sequence set forth in SEQ ID NO: 116;
(bb) the VH of the reference antibody comprises the amino acid sequence set forth in SEQ ID NO: 65 and the VL of the reference antibody comprises the amino acid sequence set forth in SEQ ID NO: 118;
(cc) the VH comprises the amino acid sequence set forth in SEQ ID NO: 65 and the VL comprises the amino acid sequence set forth in SEQ ID NO: 120;
(dd) the VH of the reference antibody comprises the amino acid sequence set forth in SEQ ID NO: 65 and the VL of the reference antibody comprises the amino acid sequence set forth in SEQ ID NO: 122;
(ee) the VH of the reference antibody comprises the amino acid sequence set forth in SEQ ID NO: 123 and the VL of the reference antibody comprises the amino acid sequence set forth in SEQ ID NO: 124;
(ff) the VH of the reference antibody comprises the amino acid sequence set forth in SEQ ID NO: 107 and the VL of the reference antibody comprises the amino acid sequence set forth in SEQ ID NO: 126;
(gg) the VH of the reference antibody comprises the amino acid sequence set forth in SEQ ID NO: 99 and the VL of the reference antibody comprises the amino acid sequence set forth in SEQ ID NO: 128;
(hh) the VH of the reference antibody comprises the amino acid sequence set forth in SEQ ID NO: 99 and the VL of the reference antibody comprises the amino acid sequence set forth in SEQ ID NO: 130;
(ii) the VH of the reference antibody comprises the amino acid sequence set forth in SEQ ID NO: 65 and the VL of the reference antibody comprises the amino acid sequence set forth in SEQ ID NO: 132;
(jj) the VH of the reference antibody comprises the amino acid sequence set forth in SEQ ID NO: 133 and the VL of the reference antibody comprises the amino acid sequence set forth in SEQ ID NO: 76;
(kk) the VH of the reference antibody comprises the amino acid sequence set forth in SEQ ID NO: 80 and the VL of the reference antibody comprises the amino acid sequence set forth in SEQ ID NO: 62;
(ll) the VH of the reference antibody comprises the amino acid sequence set forth in SEQ ID NO: 81 and the VL of the reference antibody comprises the amino acid sequence set forth in SEQ ID NO: 62;
(mm) the VH of the reference antibody comprises the amino acid sequence set forth in SEQ ID NO: 82 and the VL of the reference antibody comprises the amino acid sequence set forth in SEQ ID NO: 62;
(nn) the VH of the reference antibody comprises the amino acid sequence set forth in SEQ ID NO: 84 and the VL of the reference antibody comprises the amino acid sequence set forth in SEQ ID NO: 62; or
(oo) the VH of the reference antibody comprises the amino acid sequence set forth in SEQ ID NO: 86 and the VL of the reference antibody comprises the amino acid sequence set forth in SEQ ID NO: 62.

Some aspects of the present disclosure are directed to an isolated antibody or antigen-binding fragment thereof that specifically binds to the same cleaved human CDCP1 epitope as a reference antibody comprising a heavy chain variable region (VH) and a light chain variable region (VL), wherein:
(a) the VH of the reference antibody comprises the amino acid sequence set forth in SEQ ID NO: 61 and the VL of the reference antibody comprises the amino acid sequence set forth in SEQ ID NO: 62;
(b) the VH of the reference antibody comprises the amino acid sequence set forth in SEQ ID NO: 65 and the VL of the reference antibody comprises the amino acid sequence set forth in SEQ ID NO: 62;
(c) the VH of the reference antibody comprises the amino acid sequence set forth in SEQ ID NO 67 and the VL of the reference antibody comprises the amino acid sequence set forth in SEQ ID NO: 62;
(d) the VH of the reference antibody comprises the amino acid sequence set forth in SEQ ID NO: 69 and the VL of the reference antibody comprises the amino acid sequence set forth in SEQ ID NO: 62;
(e) the VH of the reference antibody comprises the amino acid sequence set forth in SEQ ID NO 71; and the VL of the reference antibody comprises the amino acid sequence set forth in SEQ ID NO: 62;
(f) the VH of the reference antibody comprises the amino acid sequence set forth in SEQ ID NO: 73 and the VL of the reference antibody comprises the amino acid sequence set forth in SEQ ID NO: 62;
(g) the VH of the reference antibody comprises the amino acid sequence set forth in SEQ ID NO: 75 and the VL of the reference antibody comprises the amino acid sequence set forth in SEQ ID NO: 76;
(h) the VH of the reference antibody comprises the amino acid sequence set forth in SEQ ID NO: 79 and the VL of the reference antibody comprises the amino acid sequence set forth in SEQ ID NO: 76;
(i) the VH of the reference antibody comprises the amino acid sequence set forth in SEQ ID NO: 65 and the VL of the reference antibody comprises the amino acid sequence set forth in SEQ ID NO: 76;
(j) the VH of the reference antibody comprises the amino acid sequence set forth in SEQ ID NO: 83 and the VL of the reference antibody comprises the amino acid sequence set forth in SEQ ID NO: 62;
(k) the VH of the reference antibody comprises the amino acid sequence set forth in SEQ ID NO: 85 and the VL of the reference antibody comprises the amino acid sequence set forth in SEQ ID NO: 62;
(l) the VH of the reference antibody comprises the amino acid sequence set forth in SEQ ID NO: 87 and the VL of the reference antibody comprises the amino acid sequence set forth in SEQ ID NO: 62;
(m) the VH of the reference antibody comprises the amino acid sequence set forth in SEQ ID NO: 89 and the VL of the reference antibody comprises the amino acid sequence set forth in SEQ ID NO: 62;
(n) the VH of the reference antibody comprises the amino acid sequence set forth in SEQ ID NO: 91 and the VL of the reference antibody comprises the amino acid sequence set forth in SEQ ID NO: 62;
(o) the VH of the reference antibody comprises the amino acid sequence set forth in SEQ ID NO: 65 and the VL of the reference antibody comprises the amino acid sequence set forth in SEQ ID NO: 94;
(p) the VH of the reference antibody comprises the amino acid sequence set forth in SEQ ID NO: 65 and the VL of the reference antibody comprises the amino acid sequence set forth in SEQ ID NO: 96;
(q) the VH of the reference antibody comprises the amino acid sequence set forth in SEQ ID NO: 65 and the VL of the reference antibody comprises the amino acid sequence set forth in SEQ ID NO: 98;
(r) the VH of the reference antibody comprises the amino acid sequence set forth in SEQ ID NO: 99 and the VL of the reference antibody comprises the amino acid sequence set forth in SEQ ID NO: 100;
(s) the VH of the reference antibody comprises the amino acid sequence set forth in SEQ ID NO: 65 and the VL of the reference antibody comprises the amino acid sequence set forth in SEQ ID NO: 102;
(t) the VH of the reference antibody comprises the amino acid sequence set forth in SEQ ID NO:103 and the VL of the reference antibody comprises the amino acid sequence set forth in SEQ ID NO: 104;
(u) the VH of the reference antibody comprises the amino acid sequence set forth in SEQ ID NO: 99 and the VL of the reference antibody comprises the amino acid sequence set forth in SEQ ID NO: 106;
(v) the VH of the reference antibody comprises the amino acid sequence set forth in SEQ ID NO: 107 and the VL of the reference antibody comprises the amino acid sequence set forth in SEQ ID NO: 108;
(w) the VH of the reference antibody comprises the amino acid sequence set forth in SEQ ID NO: 99 and the VL of the reference antibody comprises the amino acid sequence set forth in SEQ ID NO: 110;
(x) the VH of the reference antibody comprises the amino acid sequence set forth in SEQ ID NO: 99 and the VL of the reference antibody comprises the amino acid sequence set forth in SEQ ID NO: 112;
(y) the VH of the reference antibody comprises the amino acid sequence set forth in SEQ ID NO: 65 and the VL of the reference antibody comprises the amino acid sequence set forth in SEQ ID NO: 114;
(aa) the VH of the reference antibody comprises the amino acid sequence set forth in SEQ ID NO: 99 and the VL of the reference antibody comprises the amino acid sequence set forth in SEQ ID NO: 116;
(bb) the VH of the reference antibody comprises the amino acid sequence set forth in SEQ ID NO: 65 and the VL of the reference antibody comprises the amino acid sequence set forth in SEQ ID NO: 118;
(cc) the VH comprises the amino acid sequence set forth in SEQ ID NO: 65 and the VL comprises the amino acid sequence set forth in SEQ ID NO: 120;
(dd) the VH of the reference antibody comprises the amino acid sequence set forth in SEQ ID NO: 65 and the VL of the reference antibody comprises the amino acid sequence set forth in SEQ ID NO: 122;
(ee) the VH of the reference antibody comprises the amino acid sequence set forth in SEQ ID NO: 123 and the VL of the reference antibody comprises the amino acid sequence set forth in SEQ ID NO: 124;
(ff) the VH of the reference antibody comprises the amino acid sequence set forth in SEQ ID NO: 107 and the VL of the reference antibody comprises the amino acid sequence set forth in SEQ ID NO: 126;
(gg) the VH of the reference antibody comprises the amino acid sequence set forth in SEQ ID NO: 99 and the VL of the reference antibody comprises the amino acid sequence set forth in SEQ ID NO: 128;
(hh) the VH of the reference antibody comprises the amino acid sequence set forth in SEQ ID NO: 99 and the VL of the reference antibody comprises the amino acid sequence set forth in SEQ ID NO: 130;
(ii) the VH of the reference antibody comprises the amino acid sequence set forth in SEQ ID NO: 65 and the VL of the reference antibody comprises the amino acid sequence set forth in SEQ ID NO: 132; or
(jj) the VH of the reference antibody comprises the amino acid sequence set forth in SEQ ID NO: 133 and the VL of the reference antibody comprises the amino acid sequence set forth in SEQ ID NO: 76;
(kk) the VH of the reference antibody comprises the amino acid sequence set forth in SEQ ID NO: 80 and the VL of the reference antibody comprises the amino acid sequence set forth in SEQ ID NO: 62;
(ll) the VH of the reference antibody comprises the amino acid sequence set forth in SEQ ID NO: 81 and the VL of the reference antibody comprises the amino acid sequence set forth in SEQ ID NO: 62;
(mm) the VH of the reference antibody comprises the amino acid sequence set forth in SEQ ID NO: 82 and the VL of the reference antibody comprises the amino acid sequence set forth in SEQ ID NO: 62;
(nn) the VH of the reference antibody comprises the amino acid sequence set forth in SEQ ID NO: 84 and the VL of the reference antibody comprises the amino acid sequence set forth in SEQ ID NO: 62; or (00) the VH of the reference antibody comprises the amino acid sequence set forth in SEQ

ID NO: 86 and the VL of the reference antibody comprises the amino acid sequence set forth in SEQ ID NO: 62. Some aspects of the present disclosure are directed to an isolated antibody or antigen-binding fragment thereof that cross-competes for binding to a cleaved human CDCP1 epitope with a reference antibody comprising a heavy chain variable region (VH) and a light chain variable region (VL), wherein:
(a) the VH of the reference antibody comprises the amino acid sequence set forth in SEQ ID NO: 61 and the VL of the reference antibody comprises the amino acid sequence set forth in SEQ ID NO: 62;
(b) the VH of the reference antibody comprises the amino acid sequence set forth in SEQ ID NO: 65 and the VL of the reference antibody comprises the amino acid sequence set forth in SEQ ID NO: 62;
(c) the VH of the reference antibody comprises the amino acid sequence set forth in SEQ ID NO 67 and the VL of the reference antibody comprises the amino acid sequence set forth in SEQ ID NO: 62;
(d) the VH of the reference antibody comprises the amino acid sequence set forth in SEQ ID NO: 69 and the VL of the reference antibody comprises the amino acid sequence set forth in SEQ ID NO: 62;
(e) the VH of the reference antibody comprises the amino acid sequence set forth in SEQ ID NO 71; and the VL of the reference antibody comprises the amino acid sequence set forth in SEQ ID NO: 62;
(f) the VH of the reference antibody comprises the amino acid sequence set forth in SEQ ID NO: 73 and the VL of the reference antibody comprises the amino acid sequence set forth in SEQ ID NO: 62;
(g) the VH of the reference antibody comprises the amino acid sequence set forth in SEQ ID NO: 75 and the VL of the reference antibody comprises the amino acid sequence set forth in SEQ ID NO: 76;
(h) the VH of the reference antibody comprises the amino acid sequence set forth in SEQ ID NO: 79 and the VL of the reference antibody comprises the amino acid sequence set forth in SEQ ID NO: 76;
(i) the VH of the reference antibody comprises the amino acid sequence set forth in SEQ ID NO: 65 and the VL of the reference antibody comprises the amino acid sequence set forth in SEQ ID NO: 76;
(j) the VH of the reference antibody comprises the amino acid sequence set forth in SEQ ID NO: 83 and the VL of the reference antibody comprises the amino acid sequence set forth in SEQ ID NO: 62;
(k) the VH of the reference antibody comprises the amino acid sequence set forth in SEQ ID NO: 85 and the VL of the reference antibody comprises the amino acid sequence set forth in SEQ ID NO: 62;
(l) the VH of the reference antibody comprises the amino acid sequence set forth in SEQ ID NO: 87 and the VL of the reference antibody comprises the amino acid sequence set forth in SEQ ID NO: 62;
(m) the VH of the reference antibody comprises the amino acid sequence set forth in SEQ ID NO: 89 and the VL of the reference antibody comprises the amino acid sequence set forth in SEQ ID NO: 62;
(n) the VH of the reference antibody comprises the amino acid sequence set forth in SEQ ID NO: 91 and the VL of the reference antibody comprises the amino acid sequence set forth in SEQ ID NO: 62;
(o) the VH of the reference antibody comprises the amino acid sequence set forth in SEQ ID NO: 65 and the VL of the reference antibody comprises the amino acid sequence set forth in SEQ ID NO: 94;
(p) the VH of the reference antibody comprises the amino acid sequence set forth in SEQ ID NO: 65 and the VL of the reference antibody comprises the amino acid sequence set forth in SEQ ID NO: 96;
(q) the VH of the reference antibody comprises the amino acid sequence set forth in SEQ ID NO: 65 and the VL of the reference antibody comprises the amino acid sequence set forth in SEQ ID NO: 98;
(r) the VH of the reference antibody comprises the amino acid sequence set forth in SEQ ID NO: 99 and the VL of the reference antibody comprises the amino acid sequence set forth in SEQ ID NO: 100;
(s) the VH of the reference antibody comprises the amino acid sequence set forth in SEQ ID NO: 65 and the VL of the reference antibody comprises the amino acid sequence set forth in SEQ ID NO: 102;
(t) the VH of the reference antibody comprises the amino acid sequence set forth in SEQ ID NO:103 and the VL of the reference antibody comprises the amino acid sequence set forth in SEQ ID NO: 104;
(u) the VH of the reference antibody comprises the amino acid sequence set forth in SEQ ID NO: 99 and the VL of the reference antibody comprises the amino acid sequence set forth in SEQ ID NO: 106;
(v) the VH of the reference antibody comprises the amino acid sequence set forth in SEQ ID NO: 107 and the VL of the reference antibody comprises the amino acid sequence set forth in SEQ ID NO: 108;
(w) the VH of the reference antibody comprises the amino acid sequence set forth in SEQ ID NO: 99 and the VL of the reference antibody comprises the amino acid sequence set forth in SEQ ID NO: 110;
(x) the VH of the reference antibody comprises the amino acid sequence set forth in SEQ ID NO: 99 and the VL of the reference antibody comprises the amino acid sequence set forth in SEQ ID NO: 112;
(y) the VH of the reference antibody comprises the amino acid sequence set forth in SEQ ID NO: 65 and the VL of the reference antibody comprises the amino acid sequence set forth in SEQ ID NO: 114;
(aa) the VH of the reference antibody comprises the amino acid sequence set forth in SEQ ID NO: 99 and the VL of the reference antibody comprises the amino acid sequence set forth in SEQ ID NO: 116;
(bb) the VH of the reference antibody comprises the amino acid sequence set forth in SEQ ID NO: 65 and the VL of the reference antibody comprises the amino acid sequence set forth in SEQ ID NO: 118;
(cc) the VH comprises the amino acid sequence set forth in SEQ ID NO: 65 and the VL comprises the amino acid sequence set forth in SEQ ID NO: 120;
(dd) the VH of the reference antibody comprises the amino acid sequence set forth in SEQ ID NO: 65 and the VL of the reference antibody comprises the amino acid sequence set forth in SEQ ID NO: 122;
(ee) the VH of the reference antibody comprises the amino acid sequence set forth in SEQ ID NO: 123 and the VL of the reference antibody comprises the amino acid sequence set forth in SEQ ID NO: 124;
(ff) the VH of the reference antibody comprises the amino acid sequence set forth in SEQ ID NO: 107 and the VL of the reference antibody comprises the amino acid sequence set forth in SEQ ID NO: 126;
(gg) the VH of the reference antibody comprises the amino acid sequence set forth in SEQ ID NO: 99 and the VL of the reference antibody comprises the amino acid sequence set forth in SEQ ID NO: 128;
(hh) the VH of the reference antibody comprises the amino acid sequence set forth in SEQ ID NO: 99 and the VL of the reference antibody comprises the amino acid sequence set forth in SEQ ID NO: 130;
(ii) the VH of the reference antibody comprises the amino acid sequence set forth in SEQ ID NO: 65 and the VL of the reference antibody comprises the amino acid sequence set forth in SEQ ID NO: 132; or
(jj) the VH of the reference antibody comprises the amino acid sequence set forth in SEQ ID NO: 133 and the VL of the reference antibody comprises the amino acid sequence set forth in SEQ ID NO: 76;
(kk) the VH of the reference antibody comprises the amino acid sequence set forth in SEQ ID NO: 80 and the VL of the reference antibody comprises the amino acid sequence set forth in SEQ ID NO: 62;
(ll) the VH of the reference antibody comprises the amino acid sequence set forth in SEQ ID NO: 81 and the VL of the reference antibody comprises the amino acid sequence set forth in SEQ ID NO: 62;
(mm) the VH of the reference antibody comprises the amino acid sequence set forth in SEQ ID NO: 82 and the VL of the reference antibody comprises the amino acid sequence set forth in SEQ ID NO: 62;
(nn) the VH of the reference antibody comprises the amino acid sequence set forth in SEQ ID NO: 84 and the VL of the reference antibody comprises the amino acid sequence set forth in SEQ ID NO: 62; or
(oo) the VH of the reference antibody comprises the amino acid sequence set forth in SEQ ID NO: 86 and the VL of the reference antibody comprises the amino acid sequence set forth in SEQ ID NO: 62. In some aspects, the antibody or antigen-binding fragment thereof of the present disclosure has one or more properties selected from the group consisting of:
(a) the antibody inhibits tumor growth and/or metastasis;
(b) the antibody reduces tumor volume;
(c) the antibody increases progression-free survival;
(d) the antibody increases overall survival;
(e) the antibody promotes CDCP1 internalization and/or degradation; and
(f) any combination thereof.

In some aspects, the antibody or antigen-binding fragment thereof of the present disclosure specifically binds cleaved human CDCP1 with a K_{D} of about 1×10⁻⁴ M or less, wherein K_{D} is measured by biolayer interferometry analysis using an Octet instrument (ForteBio).

In some aspects, the antibody or antigen-binding fragment thereof of the present disclosure specifically binds cleaved human CDCP1 with an on rate (kₒₙ) of about 1×10⁻⁴ 1/Ms or more, wherein the kₒₙ rate is measured by biolayer interferometry analysis using an Octet instrument (ForteBio).

In some aspects, the antibody or antigen-binding fragment thereof of the present disclosure specifically binds cleaved human CDCP1 with an off rate (k_{off}) of about 1×10⁻⁴ M 1/s or less, wherein the k_{off} is measured by biolayer interferometry analysis using an Octet instrument (ForteBio).

In some aspects, the antibody or antigen-binding fragment thereof of the present disclosure binds to cleaved cynomolgus monkey CDCP1.

In some aspects, the antibody or antigen-binding fragment thereof of the present disclosure is selected from the group consisting of an IgG1, an IgG2, an IgG3, an IgG4 or a variant thereof.

In some aspects, the antibody or antigen-binding fragment of the present disclosure is an IgG1 antibody.

In some aspects, the antibody or antigen-binding fragment of the present disclosure is modified to remove a glycosylation site. In some aspects, the glycosylation site removal is accomplished via substitution of the asparagine (N) to Aspartic acid (D) at a position that corresponds to residue 31 in SEQ ID NO: 61.

In some aspects, the antibody or antigen-binding fragment of the present disclosure comprises substitution of methionine (M) to alanine (A), isoleucine (I), leucine (L), or valine (V) at a position that corresponds to residue 114 in SEQ ID NO: 61 or 65,

In some aspects, the antibody of the present disclosure is a human, a humanized antibody, a chimeric antibody, or antigen-binding fragment thereof.

In some aspects, the antibody or antigen-binding fragment of the present disclosure is suitable for administration to a human subject.

In some aspects, the antibody or antigen-binding fragment of the present disclosure is a full length antibody.

In some aspects, the antibody or antigen-binding fragment of the present disclosure is an antigen binding fragment. In some aspects, the antigen binding fragment is a Fab, Fab′, F(ab′)₂, single chain Fv (scFv), disulfide linked Fv, IgNar, intrabody, IgGΔCH2, minibody, F(ab′)₃, tetrabody, triabody, diabody, single-domain antibody, DVD-Ig, Fcab, mAb², (scFv)₂, or scFv-Fc.

Some aspects of the present disclosure are directed to a bispecific antibody comprising the antibody or antigen-binding fragment described herein.

Some aspects of the present disclosure are directed to a multispecific antibody comprising the bispecific antibody or the antibody or antigen-binding fragment thereof described herein.

In some aspects, the antibody or antigen-binding fragment thereof, the bispecific antibody, or the multispecific antibody of the present disclosure further comprise a detectable label.

Some aspects of the present disclosure are directed to polynucleotide or a set of polynucleotides encoding the antibody or antigen-binding fragment thereof, the bispecific antibody, or the multispecific antibody of the present disclosure.

In some aspects, a polynucleotide comprises a nucleic acid molecule encoding the heavy chain variable region or heavy chain of the antibody or antigen-binding fragment thereof of the present disclosure.

In some aspects, the nucleic acid molecule of the present disclosure encodes the VH of SEQ ID NO: 88, 92, 93, 95, 97, 163, 165, 169, 171, 173, 175, 177, 181, 183, 187, 189, 191, 193, 195, 203, 207, 211, or 227.

In some aspects, a polynucleotide comprises a nucleic acid molecule encoding the light chain variable region or light chain of the antibody or antigen-binding fragment thereof of the present disclosure.

In some aspects, the nucleic acid molecule encodes the VL of SEQ ID NO: 90, 164, 166, 180, 198, 200, 202, 204, 206, 208, 210, 212, 214, 216, 218, 220, 222, 224, 226, 228, 230, 232, 234, or 236.

In some aspects, a polynucleotide of the present disclosure comprises a first nucleic acid molecule encoding the heavy chain variable region of SEQ ID NO: 88, 92, 93, 95, 97, 163, 165, 169, 171, 173, 175, 177, 181, 183, 187, 189, 191, 193, 195, 203, 207, 211, or 227, and a second nucleic acid molecule encoding the light chain variable region of SEQ ID NO: 90, 164, 166, 180, 198, 200, 202, 204, 206, 208, 210, 212, 214, 216, 218, 220, 222, 224, 226, 228, 230, 232, 234, or 236.

In some aspects, a mixture of polynucleotides of the present disclosure comprises a first polynucleotide which comprises a nucleic acid molecule encoding the heavy chain variable region of SEQ ID NO: 88, 92, 93, 95, 97, 163, 165, 169, 171, 173, 175, 177, 181, 183, 187, 189, 191, 193, 195, 203, 207, 211, or 227, and a second polynucleotide which comprises a nucleic acid molecule encoding the light chain variable region of SEQ ID NO: 90,164,166, 180, 198, 200, 202, 204, 206, 208, 210, 212, 214, 216, 218, 220, 222, 224, 226, 228, 230, 232, 234, or 236.

In some aspects, a polynucleotide of the present disclosure comprises a nucleic acid molecule encoding the heavy chain variable region or heavy chain of the antibody or antigen-binding fragment thereof disclosed herein and the light chain variable region or light chain of the antibody or antigen-binding fragment thereof disclosed herein.

In some aspects, a vector comprises the polynucleotide disclosed herein.

In some aspects, a host cell comprises (a) the antibody or antigen-binding fragment thereof (b) the bispecific antibody, (c) the multispecific antibody, (d) the polynucleotide, (e) the vector, or (f) a first vector comprising the polynucleotide and a second vector comprising the polynucleotide disclosed herein.

In some aspects, the host cell is selected from the group consisting of E. coli, Pseudomonas, Bacillus, Streptomyces, yeast, HPAC, PL5, PL45, HPNE, Expi293F human cell, C6 (rat glioma cell line), U2OS, Chem-1, CHO, YB/20, NSO, PER-C6, HEK-293T, HEK293T-cCDCP1, NIH-3T3, HeLa, BHK, Hep G2, SP2/0, R1.1, B-W, L-M, COS 1, COS 7, BSC1, BSC40, BMT10 cell, PANC-1, Panc 03.27, Hs766T, CFPAC-1, CAPAN-1, Mia PaCa-2, CAPAN-2, BXPC3, mouse Fc1245, mouse Fc1242, mouse Fc1245-cCDCP1, mouse PyMT, mouse P53, mouse 4T1, mouse EMT6, mouse TRAMP, mouse C2, mouse MC38, mouse CT26, plant cell, insect cell, and human cell in tissue culture.

In some aspects, provided herein is an immunoconjugate comprising the antibody or antigen-binding fragment thereof, the bispecific antibody, or the multispecific antibody of the present disclosure and a therapeutic agent. In some aspects, the therapeutic agent is selected from the group consisting of a cytotoxin, a non-cytotoxic drug, a radioactive agent, a second antibody, an enzyme, an anti-neoplastic agent, and any combination thereof.

Some aspects of the present disclosure are directed to a method of producing an antibody or antigen-binding fragment thereof that binds to cleaved human CDCP1 comprising culturing the host cell disclosed herein so that the nucleic acid molecule is expressed and the antibody or antigen-binding fragment thereof is produced. In some aspects, the method further comprises isolating the antibody or antigen-binding fragment thereof from the culture.

In some aspects, an isolated antibody or antigen-binding fragment thereof that specifically binds to cleaved human CDCP1 is encoded by the polynucleotide disclosed herein or produced by the method disclosed herein.

Some aspects of the present disclosure are directed a pharmaceutical composition comprising the antibody or antigen-binding fragment, the bispecific antibody, the multispecific antibody, the polynucleotide, the vector, or the immunoconjugate of the present disclosure, and a pharmaceutically acceptable excipient.

In some aspects, the pharmaceutical composition disclosed herein is formulated for intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural, intrasternal, topical, epidermal, or mucosal administration.

Some aspects of the present disclosure are directed to a method of treating a cancer in a subject in need thereof, comprising administering to the subject the antibody or antigen binding fragment thereof, the bispecific antibody, the multispecific antibody, the polynucleotide, the vector, the immunoconjugate, or the pharmaceutical composition disclosed herein.

In some aspects, the antibody or antigen binding fragment thereof as disclosed herein reduces or inhibits metastasis of the cancer in the subject.

Some aspects of the present disclosure are directed to a method of reducing or inhibiting cancer metastasis in a subject in need thereof, comprising administering to the subject the antibody or antigen binding fragment thereof, the bispecific antibody, the multispecific antibody, the polynucleotide, the vector, the immunoconjugate, or the pharmaceutical composition disclosed herein.

In some aspects, the subject is afflicted with a cancer.

In some aspects, the cancer has the cleaved CDCP1 present on the cancer cell surface.

In some aspects, the cancer comprises a tumor.

In some aspects, the cancer is wherein the cancer is selected from the group consisting of small-cell lung cancer (SCLC), non-small cell lung cancer (NSCLC), squamous NSCLC, nonsquamous NSCLC, glioma, gastrointestinal cancer, renal cancer, clear cell carcinoma, ovarian cancer, liver cancer, colorectal cancer, endometrial cancer, kidney cancer, renal cell carcinoma (RCC), prostate cancer, hormone refractory prostate adenocarcinoma, thyroid cancer, neuroblastoma, pancreatic cancer, glioblastoma (glioblastoma multiforme), cervical cancer, stomach cancer, bladder cancer, hepatoma (hepatocellular carcinoma), breast cancer, colon carcinoma, head and neck cancer (or carcinoma), head and neck squamous cell carcinoma (HNSCC), gastric cancer, germ cell tumor, pediatric sarcoma, sinonasal natural killer, melanoma, metastatic malignant melanoma, cutaneous or intraocular malignant melanoma, mesothelioma, bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, neoplasm of the central nervous system (CNS), primary CNS lymphoma, tumor angiogenesis, spinal axis tumor, brain cancer, brain stem glioma, pituitary adenoma, Kaposi's sarcoma, epidermoid cancer, squamous cell cancer, environmentally-induced cancers including those induced by asbestos, virus-related cancers or cancers of viral origin, human papilloma virus (HPV)-related or -originating tumors, acute leukemia (ALL), acute myelogenous leukemia (AML), chronic lymphocytic leukemia (CLL), and chronic myelogenous leukemia (CML), undifferentiated AML, myeloblastic leukemia, myeloblastic leukemia, promyelocytic leukemia, myelomonocytic leukemia, monocytic leukemia, erythroleukemia, megakaryoblastic leukemia, isolated granulocytic sarcoma, chloroma, Hodgkin's lymphoma (HL), non-Hodgkin's lymphoma (NHL), B-cell lymphoma, T-cell lymphoma, lymphoplasmacytoid lymphoma, monocytoid B-cell lymphoma, mucosa-associated lymphoid tissue (MALT) lymphoma, anaplastic large-cell lymphoma, adult T-cell lymphoma/leukemia, mantle cell lymphoma, angio immunoblastic T-cell lymphoma, angiocentric lymphoma, intestinal T-cell lymphoma, primary mediastinal B-cell lymphoma, precursor T-lymphoblastic lymphoma, T-lymphoblastic; peripheral T-cell lymphoma, lymphoblastic lymphoma, post-transplantation lymphoproliferative disorder, true histiocytic lymphoma, primary central nervous system lymphoma, primary effusion lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T-cell lymphoma (CTLC), lymphoplasmacytoid lymphoma (LPL) with Waldenstrom's macroglobulinemia; myeloma, IgG myeloma, light chain myeloma, nonsecretory myeloma, smoldering myeloma (indolent myeloma), solitary plasmocytoma, multiple myeloma, chronic lymphocytic leukemia (CLL), hairy cell lymphoma; and any combinations of said cancers.

Some aspects of the present disclosure are directed to a method of killing a tumor cell in a subject in need thereof, comprising administering the antibody or antigen binding fragment thereof, the bispecific antibody, the multispecific antibody, the polynucleotide, the vector, the immunoconjugate, or the pharmaceutical composition of the present disclosure.

In some aspects, tumor cell is metastatic.

In some aspects, the methods of the present disclosure further comprise administering to the subject an additional anti-cancer therapy.

In some aspects, the additional anti-cancer therapy comprises a chemotherapy, an immunotherapy, a surgery, a radiotherapy, or any combination thereof.

In some aspects, the additional anti-cancer therapy comprises a standard of care therapy.

In some aspects, the additional anti-cancer therapy comprises a checkpoint inhibitor.

In some aspects, the additional anti-cancer therapy comprises an antibody or an antigen binding fragment thereof that specifically binds a protein selected from Inducible T cell Co-Stimulator (ICOS), CD137 (4-1BB), CD134 (OX40), NKG2A, CD27, CD96, Glucocorticoid-Induced TNFR-Related protein (GITR), and Herpes Virus Entry Mediator (HVEM), Programmed Death-1 (PD-1), Programmed Death Ligand-1 (PD-L1), CTLA-4, B and T Lymphocyte Attenuator (BTLA), T cell Immunoglobulin and Mucin domain-3 (TIM-3), Lymphocyte Activation Gene-3 (LAG-3), adenosine A2a receptor (A2aR), Killer cell Lectin-like Receptor G1 (KLRG-1), Natural Killer Cell Receptor 2B4 (CD244), CD160, T cell Immunoreceptor with Ig and ITIM domains (TIGIT), and the receptor for V-domain Ig Suppressor of T cell Activation (VISTA), KIR, TGFβ, IL-10, IL-8, B7-H4, Fas ligand, CXCR4, mesothelin, CEACAM-1, CD52, HER2, and any combination thereof.

In some aspects, the anti-PD-1 antibody comprises nivolumab or pembrolizumab.

In some aspects, the additional anti-cancer therapy comprises CAR-T cell therapy.

In some aspects, the antibody or antigen binding fragment thereof, the bispecific antibody, the multispecific antibody, the polynucleotide, the vector, the host cell, the immunoconjugate, or the pharmaceutical composition of the present disclosure is administered intravenously, intraperitoneally, intramuscularly, intraarterially, intrathecally, intralymphaticly, intralesionally, intracapsularly, intraorbitally, intracardiacly, intradermally, transtracheally, subcutaneously, subcuticularly, intraarticularly, subcapsularly, subarachnoidly, intraspinally, epidurally, intrasternally, topically, epidermally, or mucosally.

In some aspects, the subject is a human.

Some aspects of the present disclosure are directed to a method for detecting cleaved human CDCP1 in a sample comprising contacting the sample with the antibody or antigen-binding fragment thereof, the bispecific antibody, the multispecific antibody, the immunoconjugate, or the pharmaceutical composition of the present disclosure.

In some aspects, the sample is obtained from a human subject. In some aspects, the sample is a cancer sample. In some aspects, the sample is an in vitro sample.

Some aspects of the present disclosure are directed to a method of identifying a cancer drug candidate comprising generating an antibody or an antigen-binding fragment thereof that specifically binds to a cleaved CDCP1 as disclosed herein, wherein the antibody or antigen-binding fragment thereof preferentially binds to the cleaved CDCP1.

In some aspects, the cleaved CDCP1 is generated by (a) culturing a host cell comprising a first polynucleotide encoding the first cleaved domain and a second polynucleotide encoding the second cleaved domain and (b) isolating the cleaved CDCP1.

Some aspects of the present disclosure are directed to an isolated antigen consisting of or consisting essentially of a proteolytically cleaved CDCP1 protein. In some aspects, the cleaved CDCP1 is a complex of an N-terminal fragment of CDCP1 and a C-terminal fragment of CDCP1 which have the amino acid sequences as set forth in
(i) SEQ ID NOs: 63 and 64, respectively;
(ii) SEQ ID NOs: 68 and 70, respectively; or
(iii) SEQ ID NOs: 74 and 77, respectively.

Other features and advantages of the instant disclosure will be apparent from the following detailed description and examples which should not be construed as limiting. The contents of all cited references, including scientific articles, newspaper reports, GenBank entries, patents and patent applications cited throughout this application are expressly incorporated herein by reference.

### BRIEF DESCRIPTION OF THE DRAWINGS/FIGURES

FIG. 1A presents a schematic representation of the design of CDCP1 (Px)-Fc, a PreScission Protease-cleavable CDCP1 ectodomain fused to an TEV-releasable Fc domain with C-terminal Avi-tag, in accordance with Example 2.

FIG. 1B presents an image of an SDS-PAGE gel of engineered CDCP1 antigens in accordance with Example 2. Note, NTF was heavily glycosylated and thus ran as a smeared higher-molecular weight band at ˜60 kDa.

FIG. 1C presents SEC traces of CDCP1 (R368/K369A)-Fc, CDCP1 (Px)-Fc treated with PreScission Protease, and NTF (TEV released) in accordance with Example 2. Numbers denote fractions corresponding to the SDS-PAGE gel lanes in FIG. 1B.

FIG. 1D presents a graphical representation of results obtained from BLI assays performed in accordance with Example 2.

FIG. 1E presents a schematic representation of PreScission protease-cleavable CDCP1 full protein with N-terminal FLAG-tag expressed on the surface of HEK293T cells in accordance with Example 2.

FIG. 1F presents the results of flow cytometry (top) and western blot of analyses (bottom) of HEK293T-wt, HEK293T-CDCP1 (R368A/K369A), HEK293T-CDCP1 (Px) in accordance with Example 2.

FIG. 1G presents a schematic representation of a variant comprising an NTF fused to an Fc domain (NTF-Fc) that was generated in accordance with Example 2.

FIG. 1H presents a graphical representation of results obtained from BLI assays in accordance with Example 2.

FIG. 1I presents an image of an SDS-PAGE gel of thrombin protease-cleavable CDCP1-Fc (CDCP1(Tx)-Fc) in accordance with Example 2.

FIG. 1J presents a graphical representation of results obtained from BLI assays in accordance with Example 2.

FIG. 1K presents an SEC trace of CDCP1(Tx)-Fc treated or untreated with thrombin protease in accordance with Example 2.

FIG. 2A presents a schematic representation of an IP-MS strategy used to identify the endogenous proteolysis sites of CDCP1 on PDAC cells in accordance with Example 3.

FIG. 2B presents an images of a Western blot of PDAC cell lines expressing differential amounts of uncleaved and cleaved CDCP1 (top) and an IP-blot of a pulldown experiment using IgG 4A06 in accordance with Example 3 (bottom) in accordance with Example 3.

FIG. 2C presents a schematic representation of proteolysis sites of CDCP1 identified in accordance with Example 3.

FIG. 2D presents CDCP1 peptides identified by IP-MS of the PDAC cell line PL5 in accordance with Example 3.

FIG. 2E presents CDCP1 peptides identified by IP-MS of the PDAC cell line PL45 in accordance with Example 3.

FIG. 2F presents CDCP1 peptides identified by IP-MS of the HPAC cell line in accordance with Example 3.

FIG. 3A presents a schematic of a two-plasmid co-transfection strategy to generate a cleaved CDCP1 ectodomain in accordance with Example 4.

FIG. 3B presents an image of an SDS-PAGE gel of fl-CDCP1 and c-CDCP1 (Cut 1, Cut 2, Cut 3) ectodomain in accordance with Example 4. Note, NTF was heavily glycosylated and ran as a smeared band on the gel.

FIG. 3C presents a graphical representation of results obtained from BLI assays in accordance with Example 4.

FIG. 3D presents a graphical representation of results obtained from Differential Scanning Fluorimetry (DSF) of fl-CDCP1 and c-CDCP1 (Cut 1, Cut 2, Cut 3) ectodomains in accordance with Example 4.

FIG. 3E presents Circular Dichroism (CD) spectra of fl-CDCP1 and c-CDCP1 in accordance with Example 4.

FIG. 3F presents a P(r) plot of SEC-SAXS of fl-CDCP1 and c-CDCP1 ectodomains in accordance with Example 4.

FIG. 3G presents SEC-MALS traces of fl-CDCP1 and c-CDCP1 ectodomains in accordance with Example 4.

FIG. 3H presents a schematic representation of a two-plasmid co-transfection strategy to generate the cleaved CDCP1 ectodomain as an Fc fusion in accordance with Example 4.

FIG. 3I presents an image of an SDS-PAGE gel of fl-CDCP1-Fc and c-CDCP1-Fc (Cut 1, Cut 2, Cut 3) in accordance with Example 4.

FIG. 3J presents a graphical representation of results obtained from BLI assays in accordance with Example 4.

FIG. 3K presents SEC traces of fl-CDCP1-Fc and c-CDCP1-Fc (Cut 1, Cut 2, Cut 3) in accordance with Example 4.

FIG. 3L presents SEC traces of fl-CDCP1 and c-CDCP1 (Cut 1, Cut 2, Cut 3) without Fc domains

FIG. 3M presents graphical representations of SAXS profiles of uncleaved and cleaved CDCP1 ectodomain in accordance with Example 4.

FIG. 3N presents graphical representations of a normalized Kratky plot of uncleaved and cleaved CDCP1 ectodomain in accordance with Example 4.

FIG. 4A presents a schematic of a strategy to generate stably transduced HEK293T cell lines expressing uncleaved or cleaved CDCP1 in accordance with Example 5.

FIG. 4B presents a graphical representation of flow cytometry results of IgG 4A06 binding to HEK293T fl-CDCP1 and HEK293T c-CDCP1 cell lines in accordance with Example 5.

FIG. 4C presents an image of Western blot of CDCP1, CDCP1 phosphorylation, and phosphorylation of intracellular proteins associated with CDCP1 signaling in accordance with Example 5 and Example 6.

FIG. 4D presents a graphical representation of the results of a cell adhesion assay comparing HEK239T fl-CDCP1, HEK293T c-CDCP1, and HEK239T wild-type performed in accordance with Example 6. Note: **p=0.0016, ****p<0.0001, ns=not significant p>0.05. (unpaired t-test).

FIG. 4E presents a graphical representation of the results of a cell adhesion assay of HEK293T cells expressing fl-CDCP1 in accordance with Example 6.

FIG. 4F presents a graphical representation of the results of a cell adhesion assay HEK293T cells expressing c-CDCP1 variants in accordance with Example 6. Note: Individual data points are shown, and bars indicate average. **p=0.003, ****p<0.0001, ns=not significant p>0.05.

FIG. 4G presents a graphical representation of the results of a cell proliferation assay measured by MTT for HEK293T cells expressing HEK293T WT, fl-CDCP1, and c-CDCP1 in accordance with Example 6. Data were collected in triplicate and average and standard deviation are shown.

FIG. 4H presents a graphical representation of results of a cell proliferation assay measured by MTT for HEK293T cells expressing fl-CDCP1 variants in accordance with Example 6. Data were collected in triplicate and average and standard deviation are shown.

FIG. 4I presents a graphical representation of the results of a cell proliferation assay measured by MTT for HEK293T cells expressing c-CDCP1 variants in accordance with Example 6. Data were collected in triplicate and average and standard deviation are shown.

FIG. 5A-FIG. 5H present schematics and results related to generation of antibody specific to cleaved CDCP1 and in vitro and in vivo studies of this antibody in accordance with Example 7 and Example 8.

FIG. 5A presents a schematic representation of differential phage selection strategy to identify a cleaved CDCP1-specific binder in accordance with Example 7.

FIG. 5B presents a graphical representation of results obtained from BLI assays in accordance with Example 7.

FIG. 5C presents a negative-stain EM 3D reconstruction of c-CDCP1 (Cut 3) ectodomain bound to Fab CL03 and a nanobody in accordance with Example 7.

FIG. 5D presents immunofluorescence images of Alexa Fluor-488-labeled IgG CL03 on HPAC, PL5, and HPNE cells in accordance with Example 8.

FIG. 5E presents a graphical representation of flow cytometry results related to a panel of PDAC cells in accordance with Example 8. Note: n=3, data represent average and standard deviation.

FIG. 5F presents a schematic of antibody drug conjugate (ADC) cell killing assay (top), and a graphical representation of dose-dependent ADC-mediated cell killing assay (bottom) performed in accordance with Example 8. Note: **p=0.004, ***p=0.0018, unpaired T-test.

FIG. 5G presents a schematic representation of bi-specific T-cell engager (BiTE)-mediated T-cell activation assay (left), a graphical representation of dose-dependent activation of NFAT-GFP reporter Jurkat cells (middle); and a graphical representation of BiTE CL03 (1 nM) activation of NFAT-GFP reporter T-cells (right) in accordance with Example 8. ***p<0.001, unpaired T-test.

FIG. 5H presents a graphical representation of the results obtained from in vivo positron-emission tomography (PET) imaging of ⁸⁹Zr-labeled IgG CL03 in PDAC xenograft mice harboring HPAC or PL5 tumors in accordance with Example 8. Note: *p=0.0487, unpaired T-test, n=5 mice per cohort.

FIG. 5I presents an image of eluted phage from round 4 of phage selection (identification of cleaved CDCP1-specific Fab by phage selection) in accordance with Example 7.

FIG. 5J presents graphical representations of results obtained from BLI assays in accordance with Example 7.

FIG. 5K presents an image of an SDS-PAGE gel of fl-CDCP1 ectodomain treated with 0.5 μg plasmin in accordance with Example 7.

FIG. 5L presents graphical representations of results obtained from BLI assays performed in accordance with Example 7.

FIG. 5M presents an SEC trace of plasmin treated CDCP1 in accordance with Example 7.

FIG. 5N presents a graphical representation of flow cytometry results of HPAC cells treated with plasmin in accordance with Example 8.

FIG. 5O presents a graphical representation of flow cytometry results of PL5 cells treated with plasmin in accordance with Example 8.

FIG. 6A presents a schematic of NTF-Fc immobilization on a Streptavidin (SA) biosensor via a C-terminal biotinylated Avi-tag on the Fc domain (left), and further presents a graphical representation of results obtained from BLI assays (right), in accordance with Example 7.

FIG. 6B presents schematics of immobilization schemes in accordance with Example 7 (top), and a graphical representation of results obtained from BLI assays (bottom) in accordance with Example 7.

FIG. 6C presents a schematic of immobilization schemes in accordance with Example 7 (top), and graphical representations of multipoint BLI assays (middle and bottom) in accordance with Example 7.

FIG. 6D presents a schematic representation of an immobilization scheme in accordance with Example 7 (left), and presents a graphical representation of results obtained from BLI assays (right) in accordance with Example 7.

FIG. 6E presents a schematic of a proposed model of CL03 binding to cleaved CDCP1 in accordance with Example 7.

FIG. 7A presents a graphical representation of results obtained from BLI assays measuring species cross-reactivity of IgG CL03 in accordance with Example 9.

FIG. 7B presents a graphical representation of results obtained from BLI assays measuring species cross-reactivity of IgG CL03 in accordance with Example 9.

FIG. 8A presents a schematic representation of the two cut sites of cleaved mouse CDCP1 in accordance with Example 9.

FIG. 8B presents an image of an SDS-PAGE gel of mouse CDCP1 antigens: fl-CDCP1-Fc, c-CDCP1-Fc (Cut 1), c-CDCP1-Fc (Cut 2) in accordance with Example 9.

FIG. 8C presents an SEC trace of mouse CDCP1 antigens: fl-CDCP1-Fc, c-CDCP1-Fc (Cut 1), c-CDCP1-Fc (Cut 2) in accordance with Example 9.

FIG. 8D presents graphical representations of results obtained from BLI assays in accordance with Example 9.

FIG. 8E presents IgG58 binding to Fc1245 c-CDCP1 and Fc1245 WT cells, as measured by flow cytometry in accordance with Example 9. Note: n=3, error bars represent s.d.

FIG. 8F presents the dose-dependent activation of NFAT-GFP reporter Jurkat cells (% Jurkat activation) by IgG12 reformatted to a BiTE in Jurkat cells only or in the presence of Fc1245 c-CDCP1 cells or Fc1245 WT cells in accordance with Example 9. Note: n=2, error bars represent s.d.

FIG. 8G presents the dose-dependent ADC-mediated cell killing with IgG12 and a secondary antibody conjugated to MMAF observed in Fc1245 c-CDCP1 cells or Fc1245 WT cells in accordance with Example 9. Note: n=2, error bars represent s.d.; **p=0.0037, unpaired T-test.

FIG. 9A-FIG. 9G present schematics and results related to studies of a mouse cleaved CDCP1-specific antibody, IgG 58, in accordance with Example 9.

FIG. 9A presents a graphical representation of results obtained from BLI assays in accordance with Example 9.

FIG. 9B presents a graphical representation of flow cytometry results in accordance with Example 9. Note: n=3, error bars represent s.d.

FIG. 9C presents a graphical representation of dose-dependent activation of NFAT-GFP reporter Jurkat cells by Ab58 reformatted to a BiTE in accordance with Example 9. Note: n=2, error bars represent s.d.; and ***p=0.005, unpaired T-test.

FIG. 9D presents a graphical representation of dose-dependent ADC-mediated cell killing results in accordance with Example 9. Note: n=2, error bars represent s.d.; and **p=0.002, unpaired T-test.

FIG. 9E presents a representation of the results of in vivo positron-emission tomography (PET) imaging of 8°Zr-labeled IgG58 in syngeneic mice harboring Fc1245-cCDCP1 or Fc1245-WT tumors in accordance with Example 9.

FIG. 9F presents biodistribution of 8°Zr-IgG58 and 8°Zr-IgG12 in mice harboring subcutaneous Fc1245 c-CDCP1 tumors in accordance with Example 9. Note: Mice (n=5 per arm) and **p=0.003, ***p=0.0002, ****p<0.0001, unpaird T-test.

FIG. 9G presents ADC toxicity assay in non-tumor bearing mice dosed with 5, 10, 15 mg/kg either IgG12-monomethyl auorstatin F (MMAF) or IgG58-MMAF in accordance with Example 9. Note: Mice (n=5 per arm); dose 1 (day 0), dose 2 (day 7), dose 3 (day 14); (p=0.0002, ANOVA); and Tukey's multiple comparisons test 15 mg/kg dose (***p=0.0068) and 10 mg/kg dose (**p=0.0067).

FIG. 10 presents a table of in vitro binding affinities of Fab CL03 and IgG CL03 to uncleaved and cleaved CDCP1 in accordance with Example 7.

FIG. 11 presents a table of the binding affinity of mouse CDCP1 antibodies to cleaved and uncleaved forms of mCDCP1 measured in accordance with Example 9.

FIG. 12A presents negative stain EM 3D reconstruction of cleaved CDCP1 bound to 4A06 Fab. (left) 2D class averages of c-CDCP1 (Cut3)+4A06 Fab in the absence and presence of V_{H}H single domain antibody. (right) Different views of 3D negative stain EM map of c-CDCP1 (Cut3)+4A06 Fab+V_{H}H. Crystal structure of Fab with V_{H}H domain were fitted into the 3D negative stain EM map.

FIG. 12B presents a demonstrative micrograph of negatively stained c-CDCP1 (Cut3)+CL03 Fab+V_{H}H particles.

FIG. 12C presents a Fourier shell correlation plot used to determine the model resolution of 25 Å of c-CDCP1 (Cut3)+CL03 Fab+V_{H}H, as given by 0.143 criterion.

FIG. 12D presents a demonstrative micrograph of negatively stained c-CDCP1 (Cut3)+4A06 Fab+V_{H}H particles.

FIG. 12E presents a Fourier shell correlation plot used to determine the model resolution of 23 Å of c-CDCP1 (Cut3)+4A06 Fab+V_{H}H, as given by 0.143 criterion.

FIG. 13 presents a graphical representation of an ELISA assay evaluating the binding of CL03 to fl-CDCP1, c-CDCP1 cut1, c-CDCP1 cut2, c-CDCP1 cut3, His-TEV-CUB1, or His-CUB1 in accordance with Example 7. The circles represent fl-CDCP1, the squares represent c-CDCP1 cut1, the upright triangles represent c-CDCP1 cut2, the inverted triangles represent c-CDCP1 cut3, the diamonds represent His-TEV-CUB1, and the bolded circles represent His-CUB1.

FIG. 14A presents a graphical representation of a competition assay measuring the ability of CUB1 NTF to compete with the binding of CL03 on CDCP1 positive cells in accordance with Example 8. The circles represent 100 nM IgG CL03, the squares represent 50 nM IgG CL03, and the triangles represent 0 nM IgG CL03.

FIG. 14B presents a graphical representation of a competition assay measuring the ability of CUB1 NTF to compete with the binding of CL03 on CDCP1 positive cells in accordance with Example 8.

FIG. 15A presents a graphical representation of the tumor volume of Fc1245 c-CDCP1 tumor-bearing mice that were injected with either PBS (control) for a 400 uCi single injection of 177Lu-labeled IgG58 (test) (N=5 for both control and test groups) in accordance with Example 10. The arrow indicates the point at which all mice in the PBS (control) group were euthanized due to ulcerated tumors. The solid line indicates the two data points that used for the unpaired t-test. ***p=0.0001, unpaird T-test.

FIG. 15B presents a graphical representation of the weight of Fc1245 c-CDCP1 tumor-bearing mice that were injected with either PBS (control) or a 400 uCi single injection of 177Lu-labeled IgG58 (test) (N=5 for both control and test groups) in accordance with Example 10.

FIG. 15C presents a graphical representation of the probability of survival of Fc1245 c-CDCP1 tumor-bearing mice that were injected with either PBS (control) or a 400 uCi single injection of 177Lu-labeled IgG58 (test) (N=5 for both control and test groups) in accordance with Example 10.

FIG. 16A presents a graphical representation of results obtained from BLI assays measuring the binding affinity of each of the antibodies referred to as IgG87, IgG89, IgG94, IgG97, IgG101 (CL03 IgG H1-NtoD H3-MtoA), and IgG102 to c-CDCP1-Fc (Cut2) in accordance with Example 7.

FIG. 16B presents SEC traces of the antibodies referred to as IgG87, IgG89, IgG94, IgG97, IgG101 (CL03 IgG H1-NtoD H3-MtoA), and IgG102 in accordance with Example 7.

### DETAILED DESCRIPTION OF DISCLOSURE

The present disclosure relates to antibodies and antigen binding fragments thereof that specifically bind to a cleaved CDCP1 (human or mouse), wherein antibodies and antigen binding fragments preferentially bind to the cleaved CDCP1. In some aspects, antibodies and antigen binding fragments thereof disclosed herein do not bind to a full-length human CDCP1 at a detectable level.

Other aspects of the present disclosure relate to methods of treating a subject in need thereof, comprising administering to the subject an antibody, a bispecific antibody, a multispecific antibody, or antigen binding fragment thereof that specifically binds to the cleaved human CDCP1, wherein the antibody and antigen binding fragment do not bind to a full-length human CDCP1. In some aspects, the subject has a cancer, and the antibody, a bispecific antibody, a multispecific antibody, or antigen-binding fragment thereof treats the cancer in the subject.

### I. Terms

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The term “CDCP1” or “CUB domain-containing protein 1” or “complement C1r/C1s, Uegf, Bmp1 (CUB)-domain containing protein 1” as used herein is a 135-kDa, heavily glycosylated, single-pass membrane protein.

The term “cleaved CDCP1” as used herein refers to (i) an endogenous cleaved CDCP1 on the surface of cells and tissues (e.g., cancer cells) that is generated by proteolysis by proteases and (ii) recombinantly produced cleaved CDCP1. In some aspects, the cleaved CDCP1 (e.g., cleaved human CDCP1) is generated by being proteolytically cleaved between CUB1 and CUB2 ectodomains from a full-length CDCP1 (e.g., full-length human CDCP1). In some aspects, the cleaved CDCP1 comprises a membrane-bound complex. In some aspects, the membrane-bound complex comprises a cleaved CUB1 ectodomain associated with a membrane retained fragment of CDCP1. In some aspects, the membrane retained fragment of CDCP1 comprises a cleaved CUB2 ectodomain associated with an uncleaved CUB3 domain, wherein the cleaved CUB2 ectodomain is formed by the cleavage between CUB1 and CUB2 linker. In some aspects, the membrane-bound complex comprises an N-terminal fragment of CUB1 ectodomain and a C-terminal fragment of CUB2/CUB3 ectodomain. In some aspects, a protease (e.g., carboxylpeptidase) can trim the N- or C-terminal fragment of the cleaved CDCP1 (e.g., cleaved human CDCP1).

In some aspects, the cleaved human CDCP1 is generated by being cleaved at residue K365, R368, and/or K369 of SEQ ID NO: 273.

In some aspects, the cleaved CDCP1 comprises a first cleaved domain and second cleaved domain, wherein the first cleaved domain and the second cleaved domain are not linked.

The term “first cleaved domain” as used herein represents a CUB1 ectodomain of human CDCP1 consisting of the amino acid sequence as set forth in SEQ ID NOs: 63, 68, and 74.

The term “second cleaved domain” as used herein represents a CUB2/CUB3 ectodomain, a transmembrane domain (TM), and an intracellular domain (ICD) of human CDCP1 consisting of the amino acid sequence as set forth in SEQ ID NOs: 64, 70, and 77.

In some aspects, the cleaved CDCP1 (e.g., cleaved human CDCP1) is generated by (a) culturing a host cell comprising a first polynucleotide encoding a cleaved CUB1 ectodomain and a second polynucleotide encoding a CUB2/CUB3 ectodomain and (b) isolating the cleaved CDCP1 (e.g., cleaved human CDCP1). In some aspects, the second polynucleotide encoding the CUB2/CUB3 ectodomain includes the transmembrane domain (TM) and intracellular domain (ICD).

The terms “CDCP1” and “cleaved CDCP1” include any variants or isoforms of CDCP1 and cleaved CDCP1 which are naturally expressed by cells, including but not limited to tumor cells. Accordingly, antibodies described herein can cross-react with the cleaved CDCP1 from species other than human (e.g., cynomolgus CDCP1). Alternatively, the antibodies can be specific for human cleaved CDCP1 and do not exhibit any cross-reactivity with other species. CDCP1, cleaved CDCP1, or any variants and isoforms thereof, can either be isolated from cells or tissues which naturally express them or be recombinantly produced using well-known techniques in the art and/or those described herein.

Human CUB domain-containing protein 1 (CDCP1) (UniProt ID No. Q9H5V8-1; SEQ ID NO: 273) is an 836-amino acid Type I single-pass membrane protein with three CUB1 domains in its ectodomain. The intracellular region contains several tyrosine phosphorylation motifs. An extracellular protease cleaves CDCP1 between the CUB1 and CUB2 domains, which leads to its activation, phosphorylation of intracellular tyrosine residues by Src, and initiation of downstream signaling pathways through Akt. Cleaved CDCP1 can also form complexes with other key membrane proteins such as integrins to initiate complex-mediated signal transduction.

At least two additional isoforms of human CDCP1 have been identified. Isoform 2 (UniProt ID No. Q9H5V8-2; SEQ ID NO: 274) consists of 649 amino acids. Isoform 3 (UniProt ID No. Q9H5V8-3; SEQ ID NO: 275) consists of 343 amino acids. Human CDCP1 isoform 3 lacks amino acid residues 344-836 and has the following difference at amino acid residues 342-343 (NK→SE) relative to the amino acid sequence of human CDCP1 (UniProt ID No. Q9H5V8-1; SEQ ID NO: 273).

Below are the amino acid sequences of the three known human CDCP1 isoforms.

### (A) Human CDCP1 (SEQ ID NO: 273):

(SEQ ID NO: 273)
MAGLNCGVSIALLGVLLLGAARLPRGAEAFEIALPRESNITVLIKLGTP
TLLAKPCYIVISKRHITMLSIKSGERIVFTFSCQSPENHFVIEIQKNID
CMSGPCPFGEVQLQPSTSLLPTLNRTFIWDVKAHKSIGLELQFSIPRLR
QIGPGESCPDGVTHSISGRIDATVVRIGTFCSNGTVSRIKMQEGVKMAL
HLPWFHPRNVSGFSIANRSSIKRLCIIESVFEGEGSATLMSANYPEGFP
EDELMTWQFVVPAHLRASVSFLNFNLSNCERKEERVEYYIPGSTTNPEV
FKLEDKQPGNMAGNFNLSLQGCDQDAQSPGILRLQFQVLVQHPQNESNK
IYVVDLSNERAMSLTIEPRPVKQSRKFVPGCFVCLESRTCSSNLTLTSG
SKHKISFLCDDLTRLWMNVEKTISCTDHRYCQRKSYSLQVPSDILHLPV
ELHDFSWKLLVPKDRLSLVLVPAQKLQQHTHEKPCNTSFSYLVASAIPS
QDLYFGSFCPGGSIKQIQVKQNISVTLRTFAPSFQQEASRQGLTVSFIP
YFKEEGVFTVTPDTKSKVYLRTPNWDRGLPSLTSVSWNISVPRDQVACL
TFFKERSGVVCQTGRAFMIIQEQRTRAEEIFSLDEDVLPKPSFHHHSFW
VNISNCSPTSGKQLDLLFSVTLTPRTVDLTVILIAAVGGGVLLLSALGL
IICCVKKKKKKTNKGPAVGIYNDNINTEMPRQPKKFQKGRKDNDSHVYA
VIEDTMVYGHLLQDSSGSFLQPEVDTYRPFQGTMGVCPPSPPTICSRAP
TAKLATEEPPPRSPPESESEPYTFSHPNNGDVSSKDTDIPLLNTQEPME
PAE

The signal sequence of human CDCP1 corresponds to amino acids 1-29 (underlined). Thus, the mature isoform of Human CDCP1 isoform 1 consists of amino acids 30 to 836.

The extracellular domain of mature human CDCP1 consists of amino acids 30-836 of SEQ ID NO: 273 and has the amino acid sequence:

(SEQ ID NO: 276)
FEIALPRESNITVLIKLGTPTLLAKPCYIVISKRHITMLSIKSGERIVF
TFSCQSPENHFVIEIQKNIDCMSGPCPFGEVQLQPSTSLLPTLNRTFIW
DVKAHKSIGLELQFSIPRLRQIGPGESCPDGVTHSISGRIDATVVRIGT
FCSNGTVSRIKMQEGVKMALHLPWFHPRNVSGFSIANRSSIKRLCIIES
VFEGEGSATLMSANYPEGFPEDELMTWQFVVPAHLRASVSFLNFNLSNC
ERKEERVEYYIPGSTTNPEVFKLEDKQPGNMAGNFNLSLQGCDQDAQSP
GILRLQFQVLVQHPQNESNKIYVVDLSNERAMSLTIEPRPVKQSRKFVP
GCFVCLESRTCSSNLTLTSGSKHKISFLCDDLTRLWMNVEKTISCTDHR
YCQRKSYSLQVPSDILHLPVELHDFSWKLLVPKDRLSLVLVPAQKLQQH
THEKPCNTSFSYLVASAIPSQDLYFGSFCPGGSIKQIQVKQNISVTLRT
FAPSFQQEASRQGLTVSFIPYFKEEGVFTVTPDTKSKVYLRTPNWDRGL
PSLTSVSWNISVPRDQVACLTFFKERSGVVCQTGRAFMIIQEQRTRAEE
IFSLDEDVLPKPSFHHHSFWVNISNCSPTSGKQLDLLFSVTLTPRTVDL
TVILIAAVGGGVLLLSALGLIICCVKKKKKKTNKGPAVGIYNDNINTEM
PRQPKKFQKGRKDNDSHVYAVIEDTMVYGHLLQDSSGSFLQPEVDTYRP
FQGTMGVCPPSPPTICSRAPTAKLATEEPPPRSPPESESEPYTFSHPNN
GDVSSKDTDIPLLNTQEPMEPAE

(B) Human CDCP1 Isoform 2 (UniProt ID No. Q9H5V8-2; SEQ ID NO: 274):

(SEQ ID NO: 274)
MQEGVKMALHLPWFHPRNVSGFSIANRSSIKRLCIIESVFEGEGSATLM
SANYPEGFPEDELMTWQFVVPAHLRASVSFLNFNLSNCERKEERVEYYI
PGSTTNPEVFKLEDKQPGNMAGNFNLSLQGCDQDAQSPGILRLQFQVLV
QHPQNESNKIYVVDLSNERAMSLTIEPRPVKQSRKFVPGCFVCLESRTC
SSNLTLTSGSKHKISFLCDDLTRLWMNVEKTISCTDHRYCQRKSYSLQV
PSDILHLPVELHDFSWKLLVPKDRLSLVLVPAQKLQQHTHEKPCNTSFS
YLVASAIPSQDLYFGSFCPGGSIKQIQVKQNISVTLRTFAPSFQQEASR
QGLTVSFIPYFKEEGVFTVTPDTKSKVYLRTPNWDRGLPSLTSVSWNIS
VPRDQVACLTFFKERSGVVCQTGRAFMIIQEQRTRAEEIFSLDEDVLPK
PSFHHHSFWVNISNCSPTSGKQLDLLFSVTLTPRTVDLTVILIAAVGGG
VLLLSALGLIICCVKKKKKKTNKGPAVGIYNDNINTEMPRQPKKFQKGR
KDNDSHVYAVIEDTMVYGHLLQDSSGSFLQPEVDTYRPFQGTMGVCPPS
PPTICSRAPTAKLATEEPPPRSPPESESEPYTFSHPNNGDVSSKDTDIP
LLNTQEPMEPAE

(C) Human CDCP1 Isoform 3 (UniProt ID No. Q9H5V8-3; SEQ ID NO: 275):

(SEQ ID NO: 275)
MAGLNCGVSIALLGVLLLGAARLPRGAEAFEIALPRESNITVLIKLGTP
TLLAKPCYIVISKRHITMLSIKSGERIVFTFSCQSPENHFVIEIQKNID
CMSGPCPFGEVQLQPSTSLLPTLNRTFIWDVKAHKSIGLELQFSIPRLR
QIGPGESCPDGVTHSISGRIDATVVRIGTFCSNGTVSRIKMQEGVKMAL
HLPWFHPRNVSGFSIANRSSIKRLCIIESVFEGEGSATLMSANYPEGFP
EDELMTWQFVVPAHLRASVSFLNFNLSNCERKEERVEYYIPGSTTNPEV
FKLEDKQPGNMAGNFNLSLQGCDQDAQSPGILRLQFQVLVQHPQNESSE

Amino acid sequence of c-CDCP1 Cut 1 N-terminal fragment (NTF) of extracellular domain

(SEQ ID NO: 63)
FEIALPRESNITVLIKLGTPTLLAKPCYIVISKRHITMLSIKSGERIVF
TFSCQSPENHFVIEIQKNIDCMSGPCPFGEVQLQPSTSLLPTLNRTFIW
DVKAHKSIGLELQFSIPRLRQIGPGESCPDGVTHSISGRIDATVVRIGT
FCSNGTVSRIKMQEGVKMALHLPWFHPRNVSGFSIANRSSIKRLCIIES
VFEGEGSATLMSANYPEGFPEDELMTWQFVVPAHLRASVSFLNFNLSNC
ERKEERVEYYIPGSTTNPEVFKLEDKQPGNMAGNFNLSLQGCDQDAQSP
GILRLQFQVLVQHPQNESNKIYVVDLSNERAMSLTIEPRPVK

Amino acid sequence of c-CDCP1 Cut 1 C-terminal fragment (CTF) of extracellular domain, with transmembrane domain (TM) and intracellular domain (ICD)

(SEQ ID NO: 64)
QSRKFVPGCFVCLESRTCSSNLTLTSGSKHKISFLCDDLTRLWMNVEKT
ISCTDHRYCQRKSYSLQVPSDILHLPVELHDFSWKLLVPKDRLSLVLVP
AQKLQQHTHEKPCNTSFSYLVASAIPSQDLYFGSFCPGGSIKQIQVKQN
ISVTLRTFAPSFQQEASRQGLTVSFIPYFKEEGVFTVTPDTKSKVYLRT
PNWDRGLPSLTSVSWNISVPRDQVACLTFFKERSGVVCQTGRAFMIIQE
QRTRAEEIFSLDEDVLPKPSFHHHSFWVNISNCSPTSGKQLDLLFSVTL
TPRTVDLTVILIAAVGGGVLLLSALGLIICCVKKKKKKTNKGPAVGIYN
DNINTEMPRQPKKFQKGRKDNDSHVYAVIEDTMVYGHLLQDSSGSFLQP
EVDTYRPFQGTMGVCPPSPPTICSRAPTAKLATEEPPPRSPPESESEPY
TFSHPNNGDVSSKDTDIPLLNTQEPMEPAE

Amino acid sequence of c-CDCP1 Cut 1 C-terminal fragment (CTF) of extracellular domain, without transmembrane domain (TM) and intracellular domain (ICD) amino acid sequence

(SEQ ID NO: 66)
QSRKFVPGCFVCLESRTCSSNLTLTSGSKHKISFLCDDLTRLWMNVEKT
ISCTDHRYCQRKSYSLQVPSDILHLPVELHDFSWKLLVPKDRLSLVLVP
AQKLQQHTHEKPCNTSFSYLVASAIPSQDLYFGSFCPGGSIKQIQVKQN
ISVTLRTFAPSFQQEASRQGLTVSFIPYFKEEGVFTVTPDTKSKVYLRT
PNWDRGLPSLTSVSWNISVPRDQVACLTFFKERSGVVCQTGRAFMIIQE
QRTRAEEIFSLDEDVLPKPSFHHHSFWVNISNCSPTSGKQLDLLFSVTL
TPRTVDLT

Amino acid sequence of c-CDCP1 Cut 2 N-terminal fragment of extracellular domain

(SEQ ID NO: 68)
FEIALPRESNITVLIKLGTPTLLAKPCYIVISKRHITMLSIKSGERIVF
TFSCQSPENHFVIEIQKNIDCMSGPCPFGEVQLQPSTSLLPTLNRTFIW
DVKAHKSIGLELQFSIPRLRQIGPGESCPDGVTHSISGRIDATVVRIGT
FCSNGTVSRIKMQEGVKMALHLPWFHPRNVSGFSIANRSSIKRLCIIES
VFEGEGSATLMSANYPEGFPEDELMTWQFVVPAHLRASVSFLNFNLSNC
ERKEERVEYYIPGSTTNPEVFKLEDKQPGNMAGNFNLSLQGCDQDAQSP
GILRLQFQVLVQHPQNESNKIYVVDLSNERAMSLTIEPRPVKQSR

Amino acid sequence of c-CDCP1 Cut 2 C-terminal fragment of extracellular domain, with transmembrane domain (TM) and intracellular domain (ICD)

(SEQ ID NO: 70)
KFVPGCFVCLESRTCSSNLTLTSGSKHKISFLCDDLTRLWMNVEKTISC
TDHRYCQRKSYSLQVPSDILHLPVELHDFSWKLLVPKDRLSLVLVPAQK
LQQHTHEKPCNTSFSYLVASAIPSQDLYFGSFCPGGSIKQIQVKQNISV
TLRTFAPSFQQEASRQGLTVSFIPYFKEEGVFTVTPDTKSKVYLRTPNW
DRGLPSLTSVSWNISVPRDQVACLTFFKERSGVVCQTGRAFMIIQEQRT
RAEEIFSLDEDVLPKPSFHHHSFWVNISNCSPTSGKQLDLLFSVTLTPR
TVDLTVILIAAVGGGVLLLSALGLIICCVKKKKKKTNKGPAVGIYNDNI
NTEMPRQPKKFQKGRKDNDSHVYAVIEDTMVYGHLLQDSSGSFLQPEVD
TYRPFQGTMGVCPPSPPTICSRAPTAKLATEEPPPRSPPESESEPYTFS
HPNNGDVSSKDTDIPLLNTQEPMEPAE

Amino acid sequence of c-CDCP1 Cut 2 C-terminal fragment of extracellular domain, without transmembrane domain (TM) and intracellular domain (ICD)

(SEQ ID NO: 72)
KFVPGCFVCLESRTCSSNLTLTSGSKHKISFLCDDLTRLWMNVEKTISC
TDHRYCQRKSYSLQVPSDILHLPVELHDFSWKLLVPKDRLSLVLVPAQK
LQQHTHEKPCNTSFSYLVASAIPSQDLYFGSFCPGGSIKQIQVKQNISV
TLRTFAPSFQQEASRQGLTVSFIPYFKEEGVFTVTPDTKSKVYLRTPNW
DRGLPSLTSVSWNISVPRDQVACLTFFKERSGVVCQTGRAFMIIQEQRT
RAEEIFSLDEDVLPKPSFHHHSFWVNISNCSPTSGKQLDLLFSVTLTPR
TVDLT

Amino acid sequence of c-CDCP1 Cut 3 N-terminal fragment of extracellular domain

(SEQ ID NO: 74)
FEIALPRESNITVLIKLGTPTLLAKPCYIVISKRHITMLSIKSGERIVFT
FSCQSPENHFVIEIQKNIDCMSGPCPFGEVQLQPSTSLLPTLNRTFIWDV
KAHKSIGLELQFSIPRLRQIGPGESCPDGVTHSISGRIDATVVRIGTFCS
NGTVSRIKMQEGVKMALHLPWFHPRNVSGFSIANRSSIKRLCIIESVFEG
EGSATLMSANYPEGFPEDELMTWQFVVPAHLRASVSFLNFNLSNCERKEE
RVEYYIPGSTTNPEVFKLEDKQPGNMAGNFNLSLQGCDQDAQSPGILRLQ
FQVLVQHPQNESNKIYVVDLSNERAMSLTIEPRPVKQSRK

Amino acid sequence of c-CDCP1 Cut 3 C-terminal fragment of extracellular domain, with transmembrane domain (TM) and intracellular domain (ICD)

(SEQ ID NO: 77)
FVPGCFVCLESRTCSSNLTLTSGSKHKISFLCDDLTRLWMNVEKTISCTD
HRYCQRKSYSLQVPSDILHLPVELHDFSWKLLVPKDRLSLVLVPAQKLQQ
HTHEKPCNTSFSYLVASAIPSQDLYFGSFCPGGSIKQIQVKQNISVTLRT
FAPSFQQEASRQGLTVSFIPYFKEEGVFTVTPDTKSKVYLRTPNWDRGLP
SLTSVSWNISVPRDQVACLTFFKERSGVVCQTGRAFMIIQEQRTRAEEIF
SLDEDVLPKPSFHHHSFWVNISNCSPTSGKQLDLLFSVTLTPRTVDLTVI
LIAAVGGGVLLLSALGLIICCVKKKKKKTNKGPAVGIYNDNINTEMPRQP
KKFQKGRKDNDSHVYAVIEDTMVYGHLLQDSSGSFLQPEVDTYRPFQGTM
GVCPPSPPTICSRAPTAKLATEEPPPRSPPESESEPYTFSHPNNGDVSSK
DTDIPLLNTQEPMEPAE

Amino acid sequence of c-CDCP1 Cut 3 C-terminal fragment of extracellular domain, without transmembrane domain (TM) and intracellular domain (ICD)

(SEQ ID NO: 78)
FVPGCFVCLESRTCSSNLTLTSGSKHKISFLCDDLTRLWMNVEKTISCTD
HRYCQRKSYSLQVPSDILHLPVELHDFSWKLLVPKDRLSLVLVPAQKLQQ
HTHEKPCNTSFSYLVASAIPSQDLYFGSFCPGGSIKQIQVKQNISVTLRT
FAPSFQQEASRQGLTVSFIPYFKEEGVFTVTPDTKSKVYLRTPNWDRGLP
SLTSVSWNISVPRDQVACLTFFKERSGVVCQTGRAFMIIQEQRTRAEEIF
SLDEDVLPKPSFHHHSFWVNISNCSPTSGKQLDLLFSVTLTPRTVDLT

Mouse CDCP1 comprises the following amino acid sequence (including a signal sequence) (UniProt ID No. Q5U462-1; SEQ ID NO: 277):

(SEQ ID NO: 277)
MAHSACGFSVALLGALLLGTARLLRGTEASEIALPQRSGVTVSIKLGNPA
LPVKICYIVMSRQHITELIIRPGERKSFTFSCSNPEKHFVLKIEKNIDCM
SGPCPFGEVHLQPSTSELPILNRTFIWDVRAHKSIGLELQFATPRLRQIG
PGESCADGVTHSISGHIDATEVRIGTFCSNGTVSRIKMQEGVKMALHLPW
FHRRNVSGFSIANRSSIKRLCIIESVFEGEGSATLMSANYPGGFPEDELM
TWQFVVPAHLRASVSFLNFNVSNCERKEERVEYYIPGSTTNPEVFRLEDK
QPGNMAGNFNLSLQGCDQDAQSPGILRLQFQVLVQRPQDESNKTYMVDLS
RERTMSLTIEPRPVKHGRRFVPGCFVCLESRTCSTNVTLTAGSIHKISFL
CDDLTRLWVNVEKTLSCLDHRYCYRQSFKLQVPDYILQLPVQLHDFSWKL
LVPKDKLSLMLVPGQKLQQHTQERPCNTSFGYHVTSTTPGQDLYFGSFCS
GGSIEKIQVKQNSSVTLRAYAPSFQQEVSKQGLIVSYTPYFKEEGIFTVT
PDTKNKVYLRSPNWDRGLPALSSVSWNISVPSNQVACLTVLKERSGLACQ
SGRAFMIIQEQQSRAEEIFSLEEEVLPKPSFHHHSFWVNISNCSPMNGKQ
LDLLFWVTLTPRTVDLAVVIGAAGGGALLLFALVLIICFVKKKKKVDKGP
AVGIYNGNVNTQMPQTQKFPKGRKDNDSHVYAVIEDTMVYGHLLQDSGGS
FIQPEVDTYRPFQGPMGDCPPTPPPLFSRTPTAKFTAEELAPSSPPESES
EPYTFSHPNKGEIGVRETDIPLLHTQGPVETEE

Cynomolgus CDCP1 comprises the following amino acid sequence (including a signal sequence) (UniProt ID No. A0A2K5VLK2-1; SEQ ID NO: 278):

(SEQ ID NO: 278)
MAGLNCGVTIALLGVLLLGAARLPRAAEAFEIALPRESNITVLIKLGTPT
LLAKPCYIVISKRHTTMLSIKPGERILFTFSCQSPENHFVIEIQKNIDCM
SGPCPFGEVQLQPSTSLLPTLNRTFIWDVKAHKSIGLELQFSIPLLRQIG
PGESCPGGVTYSISGRIDATVVRIGTFCSNGTVSRIKMQEGVKMALHLPW
FHPRNVSGFSIANRSSIKRLCIIESVFEGEGSATLMSANYPEGFPEDELM
TWQFVIPAHLRASVSFLNFNLSNCERKEERVEYYIPGSTTNPEVFKLEDK
QPGNMAGNFNLSLQGCDQDAQNPGILRLQFQVLVQHPQNESNKIYVVDLS
NERATSLTIEPRPVKQSRKFVPGCFVCLESRTCSTNLTLTSGSKHKISFL
CDNLTRLWMNVEKNISCTDHRYCQRKSYSLQVPSDILHLPVELHDFSWKL
LVPKDRLSLVLVPAQKLQQHTHEKPCNTSFSYLVASAIPSQDLYFGSFCP
GGSIEQIQVKQNISVTLRTFAPSFRQEASRQGLTVSFIPYFKEEGVFTVT
PDTKSKVYLRTPNWDRGLPSLTSVSWNISVPRDQVACLTFFKERTGVVCQ
TGRAFMIIQEQRTRAEEIFSLDEDALPKPRFHHHSFWVNISNCSPASGKQ
LDLLFWVTLTPRTVDLTVILITVVGGGAVLLSALGLIICCVKKKKKKTNK
GPAVGVYNGNINTEMPRQPKKFQKGRKDNDSHVYAVIEDTMVYGHLLQDS
GGSFLQPEVDTYRPFQGTMGVCPPSPPTICSRAPTAKLAAEELPPCSPPE
SESEPYTFSHPNNGDINSKETDIPLLNTQEPVEPAE

The term “antibody” refers, in some aspects, to a protein comprising at least two heavy (H) chains and two light (L) chains inter-connected by disulfide bonds. Each heavy chain is comprised of a heavy chain variable region (abbreviated herein as VH) and a heavy chain constant region (abbreviated herein as CH). The term “bispecific antibody,” as used herein, refers to an antibody comprising at least two antigen-binding domains, i.e., at least two paratopes. As such, in some aspects, a bispecific antibody comprises at least two heavy chain variable regions (VH1 and VH2) and at least two light chain variable regions (VL1 and VL2. In some aspects, the at least two heavy chain variable regions are the same or different. In some aspects, the at least two light chain variable regions are the same or different. A “multispecific antibody,” as used herein, refers to an antibody comprising at least three antigen-binding domains, i.e., at least three paratopes.

In some antibodies, e.g., naturally-occurring IgG antibodies, the heavy chain constant region is comprised of a hinge and three domains, CH1, CH2 and CH3. In some antibodies, e.g., naturally-occurring IgG antibodies, each light chain is comprised of a light chain variable region (abbreviated herein as VL) and a light chain constant region. The light chain constant region is comprised of one domain (abbreviated herein as CL). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system. A heavy chain can have the C-terminal lysine or not. Unless specified otherwise herein, the amino acids in the variable regions are numbered using the Kabat numbering system and those in the constant regions are numbered using the EU system.

An “IgG antibody”, e.g., a human IgG1, IgG2, IgG3 and IgG4 antibody, as used herein has, in some aspects, the structure of a naturally-occurring IgG antibody, i.e., it has the same number of heavy and light chains and disulfide bonds as a naturally-occurring IgG antibody of the same subclass. For example, an anti-human or mouse cleaved CDCP1 IgG1, IgG2, IgG3 or IgG4 antibody consists of two heavy chains (HCs) and two light chains (LCs), wherein the two HCs and LCs are linked by the same number and location of disulfide bridges that occur in naturally-occurring IgG1, IgG2, IgG3 and IgG4 antibodies, respectively (unless the antibody has been mutated to modify the disulfide bridges).

Antibodies typically bind specifically to their cognate antigen with high affinity, reflected by a dissociation constant (K_{D}) of 10⁻⁵ to 10⁻¹¹ M or less. Any K_{D} greater than about 10+M is generally considered to indicate nonspecific binding. As used herein, an antibody that “binds specifically” to an antigen refers to an antibody that binds to the antigen and substantially identical antigens with high affinity, which means having a K_{D} of 10⁻⁷M or less, 10⁻⁸ M or less, 5×10⁻⁹M or less, or between 10⁻⁸ M and 10⁻¹⁰ M or less, but does not bind with high affinity to unrelated antigens. An antigen is “substantially identical” to a given antigen if it exhibits a high degree of sequence identity to the given antigen, for example, if it exhibits at least 80%, at least 90%, at least 95%, at least 97%, or at least 99% sequence identity to the sequence of the given antigen. By way of example, an antibody that binds specifically to cleaved human CDCP1 can, in some aspects, also have cross-reactivity with CDCP1 antigens from certain primate species (e.g., cynomolgus CDCP1), but cannot cross-react with CDCP1 antigens from other species or with an antigen other than CDCP1.

The phrase “preferentially binds” or its grammatically similar terms as used herein refer to the fact that the antibody or antigen-binding fragment thereof specifically binds to a first antigen (e.g., cleaved CDCP1) more readily than it would bind to a second antigen (e.g., full-length CDCP1). Thus, an antibody which “preferentially binds” to a given antigen (e.g., cleaved CDCP1) would more likely bind to that antigen than to a related antigen (e.g., full length CDCP1), even though such an antibody may cross-react with the related antigen. In some aspects, an antibody that preferentially binds to the first antigen (e.g., cleaved CDCP1) does not bind to the second antigen (e.g., full length CDCP1) at a detectable level. In some aspects, the antibody or antigen-binding fragment thereof as disclosed herein binds at least 10×, at least 100×, at least 1000× tighter to the cleaved CDCP1 compared to a full-length CDCP1. Exemplary method used to detect the binding between the antibody and the antigen can be, but not limited to, phase ELISA, biolayer interferometry analysis using an Octet instrument (e.g., ForteBio), and surface plasmon resonance (SPR) analysis.

An immunoglobulin can be from any of the commonly known isotypes, including but not limited to IgA, secretory IgA, IgG and IgM. The IgG isotype is divided in subclasses in certain species: IgG1, IgG2, IgG3 and IgG4 in humans, and IgG1, IgG2a, IgG2b and IgG3 in mice. In some aspects, the anti-human cleaved CDCP1 antibodies described herein are of the IgG1 subtype. Immunoglobulins, e.g., IgG1, exist in several allotypes, which differ from each other in at most a few amino acids. “Antibody” includes, by way of example, both naturally-occurring and non-naturally-occurring antibodies; monoclonal and polyclonal antibodies; chimeric and humanized antibodies; human and nonhuman antibodies and wholly synthetic antibodies.

The term “antigen-binding fragment” of an antibody, as used herein, refers to one or more fragments of an antibody that retain the ability to specifically bind to an antigen (e.g., cleaved human CDCP1). It has been shown that the antigen-binding function of an antibody can be performed by fragments of a full-length antibody. Examples of binding fragments encompassed within the term “antigen-binding fragment” of an antibody, e.g., an anti-human or mouse cleaved CDCP1 antibody described herein, include (i) a Fab fragment (fragment from papain cleavage) or a similar monovalent fragment consisting of the V_{L}, V_{H}, LC and CH1 domains; (ii) a F(ab′)2 fragment (fragment from pepsin cleavage) or a similar bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546), which consists of a V_{H} domain; (vi) an isolated complementarity determining region (CDR) and (vii) a combination of two or more isolated CDRs which can optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. These antibody fragments are obtained using conventional techniques known to those with skill in the art, and the fragments are screened for utility in the same manner as are intact antibodies. Antigen-binding fragments can be produced by recombinant DNA techniques, or by enzymatic or chemical cleavage of intact immunoglobulins.

A “bispecific” or “bifunctional antibody” is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992).

The term “monoclonal antibody,” as used herein, refers to an antibody from a population of substantially homogeneous antibodies, i.e., the individual antibodies comprised in the population are substantially similar and bind the same epitope(s) (e.g., the antibodies display a single binding specificity and affinity), except for possible variants that can arise during production of the monoclonal antibody, such variants generally being present in minor amounts. The modifier “monoclonal” indicates the character of the antibody as being obtained from a substantially homogeneous population of antibodies, and is not to be construed as requiring production of the antibody by any particular method. The term “human monoclonal antibody” refers to an antibody from a population of substantially homogeneous antibodies that display(s) a single binding specificity and which has variable and optional constant regions derived from human germline immunoglobulin sequences. In some aspects, human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell.

The term “recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom, (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies comprise variable and constant regions that utilize particular human germline immunoglobulin sequences encoded by the germline genes, but include subsequent rearrangements and mutations which occur, for example, during antibody maturation. As known in the art (see, e.g., Lonberg (2005) Nature Biotech. 23 (9): 1117-1125), the variable region contains the antigen binding domain, which is encoded by various genes that rearrange to form an antibody specific for a foreign antigen. In addition to rearrangement, the variable region can be further modified by multiple single amino acid changes (referred to as somatic mutation or hypermutation) to increase the affinity of the antibody to the foreign antigen. The constant region will change in further response to an antigen (i.e., isotype switch). Therefore, the rearranged and somatically mutated nucleic acid molecules that encode the light chain and heavy chain immunoglobulin polypeptides in response to an antigen cannot have sequence identity with the original nucleic acid molecules, but instead will be substantially identical or similar (i.e., have at least 80% identity).

A “human” antibody (HuMAb) refers to an antibody having variable regions in which both the framework and CDR regions are derived from human germline immunoglobulin sequences. Furthermore, if the antibody contains a constant region, the constant region also is derived from human germline immunoglobulin sequences. The anti-human cleaved CDCP1 antibodies described herein can include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences. The terms “human” antibodies and “fully human” antibodies are used synonymously.

A “humanized” antibody refers to an antibody in which some, most or all of the amino acids outside the CDR domains of a non-human antibody are replaced with corresponding amino acids derived from human immunoglobulins. In some aspects of a humanized form of an antibody, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

As used herein, “isotype” refers to the antibody class (e.g., IgG1, IgG2, IgG3, IgG4, IgM, IgA1, IgA2, IgD, and IgE antibody) that is encoded by the heavy chain constant region genes.

“Allotype” refers to naturally-occurring variants within a specific isotype group, which variants differ in a few amino acids (see, e.g., Jefferis et al. (2009) mAbs 1:1). Anti-human or mouse cleaved CDCP1 antibodies described herein can be of any allotype. As used herein, antibodies referred to as “IgG1f,” “IgG1.1f,” or “IgG1.3f” isotype are IgG1, effectorless IgG1.1, and effectorless IgG1.3 antibodies, respectively, of the allotype “f,” i.e., having 214R, 356E and 358M according to the EU index as in Kabat.

The phrases “an antibody recognizing an antigen” and “an antibody specific for an antigen” are used interchangeably herein with the term “an antibody which binds specifically to an antigen.”

An “isolated antibody,” as used herein, is intended to refer to an antibody which is substantially free of other proteins and cellular material.

As used herein, an antibody that “binds cleaved CDCP1” is intended to refer to an antibody that interacts with cleaved CDCP1 (e.g., human and mouse cleaved CDCP1), e.g., in binding assays using HEK293T cells transfected with cleaved human or mouse CDCP1 expressing tumor cells, with an EC₅₀ of about 25 μg/mL or less, about 23 μg/mL or less, about 20 μg/mL or less, about 15 μg/mL or less, about 10 μg/mL or less, about 5 μg/mL or less, about 3 μg/mL or less, about 2 μg/mL or less, about 1 μg/mL or less, about 0.5 μg/mL or less, about 0.45 μg/mL or less, about 0.4 μg/mL or less, about 0.35 μg/mL or less, or about 0.3 μg/mL or less, in art-recognized methods, e.g., the FACS-based binding assays described herein. In some aspects, the anti-cleaved CDCP1 antibody, e.g., described herein, or antigen-binding fragment thereof binds cleaved CDCP1 (e.g., human or mouse cleaved CDCP1) expressed on, e.g., HEK293T cells, with an EC₅₀ of about 200 nM or less, about 175 nM or less, about 160 nM or less, about 150 nM or less, about 125 nM or less, about 110 nM or less, about 100 nM or less about 80 nM or less, about 75 nM or less, about 60 nM or less, about 50 nM or less, about 40 nM or less, about 35 nM or less, about 30 nM or less, about 25 nM or less, about 20 nM or less, about 15 nM or less, about 10 nM or less, about 9 nM or less, about 8 nM or less, about 7 nM or less, about 6 nM or less, about 5 nM or less, about 4 nM or less, about 3 nM or less, about 2 nM or less, about 1.9 nM or less, about 1.8 nM or less, about 1.7 nM or less, about 1.6 nM or less, about 1.5 nM or less, about 1.4 nM or less, about 1.3 nM or less, about 1.2 nM or less, about 1.1 nM or less, about 1.0 nM or less, about 0.9 nM or less, about 0.8 nM or less, about 0.7 nM or less, about 0.6 nM or less, about 0.5 nM or less, about 0.4 nM or less, about 0.3 nM or less, about 0.2 nM or less, or about 0.1 nM or less. In certain aspects, the anti-cleaved CDCP1 antibody, e.g., described herein, or antigen-binding fragment thereof binds human or mouse cleaved CDCP1 expressed on, e.g., HEK293T cells, with an EC₅₀ of less than about 10 nM. In certain aspects, the anti-cleaved CDCP1 antibody, e.g., described herein, or antigen-binding fragment thereof binds cleaved CDCP1 (e.g., human or mouse) expressed on, e.g., HEK293T cells, with an EC₅₀ of less than about 5 nM. In certain aspects, the anti-cleaved CDCP1 antibody, e.g., described herein, or antigen-binding fragment thereof binds cleaved CDCP1 (e.g., human or mouse) expressed on, e.g., HEK293T cells, with an EC₅₀ of less than about 1.5 nM. In certain aspects, the anti-cleaved CDCP1 antibody, e.g., described herein, or antigen-binding fragment thereof binds cleaved CDCP1 (e.g., human or mouse) expressed on, e.g., HEK293T cells, with an EC₅₀ of about 1 nM or less. In certain aspects, the anti-cleaved CDCP1 antibody, e.g., described herein, or antigen-binding fragment thereof binds cleaved CDCP1 (e.g., human or mouse) expressed on, e.g., HEK293T cells, with an EC₅₀ of about 0.5 nM or less. In certain aspects, the anti-cleaved CDCP1 antibody, e.g., described herein, or antigen-binding fragment thereof binds cleaved CDCP1 (human or mouse) expressed on, e.g., HEK293T cells, with an EC₅₀ of about 0.3 nM or less. In certain aspects, the anti-cleaved CDCP1 antibody e.g., described herein, or antigen-binding fragment thereof binds cleaved CDCP1 (human or mouse) expressed on, e.g., HEK293T cells, with an EC₅₀ of about 0.2 nM or less.

As used herein, an antibody that “inhibits, prevents, or reduces shedding of cleaved CDCP1” by a cell, e.g., a tumor cell, is intended to refer to an antibody that inhibits, prevents, or reduces release of cleaved CDCP1 from the surface of the cell. Without being bound by a mechanism, the anti-cleaved CDCP1 antibodies, e.g., described herein, or antigen-binding fragments thereof disclosed herein reduce the amount of cleaved CDCP1. In some aspects, the antibody increases membrane bound cleaved CDCP1 on the surface of the cell. In some aspects, the anti-cleaved CDCP1 antibody, e.g., described herein, or antigen-binding fragment thereof increases the retention of surface cleaved CDCP1 on a cell transfected with human or mouse cleaved CDCP1 at a cleaved human or mouse CDCP1 retention EC₅₀ of about 10 nM or less, about 5 nM or less, about 1 nM or less, about 0.85 nM or less, about 0.8 nM or less, about 0.75 nM or less, about 0.7 nM or less, about 0.65 nM or less, about 0.6 nM or less, about 0.55 nM or less, about 0.5 nM or less, about 0.45 nM or less, about 0.4 nM or less, about 0.35 nM or less, about 0.3 nM or less, about 0.25 nM or less, about 0.2 nM or less, about 0.15 nM or less, or about 0.1 nM or less. In certain aspects, the anti-human or mouse cleaved CDCP1 antibody, e.g., described herein, or antigen-binding fragment thereof increases retention of surface cleaved human or mouse CDCP1 on a cell transfected with human cleaved CDCP1 by at least about 1.2 fold, at least about 1.3 fold, at least about 1.4 fold, at least about 1.5 fold, at least about 1.6 fold, at least about 1.7 fold, at least about 1.8 fold, at least about 1.9 fold, at least about 2 fold, at least about 2.1 fold, at least about 2.2 fold, at least about 2.3 fold, at least about 2.4 fold, at least about 2.5 fold, at least about 2.75 fold, at least about 3 fold, at least about 3.5 fold, at least about 4 fold, at least about 4.5 fold, at least about 5 fold, at least about 7.5 fold, at least about 10 fold, or at least about 20 fold.

An “effector function” refers to the interaction of an antibody Fc region with an Fc receptor or ligand, or a biochemical event that results therefrom. Exemplary “effector functions” include C1q binding, complement dependent cytotoxicity (CDC), Fc receptor binding, FcγR-mediated effector functions such as ADCC and antibody dependent cell-mediated phagocytosis (ADCP), and downregulation of a cell surface receptor (e.g., the B cell receptor; BCR). Such effector functions generally require the Fc region to be combined with a binding domain (e.g., an antibody variable domain).

An “Fc receptor” or “FcR” is a receptor that binds to the Fc region of an immunoglobulin. FcRs that bind to an IgG antibody comprise receptors of the FcγR family, including allelic variants and alternatively spliced forms of these receptors. The FcγR family consists of three activating (FcγRI, FcγRIII, and FcγRIV in mice; FcγRIA, FcγRIIA, and FcγRIIIA in humans) and one inhibitory (FcγRIIB) receptor. Various properties of human FcγRs are known in the art. The majority of innate effector cell types coexpress one or more activating FcγR and the inhibitory FcγRIIB, whereas natural killer (NK) cells selectively express one activating Fc receptor (FcγRIII in mice and FcγRIIIA in humans) but not the inhibitory FcγRIIB in mice and humans. Human IgG1 binds to most human Fc receptors and is considered equivalent to murine IgG2a with respect to the types of activating Fc receptors that it binds to.

An “Fc region” (fragment crystallizable region) or “Fc domain” or “Fc” refers to the C-terminal region of the heavy chain of an antibody that mediates the binding of the immunoglobulin to host tissues or factors, including binding to Fc receptors located on various cells of the immune system (e.g., effector cells) or to the first component (C1q) of the classical complement system. Thus, an Fc region comprises the constant region of an antibody excluding the first constant region immunoglobulin domain (e.g., CH1 or CL). In IgG, IgA and IgD antibody isotypes, the Fc region comprises two identical protein fragments, derived from the second (CH2) and third (CH3) constant domains of the antibody's two heavy chains; IgM and IgE Fc regions comprise three heavy chain constant domains (CH domains 2-4) in each polypeptide chain. For IgG, the Fc region comprises immunoglobulin domains CH2 and CH3 and the hinge between CH1 and CH2 domains. Although the definition of the boundaries of the Fc region of an immunoglobulin heavy chain might vary, as defined herein, the human IgG heavy chain Fc region is defined to stretch from an amino acid residue D221 for IgG1, V222 for IgG2, L221 for IgG3 and P224 for IgG4 to the carboxy-terminus of the heavy chain, wherein the numbering is according to the EU index as in Kabat. The CH2 domain of a human IgG Fc region extends from amino acid 237 to amino acid 340, and the CH3 domain is positioned on C-terminal side of a CH2 domain in an Fc region, i.e., it extends from amino acid 341 to amino acid 447 or 446 (if the C-terminal lysine residue is absent) or 445 (if the C-terminal glycine and lysine residues are absent) of an IgG. As used herein, the Fc region can be a native sequence Fc, including any allotypic variant, or a variant Fc (e.g., a non-naturally-occurring Fc).

A “native sequence Fc region” or “native sequence Fc” comprises an amino acid sequence that is identical to the amino acid sequence of an Fc region found in nature. Native sequence human Fc regions include a native sequence human IgG1 Fc region; native sequence human IgG2 Fc region; native sequence human IgG3 Fc region; and native sequence human IgG4 Fc region as well as naturally-occurring variants thereof. Native sequence Fc include the various allotypes of Fcs (see, e.g., Jefferis et al. (2009) mAbs 1:1).

The term “epitope” or “antigenic determinant” refers to a site on an antigen (e.g., cleaved CDCP1) to which an immunoglobulin or antibody specifically binds, e.g., as defined by the specific method used to identify it. Epitopes can be formed both from contiguous amino acids (usually a linear epitope) or noncontiguous amino acids juxtaposed by tertiary folding of a protein (usually a conformational epitope). Epitopes formed from contiguous amino acids are typically, but not always, retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods for determining what epitopes are bound by a given antibody (i.e., epitope mapping) are well known in the art and include, for example, immunoblotting and immunoprecipitation assays, wherein overlapping or contiguous peptides from (e.g., from cleaved CDCP1) are tested for reactivity with a given antibody (e.g., anti-cleaved CDCP1 antibody). Methods of determining spatial conformation of epitopes include techniques in the art and those described herein, for example, x-ray crystallography, x-ray co-crystallography, antigen mutational analysis, 2-dimensional nuclear magnetic resonance and HDX-MS (see, e.g., Epitope Mapping Protocols in Methods in Molecular Biology, Vol. 66, G. E. Morris, Ed. (1996)).

The term “epitope mapping” refers to the process of identification of the molecular determinants for antibody-antigen recognition.

The term “binds to the same epitope” with reference to two or more antibodies means that the antibodies bind to the same segment of amino acid residues, as determined by a given method. Techniques for determining whether antibodies bind to the “same epitope on cleaved CDCP1” with the antibodies described herein include, for example, epitope mapping methods, such as, x-ray analyses of crystals of antigen: antibody complexes which provides atomic resolution of the epitope and hydrogen/deuterium exchange mass spectrometry (HDX-MS). Other methods monitor the binding of the antibody to antigen fragments or mutated variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. Antibodies having the same VH and VL or the same CDR1, 2 and 3 sequences are expected to bind to the same epitope.

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, can be determined using known competition experiments, e.g., BIACORE® surface plasmon resonance (SPR) analysis. In some aspects, an antibody competes with, and inhibits binding of another antibody to a target by at least 50%, 60%, 70%, 80%, 90% or 100%. The level of inhibition or competition can be different depending on which antibody is the “blocking antibody” (i.e., the cold antibody that is incubated first with the target). Competition assays can be conducted as described, for example, in Ed Harlow and David Lane, Cold Spring Harb Protoc; 2006; doi: 10.1101/pdb.prot4277 or in Chapter 11 of “Using Antibodies” by Ed Harlow and David Lane, Cold Spring Harbor Laboratory Press, Cold Spring Harbor, NY, USA 1999. Two antibodies “cross-compete” if antibodies block each other both ways by at least 50%, i.e., regardless of whether one or the other antibody is contacted first with the antigen in the competition experiment.

Competitive binding assays for determining whether two antibodies compete or cross-compete for binding include: competition for binding to cells expressing cleaved CDCP1 (e.g, human or mouse cleaved CDCP1), e.g., by flow cytometry, such as described in the Examples. Other methods include: SPR (e.g., BIACORE®), solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using 1-125 label (see Morel et al., Mol. Immunol. 25 (1): 7 (1988)); solid phase direct biotin-avidin EIA (Cheung et al., Virology 176:546 (1990)); and direct labeled RIA. (Moldenhauer et al., Scand. J. Immunol. 32:77 (1990)).

As used herein, the terms “specific binding,” “selective binding,” “selectively binds,” and “specifically binds,” refer to antibody binding to an epitope on a predetermined antigen. Typically, the antibody (i) binds with an equilibrium dissociation constant (K_{D}) of approximately less than 10⁻⁷ M, such as approximately less than 10⁻⁸ M, 10⁻⁹ M or 10⁻¹⁰ M or even lower when determined by, e.g., surface plasmon resonance (SPR) technology in a BIACORE® 2000 instrument using the predetermined antigen, e.g., recombinant human cleaved CDCP1, as the analyte and the antibody as the ligand, or Scatchard analysis of binding of the antibody to antigen positive cells, and (ii) binds to the predetermined antigen with an affinity that is at least two-fold greater than its affinity for binding to a non-specific antigen (e.g., BSA, casein) other than the predetermined antigen or a closely-related antigen. Accordingly, an antibody that “specifically binds to human cleaved CDCP1” refers to an antibody that binds to cell bound human cleaved CDCP1 with a K_{D} of 10⁻⁷M or less, such as approximately less than 10⁻⁸ M, 10⁻⁹M or 10⁻¹⁰ M or even lower. An antibody that “cross-reacts with cynomolgus cleaved CDCP1” refers to an antibody that binds to cynomolgus cleaved CDCP1 with a K_{D} of 10⁻⁷M or less, such as approximately less than 10⁻⁸ M, 10⁻⁹ M or 10⁻¹⁰ M or even lower. In some aspects, such antibodies that do not cross-react with cleaved CDCP1 from a non-human species exhibit essentially undetectable binding against these proteins in standard binding assays.

The term “k_{assoc}” or “kₐ”, as used herein, is intended to refer to the association rate of a particular antibody-antigen interaction, whereas the term “k_{dis}” or “k_{d},” as used herein, is intended to refer to the dissociation rate of a particular antibody-antigen interaction. The term “K_{D}”, as used herein, is intended to refer to the dissociation constant, which is obtained from the ratio of k_{d} to kₐ (i.e., k_{d}/kₐ) and is expressed as a molar concentration (M). K_{D} values for antibodies can be determined using methods well established in the art. Available methods for determining the K_{D} of an antibody include surface plasmon resonance, a biosensor system such as a BIACORE® system or flow cytometry and Scatchard analysis.

As used herein, the term “high affinity” for an IgG antibody refers to an antibody having a K_{D} of 10⁻⁸ M or less, 10⁻⁹M or less, or 10⁻¹⁰ M or less for a target antigen. However, “high affinity” binding can vary for other antibody isotypes. For example, “high affinity” binding for an IgM isotype refers to an antibody having a K_{D} of 10⁻¹⁰ M or less, or 10⁻⁸ M or less.

The term “EC₅₀” in the context of an in vitro or in vivo assay using an antibody or antigen binding fragment thereof, refers to the concentration of an antibody or an antigen-binding fragment thereof that induces a response that is 50% of the maximal response, i.e., halfway between the maximal response and the baseline.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

A “polypeptide” refers to a chain comprising at least two consecutively linked amino acid residues, with no upper limit on the length of the chain. One or more amino acid residues in the protein can contain a modification such as, but not limited to, glycosylation, phosphorylation or disulfide bond formation. A “protein” can comprise one or more polypeptides.

The term “nucleic acid molecule,” as used herein, is intended to include DNA molecules and RNA molecules. A nucleic acid molecule can be single-stranded or double-stranded, and can be cDNA.

“Conservative amino acid substitutions” refer to substitutions of an amino acid residue with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). In some aspects, a predicted nonessential amino acid residue in an anti-human or mouse cleaved CDCP1 antibody is replaced with another amino acid residue from the same side chain family. Methods of identifying nucleotide and amino acid conservative substitutions which do not eliminate antigen binding are well-known in the art (see, e.g., Brummell et al., Biochem. 32:1180-1187 (1993); Kobayashi et al. Protein Eng. 12 (10): 879-884 (1999); and Burks et al. Proc. Natl. Acad. Sci. USA 94:412-417 (1997)).

For nucleic acids, the term “substantial homology” indicates that two nucleic acids, or designated sequences thereof, when optimally aligned and compared, are identical, with appropriate nucleotide insertions or deletions, in at least about 80% of the nucleotides, at least about 90% to 95%, or at least about 98% to 99.5% of the nucleotides. Alternatively, substantial homology exists when the segments will hybridize under selective hybridization conditions, to the complement of the strand.

For polypeptides, the term “substantial homology” indicates that two polypeptides, or designated sequences thereof, when optimally aligned and compared, are identical, with appropriate amino acid insertions or deletions, in at least about 80% of the amino acids, at least about 90% to 95%, or at least about 98% to 99.5% of the amino acids.

The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available at worldwideweb.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48): 444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences described herein can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, word length=12 to obtain nucleotide sequences homologous to the nucleic acid molecules described herein. BLAST protein searches can be performed with the XBLAST program, score=50, word length=3 to obtain amino acid sequences homologous to the protein molecules described herein. To obtain gapped alignments for comparison purposes, Gapped BLAST can be utilized as described in Altschul et al., (1997) Nucleic Acids Res. 25 (17): 3389-3402. When utilizing BLAST and Gapped BLAST programs, the default parameters of the respective programs (e.g., XBLAST and NBLAST) can be used. See worldwideweb.ncbi.nlm.nih.gov.

The nucleic acids can be present in whole cells, in a cell lysate, or in a partially purified or substantially pure form. A nucleic acid is “isolated” or “rendered substantially pure” when purified away from other cellular components or other contaminants, e.g., other cellular nucleic acids (e.g., the other parts of the chromosome) or proteins, by standard techniques, including alkaline/SDS treatment, CsCl banding, column chromatography, agarose gel electrophoresis and others well known in the art. See, F. Ausubel, et al., ed. Current Protocols in Molecular Biology, Greene Publishing and Wiley Interscience, New York (1987).

Nucleic acids, e.g., cDNA, can be mutated, in accordance with standard techniques to provide gene sequences. For coding sequences, these mutations, can affect amino acid sequence as desired. In particular, DNA sequences substantially homologous to or derived from native V, D, J, constant, switches and other such sequences described herein are contemplated (where “derived” indicates that a sequence is identical or modified from another sequence).

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments can be ligated. Another type of vector is a viral vector; wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” can be used interchangeably as the plasmid is the most commonly used form of vector. However, also included are other forms of expression vectors, such as viral vectors (e.g., replication defective retroviruses, adenoviruses and adeno-associated viruses), which serve equivalent functions.

The term “recombinant host cell” (or simply “host cell”), as used herein, is intended to refer to a cell that comprises a nucleic acid that is not naturally present in the cell, and can be a cell into which a recombinant expression vector has been introduced. It should be understood that such terms are intended to refer not only to the particular subject cell but to the progeny of such a cell. Because certain modifications can occur in succeeding generations due to either mutation or environmental influences, such progeny cannot, in fact, be identical to the parent cell, but are still included within the scope of the term “host cell” as used herein.

An “immune response” is as understood in the art, and generally refers to a biological response within a vertebrate against foreign agents or abnormal, e.g., cancerous cells, which response protects the organism against these agents and diseases caused by them. An immune response is mediated by the action of one or more cells of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell, a Th cell, a CD4⁺ cell, a CD8⁺ T cell, or a Treg cell, or activation or inhibition of any other cell of the immune system, e.g., NK cell.

An “immunomodulator” or “immunoregulator” refers to an agent, e.g., an agent targeting a component of a signaling pathway that can be involved in modulating, regulating, or modifying an immune response. “Modulating,” “regulating,” or “modifying” an immune response refers to any alteration in a cell of the immune system or in the activity of such cell (e.g., an effector T cell, such as a Th1 cell). Such modulation includes stimulation or suppression of the immune system which can be manifested by an increase or decrease in the number of various cell types, an increase or decrease in the activity of these cells, or any other changes which can occur within the immune system. Both inhibitory and stimulatory immunomodulators have been identified, some of which can have enhanced function in a tumor microenvironment. In some aspects, the immunomodulator targets a molecule on the surface of a T cell. An “immunomodulatory target” or “immunoregulatory target” is a molecule, e.g., a cell surface molecule, that is targeted for binding by, and whose activity is altered by the binding of, a substance, agent, moiety, compound or molecule. Immunomodulatory targets include, for example, receptors on the surface of a cell (“immunomodulatory receptors”) and receptor ligands (“immunomodulatory ligands”).

“Immunotherapy” refers to the treatment of a subject afflicted with, or at risk of contracting or suffering a recurrence of, a disease by a method comprising inducing, enhancing, suppressing or otherwise modifying the immune system or an immune response.

“Immuno stimulating therapy” or “immuno stimulatory therapy” refers to a therapy that results in increasing (inducing or enhancing) an immune response in a subject for, e.g., treating cancer.

“Potentiating an endogenous immune response” means increasing the effectiveness or potency of an existing immune response in a subject. This increase in effectiveness and potency can be achieved, for example, by overcoming mechanisms that suppress the endogenous host immune response or by stimulating mechanisms that enhance the endogenous host immune response.

“T effector” (“Teff”) cells refers to T cells (e.g., CD4⁺ and CD8⁺ T cells) with cytolytic activities as well as T helper (Th) cells, e.g., Th1 cells, which cells secrete cytokines and activate and direct other immune cells, but does not include regulatory T cells (Treg cells). Certain anti-cleaved CDCP1 antibodies described herein, or antigen binding fragments thereof activate T_{eff} cells, e.g., CD4⁺ and CD8⁺ T_{eff} cells and Th1 cells.

An increased ability to stimulate an immune response or the immune system, can result from an enhanced agonist activity of T cell co-stimulatory receptors and/or an enhanced antagonist activity of inhibitory receptors. An increased ability to stimulate an immune response or the immune system can be reflected by a fold increase of the EC₅₀ or maximal level of activity in an assay that measures an immune response, e.g., an assay that measures changes in cytokine or chemokine release, cytolytic activity (determined directly on target cells or indirectly via detecting CD107a or granzymes) and proliferation. The ability to stimulate an immune response or the immune system activity can be enhanced by at least 10%, 30%, 50%, 75%, 2 fold, 3 fold, 5 fold or more.

As used herein, the term “linked” refers to the association of two or more molecules. The linkage can be covalent or non-covalent. The linkage also can be genetic (i.e., recombinantly fused). Such linkages can be achieved using a wide variety of art recognized techniques, such as chemical conjugation and recombinant protein production.

As used herein, “administering” refers to the physical introduction of a composition comprising a therapeutic agent to a subject, using any of the various methods and delivery systems known to those skilled in the art. Different routes of administration for the anti-cleaved CDCP1 antibodies (e.g., anti-human cleaved CDCP1) described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, an antibody described herein can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

As used herein, the term “T cell-mediated response” refers to a response mediated by T cells, including effector T cells (e.g., CD8⁺ cells) and helper T cells (e.g., CD4⁺ cells). T cell mediated responses include, for example, T cell cytotoxicity and proliferation.

As used herein, the term “cytotoxic T lymphocyte (CTL) response” refers to an immune response induced by cytotoxic T cells. CTL responses are mediated primarily by CD8⁺ T cells.

As used herein, the phrase “inhibits growth of a tumor” includes any measurable decrease in the growth of a tumor, e.g., the inhibition of growth of a tumor by at least about 10%, for example, at least about 20%, at least about 30%, at least about 40%, at least about 50%, at least about 60%, at least about 70%, at least about 80%, at least about 90%, at least about 99%, or 100%. In some aspects, inhibition of tumor growth is measured as the percent tumor growth inhibition (TGI %). TGI % can be determined by calculating the TGI at dat “t” calculated from all treatment animals according to the formula: [1−((Tₜ/T₀)/(Cₜ/C₀))]/[(Cₜ−C₀)/Cₜ]*100 [Formula 1], where Tₜ=individual tumor size of treated animal at time ‘t’, T₀=individual tumor size of treated animal at first measurement, Cₜ=median tumors size of control animals at time ‘t’, C₀=median tumor size of control animals at first measurement.

As used herein, “cancer” refers a broad group of diseases characterized by the uncontrolled growth of abnormal cells in the body. Unregulated cell division can result in the formation of malignant tumors or cells that invade neighboring tissues and can metastasize to distant parts of the body through the lymphatic system or bloodstream.

The terms “treat,” “treating,” and “treatment,” as used herein, refer to any type of intervention or process performed on, or administering an active agent to, the subject with the objective of reversing, alleviating, ameliorating, inhibiting, or slowing down or preventing the progression, development, severity or recurrence of a symptom, complication, condition or biochemical indicia associated with a disease or enhancing overall survival. Treatment can be of a subject having a disease or a subject who does not have a disease (e.g., for prophylaxis).

The term “effective dose” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, an increase in overall survival (the length of time from either the date of diagnosis or the start of treatment for a disease, such as cancer, that patients diagnosed with the disease are still alive), or a prevention of impairment or disability due to the disease affliction. A therapeutically effective amount or dosage of a drug includes a “prophylactically effective amount” or a “prophylactically effective dosage”, which is any amount of the drug that, when administered alone or in combination with another therapeutic agent to a subject at risk of developing a disease or of suffering a recurrence of disease, inhibits the development or recurrence of the disease. The ability of a therapeutic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

By way of example, an anti-cancer agent is a drug that promotes cancer regression in a subject. In some aspects, a therapeutically effective amount of the drug promotes cancer regression to the point of eliminating the cancer. “Promoting cancer regression” means that administering an effective amount of the drug, alone or in combination with an antineoplastic agent, results in a reduction in tumor growth or size, necrosis of the tumor, a decrease in severity of at least one disease symptom, an increase in frequency and duration of disease symptom-free periods, an increase in overall survival, a prevention of impairment or disability due to the disease affliction, or otherwise amelioration of disease symptoms in the patient. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

By way of example for the treatment of tumors, a therapeutically effective amount or dosage of the drug inhibits cell growth or tumor growth by at least about 20%, by at least about 40%, by at least about 60%, or by at least about 80% relative to untreated subjects. In some aspects, a therapeutically effective amount or dosage of the drug completely inhibits cell growth or tumor growth, i.e., inhibits cell growth or tumor growth by 100%. The ability of a compound to inhibit tumor growth can be evaluated using the assays described infra. Alternatively, this property of a composition can be evaluated by examining the ability of the compound to inhibit cell growth, such inhibition can be measured in vitro by assays known to the skilled practitioner. In some aspects described herein, tumor regression can be observed and continue for a period of at least about 20 days, at least about 40 days, or at least about 60 days.

The term “patient” includes human and other mammalian subjects that receive either prophylactic or therapeutic treatment.

As used herein, the term “subject” includes any human or non-human animal. For example, the methods and compositions described herein can be used to treat a subject having cancer. The term “non-human animal” includes all vertebrates, e.g., mammals and non-mammals, such as non-human primates, sheep, dog, cow, chickens, amphibians, reptiles, etc.

As used herein, the terms “ug” and “uM” are used interchangeably with “μg” and “μM,” respectively.

Various aspects described herein are described in further detail in the following subsections.

### II. Anti-Human and Mouse Cleaved CDCP1 Antibodies

Described herein are antibodies, e.g., fully human antibodies, that are capable of specifically binding to cleaved CDCP1; that specifically bind to the same cleaved CDCP1 epitope as a reference antibody; and/or that cross-compete for binding to a cleaved CDCP1 epitope with a reference antibody. In some aspects, the anti-CDCP1 antibodies or antigen-binding fragments thereof that specifically bind to cleaved CDCP1, preferentially bind to the cleaved CDCP1, and are characterized by particular functional features or properties. For example, the antibodies specifically bind to mammalian (e.g., human and mouse) cleaved CDCP1 and exhibit one or more of the following functional properties:
(a) inhibiting tumor growth and/or metastasis;
(b) reducing tumor volume;
(c) increasing progression-free survival;
(d) increasing overall survival;
(e) promote CDCP1 internalization and/or degradation; and
(f) any combination thereof.

In some aspects, the anti-CDCP1 antibodies or antigen-binding fragments thereof described herein do not bind to a full-length human CDCP1 at a detectable level.

In some aspects, anti-human cleaved CDCP1 antibodies or antigen-binding fragments thereof described herein bind to human cleaved CDCP1 with high affinity, for example, with a K_{D} of 10⁻⁶ M or less, 10⁻⁷M or less, 10⁻⁸ M or less, 10⁻⁹M or less, 10⁻¹⁰ M or less, 10⁻¹¹ M or less, 10⁻¹² M or less, 10⁻¹² M to 10⁻⁷M, 10⁻¹¹ M to 10⁻⁷ M, 10⁻¹⁰ M to 10⁻⁷M, or 10⁻⁹ M to 10⁻⁷ M. In some aspects, the anti-CDCP1 antibody binds to human CDCP1, e.g., as determined by biolayer interferometry analysis using an Octet instrument (ForteBio), with a K_{D} of 10⁻⁶ M or less, 10⁻⁷M or less, 10⁻⁸ M or less, 10⁻⁹ M (1 nM) or less, 10⁻¹⁰ M or less, 10⁻¹² M to 10⁻⁷M, 10⁻¹¹ M to 10⁻⁷M, 10⁻¹⁰ M to 10⁻⁷M, 10⁻⁹ M to 10⁻⁷M, or 10⁻⁸ M to 10⁻⁷M. In some aspects, an anti-human cleaved CDCP1 antibody binds to human cleaved CDCP1, e.g., as determined by ELISA, with an EC₅₀ of EC₅₀ of 100 nM or less, 10 nM or less, 1 nM or less, 100 nM to 0.01 nM, 100 nM to 0.1 nM, 100 nM to 1 nM, or 10 nM to 1 nM, or 10 ug/mL or less, 5 ug/mL or less, 1 ug/mL or less, 0.9 ug/mL or less, 0.8 ug/mL or less, 0.7 ug/mL or less, 0.6 ug/mL or less, 0.5 ug/mL or less, 0.4 ug/mL or less, 0.3 ug/mL or less, 0.2 ug/mL or less, 0.1 ug/mL or less, 0.05 ug/mL or less, or 0.01 ug/mL or less. In some aspects, anti-cleaved CDCP1 antibodies described herein bind to mouse cleaved CDCP1, for example, with a K_{D} of 10⁻⁶ M or less, 10⁻⁷M or less, 10⁻⁸ M or less, 10⁻⁹ M or less, 10⁻¹⁰ M or less, 10⁻¹¹ M or less, 10⁻¹² M or less, 10⁻¹² M to 10⁻⁷M, 10⁻¹¹ M to 10⁻⁷M, 10⁻¹⁰ M to 10⁻⁷M, or 10⁻⁹ M to 10⁻⁷M. In some aspects, anti-cleaved CDCP1 antibodies or antigen-binding fragments thereof described herein bind to cyno cleaved CDCP1, for example, with a K_{D} of 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less, 10⁻¹² M to 10⁻⁷M, 10⁻¹¹ M to 10⁻⁷M, 10⁻¹⁰ M to 10⁻⁷M, or 10⁻⁹ M to 10⁻⁷M.

In some aspects, the anti-human cleaved CDCP1 antibody or antigen-binding fragment thereof specifically binds to human cleaved CDCP1 with a K_{D} of about 5×10⁻⁴ M or less, about 1×10⁻⁴ M or less, 5×10⁻⁵ M or less, about 1×10⁻⁵ M or less, about 1×10⁻⁶ M or less, about 1×10⁻⁷ M or less, or about 1×10⁻⁸ M or less, wherein K_{D} is measured by biolayer interferometry analysis using an Octet instrument (ForteBio).

In some aspects, the anti-human cleaved CDCP1 antibody or antigen-binding fragment thereof specifically binds human cleaved CDCP1 with an on rate (kₒₙ) of at least about 1×10³ ms⁻¹, at least about 5×10³ ms⁻¹, at least about 1×10⁴ ms⁻¹, at least about 5×10⁴ ms⁻¹, at least about 1×10⁵ ms⁻¹, at least about 5×10⁵ ms⁻¹, or at least about 1×10⁶ ms⁻¹, wherein kₒₙ is measured by biolayer interferometry analysis using an Octet instrument (ForteBio).

In some aspects, the anti-CDCP1 antibody or antigen-binding fragment thereof specifically binds human CDCP1 with an off rate (k_{off}) of at least about 1×10³ ms⁻¹, at least about 5×10³ ms⁻¹, at least about 1×10⁴ ms⁻¹, at least about 5×10⁴ ms⁻¹, at least about 1×10⁵ ms⁻¹, at least about 5×10⁵ ms⁻¹, or at least about 1×10⁶ ms⁻¹, wherein k_{off} is measured by biolayer interferometry analysis using an Octet instrument (ForteBio).

In some aspects, the anti-human cleaved CDCP1 antibody or antigen-binding fragment thereof comprises a light chain variable region (VL) and a heavy chain variable region (VH); wherein the VL comprises a VL complementarity determining region (CDR) 1, a VL-CDR2, and a VL-CDR3, and the VH comprises a VH-CDR1, a VH-CDR2, and a VH-CDR3 sequences of SEQ ID NOs: 1 (SVSSAVA), 2 (SASSLY), 268 (SX₁X₂X₃X₄X₅), 269 (X₆FSSX₇SI), 270 (SIYPYSGSTX₈), and 271 (X₉X₁₀X₁₂SX₁₂YSHTWWVSYGX₁₃) or 272 (X₁₄YWVX₁₅FWYGHFSYYRPAL), respectively, wherein:
X₁=Glycine (G), Serine(S), Methionine (M), Leucine (L), Valine (V), or Arginine (R);
X₂=Glutamine (Q), Serine(S), Glutamic acid (E), Asparagine (N), Lysine (K), Proline (P), Arginine (R), Leucine (L), or Histidine (H);
X₃=Arginine (R), Serine(S), Valine (V), Tryptophan (W), Leucine (L), Lysine (K), Methionine (M), Glutamine (Q), or Proline (P);
X₄=Proline (P), Leucine (L), Threonine (T), or Serine(S);
X₅=Isoleucine (I), Alanine (A), Methionine (M), Lysine (K), Valine (V), Leucine (L), Phenylalanine (F);
X₆=No Amino Acid, Aspartic acid (D), or Asparagine (N);
X₇=Serine(S) or Tyrosine (Y);
X₈=Serine(S) or Tyrosine (Y);
X₉=Glutamine (Q), Arginine (R), or Lysine (K);
X₁₀=Serine(S), Asparagine (N), Threonine (T), Glycine (G), Alanine (A), or Aspartic acid (D);
X₁₁=Glutamine (Q) or Histidine (H);
X₁₂=Tyrosine (Y) or Phenylalanine (F);
X₁₃=Methionine (M), Alanine (A), Isoleucine (I), Leucine (L), or Valine (V);
X₁₄=Threonine (T) or Isoleucine (I); and
X₁₅=Glutamine (Q) or Aspartic acid (D).

In some aspects, the anti-human cleaved CDCP1 antibody or antigen-binding fragment thereof comprises a light chain variable region (VL) and a heavy chain variable region (VH); wherein the VL comprises a VL complementarity determining region (CDR) 1 sequence of SEQ ID NO: 1 (SVSSAVA), a VL-CDR2 sequence of SEQ ID NO: 2 (SASSLY), and a VL-CDR3 sequence of SEQ ID NOs: 8 (TGQRPM), 23 (FMRPAF), 16 (TAQSPL), 11 (VELVPM), 12 (AGKRPL), or 14 (LGVRAA), and the VH comprises a VH-CDR1 sequence of SEQ ID NO: 269 (X₁FSSX₂SI), a VH-CDR2 sequence of SEQ ID NO: 270 (SIYPYSGSTX₃), and a VH-CDR3 sequence of SEQ ID NO: 271 (X₄X₅X₆SX₇YSHTWWVSYGX₈) or SEQ ID NO: 272 (X₉YWVX₁₀FWYGHFSYYRPAL), respectively, wherein:
X₁=No Amino Acid, Aspartic acid (D), or Asparagine (N);
X₂=Serine(S) or Tyrosine (Y);
X₃=Serine(S) or Tyrosine (Y);
X₄=Glutamine (Q), Arginine (R), or Lysine (K);
X₅=Serine(S), Asparagine (N), Threonine (T), Glycine (G), Alanine (A), or Aspartic acid (D);
X₆=Glutamine (Q) or Histidine (H);
X₇=Tyrosine (Y) or Phenylalanine (F);
X₈=Methionine (M), Alanine (A), Isoleucine (I), Leucine (L), or Valine (V);
X₉=Threonine (T) or Isoleucine (I); and
X₁₀=Glutamine (Q) or Aspartic acid (D).

In some aspects, the anti-human cleaved CDCP1 antibody or antigen-binding fragment thereof comprises a light chain variable region (VL) and a heavy chain variable region (VH); wherein the VL comprises a VL complementarity determining region (CDR) 1 (VL-CDR1), a VL-CDR2, and a VL-CDR3 and the VH comprises a VH-CDR1, a VH-CDR2, and a VH-CDR3; wherein the VL-CDR3 comprises an amino acid sequence having at least about 90%, at least about 95%, at least about 96%, at least about 97%, at least about 98%, or at least about 99% sequence identity to an amino acid sequence selected from the group consisting of SEQ ID NOs: 3-25. In some aspects, the anti-human cleaved CDCP1 antibody or antigen-binding fragment thereof comprises a VL-CDR3 comprising an amino acid sequence selected from the group consisting of SEQ ID NOs: 3-25, as disclosed in Table 1 below. In some aspects, the anti-CDCP1 antibody or antigen-binding fragment thereof comprises a VL-CDR3 comprising the amino acid sequence as set forth in SEQ ID NO: 3.

In some aspects, the anti-human cleaved CDCP1 antibody or antigen-binding fragment thereof comprises a VH-CDR2 comprising an amino acid sequence having at least about 90%, at least about 95%, at least about 96%, at least about 97%, at least about 98%, or at least about 99% sequence identity to an amino acid sequence of SEQ ID NO: 30 or 31. In some aspects, the anti-human cleaved CDCP1 antibody comprises a VH-CDR2 comprising an amino acid sequence of SEQ ID NO: 30 or 31, as disclosed in Table 1 below. In one aspect, the anti-human cleaved CDCP1 antibody or antigen-binding fragment thereof comprises a VH-CDR2 comprising the amino acid sequence as set forth in SEQ ID NO: 30.

In some aspects, the anti-human cleaved CDCP1 antibody or antigen-binding fragment thereof comprises a VH-CDR1 comprising an amino acid sequence having at least about 90%, at least about 95%, at least about 96%, at least about 97%, at least about 98%, or at least about 99% sequence identity to an amino acid sequence selected from the group consisting of SEQ ID NOs: 26-29, 109, and 111. In some aspects, the anti-human cleaved CDCP1 antibody or antigen-binding fragment thereof comprises a VH-CDR1 comprising an amino acid sequence selected from the group consisting of SEQ ID NOs: 26-29, 109, and 111, as disclosed in Table 1 below. In some aspects, the anti-human cleaved CDCP1 antibody or antigen-binding fragment thereof comprises a VH-CDR1 comprising the amino acid sequence as set forth in SEQ ID NO: 26.

In some aspects, the anti-human cleaved CDCP1 antibody or antigen-binding fragment thereof comprises a VL-CDR1 comprising an amino acid sequence having at least about 90%, at least about 95%, at least about 96%, at least about 97%, at least about 98%, or at least about 99% sequence identity to an amino acid sequence of SEQ ID NO: 1. In some aspects, the anti-human cleaved CDCP1 antibody or antigen-binding fragment thereof comprises a VL-CDR1 comprising an amino acid sequence of SEQ ID NO: 1, as disclosed in Table 1 below.

In some aspects, the anti-human cleaved CDCP1 antibody or antigen-binding fragment thereof comprises a VL-CDR2 comprising an amino acid sequence having at least about 90%, at least about 95%, at least about 96%, at least about 97%, at least about 98%, or at least about 99% sequence identity to an amino acid sequence of SEQ ID NO: 2. In some aspects, the anti-human cleaved CDCP1 antibody or antigen-binding fragment thereof comprises a VL-CDR2 comprising an amino acid sequence of SEQ ID NO: 2, as disclosed in Table 1 below.

In some aspects, the anti-human cleaved CDCP1 antibody or antigen-binding fragment thereof comprises a VH-CDR3 comprising an amino acid sequence having at least about 90%, at least about 95%, at least about 96%, at least about 97%, at least about 98%, or at least about 99% sequence identity to an amino acid sequence selected from the group consisting of SEQ ID NOs: 20, 32-47, and 105. In some aspects, the anti-human cleaved CDCP1 antibody or antigen-binding fragment thereof comprises a VH-CDR3 comprising an amino acid sequence selected from the group consisting of SEQ ID NOs: 20, 32-47, and 105 as disclosed in Table 1 below. In some aspects, the anti-human cleaved CDCP1 antibody or antigen-binding fragment thereof comprises a VH-CDR3 comprising the amino acid sequence as set forth in SEQ ID NO: 32. In some aspects, the anti-human cleaved CDCP1 antibody or antigen-binding fragment thereof comprises a VH-CDR3 comprising the amino acid sequence as set forth in SEQ ID NO: 33. In some aspects, the anti-human cleaved CDCP1 antibody or antigen-binding fragment thereof comprises a VH-CDR3 comprising the amino acid sequence as set forth in SEQ ID NO: 34. In some aspects, the anti-human cleaved CDCP1 antibody or antigen-binding fragment thereof comprises a VH-CDR3 comprising the amino acid sequence as set forth in SEQ ID NO: 35. In some aspects, the anti-human cleaved CDCP1 antibody or antigen-binding fragment thereof comprises a VH-CDR3 comprising the amino acid sequence as set forth in SEQ ID NO: 36.

In certain aspects, the anti-human cleaved CDCP1 antibody or antigen-binding fragment thereof comprises
(a) the VL-CDR1 comprises the amino acid sequence set forth in SEQ ID NO: 1, the VL-CDR2 comprises the amino acid sequence set forth in SEQ ID NO: 2, the VL-CDR3 comprises the amino acid sequence set forth in SEQ ID NO: 3, the VH-CDR1 comprises the amino acid sequence set forth in SEQ ID NO: 26, the VH-CDR2 comprises the amino acid sequence set forth in SEQ ID NO: 30, and the VH-CDR3 comprises the amino acid sequence set forth in SEQ ID NO: 32;
(b) the VL-CDR1 comprises the amino acid sequence set forth in SEQ ID NO: 1, the VL-CDR2 comprises the amino acid sequence set forth in SEQ ID NO: 2, the VL-CDR3 comprises the amino acid sequence set forth in SEQ ID NO: 3, the VH-CDR1 comprises the amino acid sequence set forth in SEQ ID NO: 26, the VH-CDR2 comprises the amino acid sequence set forth in SEQ ID NO: 30, and the VH-CDR3 comprises the amino acid sequence set forth in SEQ ID NO: 33;
(c) the VL-CDR1 comprises the amino acid sequence set forth in SEQ ID NO: 1, the VL-CDR2 comprises the amino acid sequence set forth in SEQ ID NO: 2, the VL-CDR3 comprises the amino acid sequence set forth in SEQ ID NO: 3, the VH-CDR1 comprises the amino acid sequence set forth in SEQ ID NO: 26, the VH-CDR2 comprises the amino acid sequence set forth in SEQ ID NO: 30, and the VH-CDR3 comprises the amino acid sequence set forth in SEQ ID NO: 34;
(d) the VL-CDR1 comprises the amino acid sequence set forth in SEQ ID NO: 1, the VL-CDR2 comprises the amino acid sequence set forth in SEQ ID NO: 2, the VL-CDR3 comprises the amino acid sequence set forth in SEQ ID NO: 3, the VH-CDR1 comprises the amino acid sequence set forth in SEQ ID NO: 26, the VH-CDR2 comprises the amino acid sequence set forth in SEQ ID NO: 30, and the VH-CDR3 comprises the amino acid sequence set forth in SEQ ID NO: 35; or
(e) the VL-CDR1 comprises the amino acid sequence set forth in SEQ ID NO: 1, the VL-CDR2 comprises the amino acid sequence set forth in SEQ ID NO: 2, the VL-CDR3 comprises the amino acid sequence set forth in SEQ ID NO: 3, the VH-CDR1 comprises the amino acid sequence set forth in SEQ ID NO: 26, the VH-CDR2 comprises the amino acid sequence set forth in SEQ ID NO: 30, and the VH-CDR3 comprises the amino acid sequence set forth in SEQ ID NO: 36.

TABLE 1
Variable Light and Heavy Chain CDR Amino Acid Sequences of Anti-Human
Cleaved CDCP1 Antibodies

| Name | CDR L1 | CDR L2 | CDR L3 | CDR H1 | CDR H2 | CDR H3 |
|---|---|---|---|---|---|---|
| CL03 Fab | SVSSAVA | SASSLY | SGQRPI | FSSYSI | SIYPYSGS | QSHSYYSHTW |
| | SEQ ID | SEQ ID | SEQ ID | SEQ ID | TY | WVSYGM |
| | NO: 1 | NO: 2 | NO: 3 | NO: 26 | SEQ ID | SEQ ID NO: 32 |
| | | | | | NO: 30 | |
| CL03 IgG | SVSSAVA | SASSLY | SGQRPI | FSSYSI | SIYPYSGS | QSHSYYSHTW |
| | SEQ ID | SEQ ID | SEQ ID | SEQ ID | TY | WVSYGM |
| | NO: 1 | NO: 2 | NO: 3 | NO: 26 | SEQ ID | SEQ ID NO: 32 |
| | | | | | NO: 30 | |
| CL03 IgG | SVSSAVA | SASSLY | SGQRPI | FSSYSI | SIYPYSGS | QSHSYYSHTW |
| H1-NtoD | SEQ ID | SEQ ID | SEQ ID | SEQ ID | TY | WVSYGM |
| | NO: 1 | NO: 2 | NO: 3 | NO: 26 | SEQ ID | SEQ ID NO: 32 |
| | | | | | NO: 30 | |
| CL03 IgG | SVSSAVA | SASSLY | SGQRPI | FSSYSI | SIYPYSGS | QSHSYYSHTW |
| H1-NtoD | SEQ ID | SEQ ID | SEQ ID | SEQ ID | TY | WVSYGA |
| H3-MtoA | NO: 1 | NO: 2 | NO: 3 | NO: 26 | SEQ ID | SEQ ID NO: 33 |
| (“IgG101”) | | | | | NO: 30 | |
| CL03 IgG | SVSSAVA | SASSLY | SGQRPI | FSSYSI | SIYPYSGS | QSHSYYSHTW |
| H1-NtoD | SEQ ID | SEQ ID | SEQ ID | SEQ ID | TY | WVSYGI |
| H3-MtoI | NO: 1 | NO: 2 | NO: 3 | NO: 26 | SEQ ID | SEQ ID NO: 34 |
| | | | | | NO: 30 | |
| CL03 IgG | SVSSAVA | SASSLY | SGQRPI | FSSYSI | SIYPYSGS | QSHSYYSHTW |
| H1-NtoD | SEQ ID | SEQ ID | SEQ ID | SEQ ID | TY | WVSYGL |
| H3-MtoL | NO: 1 | NO: 2 | NO: 3 | NO: 26 | SEQ ID | SEQ ID NO: 35 |
| | | | | | NO: 30 | |
| CL03 IgG | SVSSAVA | SASSLY | SGQRPI | FSSYSI | SIYPYSGS | QSHSYYSHTW |
| H1-NtoD | SEQ ID | SEQ ID | SEQ ID | SEQ ID | TY | WVSYGV |
| H3-Mto V | NO: 1 | NO: 2 | NO: 3 | NO: 26 | SEQ ID | SEQ ID NO: 36 |
| | | | | | NO: 30 | |
| CL07 Fab | SVSSAVA | SASSLY | SSSSLI | FSSSSI | SIYPYSGS | TYWVQFWYG |
| | SEQ ID | SEQ ID | SEQ ID | SEQ ID | TS | HFSYYRPAL |
| | NO: 1 | NO: 2 | NO: 4 | NO: 27 | SEQ ID | SEQ ID NO: 37 |
| | | | | | NO: 31 | |
| CL07 IgG | SVSSAVA | SASSLY | SSSSLI | FSSSSI | SIYPYSGS | TYWVQFWYG |
| | SEQ ID | SEQ ID | SEQ ID | SEQ ID | TS | HFSYYRPAL |
| | NO: 1 | NO: 2 | NO: 4 | NO: 27 | SEQ ID | SEQ ID NO: 37 |
| | | | | | NO: 31 | |
| CL07 IgG | SVSSAVA | SASSLY | SSSSLI | FSSSSI | SIYPYSGS | TYWVQFWYG |
| H1-NtoD | SEQ ID | SEQ ID | SEQ ID | SEQ ID | TS | HFSYYRPAL |
| | NO: 1 | NO: 2 | NO: 4 | NO: 27 | SEQ ID | SEQ ID NO: 37 |
| | | | | | NO: 31 | |
| IgG3H7L | SVSSAVA | SASSLY | SSSSLI | (D)FSSYSI | SIYPYSGS | QSHSYYSHTW |
| | SEQ ID | SEQ ID | SEQ ID | SEQ ID | TY | WVSYGM |
| | NO: 1 | NO: 2 | NO: 4 | NO: 28 | SEQ ID | SEQ ID NO: 32 |
| | | | | | NO: 30 | |
| IgGA01 | SVSSAVA | SASSLY | SGQRPI | (D)FSSYSI | SIYPYSGS | QNHSYYSHTW |
| | SEQ ID | SEQ ID | SEQ ID | SEQ ID | TY | WVSYGM |
| | NO: 1 | NO: 2 | NO: 3 | NO: 28 | SEQ ID | SEQ ID NO: 38 |
| | | | | | NO: 30 | |
| IgGA03 | SVSSAVA | SASSLY | SGQRPI | (D)FSSYSI | SIYPYSGS | QTHSYYSHTW |
| | SEQ ID | SEQ ID | SEQ ID | SEQ ID | TY | WVSYGM |
| | NO: 1 | NO: 2 | NO: 3 | NO: 28 | SEQ ID | SEQ ID NO: 39 |
| | | | | | NO: 30 | |
| IgGA04 | SVSSAVA | SASSLY | SGQRPI | (D)FSSYSI | SIYPYSGS | QGHSYYSHTW |
| | SEQ ID | SEQ ID | SEQ ID | SEQ ID | TY | WVSYGM |
| | NO: 1 | NO: 2 | NO: 3 | NO: 28 | SEQ ID | SEQ ID NO: 40 |
| | | | | | NO: 30 | |
| IgGA05 | SVSSAVA | SASSLY | SGQRPI | (D)FSSYSI | SIYPYSGS | QAHSYYSHTW |
| | SEQ ID | SEQ ID | SEQ ID | SEQ ID | TY | WVSYGM |
| | NO: 1 | NO: 2 | NO: 3 | NO: 28 | SEQ ID | SEQ ID NO: 41 |
| | | | | | NO: 30 | |
| IgGA06 | SVSSAVA | SASSLY | SGQRPI | (D)FSSYSI | SIYPYSGS | QGQSYYSHTW |
| | SEQ ID | SEQ ID | SEQ ID | SEQ ID | TY | WVSYGM |
| | NO: 1 | NO: 2 | NO: 3 | NO: 28 | SEQ ID | SEQ ID NO: 42 |
| | | | | | NO: 30 | |
| IgGA08 | SVSSAVA | SASSLY | SGERPI | (D)FSSYSI | SIYPYSGS | QSHSYYSHTW |
| | SEQ ID | SEQ ID | SEQ ID | SEQ ID | TY | WVSYGM |
| | NO: 1 | NO: 2 | NO: 5 | NO: 28 | SEQ ID NO: | SEQ ID NO: 32 |
| | | | | | 30 | |
| IgGA09 | SVSSAVA | SASSLY | SGNVP | (D)FSSYSI | SIYPYSGS | QSHSYYSHTW |
| | SEQ ID | SEQ ID | A | SEQ ID | TY | WVSYGM |
| | NO: 1 | NO: 2 | SEQ ID | NO: 28 | SEQ ID | SEQ ID NO: 32 |
| | | | NO: 6 | | NO: 30 | |
| IgGA10 | SVSSAVA | SASSLY | SGKRP | (D)FSSYSI | SIYPYSGS | QSHSYYSHTW |
| | SEQ ID | SEQ ID | M | SEQ ID | TY | WVSYGM |
| | NO: 1 | NO: 2 | SEQ ID | NO: 28 | SEQ ID | SEQ ID NO: 32 |
| | | | NO: 7 | | NO: 30 | |
| IgGA11 | SVSSAVA | SASSLY | TGQRP | (D)FSSYSI | SIYPYSGS | RSHSYYSHTW |
| | SEQ ID | SEQ ID | M | SEQ ID | TY | WVSYGM |
| | NO: 1 | NO: 2 | SEQ ID | NO: 28 | SEQ ID | SEQ ID NO: 43 |
| | | | NO: 8 | | NO: 30 | |
| IgGB01 | SVSSAVA | SASSLY | SMQWP | (D)FSSYSI | SIYPYSGS | QSHSYYSHTW |
| | SEQ ID | SEQ ID | K | SEQ ID | TY | WVSYGM |
| | NO: 1 | NO: 2 | SEQ ID | NO: 28 | SEQ ID | SEQ ID NO: 32 |
| | | | NO: 9 | | NO: 30 | |
| IgGB03 | SVSSAVA | SASSLY | SLQRP | (D)FSSYSI | SIYPYSGS | QSHSFYSHTW |
| | SEQ ID | SEQ ID | M | SEQ ID | TY | WVSYGM |
| | NO: 1 | NO: 2 | SEQ ID | NO: 28 | SEQ ID | SEQ ID NO: 45 |
| | | | NO: 10 | | NO: 30 | |
| IgGB04 | SVSSAVA | SASSLY | VELVP | (D)FSSYSI | SIYPYSGS | RSHSYYSHTW |
| | SEQ ID | SEQ ID | M | SEQ ID | TY | WVSYGM |
| | NO: 1 | NO: 2 | SEQ ID | NO: 28 | SEQ ID | SEQ ID NO: 43 |
| | | | NO: 11 | | NO: 30 | |
| IgGB07 | SVSSAVA | SASSLY | AGKRP | (D)FSSYSI | SIYPYSGS | QDHSYYSHTW |
| | SEQ ID | SEQ ID | L | SEQ ID | TY | WVSYGM |
| | NO: 1 | NO: 2 | SEQ ID | NO: 28 | SEQ ID | SEQ ID NO: 44 |
| | | | NO: 12 | | NO: 30 | |
| IgGB08 | SVSSAVA | SASSLY | SVDLT | (D)FSSYSI | SIYPYSGS | RSHSYYSHTW |
| | SEQ ID | SEQ ID | A | SEQ ID | TY | WVSYGM |
| | NO: 1 | NO: 2 | SEQ ID | NO: 28 | SEQ ID | SEQ ID NO: 43 |
| | | | NO: 13 | | NO: 30 | |
| IgGC01 | SVSSAVA | SASSLY | LGVRA | (D)FSSYSI | SIYPYSGS | RSHSYYSHTW |
| | SEQ ID | SEQ ID | A | SEQ ID | TY | WVSYGM |
| | NO: 1 | NO: 2 | SEQ ID | NO: 28 | SEQ ID | SEQ ID NO: 43 |
| | | | NO: 14 | | NO: 30 | |
| IgGC03 | SVSSAVA | SASSLY | SGRKP | (D)FSSYSI | SIYPYSGS | QSHSYYSHTW |
| | SEQ ID | SEQ ID | V | SEQ ID | TY | WVSYGM |
| | NO: 1 | NO: 2 | SEQ ID | NO: 28 | SEQ ID | SEQ ID NO: 32 |
| | | | NO: 15 | | NO: 30 | |
| IgGC04 | SVSSAVA | SASSLY | TAQSPL | (D)FSSYSI | SIYPYSGS | RSHSYYSHTW |
| | SEQ ID | SEQ ID | SEQ ID | SEQ ID | TY | WVSYGM |
| | NO: 1 | NO: 2 | NO: 16 | NO: 28 | SEQ ID | SEQ ID NO: 43 |
| | | | | | NO: 30 | |
| IgGC05 | SVSSAVA | SASSLY | SRQRPL | (D)FSSYSI | SIYPYSGS | QSHSYYSHTW |
| | SEQ ID | SEQ ID | SEQ ID | SEQ ID | TY | WVSYGM |
| | NO: 1 | NO: 2 | NO: 17 | NO: 28 | SEQ ID | SEQ ID NO: 32 |
| | | | | | NO: 30 | |
| IgGC06 | SVSSAVA | SASSLY | SGLMP | (D)FSSYSI | SIYPYSGS | QSHSYYSHTW |
| | SEQ ID | SEQ ID | L | SEQ ID | TY | WVSYGM |
| | NO: 1 | NO: 2 | SEQ ID | NO: 28 | SEQ ID | SEQ ID NO: 32 |
| | | | NO: 18 | | NO: 30 | |
| IgGC09 | SVSSAVA | SASSLY | SGHRPI | (D)FSSYSI | SIYPYSGS | QSHSYYSHTW |
| | SEQ ID | SEQ ID | SEQ ID | SEQ ID | TY | WVSYGM |
| | NO: 1 | NO: 2 | NO: 19 | NO: 28 | SEQ ID | SEQ ID NO: 32 |
| | | | | | NO: 30 | |
| IgGD02 | SVSSAVA | SASSLY | SSQRPA | (D)FSSYSI | SIYPYSGS | KYHSYYSHTW |
| | SEQ ID | SEQ ID | SEQ ID | SEQ ID | TY | WVSYGM |
| | NO: 1 | NO: 2 | NO: 21 | NO: 28 | SEQ ID | SEQ ID NO: 20 |
| | | | | | NO: 30 | |
| IgGD03 | SVSSAVA | SASSLY | SGRQPL | (D)FSSYSI | SIYPYSGS | QDHSYYSHTW |
| | SEQ ID | SEQ ID | SEQ ID | SEQ ID | TY | WVSYGM |
| | NO: 1 | NO: 2 | NO: 22 | NO: 28 | SEQ ID | SEQ ID NO: 44 |
| | | | | | NO: 30 | |
| IgGD04 | SVSSAVA | SASSLY | FMRPA | (D)FSSYSI | SIYPYSGS | RSHSYYSHTW |
| | SEQ ID | SEQ ID | F | SEQ ID | TY | WVSYGM |
| | NO: 1 | NO: 2 | SEQ ID | NO: 28 | SEQ ID | SEQ ID NO: 43 |
| | | | NO: 23 | | NO: 30 | |
| IgGD07 | SVSSAVA | SASSLY | SGQPS | (D)FSSYSI | SIYPYSGS | KSHSYYSHTW |
| | SEQ ID | SEQ ID | M | SEQ ID | TY | WVSYGM |
| | NO: 1 | NO: 2 | SEQ ID | NO: 28 | SEQ ID | SEQ ID NO: 46 |
| | | | NO: 24 | | NO: 30 | |
| IgGD08 | SVSSAVA | SASSLY | SVLRPF | (D)FSSYSI | SIYPYSGS | QSHSYYSHTW |
| | SEQ ID | SEQ ID | SEQ ID | SEQ ID | TY | WVSYGM |
| | NO: 1 | NO: 2 | NO: 25 | NO: 28 | SEQ ID | SEQ ID NO: 32 |
| | | | | | NO: 30 | |
| IgGD09 | SVSSAVA | SASSLY | SSSSLI | (N)FSSSSI | SIYPYSGS | IYWVDFWYGH |
| | SEQ ID | SEQ ID | SEQ ID | SEQ ID | TS | FSYYRPAL |
| | NO: 1 | NO: 2 | NO: 4 | NO: 29 | SEQ ID | SEQ ID NO: 47 |
| | | | | | NO: 31 | |
| IgG87 | SVSSAVA | SASSLY | SGQRPI | ASSYSI | SIYPYSGS | QSHSYYSHTW |
| | SEQ ID | SEQ ID | SEQ ID | SEQ ID | TY | WVSYGM SEQ |
| | NO: 1 | NO: 2 | NO: 3 | NO: 109 | SEQ ID | ID NO: 32 |
| | | | | | NO: 30 | |
| IgG89 | SVSSAVA | SASSLY | SGQRPI | FSSYSA | SIYPYSGS | QSHSYYSHTW |
| | SEQ ID | SEQ ID | SEQ ID | SEQ ID | TY | WVSYGM |
| | NO: 1 | NO: 2 | NO: 3 | NO: 111 | SEQ ID | SEQ ID NO: 32 |
| | | | | | NO: 30 | |
| IgG94 | SVSSAVA | SASSLY | SGQRPI | FSSYSI | SIYPYSGS | QSHSYYSHTW |
| | SEQ ID | SEQ ID | SEQ ID | SEQ ID | SEQ ID | WVSYGM SEQ |
| | NO: 1 | NO: 2 | NO: 3 | NO: 26 | NO: 31 | ID NO: |
| | | | | | | 32 |
| IgG97 | SVSSAVA | SASSLY | SGQRPI | FSSYSI | SIYPYSGS | QSHSYYSHTA |
| | SEQ ID | SEQ ID | SEQ ID | SEQ ID | TY | WVSYGM |
| | NO: 1 | NO: 2 | NO: 3 | NO: 26 | SEQ ID | SEQ ID NO: |
| | | | | | NO: 30 | 105 |
| IgG102 | SVSSAVA | SASSLY | SGQRPI | FSSYSI | SSYPYSG | QSHSYYSHTW |
| | SEQ ID | SEQ ID | SEQ ID | SEQ ID | STY | WVSYGM |
| | NO: 1 | NO: 2 | NO: 3 | NO: 26 | SEQ ID | SEQ ID NO: 32 |
| | | | | | NO: 101 | |

In some aspects, the anti-mouse cleaved CDCP1 antibody or antigen-binding fragment thereof comprises a light chain variable region (VL) and a heavy chain variable region (VH); wherein the VL comprises a VL complementarity determining region (CDR) 1 (VL-CDR1), a VL-CDR2, and a VL-CDR3 and the VH comprises a VH-CDR1, a VH-CDR2, and a VH-CDR3; wherein the VL-CDR3 comprises an amino acid sequence having at least about 90%, at least about 95%, at least about 96%, at least about 97%, at least about 98%, or at least about 99% sequence identity to an amino acid sequence selected of SEQ ID NO: 48 or 49. In some aspects, the anti-mouse cleaved CDCP1 antibody or antigen-binding fragment thereof comprises a VL-CDR3 comprising an amino acid of SEQ ID NO: 48 or 49, as disclosed in Table 2 below.

In some aspects, the anti-mouse cleaved CDCP1 antibody or antigen-binding fragment thereof comprises a VH-CDR2 comprising an amino acid sequence having at least about 90%, at least about 95%, at least about 96%, at least about 97%, at least about 98%, or at least about 99% sequence identity to an amino acid sequence selected from the group consisting of SEQ ID Nos: 52-55. In some aspects, the anti-mouse cleaved CDCP1 antibody comprises a VH-CDR2 comprising an amino acid sequence selected from the group consisting of SEQ ID NOs: 52-55, as disclosed in Table 2 below.

In some aspects, the anti-mouse cleaved CDCP1 antibody or antigen-binding fragment thereof comprises a VH-CDR1 comprising an amino acid sequence having at least about 90%, at least about 95%, at least about 96%, at least about 97%, at least about 98%, or at least about 99% sequence identity to an amino acid sequence of SEQ ID NO: 50 or 51. In some aspects, the anti-mouse cleaved CDCP1 antibody or antigen-binding fragment thereof comprises a VH-CDR1 comprising an amino acid sequence of SEQ ID NO: 50 or 51, as disclosed in Table 2 below.

In some aspects, the anti-mouse cleaved CDCP1 antibody or antigen-binding fragment thereof comprises a VL-CDR1 comprising an amino acid sequence having at least about 90%, at least about 95%, at least about 96%, at least about 97%, at least about 98%, or at least about 99% sequence identity to an amino acid sequence of SEQ ID NO: 1. In some aspects, the anti-mouse cleaved CDCP1 antibody or antigen-binding fragment thereof comprises a VL-CDR1 comprising an amino acid sequence of SEQ ID NO: 1, as disclosed in Table 2 below.

In some aspects, the anti-mouse cleaved CDCP1 antibody or antigen-binding fragment thereof comprises a VL-CDR2 comprising an amino acid sequence having at least about 90%, at least about 95%, at least about 96%, at least about 97%, at least about 98%, or at least about 99% sequence identity to an amino acid sequence of SEQ ID NO: 2. In some aspects, the anti-mouse cleaved CDCP1 antibody or antigen-binding fragment thereof comprises a VL-CDR2 comprising an amino acid sequence of SEQ ID NO: 2, as disclosed in Table 2 below.

In some aspects, the anti-mouse cleaved CDCP1 antibody or antigen-binding fragment thereof comprises a VH-CDR3 comprising an amino acid sequence having at least about 90%, at least about 95%, at least about 96%, at least about 97%, at least about 98%, or at least about 99% sequence identity to an amino acid sequence selected from the group consisting of SEQ ID NOs: 56-60. In some aspects, the anti-mouse cleaved CDCP1 antibody or antigen-binding fragment thereof comprises a VH-CDR3 comprising an amino acid sequence selected from the group consisting of SEQ ID NOs: 56-60, as disclosed in Table 2 below.

TABLE 2
Variable Light and Heavy Chain CDR Amino Acid Sequences of Anti-Mouse
Cleaved CDCP1 Antibodies

| Name | CDR L1 | CDR L2 | CDR L3 | CDR H1 | CDR H2 | CDR H3 |
|---|---|---|---|---|---|---|
| Fab 0114 | SVSSAVA | SASSLY | SSWGLF | VSYSSI | SISSSSG | QEGDWWVA |
| | SEQ ID | SEQ ID | SEQ ID | SEQ ID | YTY | YWPAL |
| | NO: 1 | NO: 2 | NO: 48 | NO: 50 | SEQ ID | SEQ ID NO: 56 |
| | | | | | NO: 52 | |
| IgG 0029 | SVSSAVA | SASSLY | SSWGLF | VSYSSI | SISSSSG | QEGDWWVA |
| (Fab 0114) | SEQ ID | SEQ ID | SEQ ID | SEQ ID | YTY | YWPAL |
| | NO: 1 | NO: 2 | NO: 48 | NO: 50 | SEQ ID | SEQ ID NO: 56 |
| | | | | | NO: 52 | |
| Fab 0123 | SVSSAVA | SASSLY | TYSGPI | VYSYSI | SISSSSG | YYWDPWQY |
| | SEQ ID | SEQ ID | SEQ ID | SEQ ID | YTY | WYYSYLAF |
| | NO: 1 | NO: 2 | NO: 49 | NO: 51 | SEQ ID | SEQ ID NO: 57 |
| | | | | | NO: 52 | |
| IgG 0030 | SVSSAVA | SASSLY | TYSGPI | VYSYSI | SISSSSG | YYWDPWQY |
| (Fab 0123) | SEQ ID | SEQ ID | SEQ ID | SEQ ID | YTY | WYYSYLAF |
| | NO: 1 | NO: 2 | NO: 49 | NO: 51 | SEQ ID | SEQ ID NO: 57 |
| | | | | | NO: 52 | |
| IgG 0046 | SVSSAVA | SASSLY | TYSGPI | VYSYSI | SISSSSG | YYWDPWQY |
| | SEQ ID | SEQ ID | SEQ ID | SEQ ID | YTY | AYYSYLAF |
| | NO: 1 | NO: 2 | NO: 49 | NO: 51 | SEQ ID | SEQ ID NO: 58 |
| | | | | | NO: 52 | |
| IgG 0050 | SVSSAVA | SASSLY | TYSGPI | VYSYSI | SISSSSG | YYWDPWQY |
| | SEQ ID | SEQ ID | SEQ ID | SEQ ID | STY | AYYSYLAF |
| | NO: 1 | NO: 2 | NO: 49 | NO: 51 | SEQ ID | SEQ ID NO: 58 |
| | | | | | NO: 53 | |
| IgG 0051 | SVSSAVA | SASSLY | TYSGPI | VYSYSI | SISSSSG | YYWDPWQY |
| | SEQ ID | SEQ ID | SEQ ID | SEQ ID | YTS | AYYSYLAF |
| | NO: 1 | NO: 2 | NO: 49 | NO: 51 | SEQ ID | SEQ ID NO: 58 |
| | | | | | NO: 54 | |
| IgG 0055 | SVSSAVA | SASSLY | TYSGPI | VYSYSI | SISSSSG | YYWDPWQY |
| | SEQ ID | SEQ ID | SEQ ID | SEQ ID | YTY | ASYSYLAF |
| | NO: 1 | NO: 2 | NO: 49 | NO: 51 | SEQ ID | SEQ ID NO: 59 |
| | | | | | NO: 52 | |
| IgG 0058 | SVSSAVA | SASSLY | TYSGPI | VYSYSI | SISSSSG | YYWDPWQY |
| | SEQ ID | SEQ ID | SEQ ID | SEQ ID | STS | ASYSYLAF |
| | NO: 1 | NO: 2 | NO: 49 | NO: 51 | SEQ ID | SEQ ID NO: 59 |
| | | | | | NO: 55 | |
| IgG 0059 | SVSSAVA | SASSLY | TYSGPI | VYSYSI | SISSSSG | YYWDPWQY |
| | SEQ ID | SEQ ID | SEQ ID | SEQ ID | STY | ASYSYLAF |
| | NO: 1 | NO: 2 | NO: 49 | NO: 51 | SEQ ID | SEQ ID NO: 59 |
| | | | | | NO: 53 | |
| IgG 0060 | SVSSAVA | SASSLY | TYSGPI | VYSYSI | SISSSSG | YYWDPWQY |
| | SEQ ID | SEQ ID | SEQ ID | SEQ ID | YTS | ASYSYLAF |
| | NO: 1 | NO: 2 | NO: 49 | NO: 51 | SEQ ID | SEQ ID NO: 59 |
| | | | | | NO: 54 | |
| IgG 0062 | SVSSAVA | SASSLY | TYSGPI | VYSYSI | SISSSSG | YYWDPWQYS |
| | SEQ ID | SEQ ID | SEQ ID | SEQ ID | STY | SYSYLAF |
| | NO: 1 | NO: 2 | NO: 49 | NO: 51 | SEQ ID | SEQ ID NO: 60 |
| | | | | | NO: 53 | |
| IgG 0063 | SVSSAVA | SASSLY | TYSGPI | VYSYSI | SISSSSG | YYWDPWQYS |
| | SEQ ID | SEQ ID | SEQ ID | SEQ ID | YTS | SYSYLAF |
| | NO: 1 | NO: 2 | NO: 49 | NO: 51 | SEQ ID | SEQ ID NO: 60 |
| | | | | | NO: 54 | |
| IgG 0065 | SVSSAVA | SASSLY | TYSGPI | VYSYSI | SISSSSG | YYWDPWQYS |
| | SEQ ID | SEQ ID | SEQ ID | SEQ ID | STS | SYSYLAF |
| | NO: 1 | NO: 2 | NO: 49 | NO: 51 | SEQ ID | SEQ ID NO: 60 |
| | | | | | NO: 55 | |

In some aspects, the anti-human cleaved CDCP1 antibody or antigen-binding fragment thereof comprises a VH comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to an amino acid sequence selected from the group consisting of SEQ ID NOs: 61, 65, 67, 69, 71, 73, 75, 79, 80, 81, 82, 83, 84, 85, 86, 87, 89, 91, 99, 103, 107, 123, and 133. In certain aspects, the VH comprises an amino acid sequence selected from the group consisting of SEQ ID NOs: 61, 65, 67, 69, 71, 73, 75, 79, 80, 81, 82, 83, 84, 85, 86, 87, 89, 91, 99, 103, 107, 123, and 133, as disclosed in Table 3 below.

In some aspects, the anti-human cleaved CDCP1 antibody or antigen-binding fragment thereof comprises a VL comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to an amino acid sequence selected from the group consisting of SEQ ID NOs: 62, 76, 94, 96, 98, 100, 102, 104, 106, 108, 110, 112, 114, 116, 118, 120, 122, 124, 126, 128, 130, and 132. In certain aspects, the VL comprises an amino acid sequence selected from the group consisting of SEQ ID NOs: 62, 76, 94, 96, 98, 100, 102, 104, 106, 108, 110, 112, 114, 116, 118, 120, 122, 124, 126, 128, 130, and 132, as disclosed in Table 3 below.

In some aspects, the anti-human cleaved CDCP1 antibody or antigen-binding fragment thereof comprises a VH comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO: 61. In certain aspects, the VH comprises the amino acid sequence set forth in SEQ ID NO:61. In some aspects, the anti-human cleaved CDCP1 about 90%, at least about 95%, at least about 96%, at least about 97%, at least about 98%, or at least about 99% sequence identity to SEQ ID NO:62. In certain aspects, the VL comprises the amino acid sequence set forth in SEQ ID NO:62. In some aspects, the VH comprises the amino acid sequence set forth in SEQ ID NO:61 and the VL comprises the amino acid sequence set forth in SEQ ID NO:62. In another embodiment, the anti-CDCP1 antibody or antigen-binding fragment thereof cross competes for binding to human cleaved CDCP1 with an antibody or antigen-binding fragment thereof comprising a VH comprising the amino acid sequence set forth in SEQ ID NO:61 and a VL comprising the amino acid sequence set forth in SEQ ID NO:62.

In some aspects, the anti-human cleaved CDCP1 antibody or antigen-binding fragment thereof comprises a VH comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:65. In certain aspects, the VH comprises the amino acid sequence set forth in SEQ ID NO:65. In some aspects, the anti-human cleaved CDCP1 antibody comprises a VL comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:62. In certain aspects, the VL comprises the amino acid sequence set forth in SEQ ID NO:62. In some aspects, the VH comprises the amino acid sequence set forth in SEQ ID NO: 65 and the VL comprises the amino acid sequence set forth in SEQ ID NO:62. In another embodiment, the anti-CDCP1 antibody or antigen-binding fragment thereof cross competes for binding to human cleaved CDCP1 with an antibody or antigen-binding fragment thereof comprising a VH comprising the amino acid sequence set forth in SEQ ID NO:65 and a VL comprising the amino acid sequence set forth in SEQ ID NO:62.

In some aspects, the anti-human cleaved CDCP1 antibody or antigen-binding fragment thereof comprises a VH comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:67. In certain aspects, the VH comprises the amino acid sequence set forth in SEQ ID NO:67. In some aspects, the anti-human cleaved CDCP1 about 90%, at least about 95%, at least about 96%, at least about 97%, at least about 98%, or at least about 99% sequence identity to SEQ ID NO:62. In certain aspects, the VL comprises the amino acid sequence set forth in SEQ ID NO:62. In some aspects, the VH comprises the amino acid sequence set forth in SEQ ID NO:67 and the VL comprises the amino acid sequence set forth in SEQ ID NO:62. In another embodiment, the anti-CDCP1 antibody or antigen-binding fragment thereof cross competes for binding to human cleaved CDCP1 with an antibody or antigen-binding fragment thereof comprising a VH comprising the amino acid sequence set forth in SEQ ID NO:67 and a VL comprising the amino acid sequence set forth in SEQ ID NO:62.

In some aspects, the anti-human cleaved CDCP1 antibody or antigen-binding fragment thereof comprises a VH comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:69. In certain aspects, the VH comprises the amino acid sequence set forth in SEQ ID NO:69. In some aspects, the anti-human cleaved CDCP1 antibody comprises a VL comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:62. In certain aspects, the VL comprises the amino acid sequence set forth in SEQ ID NO:62. In some aspects, the VH comprises the amino acid sequence set forth in SEQ ID NO:69 and the VL comprises the amino acid sequence set forth in SEQ ID NO:62. In another embodiment, the anti-CDCP1 antibody or antigen-binding fragment thereof cross competes for binding to human cleaved CDCP1 with an antibody or antigen-binding fragment thereof comprising a VH comprising the amino acid sequence set forth in SEQ ID NO:69 and a VL comprising the amino acid sequence set forth in SEQ ID NO:62.

In some aspects, the anti-human cleaved CDCP1 antibody or antigen-binding fragment thereof comprises a VH comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:71. In certain aspects, the VH comprises the amino acid sequence set forth in SEQ ID NO:71. In some aspects, the anti-human cleaved CDCP1 about 90%, at least about 95%, at least about 96%, at least about 97%, at least about 98%, or at least about 99% sequence identity to SEQ ID NO:62. In certain aspects, the VL comprises the amino acid sequence set forth in SEQ ID NO:62. In some aspects, the VH comprises the amino acid sequence set forth in SEQ ID NO:71 and the VL comprises the amino acid sequence set forth in SEQ ID NO:62. In another embodiment, the anti-CDCP1 antibody or antigen-binding fragment thereof cross competes for binding to human cleaved CDCP1 with an antibody or antigen-binding fragment thereof comprising a VH comprising the amino acid sequence set forth in SEQ ID NO:71 and a VL comprising the amino acid sequence set forth in SEQ ID NO:62.

In some aspects, the anti-human cleaved CDCP1 antibody or antigen-binding fragment thereof comprises a VH comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:73. In certain aspects, the VH comprises the amino acid sequence set forth in SEQ ID NO:73. In some aspects, the anti-human cleaved CDCP1 antibody comprises a VL comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:62. In certain aspects, the VL comprises the amino acid sequence set forth in SEQ ID NO:74. In some aspects, the VH comprises the amino acid sequence set forth in SEQ ID NO:73 and the VL comprises the amino acid sequence set forth in SEQ ID NO:62. In another embodiment, the anti-CDCP1 antibody or antigen-binding fragment thereof cross competes for binding to human cleaved CDCP1 with an antibody or antigen-binding fragment thereof comprising a VH comprising the amino acid sequence set forth in SEQ ID NO: 73 and a VL comprising the amino acid sequence set forth in SEQ ID NO:62.

In some aspects, the anti-human cleaved CDCP1 antibody or antigen-binding fragment thereof comprises a VH comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:75. In certain aspects, the VH comprises the amino acid sequence set forth in SEQ ID NO:75. In some aspects, the anti-human cleaved CDCP1 about 90%, at least about 95%, at least about 96%, at least about 97%, at least about 98%, or at least about 99% sequence identity to SEQ ID NO:76. In certain aspects, the VL comprises the amino acid sequence set forth in SEQ ID NO:76. In some aspects, the VH comprises the amino acid sequence set forth in SEQ ID NO:75 and the VL comprises the amino acid sequence set forth in SEQ ID NO:76. In another embodiment, the anti-CDCP1 antibody or antigen-binding fragment thereof cross competes for binding to human cleaved CDCP1 with an antibody or antigen-binding fragment thereof comprising a VH comprising the amino acid sequence set forth in SEQ ID NO: 75 and a VL comprising the amino acid sequence set forth in SEQ ID NO:76.

In some aspects, the anti-human cleaved CDCP1 antibody or antigen-binding fragment thereof comprises a VH comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:79. In certain aspects, the VH comprises the amino acid sequence set forth in SEQ ID NO:79. In some aspects, the anti-human cleaved CDCP1 antibody comprises a VL comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:76. In certain aspects, the VL comprises the amino acid sequence set forth in SEQ ID NO:76. In some aspects, the VH comprises the amino acid sequence set forth in SEQ ID NO:79 and the VL comprises the amino acid sequence set forth in SEQ ID NO:76. In another embodiment, the anti-CDCP1 antibody or antigen-binding fragment thereof cross competes for binding to human cleaved CDCP1 with an antibody or antigen-binding fragment thereof comprising a VH comprising the amino acid sequence set forth in SEQ ID NO:79 and a VL comprising the amino acid sequence set forth in SEQ ID NO:76.

In some aspects, the anti-human cleaved CDCP1 antibody or antigen-binding fragment thereof comprises a VH comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:65. In certain aspects, the VH comprises the amino acid sequence set forth in SEQ ID NO:65. In some aspects, the anti-human cleaved CDCP1 about 90%, at least about 95%, at least about 96%, at least about 97%, at least about 98%, or at least about 99% sequence identity to SEQ ID NO:76. In certain aspects, the VL comprises the amino acid sequence set forth in SEQ ID NO:76. In some aspects, the VH comprises the amino acid sequence set forth in SEQ ID NO:65 and the VL comprises the amino acid sequence set forth in SEQ ID NO:76. In another embodiment, the anti-CDCP1 antibody or antigen-binding fragment thereof cross competes for binding to human cleaved CDCP1 with an antibody or antigen-binding fragment thereof comprising a VH comprising the amino acid sequence set forth in SEQ ID NO:65 and a VL comprising the amino acid sequence set forth in SEQ ID NO:76.

In some aspects, the anti-human cleaved CDCP1 antibody or antigen-binding fragment thereof comprises a VH comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:83. In certain aspects, the VH comprises the amino acid sequence set forth in SEQ ID NO:83. In some aspects, the anti-human cleaved CDCP1 antibody comprises a VL comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:62. In certain aspects, the VL comprises the amino acid sequence set forth in SEQ ID NO:62. In some aspects, the VH comprises the amino acid sequence set forth in SEQ ID NO:83 and the VL comprises the amino acid sequence set forth in SEQ ID NO:62. In another embodiment, the anti-CDCP1 antibody or antigen-binding fragment thereof cross competes for binding to human cleaved CDCP1 with an antibody or antigen-binding fragment thereof comprising a VH comprising the amino acid sequence set forth in SEQ ID NO:83 and a VL comprising the amino acid sequence set forth in SEQ ID NO:62.

In some aspects, the anti-human cleaved CDCP1 antibody or antigen-binding fragment thereof comprises a VH comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:85. In certain aspects, the VH comprises the amino acid sequence set forth in SEQ ID NO:85. In some aspects, the anti-human cleaved CDCP1 about 90%, at least about 95%, at least about 96%, at least about 97%, at least about 98%, or at least about 99% sequence identity to SEQ ID NO:62. In certain aspects, the VL comprises the amino acid sequence set forth in SEQ ID NO:62. In some aspects, the VH comprises the amino acid sequence set forth in SEQ ID NO:85 and the VL comprises the amino acid sequence set forth in SEQ ID NO:62. In another embodiment, the anti-CDCP1 antibody or antigen-binding fragment thereof cross competes for binding to human cleaved CDCP1 with an antibody or antigen-binding fragment thereof comprising a VH comprising the amino acid sequence set forth in SEQ ID NO:85 and a VL comprising the amino acid sequence set forth in SEQ ID NO:62.

In some aspects, the anti-human cleaved CDCP1 antibody or antigen-binding fragment thereof comprises a VH comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:87. In certain aspects, the VH comprises the amino acid sequence set forth in SEQ ID NO:87. In some aspects, the anti-human cleaved CDCP1 antibody comprises a VL comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:62. In certain aspects, the VL comprises the amino acid sequence set forth in SEQ ID NO:62. In some aspects, the VH comprises the amino acid sequence set forth in SEQ ID NO:87 and the VL comprises the amino acid sequence set forth in SEQ ID NO:62. In another embodiment, the anti-CDCP1 antibody or antigen-binding fragment thereof cross competes for binding to human cleaved CDCP1 with an antibody or antigen-binding fragment thereof comprising a VH comprising the amino acid sequence set forth in SEQ ID NO:87 and a VL comprising the amino acid sequence set forth in SEQ ID NO:62.

In some aspects, the anti-human cleaved CDCP1 antibody or antigen-binding fragment thereof comprises a VH comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:89. In certain aspects, the VH comprises the amino acid sequence set forth in SEQ ID NO:89. In some aspects, the anti-human cleaved CDCP1 about 90%, at least about 95%, at least about 96%, at least about 97%, at least about 98%, or at least about 99% sequence identity to SEQ ID NO:62. In certain aspects, the VL comprises the amino acid sequence set forth in SEQ ID NO:62. In some aspects, the VH comprises the amino acid sequence set forth in SEQ ID NO:89 and the VL comprises the amino acid sequence set forth in SEQ ID NO:62. In another embodiment, the anti-CDCP1 antibody or antigen-binding fragment thereof cross competes for binding to human cleaved CDCP1 with an antibody or antigen-binding fragment thereof comprising a VH comprising the amino acid sequence set forth in SEQ ID NO:89 and a VL comprising the amino acid sequence set forth in SEQ ID NO:62.

In some aspects, the anti-human cleaved CDCP1 antibody or antigen-binding fragment thereof comprises a VH comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:91. In certain aspects, the VH comprises the amino acid sequence set forth in SEQ ID NO:91. In some aspects, the anti-human cleaved CDCP1 antibody comprises a VL comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:62. In certain aspects, the VL comprises the amino acid sequence set forth in SEQ ID NO:96. In some aspects, the VH comprises the amino acid sequence set forth in SEQ ID NO:91 and the VL comprises the amino acid sequence set forth in SEQ ID NO:62. In another embodiment, the anti-CDCP1 antibody or antigen-binding fragment thereof cross competes for binding to human cleaved CDCP1 with an antibody or antigen-binding fragment thereof comprising a VH comprising the amino acid sequence set forth in SEQ ID NO:91 and a VL comprising the amino acid sequence set forth in SEQ ID NO:62.

In some aspects, the anti-human cleaved CDCP1 antibody or antigen-binding fragment thereof comprises a VH comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:65. In certain aspects, the VH comprises the amino acid sequence set forth in SEQ ID NO:65. In some aspects, the anti-human cleaved CDCP1 about 90%, at least about 95%, at least about 96%, at least about 97%, at least about 98%, or at least about 99% sequence identity to SEQ ID NO:94. In certain aspects, the VL comprises the amino acid sequence set forth in SEQ ID NO:94. In some aspects, the VH comprises the amino acid sequence set forth in SEQ ID NO:65 and the VL comprises the amino acid sequence set forth in SEQ ID NO:94. In another embodiment, the anti-CDCP1 antibody or antigen-binding fragment thereof cross competes for binding to human cleaved CDCP1 with an antibody or antigen-binding fragment thereof comprising a VH comprising the amino acid sequence set forth in SEQ ID NO:65 and a VL comprising the amino acid sequence set forth in SEQ ID NO:94.

In some aspects, the anti-human cleaved CDCP1 antibody or antigen-binding fragment thereof comprises a VH comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:65. In certain aspects, the VH comprises the amino acid sequence set forth in SEQ ID NO:65. In some aspects, the anti-human cleaved CDCP1 antibody comprises a VL comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:96. In certain aspects, the VL comprises the amino acid sequence set forth in SEQ ID NO:96. In some aspects, the VH comprises the amino acid sequence set forth in SEQ ID NO:65 and the VL comprises the amino acid sequence set forth in SEQ ID NO:96. In another embodiment, the anti-CDCP1 antibody or antigen-binding fragment thereof cross competes for binding to human cleaved CDCP1 with an antibody or antigen-binding fragment thereof comprising a VH comprising the amino acid sequence set forth in SEQ ID NO:65 and a VL comprising the amino acid sequence set forth in SEQ ID NO:96.

In some aspects, the anti-human cleaved CDCP1 antibody or antigen-binding fragment thereof comprises a VH comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:65. In certain aspects, the VH comprises the amino acid sequence set forth in SEQ ID NO:65. In some aspects, the anti-human cleaved CDCP1 about 90%, at least about 95%, at least about 96%, at least about 97%, at least about 98%, or at least about 99% sequence identity to SEQ ID NO:98. In certain aspects, the VL comprises the amino acid sequence set forth in SEQ ID NO:98. In some aspects, the VH comprises the amino acid sequence set forth in SEQ ID NO:65 and the VL comprises the amino acid sequence set forth in SEQ ID NO:98. In another embodiment, the anti-CDCP1 antibody or antigen-binding fragment thereof cross competes for binding to human cleaved CDCP1 with an antibody or antigen-binding fragment thereof comprising a VH comprising the amino acid sequence set forth in SEQ ID NO:65 and a VL comprising the amino acid sequence set forth in SEQ ID NO:98.

In some aspects, the anti-human cleaved CDCP1 antibody or antigen-binding fragment thereof comprises a VH comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:99. In certain aspects, the VH comprises the amino acid sequence set forth in SEQ ID NO:99. In some aspects, the anti-human cleaved CDCP1 antibody comprises a VL comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO: 100. In certain aspects, the VL comprises the amino acid sequence set forth in SEQ ID NO: 100. In some aspects, the VH comprises the amino acid sequence set forth in SEQ ID NO:99 and the VL comprises the amino acid sequence set forth in SEQ ID NO: 100. In another embodiment, the anti-CDCP1 antibody or antigen-binding fragment thereof cross competes for binding to human cleaved CDCP1 with an antibody or antigen-binding fragment thereof comprising a VH comprising the amino acid sequence set forth in SEQ ID NO:99 and a VL comprising the amino acid sequence set forth in SEQ ID NO:100.

In some aspects, the anti-human cleaved CDCP1 antibody or antigen-binding fragment thereof comprises a VH comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:65. In certain aspects, the VH comprises the amino acid sequence set forth in SEQ ID NO:65. In some aspects, the anti-human cleaved CDCP1 about 90%, at least about 95%, at least about 96%, at least about 97%, at least about 98%, or at least about 99% sequence identity to SEQ ID NO: 102. In certain aspects, the VL comprises the amino acid sequence set forth in SEQ ID NO:102. In some aspects, the VH comprises the amino acid sequence set forth in SEQ ID NO:65 and the VL comprises the amino acid sequence set forth in SEQ ID NO:102. In another embodiment, the anti-CDCP1 antibody or antigen-binding fragment thereof cross competes for binding to human cleaved CDCP1 with an antibody or antigen-binding fragment thereof comprising a VH comprising the amino acid sequence set forth in SEQ ID NO:65 and a VL comprising the amino acid sequence set forth in SEQ ID NO:102.

In some aspects, the anti-human cleaved CDCP1 antibody or antigen-binding fragment thereof comprises a VH comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:103. In certain aspects, the VH comprises the amino acid sequence set forth in SEQ ID NO: 103. In some aspects, the anti-human cleaved CDCP1 antibody comprises a VL comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:104. In certain aspects, the VL comprises the amino acid sequence set forth in SEQ ID NO:104. In some aspects, the VH comprises the amino acid sequence set forth in SEQ ID NO: 103 and the VL comprises the amino acid sequence set forth in SEQ ID NO: 104. In another embodiment, the anti-CDCP1 antibody or antigen-binding fragment thereof cross competes for binding to human cleaved CDCP1 with an antibody or antigen-binding fragment thereof comprising a VH comprising the amino acid sequence set forth in SEQ ID NO: 103 and a VL comprising the amino acid sequence set forth in SEQ ID NO:104.

In some aspects, the anti-human cleaved CDCP1 antibody or antigen-binding fragment thereof comprises a VH comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:99. In certain aspects, the VH comprises the amino acid sequence set forth in SEQ ID NO:99. In some aspects, the anti-human cleaved CDCP1 about 90%, at least about 95%, at least about 96%, at least about 97%, at least about 98%, or at least about 99% sequence identity to SEQ ID NO:106. In certain aspects, the VL comprises the amino acid sequence set forth in SEQ ID NO:106. In some aspects, the VH comprises the amino acid sequence set forth in SEQ ID NO:99 and the VL comprises the amino acid sequence set forth in SEQ ID NO: 106. In another embodiment, the anti-CDCP1 antibody or antigen-binding fragment thereof cross competes for binding to human cleaved CDCP1 with an antibody or antigen-binding fragment thereof comprising a VH comprising the amino acid sequence set forth in SEQ ID NO:99 and a VL comprising the amino acid sequence set forth in SEQ ID NO: 106.

In some aspects, the anti-human cleaved CDCP1 antibody or antigen-binding fragment thereof comprises a VH comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:107. In certain aspects, the VH comprises the amino acid sequence set forth in SEQ ID NO: 107. In some aspects, the anti-human cleaved CDCP1 antibody comprises a VL comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:108. In certain aspects, the VL comprises the amino acid sequence set forth in SEQ ID NO:108. In some aspects, the VH comprises the amino acid sequence set forth in SEQ ID NO: 107 and the VL comprises the amino acid sequence set forth in SEQ ID NO: 108. In another embodiment, the anti-CDCP1 antibody or antigen-binding fragment thereof cross competes for binding to human cleaved CDCP1 with an antibody or antigen-binding fragment thereof comprising a VH comprising the amino acid sequence set forth in SEQ ID NO: 107 and a VL comprising the amino acid sequence set forth in SEQ ID NO:108.

In some aspects, the anti-human cleaved CDCP1 antibody or antigen-binding fragment thereof comprises a VH comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:99. In certain aspects, the VH comprises the amino acid sequence set forth in SEQ ID NO:99. In some aspects, the anti-human cleaved CDCP1 about 90%, at least about 95%, at least about 96%, at least about 97%, at least about 98%, or at least about 99% sequence identity to SEQ ID NO: 110. In certain aspects, the VL comprises the amino acid sequence set forth in SEQ ID NO:110. In some aspects, the VH comprises the amino acid sequence set forth in SEQ ID NO:99 and the VL comprises the amino acid sequence set forth in SEQ ID NO: 110. In another embodiment, the anti-CDCP1 antibody or antigen-binding fragment thereof cross competes for binding to human cleaved CDCP1 with an antibody or antigen-binding fragment thereof comprising a VH comprising the amino acid sequence set forth in SEQ ID NO:99 and a VL comprising the amino acid sequence set forth in SEQ ID NO:110.

In some aspects, the anti-human cleaved CDCP1 antibody or antigen-binding fragment thereof comprises a VH comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:99. In certain aspects, the VH comprises the amino acid sequence set forth in SEQ ID NO:99. In some aspects, the anti-human cleaved CDCP1 antibody comprises a VL comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO: 112. In certain aspects, the VL comprises the amino acid sequence set forth in SEQ ID NO:112. In some aspects, the VH comprises the amino acid sequence set forth in SEQ ID NO:99 and the VL comprises the amino acid sequence set forth in SEQ ID NO: 112. In another embodiment, the anti-CDCP1 antibody or antigen-binding fragment thereof cross competes for binding to human cleaved CDCP1 with an antibody or antigen-binding fragment thereof comprising a VH comprising the amino acid sequence set forth in SEQ ID NO:99 and a VL comprising the amino acid sequence set forth in SEQ ID NO:112.

In some aspects, the anti-human cleaved CDCP1 antibody or antigen-binding fragment thereof comprises a VH comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:65. In certain aspects, the VH comprises the amino acid sequence set forth in SEQ ID NO:65. In some aspects, the anti-human cleaved CDCP1 about 90%, at least about 95%, at least about 96%, at least about 97%, at least about 98%, or at least about 99% sequence identity to SEQ ID NO: 114. In certain aspects, the VL comprises the amino acid sequence set forth in SEQ ID NO:114. In some aspects, the VH comprises the amino acid sequence set forth in SEQ ID NO:65 and the VL comprises the amino acid sequence set forth in SEQ ID NO: 114. In another embodiment, the anti-CDCP1 antibody or antigen-binding fragment thereof cross competes for binding to human cleaved CDCP1 with an antibody or antigen-binding fragment thereof comprising a VH comprising the amino acid sequence set forth in SEQ ID NO:65 and a VL comprising the amino acid sequence set forth in SEQ ID NO:114.

In some aspects, the anti-human cleaved CDCP1 antibody or antigen-binding fragment thereof comprises a VH comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:99. In certain aspects, the VH comprises the amino acid sequence set forth in SEQ ID NO:99. In some aspects, the anti-human cleaved CDCP1 antibody comprises a VL comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO: 116. In certain aspects, the VL comprises the amino acid sequence set forth in SEQ ID NO:116. In some aspects, the VH comprises the amino acid sequence set forth in SEQ ID NO:99 and the VL comprises the amino acid sequence set forth in SEQ ID NO: 116. In another embodiment, the anti-CDCP1 antibody or antigen-binding fragment thereof cross competes for binding to human cleaved CDCP1 with an antibody or antigen-binding fragment thereof comprising a VH comprising the amino acid sequence set forth in SEQ ID NO:99 and a VL comprising the amino acid sequence set forth in SEQ ID NO:116.

In some aspects, the anti-human cleaved CDCP1 antibody or antigen-binding fragment thereof comprises a VH comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:65. In certain aspects, the VH comprises the amino acid sequence set forth in SEQ ID NO:65. In some aspects, the anti-human cleaved CDCP1 about 90%, at least about 95%, at least about 96%, at least about 97%, at least about 98%, or at least about 99% sequence identity to SEQ ID NO: 118. In certain aspects, the VL comprises the amino acid sequence set forth in SEQ ID NO:118. In some aspects, the VH comprises the amino acid sequence set forth in SEQ ID NO:65 and the VL comprises the amino acid sequence set forth in SEQ ID NO:118. In another embodiment, the anti-CDCP1 antibody or antigen-binding fragment thereof cross competes for binding to human cleaved CDCP1 with an antibody or antigen-binding fragment thereof comprising a VH comprising the amino acid sequence set forth in SEQ ID NO:65 and a VL comprising the amino acid sequence set forth in SEQ ID NO:118.

In some aspects, the anti-human cleaved CDCP1 antibody or antigen-binding fragment thereof comprises a VH comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:65. In certain aspects, the VH comprises the amino acid sequence set forth in SEQ ID NO:65. In some aspects, the anti-human cleaved CDCP1 antibody comprises a VL comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:120. In certain aspects, the VL comprises the amino acid sequence set forth in SEQ ID NO: 120. In some aspects, the VH comprises the amino acid sequence set forth in SEQ ID NO:65 and the VL comprises the amino acid sequence set forth in SEQ ID NO: 120. In another embodiment, the anti-CDCP1 antibody or antigen-binding fragment thereof cross competes for binding to human cleaved CDCP1 with an antibody or antigen-binding fragment thereof comprising a VH comprising the amino acid sequence set forth in SEQ ID NO:65 and a VL comprising the amino acid sequence set forth in SEQ ID NO:120.

In some aspects, the anti-human cleaved CDCP1 antibody or antigen-binding fragment thereof comprises a VH comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:65. In certain aspects, the VH comprises the amino acid sequence set forth in SEQ ID NO:65. In some aspects, the anti-human cleaved CDCP1 about 90%, at least about 95%, at least about 96%, at least about 97%, at least about 98%, or at least about 99% sequence identity to SEQ ID NO:122. In certain aspects, the VL comprises the amino acid sequence set forth in SEQ ID NO:122. In some aspects, the VH comprises the amino acid sequence set forth in SEQ ID NO:65 and the VL comprises the amino acid sequence set forth in SEQ ID NO:122. In another embodiment, the anti-CDCP1 antibody or antigen-binding fragment thereof cross competes for binding to human cleaved CDCP1 with an antibody or antigen-binding fragment thereof comprising a VH comprising the amino acid sequence set forth in SEQ ID NO:65 and a VL comprising the amino acid sequence set forth in SEQ ID NO: 122.

In some aspects, the anti-human cleaved CDCP1 antibody or antigen-binding fragment thereof comprises a VH comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:123. In certain aspects, the VH comprises the amino acid sequence set forth in SEQ ID NO: 123. In some aspects, the anti-human cleaved CDCP1 antibody comprises a VL comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:124. In certain aspects, the VL comprises the amino acid sequence set forth in SEQ ID NO: 124. In some aspects, the VH comprises the amino acid sequence set forth in SEQ ID NO: 123 and the VL comprises the amino acid sequence set forth in SEQ ID NO: 124. In another embodiment, the anti-CDCP1 antibody or antigen-binding fragment thereof cross competes for binding to human cleaved CDCP1 with an antibody or antigen-binding fragment thereof comprising a VH comprising the amino acid sequence set forth in SEQ ID NO: 123 and a VL comprising the amino acid sequence set forth in SEQ ID NO: 124.

In some aspects, the anti-human cleaved CDCP1 antibody or antigen-binding fragment thereof comprises a VH comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:107. In certain aspects, the VH comprises the amino acid sequence set forth in SEQ ID NO: 107. In some aspects, the anti-human cleaved CDCP1 about 90%, at least about 95%, at least about 96%, at least about 97%, at least about 98%, or at least about 99% sequence identity to SEQ ID NO: 126. In certain aspects, the VL comprises the amino acid sequence set forth in SEQ ID NO:126. In some aspects, the VH comprises the amino acid sequence set forth in SEQ ID NO: 107 and the VL comprises the amino acid sequence set forth in SEQ ID NO:126. In another embodiment, the anti-CDCP1 antibody or antigen-binding fragment thereof cross competes for binding to human cleaved CDCP1 with an antibody or antigen-binding fragment thereof comprising a VH comprising the amino acid sequence set forth in SEQ ID NO: 107 and a VL comprising the amino acid sequence set forth in SEQ ID NO: 126.

In some aspects, the anti-human cleaved CDCP1 antibody or antigen-binding fragment thereof comprises a VH comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:99. In certain aspects, the VH comprises the amino acid sequence set forth in SEQ ID NO:99. In some aspects, the anti-human cleaved CDCP1 antibody comprises a VL comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:128. In certain aspects, the VL comprises the amino acid sequence set forth in SEQ ID NO:128. In some aspects, the VH comprises the amino acid sequence set forth in SEQ ID NO:99 and the VL comprises the amino acid sequence set forth in SEQ ID NO: 128. In another embodiment, the anti-CDCP1 antibody or antigen-binding fragment thereof cross competes for binding to human cleaved CDCP1 with an antibody or antigen-binding fragment thereof comprising a VH comprising the amino acid sequence set forth in SEQ ID NO:99 and a VL comprising the amino acid sequence set forth in SEQ ID NO:128.

In some aspects, the anti-human cleaved CDCP1 antibody or antigen-binding fragment thereof comprises a VH comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:99. In certain aspects, the VH comprises the amino acid sequence set forth in SEQ ID NO:99. In some aspects, the anti-human cleaved CDCP1 about 90%, at least about 95%, at least about 96%, at least about 97%, at least about 98%, or at least about 99% sequence identity to SEQ ID NO:130. In certain aspects, the VL comprises the amino acid sequence set forth in SEQ ID NO:130. In some aspects, the VH comprises the amino acid sequence set forth in SEQ ID NO:99 and the VL comprises the amino acid sequence set forth in SEQ ID NO:130. In another embodiment, the anti-CDCP1 antibody or antigen-binding fragment thereof cross competes for binding to human cleaved CDCP1 with an antibody or antigen-binding fragment thereof comprising a VH comprising the amino acid sequence set forth in SEQ ID NO:99 and a VL comprising the amino acid sequence set forth in SEQ ID NO: 130.

In some aspects, the anti-human cleaved CDCP1 antibody or antigen-binding fragment thereof comprises a VH comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:65. In certain aspects, the VH comprises the amino acid sequence set forth in SEQ ID NO:65. In some aspects, the anti-human cleaved CDCP1 antibody comprises a VL comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO: 132. In certain aspects, the VL comprises the amino acid sequence set forth in SEQ ID NO:132. In some aspects, the VH comprises the amino acid sequence set forth in SEQ ID NO:65 and the VL comprises the amino acid sequence set forth in SEQ ID NO: 132. In another embodiment, the anti-CDCP1 antibody or antigen-binding fragment thereof cross competes for binding to human cleaved CDCP1 with an antibody or antigen-binding fragment thereof comprising a VH comprising the amino acid sequence set forth in SEQ ID NO:65 and a VL comprising the amino acid sequence set forth in SEQ ID NO:132.

In some aspects, the anti-human cleaved CDCP1 antibody or antigen-binding fragment thereof comprises a VH comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:133. In certain aspects, the VH comprises the amino acid sequence set forth in SEQ ID NO: 133. In some aspects, the anti-human cleaved CDCP1 about 90%, at least about 95%, at least about 96%, at least about 97%, at least about 98%, or at least about 99% sequence identity to SEQ ID NO:76. In certain aspects, the VL comprises the amino acid sequence set forth in SEQ ID NO:76. In some aspects, the VH comprises the amino acid sequence set forth in SEQ ID NO: 133 and the VL comprises the amino acid sequence set forth in SEQ ID NO:76. In another embodiment, the anti-CDCP1 antibody or antigen-binding fragment thereof cross competes for binding to human cleaved CDCP1 with an antibody or antigen-binding fragment thereof comprising a VH comprising the amino acid sequence set forth in SEQ ID NO: 133 and a VL comprising the amino acid sequence set forth in SEQ ID NO:76.

In some aspects, the anti-human cleaved CDCP1 antibody or antigen-binding fragment thereof comprises a VH comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:80. In certain aspects, the VH comprises the amino acid sequence set forth in SEQ ID NO:80. In some aspects, the anti-human cleaved CDCP1 antibody comprises a VL comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:62. In certain aspects, the VL comprises the amino acid sequence set forth in SEQ ID NO:62. In some aspects, the VH comprises the amino acid sequence set forth in SEQ ID NO:80 and the VL comprises the amino acid sequence set forth in SEQ ID NO:62. In another embodiment, the anti-CDCP1 antibody or antigen-binding fragment thereof cross competes for binding to human cleaved CDCP1 with an antibody or antigen-binding fragment thereof comprising a VH comprising the amino acid sequence set forth in SEQ ID NO:80 and a VL comprising the amino acid sequence set forth in SEQ ID NO:62.

In some aspects, the anti-human cleaved CDCP1 antibody or antigen-binding fragment thereof comprises a VH comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:81. In certain aspects, the VH comprises the amino acid sequence set forth in SEQ ID NO:81. In some aspects, the anti-human cleaved CDCP1 about 90%, at least about 95%, at least about 96%, at least about 97%, at least about 98%, or at least about 99% sequence identity to SEQ ID NO:62. In certain aspects, the VL comprises the amino acid sequence set forth in SEQ ID NO:62. In some aspects, the VH comprises the amino acid sequence set forth in SEQ ID NO:81 and the VL comprises the amino acid sequence set forth in SEQ ID NO:62. In another embodiment, the anti-CDCP1 antibody or antigen-binding fragment thereof cross competes for binding to human cleaved CDCP1 with an antibody or antigen-binding fragment thereof comprising a VH comprising the amino acid sequence set forth in SEQ ID NO:81 and a VL comprising the amino acid sequence set forth in SEQ ID NO:62.

In some aspects, the anti-human cleaved CDCP1 antibody or antigen-binding fragment thereof comprises a VH comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:82. In certain aspects, the VH comprises the amino acid sequence set forth in SEQ ID NO:82. In some aspects, the anti-human cleaved CDCP1 antibody comprises a VL comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:62. In certain aspects, the VL comprises the amino acid sequence set forth in SEQ ID NO:62. In some aspects, the VH comprises the amino acid sequence set forth in SEQ ID NO:82 and the VL comprises the amino acid sequence set forth in SEQ ID NO:62. In another embodiment, the anti-CDCP1 antibody or antigen-binding fragment thereof cross competes for binding to human cleaved CDCP1 with an antibody or antigen-binding fragment thereof comprising a VH comprising the amino acid sequence set forth in SEQ ID NO:82 and a VL comprising the amino acid sequence set forth in SEQ ID NO:62.

In some aspects, the anti-human cleaved CDCP1 antibody or antigen-binding fragment thereof comprises a VH comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:84. In certain aspects, the VH comprises the amino acid sequence set forth in SEQ ID NO:84. In some aspects, the anti-human cleaved CDCP1 about 90%, at least about 95%, at least about 96%, at least about 97%, at least about 98%, or at least about 99% sequence identity to SEQ ID NO:62. In certain aspects, the VL comprises the amino acid sequence set forth in SEQ ID NO:62. In some aspects, the VH comprises the amino acid sequence set forth in SEQ ID NO:84 and the VL comprises the amino acid sequence set forth in SEQ ID NO:62. In another embodiment, the anti-CDCP1 antibody or antigen-binding fragment thereof cross competes for binding to human cleaved CDCP1 with an antibody or antigen-binding fragment thereof comprising a VH comprising the amino acid sequence set forth in SEQ ID NO:84 and a VL comprising the amino acid sequence set forth in SEQ ID NO:62.

In some aspects, the anti-human cleaved CDCP1 antibody or antigen-binding fragment thereof comprises a VH comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:86. In certain aspects, the VH comprises the amino acid sequence set forth in SEQ ID NO:86. In some aspects, the anti-human cleaved CDCP1 antibody comprises a VL comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO:62. In certain aspects, the VL comprises the amino acid sequence set forth in SEQ ID NO:62. In some aspects, the VH comprises the amino acid sequence set forth in SEQ ID NO:86 and the VL comprises the amino acid sequence set forth in SEQ ID NO:62. In another embodiment, the anti-CDCP1 antibody or antigen-binding fragment thereof cross competes for binding to human cleaved CDCP1 with an antibody or antigen-binding fragment thereof comprising a VH comprising the amino acid sequence set forth in SEQ ID NO:86 and a VL comprising the amino acid sequence set forth in SEQ ID NO:62.

TABLE 3
Variable Heavy Chain (VH) and Variable Light Chain (VL) Amino Acid
Sequences of Anti-Human Cleaved CDCP1 Antibodies

| | VH Amino Acid Sequence | VL Amino Acid Sequence |
|---|---|---|
| Antibody | (SEQ ID NO) | (SEQ ID NO) |
| CL03 Fab | EVQLVESGGGLVQPGGSLRLSCAASGF | DIQMTQSPSSLSASVGDRVTITCRASQS |
| | NFSSYSIHWVRQAPGKGLEWVASIYPYS | VSSAVAWYQQKPGKAPKLLIYSASSLY |
| | GSTYYADSVKGRFTISADTSKNTAYLQ | SGVPSRFSGSRSGTDFTLTISSLQPEDFA |
| | MNSLRAEDTAVYYCARQSHSYYSHTW | TYYCQQSGQRPITFGQGTKVEIK |
| | WVSYGMDYWGQGTLVTVSS | (SEQ ID NO: 62) |
| | (SEQ ID NO: 61) | |
| CL03 IgG | EVQLVESGGGLVQPGGSLRLSCAASGF | DIQMTQSPSSLSASVGDRVTITCRASQS |
| | NFSSYSIHWVRQAPGKGLEWVASIYPYS | VSSAVAWYQQKPGKAPKLLIYSASSLY |
| | GSTYYADSVKGRFTISADTSKNTAYLQ | SGVPSRFSGSRSGTDFTLTISSLQPEDFA |
| | MNSLRAEDTAVYYCARQSHSYYSHTW | TYYCQQSGQRPITFGQGTKVEIK |
| | WVSYGMDYWGQGTLVTVSS | (SEQ ID NO: 62) |
| | (SEQ ID NO: 61) | |
| CL03 IgG H1- | EVQLVESGGGLVQPGGSLRLSCAASGF | DIQMTQSPSSLSASVGDRVTITCRASQS |
| NtoD | DFSSYSIHWVRQAPGKGLEWVASIYPYS | VSSAVAWYQQKPGKAPKLLIYSASSLY |
| | GSTYYADSVKGRFTISADTSKNTAYLQ | SGVPSRFSGSRSGTDFTLTISSLQPEDFA |
| | MNSLRAEDTAVYYCARQSHSYYSHTW | TYYCQQSGQRPITFGQGTKVEIK |
| | WVSYGMDYWGQGTLVTVSS | (SEQ ID NO: 62) |
| | (SEQ ID NO: 65) | |
| CL03 IgG H1- | EVQLVESGGGLVQPGGSLRLSCAASGF | DIQMTQSPSSLSASVGDRVTITCRASQS |
| NtoD H3-MtoA | DFSSYSIHWVRQAPGKGLEWVASIYPYS | VSSAVAWYQQKPGKAPKLLIYSASSLY |
| (“IgG101”) | GSTYYADSVKGRFTISADTSKNTAYLQ | SGVPSRFSGSRSGTDFTLTISSLQPEDFA |
| | MNSLRAEDTAVYYCARQSHSYYSHTW | TYYCQQSGQRPITFGQGTKVEIK |
| | WVSYGADYWGQGTLVTVSS | (SEQ ID NO: 62) |
| | (SEQ ID NO: 67) | |
| CL03 IgG H1- | EVQLVESGGGLVQPGGSLRLSCAASGF | DIQMTQSPSSLSASVGDRVTITCRASQS |
| NtoD H3-MtoI | DFSSYSIHWVRQAPGKGLEWVASIYPYS | VSSAVAWYQQKPGKAPKLLIYSASSLY |
| | GSTYYADSVKGRFTISADTSKNTAYLQ | SGVPSRFSGSRSGTDFTLTISSLQPEDFA |
| | MNSLRAEDTAVYYCARQSHSYYSHTW | TYYCQQSGQRPITFGQGTKVEIK |
| | WVSYGIDYWGQGTLVTVSS | (SEQ ID NO: 62) |
| | (SEQ ID NO: 69) | |
| CL03 IgG H1- | EVQLVESGGGLVQPGGSLRLSCAASGF | DIQMTQSPSSLSASVGDRVTITCRASQS |
| NtoD H3-MtoL | DFSSYSIHWVRQAPGKGLEWVASIYPYS | VSSAVAWYQQKPGKAPKLLIYSASSLY |
| | GSTYYADSVKGRFTISADTSKNTAYLQ | SGVPSRFSGSRSGTDFTLTISSLQPEDFA |
| | MNSLRAEDTAVYYCARQSHSYYSHTW | TYYCQQSGQRPITFGQGTKVEIK |
| | WVSYGLDYWGQGTLVTVSS | (SEQ ID NO: 62) |
| | (SEQ ID NO: 71) | |
| CL03 IgG H1- | EVQLVESGGGLVQPGGSLRLSCAASGF | DIQMTQSPSSLSASVGDRVTITCRASQS |
| NtoD H3-MtoV | DFSSYSIHWVRQAPGKGLEWVASIYPYS | VSSAVAWYQQKPGKAPKLLIYSASSLY |
| | GSTYYADSVKGRFTISADTSKNTAYLQ | SGVPSRFSGSRSGTDFTLTISSLQPEDFA |
| | MNSLRAEDTAVYYCARQSHSYYSHTW | TYYCQQSGQRPITFGQGTKVEIK |
| | WVSYGVDYWGQGTLVTVSS | (SEQ ID NO: 62) |
| | (SEQ ID NO: 73) | |
| CL07 Fab | EVQLVESGGGLVQPGGSLRLSCAASGF | DIQMTQSPSSLSASVGDRVTITCRASQS |
| | NFSSSSIHWVRQAPGKGLEWVASIYPYS | VSSAVAWYQQKPGKAPKLLIYSASSLY |
| | GSTSYADSVKGRFTISADTSKNTAYLQ | SGVPSRFSGSRSGTDFTLTISSLQPEDFA |
| | MNSLRAEDTAVYYCARTYWVQFWYG | TYYCQQSSSSLITFGQGTKVEIK |
| | HFSYYRPALDYWGQGTLVTVSS | (SEQ ID NO: 76) |
| | (SEQ ID NO: 75) | |
| CL07 IgG | EVQLVESGGGLVQPGGSLRLSCAASGF | DIQMTQSPSSLSASVGDRVTITCRASQS |
| | NFSSSSIHWVRQAPGKGLEWVASIYPYS | VSSAVAWYQQKPGKAPKLLIYSASSLY |
| | GSTSYADSVKGRFTISADTSKNTAYLQ | SGVPSRFSGSRSGTDFTLTISSLQPEDFA |
| | MNSLRAEDTAVYYCARTYWVQFWYG | TYYCQQSSSSLITFGQGTKVEIK |
| | HFSYYRPALDYWGQGTLVTVSS | (SEQ ID NO: 76) |
| | (SEQ ID NO: 75) | |
| CL07 IgG H1- | EVQLVESGGGLVQPGGSLRLSCAASGF | DIQMTQSPSSLSASVGDRVTITCRASQS |
| NtoD | DFSSSSIHWVRQAPGKGLEWVASIYPYS | VSSAVAWYQQKPGKAPKLLIYSASSLY |
| | GSTSYADSVKGRFTISADTSKNTAYLQ | SGVPSRFSGSRSGTDFTLTISSLQPEDFA |
| | MNSLRAEDTAVYYCARTYWVQFWYG | TYYCQQSSSSLITFGQGTKVEIK |
| | HFSYYRPALDYWGQGTLVTVSS | (SEQ ID NO: 76) |
| | (SEQ ID NO: 79) | |
| IgG3H7L | EVQLVESGGGLVQPGGSLRLSCAASGF | DIQMTQSPSSLSASVGDRVTITCRASQS |
| | DFSSYSIHWVRQAPGKGLEWVASIYPYS | VSSAVAWYQQKPGKAPKLLIYSASSLY |
| | GSTYYADSVKGRFTISADTSKNTAYLQ | SGVPSRFSGSRSGTDFTLTISSLQPEDFA |
| | MNSLRAEDTAVYYCARQSHSYYSHTW | TYYCQQSSSSLITFGQGTKVEIK |
| | WVSYGMDYWGQGTLVTVSS | (SEQ ID NO: 76) |
| | (SEQ ID NO: 65) | |
| IgGA01 | EVQLVESGGGLVQPGGSLRLSCAASGF | DIQMTQSPSSLSASVGDRVTITCRASQS |
| | DFSSYSIHWVRQAPGKGLEWVASIYPYS | VSSAVAWYQQKPGKAPKLLIYSASSLY |
| | GSTYYADSVKGRFTISADTSKNTAYLQ | SGVPSRFSGSRSGTDFTLTISSLQPEDFA |
| | MNSLRAEDTAVYYCARQNHSYYSHTW | TYYCQQSGQRPITFGQGTKVEIK |
| | WVSYGMDYWGQGTLVTVSS | (SEQ ID NO: 62) |
| | (SEQ ID NO: 83) | |
| IgGA03 | EVQLVESGGGLVQPGGSLRLSCAASGF | DIQMTQSPSSLSASVGDRVTITCRASQS |
| | DFSSYSIHWVRQAPGKGLEWVASIYPYS | VSSAVAWYQQKPGKAPKLLIYSASSLY |
| | GSTYYADSVKGRFTISADTSKNTAYLQ | SGVPSRFSGSRSGTDFTLTISSLQPEDFA |
| | MNSLRAEDTAVYYCARQTHSYYSHTW | TYYCQQSGQRPITFGQGTKVEIK |
| | WVSYGMDYWGQGTLVTVSS | (SEQ ID NO: 62) |
| | (SEQ ID NO: 85) | |
| IgGA04 | EVQLVESGGGLVQPGGSLRLSCAASGF | DIQMTQSPSSLSASVGDRVTITCRASQS |
| | DFSSYSIHWVRQAPGKGLEWVASIYPYS | VSSAVAWYQQKPGKAPKLLIYSASSLY |
| | GSTYYADSVKGRFTISADTSKNTAYLQ | SGVPSRFSGSRSGTDFTLTISSLQPEDFA |
| | MNSLRAEDTAVYYCARQGHSYYSHTW | TYYCQQSGQRPITFGQGTKVEIK |
| | WVSYGMDYWGQGTLVTVSS | (SEQ ID NO: 62) |
| | (SEQ ID NO: 87) | |
| IgGA05 | EVQLVESGGGLVQPGGSLRLSCAASGF | DIQMTQSPSSLSASVGDRVTITCRASQS |
| | DFSSYSIHWVRQAPGKGLEWVASIYPYS | VSSAVAWYQQKPGKAPKLLIYSASSLY |
| | GSTYYADSVKGRFTISADTSKNTAYLQ | SGVPSRFSGSRSGTDFTLTISSLQPEDFA |
| | MNSLRAEDTAVYYCARQAHSYYSHTW | TYYCQQSGQRPITFGQGTKVEIK |
| | WVSYGMDYWGQGTLVTVSS | (SEQ ID NO: 62) |
| | (SEQ ID NO: 89) | |
| IgGA06 | EVQLVESGGGLVQPGGSLRLSCAASGF | DIQMTQSPSSLSASVGDRVTITCRASQS |
| | DFSSYSIHWVRQAPGKGLEWVASIYPYS | VSSAVAWYQQKPGKAPKLLIYSASSLY |
| | GSTYYADSVKGRFTISADTSKNTAYLQ | SGVPSRFSGSRSGTDFTLTISSLQPEDFA |
| | MNSLRAEDTAVYYCARQGQSYYSHTW | TYYCQQSGQRPITFGQGTKVEIK |
| | WVSYGMDYWGQGTLVTVSS | (SEQ ID NO: 62) |
| | (SEQ ID NO: 91) | |
| IgGA08 | EVQLVESGGGLVQPGGSLRLSCAASGF | DIQMTQSPSSLSASVGDRVTITCRASQS |
| | DFSSYSIHWVRQAPGKGLEWVASIYPYS | VSSAVAWYQQKPGKAPKLLIYSASSLY |
| | GSTYYADSVKGRFTISADTSKNTAYLQ | SGVPSRFSGSRSGTDFTLTISSLQPEDFA |
| | MNSLRAEDTAVYYCARQSHSYYSHTW | TYYCQQSGERPITFGQGTKVEIK |
| | WVSYGMDYWGQGTLVTVSS | (SEQ ID NO: 94) |
| | (SEQ ID NO: 65) | |
| IgGA09 | EVQLVESGGGLVQPGGSLRLSCAASGF | DIQMTQSPSSLSASVGDRVTITCRASQS |
| | DFSSYSIHWVRQAPGKGLEWVASIYPYS | VSSAVAWYQQKPGKAPKLLIYSASSLY |
| | GSTYYADSVKGRFTISADTSKNTAYLQ | SGVPSRFSGSRSGTDFTLTISSLQPEDFA |
| | MNSLRAEDTAVYYCARQSHSYYSHTW | TYYCQQSGNVPATFGQGTKVEIK |
| | WVSYGMDYWGQGTLVTVSS | (SEQ ID NO: 96) |
| | (SEQ ID NO: 65) | |
| IgGA10 | EVQLVESGGGLVQPGGSLRLSCAASGF | DIQMTQSPSSLSASVGDRVTITCRASQS |
| | DFSSYSIHWVRQAPGKGLEWVASIYPYS | VSSAVAWYQQKPGKAPKLLIYSASSLY |
| | GSTYYADSVKGRFTISADTSKNTAYLQ | SGVPSRFSGSRSGTDFTLTISSLQPEDFA |
| | MNSLRAEDTAVYYCARQSHSYYSHTW | TYYCQQSGKRPMTFGQGTKVEIK |
| | WVSYGMDYWGQGTLVTVSS | (SEQ ID NO: 98) |
| | (SEQ ID NO: 65) | |
| IgGA11 | EVQLVESGGGLVQPGGSLRLSCAASGF | DIQMTQSPSSLSASVGDRVTITCRASQS |
| | DFSSYSIHWVRQAPGKGLEWVASIYPYS | VSSAVAWYQQKPGKAPKLLIYSASSLY |
| | GSTYYADSVKGRFTISADTSKNTAYLQ | SGVPSRFSGSRSGTDFTLTISSLQPEDFA |
| | MNSLRAEDTAVYYCARRSHSYYSHTW | TYYCQQTGQRPMTFGQGTKVEIK |
| | WVSYGMDYWGQGTLVTVSS | (SEQ ID NO: 100) |
| | (SEQ ID NO: 99) | |
| IgGB01 | EVQLVESGGGLVQPGGSLRLSCAASGF | DIQMTQSPSSLSASVGDRVTITCRASQS |
| | DFSSYSIHWVRQAPGKGLEWVASIYPYS | VSSAVAWYQQKPGKAPKLLIYSASSLY |
| | GSTYYADSVKGRFTISADTSKNTAYLQ | SGVPSRFSGSRSGTDFTLTISSLQPEDFA |
| | MNSLRAEDTAVYYCARQSHSYYSHTW | TYYCQQSMQWPKTFGQGTKVEIK |
| | WVSYGMDYWGQGTLVTVSS | (SEQ ID NO: 102) |
| | (SEQ ID NO: 65) | |
| IgGB03 | EVQLVESGGGLVQPGGSLRLSCAASGF | DIQMTQSPSSLSASVGDRVTITCRASQS |
| | DFSSYSIHWVRQAPGKGLEWVASIYPYS | VSSAVAWYQQKPGKAPKLLIYSASSLY |
| | GSTYYADSVKGRFTISADTSKNTAYLQ | SGVPSRFSGSRSGTDFTLTISSLQPEDFA |
| | MNSLRAEDTAVYYCARQSHSFYSHTW | TYYCQQSLQRPMTFGQGTKVEIK |
| | WVSYGMDYWGQGTLVTVSS | (SEQ ID NO: 104) |
| | (SEQ ID NO: 103) | |
| IgGB04 | EVQLVESGGGLVQPGGSLRLSCAASGF | DIQMTQSPSSLSASVGDRVTITCRASQS |
| | DFSSYSIHWVRQAPGKGLEWVASIYPYS | VSSAVAWYQQKPGKAPKLLIYSASSLY |
| | GSTYYADSVKGRFTISADTSKNTAYLQ | SGVPSRFSGSRSGTDFTLTISSLQPEDFA |
| | MNSLRAEDTAVYYCARRSHSYYSHTW | TYYCQQVELVPMTFGQGTKVEIK |
| | WVSYGMDYWGQGTLVTVSS | (SEQ ID NO: 106) |
| | (SEQ ID NO: 99) | |
| IgGB07 | EVQLVESGGGLVQPGGSLRLSCAASGF | DIQMTQSPSSLSASVGDRVTITCRASQS |
| | DFSSYSIHWVRQAPGKGLEWVASIYPYS | VSSAVAWYQQKPGKAPKLLIYSASSLY |
| | GSTYYADSVKGRFTISADTSKNTAYLQ | SGVPSRFSGSRSGTDFTLTISSLQPEDFA |
| | MNSLRAEDTAVYYCARQDHSYYSHTW | TYYCQQAGKRPLTFGQGTKVEIK |
| | WVSYGMDYWGQGTLVTVSS | (SEQ ID NO: 108) |
| | (SEQ ID NO: 107) | |
| IgGB08 | EVQLVESGGGLVQPGGSLRLSCAASGF | DIQMTQSPSSLSASVGDRVTITCRASQS |
| | DFSSYSIHWVRQAPGKGLEWVASIYPYS | VSSAVAWYQQKPGKAPKLLIYSASSLY |
| | GSTYYADSVKGRFTISADTSKNTAYLQ | SGVPSRFSGSRSGTDFTLTISSLQPEDFA |
| | MNSLRAEDTAVYYCARRSHSYYSHTW | TYYCQQSVDLTATFGQGTKVEIK |
| | WVSYGMDYWGQGTLVTVSS | (SEQ ID NO: 110) |
| | (SEQ ID NO: 99) | |
| IgGC01 | EVQLVESGGGLVQPGGSLRLSCAASGF | DIQMTQSPSSLSASVGDRVTITCRASQS |
| | DFSSYSIHWVRQAPGKGLEWVASIYPYS | VSSAVAWYQQKPGKAPKLLIYSASSLY |
| | GSTYYADSVKGRFTISADTSKNTAYLQ | SGVPSRFSGSRSGTDFTLTISSLQPEDFA |
| | MNSLRAEDTAVYYCARRSHSYYSHTW | TYYCQQLGVRAATFGQGTKVEIK |
| | WVSYGMDYWGQGTLVTVSS | (SEQ ID NO: 112) |
| | (SEQ ID NO: 99) | |
| IgGC03 | EVQLVESGGGLVQPGGSLRLSCAASGF | DIQMTQSPSSLSASVGDRVTITCRASQS |
| | DFSSYSIHWVRQAPGKGLEWVASIYPYS | VSSAVAWYQQKPGKAPKLLIYSASSLY |
| | GSTYYADSVKGRFTISADTSKNTAYLQ | SGVPSRFSGSRSGTDFTLTISSLQPEDFA |
| | MNSLRAEDTAVYYCARQSHSYYSHTW | TYYCQQSGRKPVTFGQGTKVEIK |
| | WVSYGMDYWGQGTLVTVSS | (SEQ ID NO: 114) |
| | (SEQ ID NO: 65) | |
| IgGC04 | EVQLVESGGGLVQPGGSLRLSCAASGF | DIQMTQSPSSLSASVGDRVTITCRASQS |
| | DFSSYSIHWVRQAPGKGLEWVASIYPYS | VSSAVAWYQQKPGKAPKLLIYSASSLY |
| | GSTYYADSVKGRFTISADTSKNTAYLQ | SGVPSRFSGSRSGTDFTLTISSLQPEDFA |
| | MNSLRAEDTAVYYCARRSHSYYSHTW | TYYCQQTAQSPLTFGQGTKVEIK |
| | WVSYGMDYWGQGTLVTVSS | (SEQ ID NO: 116) |
| | (SEQ ID NO: 99) | |
| IgGC05 | EVQLVESGGGLVQPGGSLRLSCAASGF | DIQMTQSPSSLSASVGDRVTITCRASQS |
| | DFSSYSIHWVRQAPGKGLEWVASIYPYS | VSSAVAWYQQKPGKAPKLLIYSASSLY |
| | GSTYYADSVKGRFTISADTSKNTAYLQ | SGVPSRFSGSRSGTDFTLTISSLQPEDFA |
| | MNSLRAEDTAVYYCARQSHSYYSHTW | TYYCQQSRQRPLTFGQGTKVEIK |
| | WVSYGMDYWGQGTLVTVSS | (SEQ ID NO: 118) |
| | (SEQ ID NO: 65) | |
| IgGC06 | EVQLVESGGGLVQPGGSLRLSCAASGF | DIQMTQSPSSLSASVGDRVTITCRASQS |
| | DFSSYSIHWVRQAPGKGLEWVASIYPYS | VSSAVAWYQQKPGKAPKLLIYSASSLY |
| | GSTYYADSVKGRFTISADTSKNTAYLQ | SGVPSRFSGSRSGTDFTLTISSLQPEDFA |
| | MNSLRAEDTAVYYCARQSHSYYSHTW | TYYCQQSGLMPLTFGQGTKVEIK |
| | WVSYGMDYWGQGTLVTVSS | (SEQ ID NO: 120) |
| | (SEQ ID NO: 65) | |
| IgGC09 | EVQLVESGGGLVQPGGSLRLSCAASGF | DIQMTQSPSSLSASVGDRVTITCRASQS |
| | DFSSYSIHWVRQAPGKGLEWVASIYPYS | VSSAVAWYQQKPGKAPKLLIYSASSLY |
| | GSTYYADSVKGRFTISADTSKNTAYLQ | SGVPSRFSGSRSGTDFTLTISSLQPEDFA |
| | MNSLRAEDTAVYYCARQSHSYYSHTW | TYYCQQSGHRPITFGQGTKVEIK |
| | WVSYGMDYWGQGTLVTVSS | (SEQ ID NO: 122) |
| | (SEQ ID NO: 65) | |
| IgGD02 | EVQLVESGGGLVQPGGSLRLSCAASGF | DIQMTQSPSSLSASVGDRVTITCRASQS |
| | DFSSYSIHWVRQAPGKGLEWVASIYPYS | VSSAVAWYQQKPGKAPKLLIYSASSLY |
| | GSTYYADSVKGRFTISADTSKNTAYLQ | SGVPSRFSGSRSGTDFTLTISSLQPEDFA |
| | MNSLRAEDTAVYYCARKYHSYYSHTW | TYYCQQSSQRPATFGQGTKVEIK |
| | WVSYGMDYWGQGTLVTVSS | (SEQ ID NO: 124) |
| | (SEQ ID NO: 123) | |
| IgGD03 | EVQLVESGGGLVQPGGSLRLSCAASGF | DIQMTQSPSSLSASVGDRVTITCRASQS |
| | DFSSYSIHWVRQAPGKGLEWVASIYPYS | VSSAVAWYQQKPGKAPKLLIYSASSLY |
| | GSTYYADSVKGRFTISADTSKNTAYLQ | SGVPSRFSGSRSGTDFTLTISSLQPEDFA |
| | MNSLRAEDTAVYYCARQDHSYYSHTW | TYYCQQSGRQPLTFGQGTKVEIK |
| | WVSYGMDYWGQGTLVTVSS | (SEQ ID NO: 126) |
| | (SEQ ID NO: 107) | |
| IgGD04 | EVQLVESGGGLVQPGGSLRLSCAASGF | DIQMTQSPSSLSASVGDRVTITCRASQS |
| | DFSSYSIHWVRQAPGKGLEWVASIYPYS | VSSAVAWYQQKPGKAPKLLIYSASSLY |
| | GSTYYADSVKGRFTISADTSKNTAYLQ | SGVPSRFSGSRSGTDFTLTISSLQPEDFA |
| | MNSLRAEDTAVYYCARRSHSYYSHTW | TYYCQQFMRPAFTFGQGTKVEIK |
| | WVSYGMDYWGQGTLVTVSS | (SEQ ID NO: 128) |
| | (SEQ ID NO: 99) | |
| IgGD07 | EVQLVESGGGLVQPGGSLRLSCAASGF | DIQMTQSPSSLSASVGDRVTITCRASQS |
| | DFSSYSIHWVRQAPGKGLEWVASIYPYS | VSSAVAWYQQKPGKAPKLLIYSASSLY |
| | GSTYYADSVKGRFTISADTSKNTAYLQ | SGVPSRFSGSRSGTDFTLTISSLQPEDFA |
| | MNSLRAEDTAVYYCARRSHSYYSHTW | TYYCQQSGQPSMTFGQGTKVEIK |
| | WVSYGMDYWGQGTLVTVSS | (SEQ ID NO: 130) |
| | (SEQ ID NO: 99) | |
| IgGD08 | EVQLVESGGGLVQPGGSLRLSCAASGF | DIQMTQSPSSLSASVGDRVTITCRASQS |
| | DFSSYSIHWVRQAPGKGLEWVASIYPYS | VSSAVAWYQQKPGKAPKLLIYSASSLY |
| | GSTYYADSVKGRFTISADTSKNTAYLQ | SGVPSRFSGSRSGTDFTLTISSLQPEDFA |
| | MNSLRAEDTAVYYCARQSHSYYSHTW | TYYCQQSVLRPFTFGQGTKVEIK |
| | WVSYGMDYWGQGTLVTVSS | (SEQ ID NO: 132) |
| | (SEQ ID NO: 65) | |
| IgGD09 | EVQLVESGGGLVQPGGSLRLSCAASGF | DIQMTQSPSSLSASVGDRVTITCRASQS |
| | NFSSSSIHWVRQAPGKGLEWVASIYPYS | VSSAVAWYQQKPGKAPKLLIYSASSLY |
| | GSTSYADSVKGRFTISADTSKNTAYLQ | SGVPSRFSGSRSGTDFTLTISSLQPEDFA |
| | MNSLRAEDTAVYYCARIYWVDFWYGH | TYYCQQSSSSLITFGQGTKVEIK |
| | FSYYRPALDYWGQGTLVTVSS | (SEQ ID NO: 76) |
| | (SEQ ID NO: 133) | |
| IgG87 | EVQLVESGGGLVQPGGSLRLSCAASGF | DIQMTQSPSSLSASVGDRVTITCRASQS |
| | DASSYSIHWVRQAPGKGLEWVASIYPY | VSSAVAWYQQKPGKAPKLLIYSASSLY |
| | SGSTYYADSVKGRFTISADTSKNTAYLQ | SGVPSRFSGSRSGTDFTLTISSLQPEDFA |
| | MNSLRAEDTAVYYCARQSHSYYSHTW | TYYCQQSGQRPITFGQGTKVEIK (SEQ |
| | WVSYGMDYWGQGTLVTVSS (SEQ ID | ID NO: 62) |
| | NO: 80) | |
| IgG89 | EVQLVESGGGLVQPGGSLRLSCAASGF | DIQMTQSPSSLSASVGDRVTITCRASQS |
| | DFSSYSAHWVRQAPGKGLEWVASIYPY | VSSAVAWYQQKPGKAPKLLIYSASSLY |
| | SGSTYYADSVKGRFTISADTSKNTAYLQ | SGVPSRFSGSRSGTDFTLTISSLQPEDFA |
| | MNSLRAEDTAVYYCARQSHSYYSHTW | TYYCQQSGQRPITFGQGTKVEIK (SEQ |
| | WVSYGMDYWGQGTLVTVSS (SEQ ID | ID NO: 62) |
| | NO: 81) | |
| IgG94 | EVQLVESGGGLVQPGGSLRLSCAASGF | DIQMTQSPSSLSASVGDRVTITCRASQS |
| | DFSSYSIHWVRQAPGKGLEWVASIYPYS | VSSAVAWYQQKPGKAPKLLIYSASSLY |
| | GSTSYADSVKGRFTISADTSKNTAYLQ | SGVPSRFSGSRSGTDFTLTISSLQPEDFA |
| | MNSLRAEDTAVYYCARQSHSYYSHTW | TYYCQQSGQRPITFGQGTKVEIK (SEQ |
| | WVSYGMDYWGQGTLVTVSS (SEQ ID | ID NO: 62) |
| | NO: 82) | |
| IgG97 | EVQLVESGGGLVQPGGSLRLSCAASGF | DIQMTQSPSSLSASVGDRVTITCRASQS |
| | DFSSYSIHWVRQAPGKGLEWVASIYPYS | VSSAVAWYQQKPGKAPKLLIYSASSLY |
| | GSTYYADSVKGRFTISADTSKNTAYLQ | SGVPSRFSGSRSGTDFTLTISSLQPEDFA |
| | MNSLRAEDTAVYYCARQSHSYYSHTA | TYYCQQSGQRPITFGQGTKVEIK (SEQ |
| | WVSYGMDYWGQGTLVTVSS (SEQ ID | ID NO: 62) |
| | NO: 84) | |
| IgG102 | EVQLVESGGGLVQPGGSLRLSCAASGF | DIQMTQSPSSLSASVGDRVTITCRASQS |
| | DFSSYSIHWVRQAPGKGLEWVASSYPY | VSSAVAWYQQKPGKAPKLLIYSASSLY |
| | SGSTYYADSVKGRFTISADTSKNTAYLQ | SGVPSRFSGSRSGTDFTLTISSLQPEDFA |
| | MNSLRAEDTAVYYCARQSHSYYSHTW | TYYCQQSGQRPITFGQGTKVEIK (SEQ |
| | WVSYGMDYWGQGTLVTVSS (SEQ ID | ID NO: 62) |
| | NO: 86) | |

In some aspects, the anti-mouse cleaved CDCP1 antibody or antigen-binding fragment thereof comprises a VH comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to an amino acid sequence selected from the group consisting of SEQ ID NOs: 135, 139, 143, 145, 147, 149, 151, 153, 155, 157, 159, and 161. In certain aspects, the VH comprises an amino acid sequence selected from the group consisting of SEQ ID NOs: 135, 139, 143, 145, 147, 149, 151, 153, 155, 157, 159, and 161, as disclosed in Table 4, below.

In some aspects, the anti-mouse cleaved CDCP1 antibody or antigen-binding fragment thereof comprises a VL comprising an amino acid sequence having at least about 60%, at least about 90%, at least about 95%, at least about 96%, at least about 97%, at least about 98%, or at least about 99% sequence identity to an amino acid sequence selected from the group consisting of SEQ ID NOs: 136 and 140. In certain aspects, the VL comprises an amino acid sequence selected from the group consisting of SEQ ID NOs: 136 and 140, as disclosed in Table 4, below.

TABLE 4
Variable Heavy Chain (VH) and Variable Light Chain (VL) Amino Acid Sequences
of Anti-Mouse Cleaved CDCP1 Antibodies

| | VH Amino Acid Sequence | VL Amino Acid Sequence |
|---|---|---|
| Antibody | (SEQ ID NQ) | (SEQ ID NQ) |
| Fab 0114 | EVQLVESGGGLVQPGGSLRLSCAASG | DIQMTQSPSSLSASVGDRVTITCRASQS |
| | FNVSYSSIHWVRQAPGKGLEWVASISS | VSSAVAWYQQKPGKAPKLLIYSASSLY |
| | SSGYTYYADSVKGRFTISADTSKNTA | SGVPSRFSGSRSGTDFTLTISSLQPEDFA |
| | YLQMNSLRAEDTAVYYCARQEGDW | TYYCQQSSWGLFTFGQGTKVEIK |
| | WVAYWPALDYWGQGTLVTVSS | (SEQ ID NO: 136) |
| | (SEQ ID NO: 135) | |
| IgG 0029 (Fab | EVQLVESGGGLVQPGGSLRLSCAASG | DIQMTQSPSSLSASVGDRVTITCRASQS |
| 0114) | FNVSYSSIHWVRQAPGKGLEWVASISS | VSSAVAWYQQKPGKAPKLLIYSASSLY |
| | SSGYTYYADSVKGRFTISADTSKNTA | SGVPSRFSGSRSGTDFTLTISSLQPEDFA |
| | YLQMNSLRAEDTAVYYCARQEGDW | TYYCQQSSWGLFTFGQGTKVEIK |
| | WVAYWPALDYWGQGTLVTVSS | (SEQ ID NO: 136) |
| | (SEQ ID NO: 135) | |
| Fab 0123 | EVQLVESGGGLVQPGGSLRLSCAASG | DIQMTQSPSSLSASVGDRVTITCRASQS |
| | FNVYSYSIHWVRQAPGKGLEWVASIS | VSSAVAWYQQKPGKAPKLLIYSASSLY |
| | SSSGYTYYADSVKGRFTISADTSKNTA | SGVPSRFSGSRSGTDFTLTISSLQPEDFA |
| | YLQMNSLRAEDTAVYYCARYYWDP | TYYCQQTYSGPITFGQGTKVEIK |
| | WQYWYYSYLAFDYWGQGTLVTVSS | (SEQ ID NO: 140) |
| | (SEQ ID NO: 139) | |
| IgG 0030 (Fab | EVQLVESGGGLVQPGGSLRLSCAASG | DIQMTQSPSSLSASVGDRVTITCRASQS |
| 0123) | FNVYSYSIHWVRQAPGKGLEWVASIS | VSSAVAWYQQKPGKAPKLLIYSASSLY |
| | SSSGYTYYADSVKGRFTISADTSKNTA | SGVPSRFSGSRSGTDFTLTISSLQPEDFA |
| | YLQMNSLRAEDTAVYYCARYYWDP | TYYCQQTYSGPITFGQGTKVEIK |
| | WQYWYYSYLAFDYWGQGTLVTVSS | (SEQ ID NO: 140) |
| | (SEQ ID NO: 139) | |
| IgG 0046 | EVQLVESGGGLVQPGGSLRLSCAASG | DIQMTQSPSSLSASVGDRVTITCRASQS |
| | FNVYSYSIHWVRQAPGKGLEWVASIS | VSSAVAWYQQKPGKAPKLLIYSASSLY |
| | SSSGYTYYADSVKGRFTISADTSKNTA | SGVPSRFSGSRSGTDFTLTISSLQPEDFA |
| | YLQMNSLRAEDTAVYYCARYYWDP | TYYCQQTYSGPITFGQGTKVEIK |
| | WQYAYYSYLAFDYWGQGTLVTVSS | (SEQ ID NO: 140) |
| | (SEQ ID NO: 143) | |
| IgG 0050 | EVQLVESGGGLVQPGGSLRLSCAASG | DIQMTQSPSSLSASVGDRVTITCRASQS |
| | FNVYSYSIHWVRQAPGKGLEWVASIS | VSSAVAWYQQKPGKAPKLLIYSASSLY |
| | SSSGSTYYADSVKGRFTISADTSKNTA | SGVPSRFSGSRSGTDFTLTISSLQPEDFA |
| | YLQMNSLRAEDTAVYYCARYYWDP | TYYCQQTYSGPITFGQGTKVEIK |
| | WQYAYYSYLAFDYWGQGTLVTVSS | (SEQ ID NO: 140) |
| | (SEQ ID NO: 145) | |
| IgG 0051 | EVQLVESGGGLVQPGGSLRLSCAASG | DIQMTQSPSSLSASVGDRVTITCRASQS |
| | FNVYSYSIHWVRQAPGKGLEWVASIS | VSSAVAWYQQKPGKAPKLLIYSASSLY |
| | SSSGYTSYADSVKGRFTISADTSKNTA | SGVPSRFSGSRSGTDFTLTISSLQPEDFA |
| | YLQMNSLRAEDTAVYYCARYYWDP | TYYCQQTYSGPITFGQGTKVEIK |
| | WQYAYYSYLAFDYWGQGTLVTVSS | (SEQ ID NO: 140) |
| | (SEQ ID NO: 147) | |
| IgG 0055 | EVQLVESGGGLVQPGGSLRLSCAASG | DIQMTQSPSSLSASVGDRVTITCRASQS |
| | FNVYSYSIHWVRQAPGKGLEWVASIS | VSSAVAWYQQKPGKAPKLLIYSASSLY |
| | SSSGYTYYADSVKGRFTISADTSKNTA | SGVPSRFSGSRSGTDFTLTISSLQPEDFA |
| | YLQMNSLRAEDTAVYYCARYYWDP | TYYCQQTYSGPITFGQGTKVEIK |
| | WQYASYSYLAFDYWGQGTLVTVSS | (SEQ ID NO: 140) |
| | (SEQ ID NO: 149) | |
| IgG 0058 | EVQLVESGGGLVQPGGSLRLSCAASG | DIQMTQSPSSLSASVGDRVTITCRASQS |
| | FNVYSYSIHWVRQAPGKGLEWVASIS | VSSAVAWYQQKPGKAPKLLIYSASSLY |
| | SSSGSTSYADSVKGRFTISADTSKNTA | SGVPSRFSGSRSGTDFTLTISSLQPEDFA |
| | YLQMNSLRAEDTAVYYCARYYWDP | TYYCQQTYSGPITFGQGTKVEIK |
| | WQYASYSYLAFDYWGQGTLVTVSS | (SEQ ID NO: 140) |
| | (SEQ ID NO: 151) | |
| IgG 0059 | EVQLVESGGGLVQPGGSLRLSCAASG | DIQMTQSPSSLSASVGDRVTITCRASQS |
| | FNVYSYSIHWVRQAPGKGLEWVASIS | VSSAVAWYQQKPGKAPKLLIYSASSLY |
| | SSSGSTYYADSVKGRFTISADTSKNTA | SGVPSRFSGSRSGTDFTLTISSLQPEDFA |
| | YLQMNSLRAEDTAVYYCARYYWDP | TYYCQQTYSGPITFGQGTKVEIK |
| | WQYASYSYLAFDYWGQGTLVTVSS | (SEQ ID NO: 140) |
| | (SEQ ID NO: 153) | |
| IgG 0060 | EVQLVESGGGLVQPGGSLRLSCAASG | DIQMTQSPSSLSASVGDRVTITCRASQS |
| | FNVYSYSIHWVRQAPGKGLEWVASIS | VSSAVAWYQQKPGKAPKLLIYSASSLY |
| | SSSGYTSYADSVKGRFTISADTSKNTA | SGVPSRFSGSRSGTDFTLTISSLQPEDFA |
| | YLQMNSLRAEDTAVYYCARYYWDP | TYYCQQTYSGPITFGQGTKVEIK |
| | WQYASYSYLAFDYWGQGTLVTVSS | (SEQ ID NO: 140) |
| | (SEQ ID NO: 155) | |
| IgG 0062 | EVQLVESGGGLVQPGGSLRLSCAASG | DIQMTQSPSSLSASVGDRVTITCRASQS |
| | FNVYSYSIHWVRQAPGKGLEWVASIS | VSSAVAWYQQKPGKAPKLLIYSASSLY |
| | SSSGSTYYADSVKGRFTISADTSKNTA | SGVPSRFSGSRSGTDFTLTISSLQPEDFA |
| | YLQMNSLRAEDTAVYYCARYYWDP | TYYCQQTYSGPITFGQGTKVEIK |
| | WQYSSYSYLAFDYWGQGTLVTVSS | (SEQ ID NO: 140) |
| | (SEQ ID NO: 157) | |
| IgG 0063 | EVQLVESGGGLVQPGGSLRLSCAASG | DIQMTQSPSSLSASVGDRVTITCRASQS |
| | FNVYSYSIHWVRQAPGKGLEWVASIS | VSSAVAWYQQKPGKAPKLLIYSASSLY |
| | SSSGYTSYADSVKGRFTISADTSKNTA | SGVPSRFSGSRSGTDFTLTISSLQPEDFA |
| | YLQMNSLRAEDTAVYYCARYYWDP | TYYCQQTYSGPITFGQGTKVEIK |
| | WQYSSYSYLAFDYWGQGTLVTVSS | (SEQ ID NO: 140) |
| | (SEQ ID NO: 159) | |
| IgG 0065 | EVQLVESGGGLVQPGGSLRLSCAASG | DIQMTQSPSSLSASVGDRVTITCRASQS |
| | FNVYSYSIHWVRQAPGKGLEWVASIS | VSSAVAWYQQKPGKAPKLLIYSASSLY |
| | SSSGSTSYADSVKGRFTISADTSKNTA | SGVPSRFSGSRSGTDFTLTISSLQPEDFA |
| | YLQMNSLRAEDTAVYYCARYYWDP | TYYCQQTYSGPITFGQGTKVEIK |
| | WQYSSYSYLAFDYWGQGTLVTVSS | (SEQ ID NO: 140) |
| | (SEQ ID NO: 161) | |

In some aspects, the anti-human cleaved CDCP1 antibody or antigen-binding fragment thereof comprises a heavy chain (HC) comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to an amino acid sequence selected from the group consisting of SEQ ID NOs: 279, 280, 281, 282, 283, 284, 285, 286, 287, 288, 290, 291, 292, 293, 294, 298, 300, 302, 310, and 315. In certain aspects, the HC comprises an amino acid sequence selected from the group consisting of SEQ ID NOs: 279, 280, 281, 282, 283, 284, 285, 286, 287, 288, 290, 291, 292, 293, 294, 298, 300, 302, 310, and 315, as disclosed in Table 5, below. In certain aspects, the heavy chain amino acid sequence comprises one or more deletions, substitutions, or mutations within the immunoglobulin constant region, e.g., within the CH1 domain, the CH2 domain, the CH3 domain, or the hinge region.

In some aspects, the anti-human cleaved CDCP1 antibody or antigen-binding fragment thereof comprises a light chain (LC) comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to an amino acid sequence selected from the group consisting of SEQ ID Nos: 316, 317, 323, 324, 326, 327, 332, 333, 334, 335, 336, 337, 338, 339, 340, 341, 342, 343, 344, 345, 346, 347, 348, 349, 350, 351, and 352. In certain aspects, the LC comprises an amino acid sequence selected from the group consisting of SEQ ID NOs: 316, 317, 323, 324, 326, 327, 332, 333, 334, 335, 336, 337, 338, 339, 340, 341, 342, 343, 344, 345, 346, 347, 348, 349, 350, 351, and 352, as disclosed in Table 5, below.

In some aspects, the anti-human cleaved CDCP1 antibody or antigen-binding fragment thereof comprises a heavy chain (HC) and a light chain (LC), wherein the HC of the antibody or antigen-binding fragment thereof comprises an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to an amino acid sequence selected from the group consisting of SEQ ID NOs: 279, 280, 281, 282, 283, 284, 285, 286, 287, 288, 290, 291, 292, 293, 294, 298, 300, 302, 310, and 315 and the LC comprises an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to an amino acid sequence selected from the group consisting of SEQ ID NOs: 316, 317, 323, 324, 326, 327, 332, 333, 334, 335, 336, 337, 338, 339, 340, 341, 342, 343, 344, 345, 346, 347, 348, 349, 350, 351, and 352.

In some aspects, the anti-human cleaved CDCP1 antibody or antigen-binding fragment thereof comprises a heavy chain (HC) and a light chain (LC), wherein the HC of the antibody or antigen-binding fragment thereof comprises the HC comprises an amino acid sequence selected from the group consisting of SEQ ID NOs: 279, 280, 281, 282, 283, 284, 285, 286, 287, 288, 290, 291, 292, 293, 294, 298, 300, 302, 310, and 315 and the LC comprises an amino acid sequence selected from the group consisting of SEQ ID NOs: 316, 317, 323, 324, 326, 327, 332, 333, 334, 335, 336, 337, 338, 339, 340, 341, 342, 343, 344, 345, 346, 347, 348, 349, 350, 351, and 352.

TABLE 5
Heavy Chain (HC) and Light Chain (LC) Amino Acid
Sequences of Anti-Human Cleaved CDCP1 Antibodies

| | HC Amino Acid Sequence | LC Amino Acid Sequence |
|---|---|---|
| Antibody | (SEQ ID NO) | (SEQ ID NO) |
| CL03 Fab | (SEQ ID NO: 279) | (SEQ ID NO: 316) |
| CL03 IgG | (SEQ ID NO: 280) | (SEQ ID NO: 317) |
| CL03 IgG | (SEQ ID NO: 281) | (SEQ ID NO: 317) |
| H1-NtoD | | |
| CL03 IgG | (SEQ ID NO: 282) | (SEQ ID NO: 317) |
| H1-NtoD | | |
| H3-MtoA | | |
| (“IgG101) | | |
| CL03 IgG | (SEQ ID NO: 283) | (SEQ ID NO: 317) |
| H1-NtoD | | |
| H3-MtoI | | |
| CL03 IgG | (SEQ ID NO: 284) | (SEQ ID NO: 317) |
| H1-NtoD | | |
| H3-MtoL | | |
| CL03 IgG | (SEQ ID NO: 285) | (SEQ ID NO: 317) |
| H1-NtoD | | |
| H3-MtoV | | |
| CL07 Fab | (SEQ ID NO: 286) | (SEQ ID NO: 323) |
| CL07 IgG | (SEQ ID NO: 287) | (SEQ ID NO: 324) |
| CL07 IgG | (SEQ ID NO: 288) | (SEQ ID NO: 324) |
| H1-NtoD | | |
| IgG3H7L | (SEQ ID NO: 281) | (SEQ ID NO: 326) |
| IgGA01 | (SEQ ID NO: 290) | (SEQ ID NO: 327) |
| IgGA03 | (SEQ ID NO: 291) | (SEQ ID NO: 327) |
| IgGA04 | (SEQ ID NO: 292) | (SEQ ID NO: 327) |
| IgGA05 | (SEQ ID NO: 293) | (SEQ ID NO: 327) |
| IgGA06 | (SEQ ID NO: 294) | (SEQ ID NO: 327) |
| IgGA08 | (SEQ ID NO: 281) | (SEQ ID NO: 332) |
| IgGA09 | (SEQ ID NO: 281) | (SEQ ID NO: 333) |
| IgGA10 | (SEQ ID NO: 281) | (SEQ ID NO: 334) |
| IgGA11 | (SEQ ID NO: 298) | (SEQ ID NO: 335) |
| IgGB01 | (SEQ ID NO: 281) | (SEQ ID NO: 336) |
| IgGB03 | (SEQ ID NO: 300) | (SEQ ID NO: 337) |
| IgGB04 | (SEQ ID NO: 298) | (SEQ ID NO: 338) |
| IgGB07 | (SEQ ID NO: 302) | (SEQ ID NO: 339) |
| IgGB08 | (SEQ ID NO: 298) | (SEQ ID NO: 340) |
| IgGC01 | (SEQ ID NO: 298) | (SEQ ID NO: 341) |
| IgGC03 | (SEQ ID NO: 281) | (SEQ ID NO: 342) |
| IgGC04 | (SEQ ID NO: 298) | (SEQ ID NO: 343) |
| IgGC05 | (SEQ ID NO: 281) | (SEQ ID NO: 344) |
| IgGC06 | (SEQ ID NO: 281) | (SEQ ID NO: 345) |
| IgGC09 | (SEQ ID NO: 281) | (SEQ ID NO: 346) |
| IgGD02 | (SEQ ID NO: 310) | (SEQ ID NO: 347) |
| IgGD03 | (SEQ ID NO: 302) | (SEQ ID NO: 348) |
| IgGD04 | (SEQ ID NO: 298) | (SEQ ID NO: 349) |
| IgGD07 | (SEQ ID NO: 298) | (SEQ ID NO: 350) |
| IgGD08 | (SEQ ID NO: 281) | (SEQ ID NO: 351) |
| IgGD09 | (SEQ ID NO: 315) | (SEQ ID NO: 352) |

In some aspects, the anti-mouse cleaved CDCP1 antibody or antigen-binding fragment thereof comprises a heavy chain (HC) comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to an amino acid sequence selected from the group consisting of SEQ ID NOs: 353-366. In certain aspects, the HC comprises an amino acid sequence selected from the group consisting of SEQ ID NOs: 353-366, as disclosed in Table 6, below. In certain aspects, the heavy chain amino acid sequence comprises one or more deletions, substitutions, or mutations within the immunoglobulin constant region, e.g., within the CH1 domain, the CH2 domain, the CH3 domain, or the hinge region.

In some aspects, the anti-mouse cleaved CDCP1 antibody or antigen-binding fragment thereof comprises a light chain (LC) comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to an amino acid sequence selected from the group consisting of SEQ ID NOs: 367-372. In certain aspects, the LC comprises an amino acid sequence selected from the group consisting of SEQ ID NOs: 367-372, as disclosed in Table 6, below.

In some aspects, the anti-mouse cleaved CDCP1 antibody or antigen-binding fragment thereof comprises a heavy chain (HC) and a light chain (LC), wherein the HC of the antibody or antigen-binding fragment thereof comprises an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to an amino acid sequence selected from the group consisting of SEQ ID NOs: 353-366 and the LC comprises an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to an amino acid sequence selected from the group consisting of SEQ ID NOs: 367-372.

In some aspects, the anti-mouse cleaved CDCP1 antibody or antigen-binding fragment thereof comprises a heavy chain (HC) and a light chain (LC), wherein the HC of the antibody or antigen-binding fragment thereof comprises the HC comprises an amino acid sequence selected from the group consisting of SEQ ID NOs: 353-366 and the LC comprises an amino acid sequence selected from the group consisting of SEQ ID NOs: 367-372.

TABLE 6
Heavy Chain HC) and Light Chain (LC) Amino Acid
Sequences of Anti-Mouse Cleaved CDCP1 Antibodies

| | HC Amino Acid Sequence | LC Amino Acid Sequence |
|---|---|---|
| Antibody | (SEQ ID NO) | (SEQ ID NO) |
| Fab 0114 | (SEQ ID NO: 353) | (SEQ ID NO: 367) |
| IgG 0029 | (SEQ ID NO: 354) | (SEQ ID NO: 368) |
| (Fab 0114) | | |
| Fab 0123 | (SEQ ID NO: 355) | (SEQ ID NO: 369) |
| IgG 0030 | (SEQ ID NO: 356) | (SEQ ID NO: 370) |
| (Fab 0123) | | |
| IgG 0046 | (SEQ ID NO: 357) | (SEQ ID NO: 371) |
| IgG 0050 | (SEQ ID NO: 358) | (SEQ ID NO: 372) |
| IgG 0051 | (SEQ ID NO: 359) | (SEQ ID NO: 371) |
| IgG 0055 | (SEQ ID NO: 360) | (SEQ ID NO: 371) |
| IgG 0058 | (SEQ ID NO: 361) | (SEQ ID NO: 371) |
| IgG 0059 | (SEQ ID NO: 362) | (SEQ ID NO: 371) |
| IgG 0060 | (SEQ ID NO: 363) | (SEQ ID NO: 371) |
| IgG 0062 | (SEQ ID NO: 364) | (SEQ ID NO: 371) |
| IgG 0063 | (SEQ ID NO: 365) | (SEQ ID NO: 371) |
| IgG 0065 | (SEQ ID NO: 366) | (SEQ ID NO: 371) |

A VH domain, or one or more CDRs thereof, described herein can be linked to a constant domain for forming a heavy chain, e.g., a full length heavy chain. Similarly, a VL domain, or one or more CDRs thereof, described herein can be linked to a constant domain for forming a light chain, e.g., a full length light chain. A full length heavy chain (optionally with the exception of the C-terminal lysine (K) or with the exception of the C-terminal glycine and lysine (GK), which can be absent) and full length light chain can combine to form a full length antibody.

A VH domain described herein can be fused to the constant domain of a human IgG, e.g., IgG1, IgG2, IgG3 or IgG4, which are either naturally-occurring or modified, e.g., as further described herein. For example, a VH domain can comprise the amino acid sequence of any VH domain described herein fused to a human IgG, e.g., an IgG1, constant region, such as the following wild-type human IgG1 constant domain amino acid sequence:

(SEQ ID NO: 381)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK

or that of an allotypic variant of SEQ ID NO: 381 and have the following amino acid sequences:

ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK
(SEQ ID NO: 382; allotype specific amino acid
residues are in bold and underlined).

A VH domain of an anti-human cleaved CDCP1 antibody can comprise the amino acid sequence of any VH domain described herein fused to an effectorless constant region, e.g., the following effectorless human IgG1 constant domain amino acid sequences:

ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPEAEGAPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPSSIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK
(SEQ ID NO: 383; “IgG1.1f,” comprising substitu-
tions L234A, L235E, G237A, A330S and P331S, which
are underlined)
or
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPEAEGAPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK
(SEQ ID NO: 384; “IgG1.3f”, comprising substitu-
tions L234A, L235E and G237A, which are
underlined).

For example, an allotypic variant of IgG1 comprises K97R, D239E, and/or L241M (underlined and bolded above) as numbered in SEQ ID NO: 381. Within the full length heavy chain constant region, and according to EU numbering, these amino acid substitutions are numbered K214R, D356E, and L358M. In some aspects, the constant region of an anti-cleaved CDCP1 antibody (e.g., anti-human cleaved CDCP1 antibody) can further comprises one or more mutations or substitutions at amino acids L117, A118, G120, A213, and P214 (underlined above) as numbered in SEQ ID NO: 382, 383, and 384, or L234, A235, G237, A330 and P331, per EU numbering. In some aspects, the constant region of an anti-cleaved CDCP1 antibody (e.g., anti-human cleaved CDCP1 antibody) can comprise one or more mutations or substitutions at amino acids L117A, A118E, G120A, A213S, and P214S of SEQ ID NO: 381, or L234A, L235E, G237A, A330S and P331S, per EU numbering. The constant region of an anti-cleaved CDCP1 antibody (e.g., anti-human cleaved CDCP1 antibody) can also comprise one or more mutations or substitutions L117A, A118E and G120A of SEQ ID NO: 381, or L234A, L235E and G237A, per EU numbering.

Alternatively, a VH domain of an anti-cleaved CDCP1 antibody (e.g., anti-human cleaved CDCP1) antibody can comprise the amino acid sequence of any VH domain described herein fused to a human IgG4 constant region, e.g., the following human IgG4 amino acid sequence or variants thereof:

ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPSCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK (SEQ ID NO: 385,
comprising S228P).

A VL domain described herein can be fused to the constant domain of a human Kappa or Lambda light chain. For example, a VL domain of an anti-cleaved CDCP1 antibody (e.g., anti-human cleaved CDCP1 antibody) can comprise the amino acid sequence of any VL domain described herein fused to the following human IgG1 kappa light chain amino acid sequence:

(SEQ ID NO: 386)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC

In some aspects, the heavy chain constant region comprises a lysine or another amino acid at the C-terminus, e.g., it comprises the following last amino acids: LSPGK (SEQ ID NO: 387) in the heavy chain. In some aspects, the heavy chain constant region is lacking one or more amino acids at the C-terminus, and has, e.g., the C-terminal sequence LSPG (SEQ ID NO: 388) or LSP (SEQ ID NO: 389).

The amino acid sequences of exemplary heavy and light chains are described herein.

In some aspects, an anti-cleaved CDCP1 antibody (e.g., anti-human cleaved CDCP1 antibody) comprises a combination of a heavy and light chain sequences set forth herein, e.g., in the preceding paragraph, wherein the antibody comprises two heavy chains and two light chains, and can further comprise at least one disulfide bond linking the two heavy chains together. The antibodies can also comprise disulfide bonds linking each of the light chains to each of the heavy chains.

Heavy and light chains comprising an amino acid sequence that is at least 99%, 98%, 97%, 96%, 95%, 90%, 85%, 80%, 75% or 70% identical to any of the heavy or light chains set forth herein (or their variable regions) can be used for forming anti-human cleaved CDCP1 antibodies having the desired characteristics, e.g., those described herein.

In some aspects, an anti-cleaved CDCP1 antibody (e.g., anti-human cleaved CDCP1 antibody) comprises a heavy chain variable region from a particular germline heavy chain immunoglobulin gene and/or a light chain variable region from a particular germline light chain immunoglobulin gene.

As used herein, a human antibody comprises heavy and light chain variable regions that are “the product of” or “derived from” a particular germline sequence if the variable regions of the antibody are obtained from a system that uses human germline immunoglobulin genes. Such systems include immunizing a transgenic mouse carrying human immunoglobulin genes with the antigen of interest or screening a human immunoglobulin gene library displayed on phage with the antigen of interest. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody. A human antibody that is “the product of” or “derived from” a particular human germline immunoglobulin sequence can contain amino acid differences as compared to the germline sequence, due to, for example, naturally-occurring somatic mutations or intentional introduction of site-directed mutation. However, a selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In some cases, a human antibody can be at least 95%, or even at least 96%, 97%, 98%, or 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody can display no more than 5, or even no more than 4, 3, 2, or 1 amino acid difference from the amino acid sequence encoded by the germline immunoglobulin gene.

Anti-cleaved CDCP1 antibodies or antigen-binding fragments thereof can comprise a heavy chain variable region comprising CDR1, CDR2 and CDR3 sequences and a light chain variable region comprising CDR1, CDR2 and CDR3 sequences, wherein one or more of these CDR sequences comprise specified amino acid sequences based on the anti-cleaved CDCP1 antibodies antigen-binding fragments thereof described herein, or conservative modifications thereof, and wherein the antibodies retain the desired functional properties of the anti-cleaved CDCP1 antibodies described herein.

Conservative amino acid substitutions can be made in portions of the antibodies other than, or in addition to, the CDRs. For example, conservative amino acid modifications can be made in a framework region or in the Fc region. A variable region or a heavy or light chain can comprise 1, 2, 3, 4, 5, 1-2, 1-3, 1-4, 1-5, 1-10, 1-15, 1-20, 1-25, or 1-50 conservative amino acid substitutions relative to the anti-cleaved CDCP1 antibody sequences provided herein. In some aspects, an anti-cleaved CDCP1 antibody antigen-binding fragment thereof comprises a combination of conservative and non-conservative amino acid modification.

Also provided are engineered and modified antibodies that can be prepared using an antibody having one or more of the VH and/or VL sequences disclosed herein as starting material to engineer a modified antibody, which modified antibody can have altered properties from the starting antibody. An antibody can be engineered by modifying one or more residues within one or both variable regions (i.e., VH and/or VL), for example within one or more CDR regions and/or within one or more framework regions. Additionally, or alternatively, an antibody can be engineered by modifying residues within the constant region(s), for example to alter the effector function(s) of the antibody.

One type of variable region engineering that can be performed is CDR grafting. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally-occurring antibodies by constructing expression vectors that include CDR sequences from the specific naturally-occurring antibody grafted onto framework sequences from a different antibody with different properties (see, e.g., Riechmann, L. et al. (1998) Nature 332:323-327; Jones, P. et al. (1986) Nature 321:522-525; Queen, C. et al. (1989) Proc. Natl. Acad. Sci. U.S.A. 86:10029-10033; U.S. Pat. No. 5,225,539 to Winter, and U.S. Pat. Nos. 5,530,101; 5,585,089; 5,693,762 and 6,180,370 to Queen et al.).

Accordingly, some aspects described herein pertain to an isolated monoclonal antibody, or antigen binding fragment thereof, comprising a heavy chain variable region comprising CDR1, CDR2, and CDR3 sequences described herein, and a light chain variable region comprising CDR1, CDR2, and CDR3 sequences described herein. Thus, such antibodies contain the VH and VL CDR sequences of monoclonal antibodies described herein yet can contain different framework sequences from these antibodies.

Such framework sequences can be obtained from public DNA databases or published references that include germline antibody gene sequences. For example, germline DNA sequences for human heavy and light chain variable region genes can be found in the “VBase” human germline sequence database (available on the Internet at www.mrc-cpe.cam.ac.uk/vbase), as well as in Kabat, E. A., et al. (1991) Sequences of Proteins of Immunological Interest, Fifth Edition, U.S. Department of Health and Human Services, NIH Publication No. 91-3242; Tomlinson, I. M., et al. (1992) “The Repertoire of Human Germline V_{H} Sequences Reveals about Fifty Groups of V_{H} Segments with Different Hypervariable Loops”/. Mol. Biol. 227:776-798; and Cox, J. P. L. et al. (1994) “A Directory of Human Germ-line VH Segments Reveals a Strong Bias in their Usage” Eur. J. Immunol. 24:827-836; the contents of each of which are expressly incorporated herein by reference.

In some aspects, the framework sequences for use in the anti-cleaved CDCP1 antibodies described herein are those that are structurally similar to the framework sequences used by the anti-cleaved CDCP1 antibodies described herein. The VH CDR1, CDR2 and CDR3 sequences, and the VL CDR1, CDR2 and CDR3 sequences, can be grafted onto framework regions that have the identical sequence as that found in the germline immunoglobulin gene from which the framework sequence derives, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see, e.g., U.S. Pat. Nos. 5,530,101; 5,585,089; 5,693,762; and U.S. Pat. No. 6,180,370 to Queen et al.).

Engineered anti-cleaved CDCP1 antibodies described herein include those in which modifications have been made to framework residues within VH and/or VL, e.g., to improve the properties of the antibody. Typically, such framework modifications are made to decrease the immunogenicity of the antibody. For example, one approach is to “backmutate” one or more framework residues to the corresponding germline sequence. More specifically, an antibody that has undergone somatic mutation can contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis or PCR-mediated mutagenesis. Such “backmutated” antibodies are also intended to be encompassed. Another type of framework modification involves mutating one or more residues within the framework region, or even within one or more CDR regions, to remove T cell epitopes to thereby reduce the potential immunogenicity of the antibody. This approach is also referred to as “deimmunization” and is described in further detail in U.S. Patent Publication No. 20030153043 by Carr et al.

Another type of variable region modification is to mutate amino acid residues within the VH and/or VL CDR1, CDR2 and/or CDR3 regions to thereby improve one or more binding properties (e.g., affinity) of the antibody of interest. Site-directed mutagenesis or PCR-mediated mutagenesis can be performed to introduce the mutation(s) and the effect on antibody binding, or other functional property of interest, can be evaluated in in vitro or in vivo assays as described herein and provided in the Examples. In some aspects, conservative modifications (as discussed above) are introduced. The mutations can be amino acid substitutions, additions or deletions. Moreover, typically no more than one, two, three, four or five residues within a CDR region are altered.

Methionine residues in CDRs of antibodies can be oxidized, resulting in potential chemical degradation and consequent reduction in potency of the antibody. Accordingly, also provided are anti-cleaved CDCP1 antibodies which have one or more methionine residues in the heavy and/or light chain CDRs replaced with amino acid residues which do not undergo oxidative degradation. In some aspects, the methionine residues in the CDRs of the anti-cleaved CDCP1 antibodies of the present disclosure, or antigen-binding fragments thereof are replaced with amino acid residues which do not undergo oxidative degradation.

Similarly, deamidation sites can be removed from anti-cleaved CDCP1 antibodies, particularly in the CDRs.

Anti-cleaved CDCP1 variable regions described herein can be linked (e.g., covalently linked or fused) to an Fc, e.g., an IgG1, IgG2, IgG3 or IgG4 Fc, which can be of any allotype or isoallotype, e.g., for IgG1: G1m, G1m1(a), G1m2(x), G1m3(f), G1m17(z); for IgG2: G2m, G2m23(n); for IgG3: G3m, G3m21(g1), G3m28(g5), G3ml l(b0), G3m5(b1), G3ml3(b3), G3ml4(b4), G3ml0(b5), G3ml5(s), G3ml6(t), G3m6(c3), G3m24(c5), G3m26(u), G3m27(v); and for K: Km, Km1, Km2, Km3 (see, e.g., Jefferies et al. (2009) mAbs 1:1).

Generally, variable regions described herein can be linked to an Fc comprising one or more modification, typically to alter one or more functional properties of the antibody, such as serum half-life, complement fixation, Fc receptor binding, antigen-dependent cellular cytotoxicity, and/or antibody-dependent cellular phagocytosis. Furthermore, an antibody described herein can be chemically modified (e.g., one or more chemical moieties can be attached to the antibody) or be modified to alter its glycosylation, to alter one or more functional properties of the antibody. Each of these aspects is described in further detail below. The numbering of residues in the Fc region is that of the EU index of Kabat.

The Fc region encompasses domains derived from the constant region of an immunoglobulin, including a fragment, analog, variant, mutant or derivative of the constant region. Suitable immunoglobulins include IgG1, IgG2, IgG3, IgG4, and other classes such as IgA, IgD, IgE and IgM, The constant region of an immunoglobulin is defined as a naturally-occurring or synthetically-produced polypeptide homologous to the immunoglobulin C-terminal region, and can include a CH1 domain, a hinge, a CH2 domain, a CH3 domain, or a CH4 domain, separately or in combination.

Ig molecules interact with multiple classes of cellular receptors. For example IgG molecules interact with three classes of Fcγ receptors (FcγR) specific for the IgG class of antibody, namely FcγRI, FcγRII, and FcγRIII. The important sequences for the binding of IgG to the FcγR receptors have been reported to be located in the CH2 and CH3 domains. The serum half-life of an antibody is influenced by the ability of that antibody to bind to an Fc receptor (FcR).

In some aspects, the Fc region is a variant Fc region, e.g., an Fc sequence that has been modified (e.g., by amino acid substitution, deletion and/or insertion) relative to a parent Fc sequence (e.g., an unmodified Fc polypeptide that is subsequently modified to generate a variant), to provide desirable structural features and/or biological activity,

Generally, variants of the constant region or portions thereof, e.g., CH1, CL, hinge, CH2 or CH3 domains can comprise 1, 2, 3, 4, 5, 6, 7, 8, 9, 10 or more mutations, and/or at most 10, 9, 8, 7, 6, 5, 4, 3, 2 or 1 mutation, or 1-10 or 1-5 mutations, or comprise an amino acid sequence that is at least about 75%, 80%, 85%, 90%, 95%, 96%, 97%, 98%, or 99% identical to that of the corresponding wild-type region or domain (CH1, CL, hinge, CH2, or CH3 domain, respectively), provided that the heavy chain constant region comprising the specific variant retains the necessary biological activity.

For example, one can make modifications in the Fc region in order to generate an Fc variant that (a) mediates increased or decreased antibody-dependent cell-mediated cytotoxicity (ADCC) and/or antibody-dependent cellular phagocytosis (ADCP), (b) mediates increased or decreased complement mediated cytotoxicity (CDC), (c) has increased or decreased affinity for C1q and/or (d) has increased or decreased affinity for a Fc receptor relative to the parent Fc. Such Fc region variants will generally comprise at least one amino acid modification in the Fc region. Combining amino acid modifications is thought to be particularly desirable. For example, the variant Fc region can include two, three, four, five, etc. substitutions therein, e.g., of the specific Fc region positions identified herein.

A variant Fc region can also comprise a sequence alteration wherein amino acids involved in disulfide bond formation are removed or replaced with other amino acids. Such removal can avoid reaction with other cysteine-containing proteins present in the host cell used to produce the anti-cleaved CDCP1 antibodies described herein. Even when cysteine residues are removed, single chain Fc domains can still form a dimeric Fc domain that is held together non-covalently. In some aspects, the Fc region can be modified to make it more compatible with a selected host cell. For example, one can remove the PA sequence near the N-terminus of a typical native Fc region, which can be recognized by a digestive enzyme in E. coli such as proline iminopeptidase. In some aspects, one or more glycosylation sites within the Fc domain can be removed. Residues that are typically glycosylated (e.g., asparagine) can confer cytolytic response. Such residues can be deleted or substituted with unglycosylated residues (e.g., alanine). In some aspects, sites involved in interaction with complement, such as the C1q binding site, can be removed from the Fc region. For example, one can delete or substitute the EKK sequence of human IgG1. In some aspects, sites that affect binding to Fc receptors can be removed, preferably sites other than salvage receptor binding sites. In some aspects, an Fc region can be modified to remove an ADCC site. ADCC sites are known in the art; see, for example, Molec. Immunol. 29 (5): 633-9 (1992) with regard to ADCC sites in IgG1. Specific examples of variant Fc domains are disclosed for example, in WO 97/34631, WO 96/32478 and WO07/041635.

In some aspects, the hinge region of Fc is modified such that the number of cysteine residues in the hinge region is altered, e.g., increased or decreased. This approach is described further in U.S. Pat. No. 5,677,425 by Bodmer et al. The number of cysteine residues in the hinge region of Fc is altered to, for example, facilitate assembly of the light and heavy chains or to increase or decrease the stability of the antibody. In some aspects, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In some aspects, the Fc region is altered by replacing at least one amino acid residue with a different amino acid residue to alter the effector function(s) of the antibody. For example, one or more amino acids selected from amino acid residues 234, 235, 236, 237, 297, 318, 320, 322, 330, and/or 331 can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In another example, one or more amino acids selected from amino acid residues 329, 331, and 322 can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al.

In another example, one or more amino acid residues within amino acid positions 231 and 239 are altered to thereby alter the ability of the antibody to fix complement. This approach is described further in PCT Publication WO 94/29351 by Bodmer et al.

In another example, the Fc region can be modified to enhance affinity for an Fcγ and increase macrophage-mediated phagocytosis. See, e.g., Richard et al., Mo. Cancer. Ther. 7 (8): 2517-27 (2008), which is incorporated by reference herein in its entirety. In certain aspects, the Fc region can be modified to increase affinity for FcγRIIa relative to inhibitory FcγRIIb. One particular point mutation, G236A (whose numbering is according to the EU index), has been identified as having increased affinity for FcγRIIa relative to inhibitory FcγRIIb. This increased affinity for FcRIIa correlated with increased macrophage-mediated phagocytosis, relative to native IgG1. In some aspects, the Fc region of the anti-cleaved CDCP1 antibody comprises one or more mutation or combination of mutations selected from G236A, I332E, S239/I332E, I332E/G236A, and S239D/I332E/G236A. Other modifications to the Fc region can increase antibody dependent cellular cytotoxicity (ADCC), e.g., by increasing affinity for activating receptors such as FcγRI and/or FcγRIIIa. For example, the G236A substitution, and combination of the G236A substitution with modifications that improve affinity for activating receptors (e.g., FcγRI and/or FcγRIIIa), for example including but not limited to substitutions at 332 and 239, provide substantially improved ADCC relative to the parent WT antibody. See U.S. Pat. No. 9,040,041, which is incorporated by reference herein in its entirety.

In another example, the Fc region can be modified to decrease antibody dependent cellular cytotoxicity (ADCC) and/or to decrease the affinity for an Fcγ receptor by modifying one or more amino acids at the following positions: 234, 235, 236, 238, 239, 240, 241, 243, 244, 245, 247, 248, 249, 252, 254, 255, 256, 258, 262, 263, 264, 265, 267, 268, 269, 270, 272, 276, 278, 280, 283, 285, 286, 289, 290, 292, 293, 294, 295, 296, 298, 299, 301, 303, 305, 307, 309, 312, 313, 315, 320, 322, 324, 325, 326, 327, 329, 330, 331, 332, 333, 334, 335, 337, 338, 340, 360, 373, 376, 378, 382, 388, 389, 398, 414, 416, 419, 430, 433, 434, 435, 436, 437, 438 or 439. Exemplary substitutions include 236A, 239D, 239E, 268D, 267E, 268E, 268F, 324T, 332D, and 332E. Exemplary variants include 239D/332E, 236A/332E, 236A/239D/332E, 268F/324T, 267E/268F, 267E/324T, and 267E/268F7324T. Other modifications for enhancing FcγR and complement interactions include but are not limited to substitutions 298A, 333A, 334A, 326A, 2471, 339D, 339Q, 280H, 290S, 298D, 298V, 243L, 292P, 300L, 396L, 3051, and 396L. These and other modifications are reviewed in Strohl, 2009, Current Opinion in Biotechnology 20:685-691.

Fc modifications that increase binding to an Fcγ receptor include amino acid modifications at any one or more of amino acid positions 238, 239, 248, 249, 252, 254, 255, 256, 258, 265, 267, 268, 269, 270, 272, 279, 280, 283, 285, 298, 289, 290, 292, 293, 294, 295, 296, 298, 301, 303, 305, 307, 312, 315, 324, 327, 329, 330, 335, 337, 338, 340, 360, 373, 376, 379, 382, 388, 389, 398, 414, 416, 419, 430, 434, 435, 437, 438 or 439 of the Fc region, wherein the numbering of the residues in the Fc region is that of the EU index as in Kabat (WO00/42072).

Optionally, the Fc region can comprise a non-naturally-occurring amino acid residue at additional and/or alternative positions known to one skilled in the art (see, e.g., U.S. Pat. Nos. 5,624,821; 6,277,375; 6,737,056; 6,194,551; 7,317,091; 8,101,720; 9,040,041; PCX Patent Publications WO 00/42072; WO 01/58957; WO 02/06919; WO 04/016750; WO 04/029207; WO 04/035752; WO 04/074455; WO 04/099249; WO 04/063351; WO 05/070963; WO 05/040217; WO 05/092925; and WO 06/0201 14).

The affinities and binding properties of an Fc region for its ligand can be determined by a variety of in vitro assay methods (biochemical or immunological based assays) known in the art including but not limited to, equilibrium methods (e.g., enzyme-linked immunosorbent assay (ELISA), or radioimmunoassay (RIA)), or kinetics (e.g., BIACORE analysis), and other methods such as indirect binding assays, competitive inhibition assays, fluorescence resonance energy transfer (FRET), gel electrophoresis and chromatography (e.g., gel filtration). These and other methods can utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental immunology, 4th Ed., Lippincott-Raven, Philadelphia (1999), which focuses on antibody-immunogen interactions.

In some aspects, the antibody is modified to increase its biological half-life. Various approaches are possible. For example, this can be done by increasing the binding affinity of the Fc region for FcRn, for example, one or more of more of following residues can be mutated: 252, 254, 256, 433, 435, 436, as described in U.S. Pat. No. 6,277,375. Specific exemplary substitutions include one or more of the following: T252L, T254S, and/or T256F. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al. Other exemplary variants that increase binding to FcRn and/or improve pharmacokinetic properties include substitutions at positions 259, 308, 428, and 434, including for Example 3591, 308F, 428L, 428M, 434S, 4341 1. 434F, 434Y, and 434X1. Other variants that increase Fc binding to FcRn include: 250E, 250Q, 428 L, 428F, 250Q/428L (Hinton et al. 2004, J. Biol. Chem. 279 (8): 6213-6216, Hinton et al. 2006 Journal of Immunology 176:346-356), 256A, 272A, 286A, 305A, 307A, 307Q, 31 1A, 312A, 376A, 378Q, 380A, 382A, 434A (Shields et al., Journal of Biological Chemistry, 2001, 276 (9): 6591-6604), 252F, 252T, 252Y, 252W, 254T, 256S, 256R, 256Q, 256E, 256D, 256T, 309P, 31 1 S, 433R, 433S, 4331, 433P, 433Q, 434H, 434F, 434Y, 252Y/254T/256E, 433K/434F/436H, 308T/309P/311S (Dall Acqua et al. Journal of Immunology, 2002, 169:5171-5180, Dall'Acqua et al., 2006, Journal of Biological Chemistry 281:23514-23524). Other modifications for modulating FcRn binding are described in Yeung et al., 2010, J Immunol, 182:7663-7671.

In some aspects, hybrid IgG isotypes with particular biological characteristics can be used. For example, an IgG1/IgG3 hybrid variant can be constructed by substituting IgG1 positions in the CH2 and/or CH3 region with the amino acids from IgG3 at positions where the two isotypes differ. Thus a hybrid variant IgG antibody can be constructed that comprises one or more substitutions, e.g., 274Q, 276K, 300F, 339T, 356E, 358M, 384S, 392N, 397M, 4221, 435R, and 436F. In some aspects described herein, an IgG1/IgG2 hybrid variant can be constructed by substituting IgG2 positions in the CH2 and/or CH3 region with amino acids from IgG1 at positions where the two isotypes differ. Thus a hybrid variant IgG antibody can be constructed that comprises one or more substitutions, e.g., one or more of the following amino acid substitutions: 233E, 234L, 235L, −236G (referring to an insertion of a glycine at position 236), and 327A.

Moreover, the binding sites on human IgG1 for FcγRI, FcγRII, FcγRIII and FcRn have been mapped and variants with improved binding have been described (see Shields, R. L. et al. (2001) J. Biol. Chem. 276:6591-6604). Specific mutations at positions 256, 290, 298, 333, 334 and 339 were shown to improve binding to FcγRIII. Additionally, the following combination mutants were shown to improve FcγRIII binding: T256A/S298A, S298A/E333A, S298A/K224A and S298A/E333A/K334A, which has been shown to exhibit enhanced FcγRIIIa binding and ADCC activity (Shields et al., 2001). Other IgG1 variants with strongly enhanced binding to FcγRIIIa have been identified, including variants with S239D/I332E and S239D/I332E/A330L mutations which showed the greatest increase in affinity for FcγRIIIa, a decrease in FcγRIIb binding, and strong cytotoxic activity in cynomolgus monkeys (Lazar et al., 2006). Introduction of the triple mutations into antibodies such as alemtuzumab (CD52-specific), trastuzumab (HER2/neu-specific), rituximab (CD20-specific), and cetuximab (EGFR-specific) translated into greatly enhanced ADCC activity in vitro, and the S239D/I332E variant showed an enhanced capacity to deplete B cells in monkeys (Lazar et al., 2006). In addition, IgG1 mutants containing L235V, F243L, R292P, Y300L and P396L mutations which exhibited enhanced binding to FcγRIIIa and concomitantly enhanced ADCC activity in transgenic mice expressing human FcγRIIIa in models of B cell malignancies and breast cancer have been identified (Stavenhagen et al., 2007; Nordstrom et al., 2011). Other Fc mutants that can be used include: S298A/E333A/L334A, S239D/I332E, S239D/I332E/A330L, L235V/F243L/R292P/Y300L/P396L, and M428L/N434S.

Specific mutations at positions 234, 235, 236, 239, 267, 268, 293, 295, 324, 327, 328, 330, and 332 were shown to improve binding to FcγRIIa and/or reduce binding to FcγRIIb, resulting in enhanced ADCC and/or ADCP activity (Richards et al., Mol. Cancer Ther. 7 (8): 2517-2527; U.S. Pat. No. 9,040,041). In particular, Fc variants that selectively improve binding to one or more human activating receptors relative to FcγRIIb, or selectively improve binding to FcγRIIb relative to one or more activating receptors, can comprise a substitution selected from the group consisting of 234G, 234I, 235D, 235E, 235I, 235Y, 236A, 236S, 239D, 267D, 267E, 267Q, 268D, 268E, 293R, 295E, 324G, 324I, 327H, 328A, 328F, 328I, 3301, 330L, 330Y, 332D, and 332E. Additional substitutions that can also be combined include other substitutions that modulate FcγR affinity and complement activity, including but not limited to 298A, 298T, 326A, 326D, 326E, 326W, 326Y, 333A, 333S, 334L, and 334A (U.S. Pat. No. 6,737,056; Shields et al, Journal of Biological Chemistry, 2001, 276 (9): 6591-6604; U.S. Pat. No. 6,528,624; Idusogie et al., 2001, J. Immunology 166:2571-2572). Preferred variants that can be particularly useful to combine with other Fc variants include those that comprise the substitutions 298A, 326A, 333A, and 334A. Additional substitutions that can be combined with the FcγR selective variants include 247L, 255L, 270E, 392T, 396L, and 421K (U.S. Ser. No. 10/754,922; U.S. Ser. No. 10/902,588); and 280H, 280Q, and 280Y (U.S. Ser. No. 10/370,749).

When using an IgG4 constant domain, it can include the substitution S228P, which mimics the hinge sequence in IgG1 and thereby stabilizes IgG4 molecules.

### III. Antibody Physical Properties

Anti-cleaved CDCP1 antibodies or antigen binding fragments thereof, e.g., those described herein, have some or all of the physical characteristics of the specific anti-cleaved CDCP1 antibodies described herein, such as the characteristics described in the Examples.

Anti-cleaved CDCP1 antibodies or antigen binding fragments thereof described herein can contain one or more glycosylation sites in either the light or heavy chain variable region. Such glycosylation sites can result in increased immunogenicity of the antibody or an alteration of the pK of the antibody due to altered antigen binding (Marshall et al., (1972) Annu Rev Biochem 41:673-702; Gala and Morrison (2004) J. Immunol 172:5489-94; Wallick et al., (1988) J Exp Med 168:1099-109; Spiro (2002) Glycobiology 12: 43R-56R; Parekh et al., (1985) Nature 316:452-7; Mimura et al., (2000) Mol Immunol 37:697-706). Glycosylation has been known to occur at motifs containing an N-X-S/T sequence. In some instances, an anti-cleaved CDCP1 antibody does not contain variable region glycosylation. This can be achieved either by selecting antibodies that do not contain the glycosylation motif in the variable region or by mutating residues within the glycosylation region.

In some aspects, the anti-cleaved CDCP1 antibodies or antigen binding fragments are modified to remove a glycosylation site. In some aspects, the glycosylation site removal is accomplished via substitution of the asparagine (N) to Aspartic acid (D) at a position that corresponds to residue 31 in SEQ ID NO: 61. In some aspects, the antibody or antigen-binding fragment described herein comprises substitution of methionine (M) to alanine (A), isoleucine (I), leucine (L), or valine (V) at a position that corresponds to residue 114 in SEQ ID NO: 61 or 65.

In some aspects, the anti-cleaved CDCP1 antibodies or antigen binding fragments thereof described herein do not contain asparagine isomerism sites. The deamidation of asparagine can occur on N-G or D-G sequences and result in the creation of an isoaspartic acid residue that introduces a kink into the polypeptide chain and decreases its stability (isoaspartic acid effect).

Each antibody will have a unique isoelectric point (pi), which generally falls in the pH range between 6 and 9.5. The pi for an IgG1 antibody typically falls within the pH range of 7-9.5 and the pi for an IgG4 antibody typically falls within the pH range of 6-8. There is speculation that antibodies with a pi outside the normal range can have some unfolding and instability under in vivo conditions. Thus, an anti-cleaved CDCP1 antibody can contain a pi value that falls in the normal range. This can be achieved either by selecting antibodies with a pi in the normal range or by mutating charged surface residues.

Each antibody will have a characteristic melting temperature, with a higher melting temperature indicating greater overall stability in vivo (Krishnamurthy R and Manning M C (2002) Curr Pharm Biotechnol 3:361-71). Generally, the T_{M}i (the temperature of initial unfolding) can be greater than 60° C., greater than 65° C., or greater than 70° C. The melting point of an antibody can be measured using differential scanning calorimetry (Chen et al., (2003) Pharm Res 20:1952-60; Ghirlando et al., (1999) Immunol Lett 68:47-52) or circular dichroism (Murray et al., (2002) J. Chromatogr Sci 40:343-9).

In some aspects, antibodies are selected that do not degrade rapidly. Degradation of an antibody can be measured using capillary electrophoresis (CE) and MALDI-MS (Alexander A J and Hughes D E (1995) Anal Chem 67:3626-32).

In some aspects, antibodies are selected that have minimal aggregation effects, which can lead to the triggering of an unwanted immune response and/or altered or unfavorable pharmacokinetic properties. Generally, antibodies are acceptable with aggregation of 25% or less, 20% or less, 15% or less, 10% or less, or 5% or less. Aggregation can be measured by several techniques, including size-exclusion column (SEC), high performance liquid chromatography (HPLC), and light scattering. In some aspects, the antibodies display a desirable solubility, e.g., solubility that allows commercial manufacturing. In some aspects, the solubility of the antibodies described herein was at least 10 mg/ml, at least 15 mg/ml, at least 20 mg/ml, at least 25 mg/ml, at least 30 mg/ml, at least 40 mg/ml, at least 50 mg/ml, at least 60 mg/ml, or at least 70 mg/ml, at neutral or slightly acidic pH.

In some aspects, the antibodies described herein have higher stability than a reference antibody. In some aspects, the antibodies described herein have a higher melting temperature than a reference antibody. In some aspects, the antibodies described herein have a lower tendency for aggregation than a reference antibody. In some aspects, the antibodies described herein have a higher solubility than a reference antibody. In some aspects, the antibodies described herein have a higher rate of absorption, lower toxicity, higher biological activity and/or target selectivity, better manufacturability, and/or lower immunogenicity than a reference antibody. The reference antibody can be another antibody or fragments thereof, or conjugate thereof, that binds to e.g., human cleaved CDCP1.

In some aspects, antibodies or antigen-binding fragments thereof that specifically bind to a cleaved CDCP1, described herein, are less toxic than the antibodies or antigen-binding fragments thereof that bind to a full-length CDCP1 (e.g., human full-length CDCP1) at a detectable level.

### IV. Methods of Engineering Antibodies

As discussed above, the anti-cleaved CDCP1 antibodies or antigen-binding fragments thereof having VH and VL sequences disclosed herein can be used to create new anti-cleaved CDCP1 antibodies by modifying the VH and/or VL sequences, or the constant region(s) attached thereto. Thus, in another aspect described herein, the structural features of an anti-cleaved CDCP1 antibody antigen-binding fragment thereof described herein are used to create structurally related anti-cleaved CDCP1 antibodies antigen-binding fragments thereof that retain at least one functional property of the anti-cleaved CDCP1 antibodies or antigen-binding fragments thereof described herein, such as binding to human CDCP1 and cynomolgus CDCP1. For example, one or more CDR regions of an anti-cleaved CDCP1 antibody or antigen-binding fragment thereof described herein can be combined recombinantly with known framework regions and/or other CDRs to create additional, recombinantly-engineered, anti-cleaved CDCP1 antibodies described herein, as discussed above. Other types of modifications include those described in the previous section. The starting material for the engineering method is one or more of the VH and/or VL sequences provided herein, or one or more CDR regions thereof. To create the engineered antibody, it is not necessary to actually prepare (i.e., express as a protein) an antibody having one or more of the VH and/or VL sequences provided herein, or one or more CDR regions thereof. Rather, the information contained in the sequence(s) is used as the starting material to create a “second generation” sequence(s) derived from the original sequence(s) and then the “second generation” sequence(s) is prepared and expressed as a protein.

Accordingly, provided herein are methods for preparing an anti-cleaved CDCP1 antibody antigen-binding fragment thereof described herein.

The altered antibody can exhibit at least one of the functional properties set herein. The functional properties of the altered antibodies can be assessed using standard assays available in the art and/or described herein, such as those set forth in the Examples (e.g., ELISAs, FACS).

In some aspects of the methods of engineering the anti-cleaved CDCP1 antibodies or antigen-binding fragments thereof described herein, mutations can be introduced randomly or selectively along all or part of an anti-cleaved CDCP1 antibody or antigen-binding fragment coding sequence and the resulting modified anti-cleaved CDCP1 antibodies or antigen-binding fragments thereof be screened for binding activity and/or other functional properties as described herein. Mutational methods have been described in the art. For example, PCT Publication WO 02/092780 by Short describes methods for creating and screening antibody mutations using saturation mutagenesis, synthetic ligation assembly, or a combination thereof. Alternatively, PCT Publication WO 03/074679 by Lazar et al. describes methods of using computational screening methods to optimize physiochemical properties of antibodies.

### V. Nucleic Acid Molecules

Another aspect described herein pertains to nucleic acid molecules that encode the anti-cleaved CDCP1 antibodies or antigen-binding fragments described herein. The nucleic acids can be present in whole cells, in a cell lysate, or in a partially purified or substantially pure form. A nucleic acid is “isolated” or “rendered substantially pure” when purified away from other cellular components or other contaminants, e.g., other cellular nucleic acids (e.g., other chromosomal DNA, e.g., the chromosomal DNA that is linked to the isolated DNA in nature) or proteins, by standard techniques, including alkaline/SDS treatment, CsCl banding, column chromatography, restriction enzymes, agarose gel electrophoresis and others well known in the art. See, F. Ausubel, et al., ed. (1987) Current Protocols in Molecular Biology, Greene Publishing and Wiley Interscience, New York. A nucleic acid described herein can be, for example, DNA or RNA and can or cannot contain intronic sequences. In some aspects, the nucleic acid is a cDNA molecule.

Nucleic acids described herein can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), nucleic acid encoding the antibody can be recovered from the library.

In some aspects, a polynucleotide described herein comprises a nucleic acid molecule encoding the heavy chain variable region or heavy chain of the antibody or antigen-binding fragment thereof of the present disclosure.

In some aspects, the nucleic acid molecule of the present disclosure encodes the VH of SEQ ID NO: 88, 92, 93, 95, 97, 163, 165, 169, 171, 173, 175, 177, 181, 183, 187, 189, 191, 193, 195, 203, 207, 211, or 227, as shown in Table 7 below.

In some aspects, a polynucleotide comprises a nucleic acid molecule encoding the light chain variable region or light chain of the antibody or antigen-binding fragment thereof of the present disclosure.

In some aspects, the nucleic acid molecule encodes the VL of SEQ ID NO: 90, 164, 166, 180, 198, 200, 202, 204, 206, 208, 210, 212, 214, 216, 218, 220, 222, 224, 226, 228, 230, 232, 234, or 236, as, as shown in Table 7 below.

In some aspects, a polynucleotide of the present disclosure comprises a first nucleic acid molecule encoding the heavy chain variable region of SEQ ID NO: 88, 92, 93, 95, 97, 163, 165, 169, 171, 173, 175, 177, 181, 183, 187, 189, 191, 193, 195, 203, 207, 211, or 227, and a second nucleic acid molecule encoding the light chain variable region of SEQ ID NO: 90, 164, 166, 180, 198, 200, 202, 204, 206, 208, 210, 212, 214, 216, 218, 220, 222, 224, 226, 228, 230, 232, 234, or 236.

In some aspects, a mixture of polynucleotides of the present disclosure comprises a first polynucleotide which comprises a nucleic acid molecule encoding the heavy chain variable region of SEQ ID NO: 88, 92, 93, 95, 97, 163, 165, 169, 171, 173, 175, 177, 181, 183, 187, 189, 191, 193, 195, 203, 207, 211, or 227, and a second polynucleotide which comprises a nucleic acid molecule encoding the light chain variable region of SEQ ID NO: 90,164,166, 180, 198, 200, 202, 204, 206, 208, 210, 212, 214, 216, 218, 220, 222, 224, 226, 228, 230, 232, 234, or 236.

TABLE 7
Variable Heavy Chain (VH) and Variable Light
Chain (VL) Nucleic Acid Sequences of
Anti-Human Cleaved CDCP1 Antibodies

| | VH Nucleic | VL Nucleic |
|---|---|---|
| | Acid Sequence | Acid Sequence |
| Antibody | (SEQ ID NO) | (SEQ ID NO) |
| CL03 Fab | (SEQ ID NO: 163) | (SEQ ID NO: 164) |
| IgGD09 | (SEQ ID NO: 165) | (SEQ ID NO: 166) |
| CL03 IgG | (SEQ ID NO: 163) | (SEQ ID NO: 164) |
| CL03 IgG H1- | (SEQ ID NO: 169) | (SEQ ID NO: 164) |
| NtoD | | |
| CL03 IgG H1- | (SEQ ID NO: 171) | (SEQ ID NO: 164) |
| NtoD H3-MtoA | | |
| (“IgG101”) | | |
| CL03 IgG H1- | (SEQ ID NO: 173) | (SEQ ID NO: 164) |
| NtoD H3-MtoI | | |
| CL03 IgG H1- | (SEQ ID NO: 175) | (SEQ ID NO: 164) |
| NtoD H3-MtoL | | |
| CL03 IgG H1- | (SEQ ID NO: 177) | (SEQ ID NO: 164) |
| NtoD H3-MtoV | | |
| CL07 Fab | (SEQ ID NO: 179) | (SEQ ID NO: 180) |
| CL07 IgG | (SEQ ID NO: 179) | (SEQ ID NO: 180) |
| CL07 IgG H1- | (SEQ ID NO: 183) | (SEQ ID NO: 180) |
| NtoD | | |
| IgG3H7L | (SEQ ID NO: 169) | (SEQ ID NO: 180) |
| IgGA01 | (SEQ ID NO: 187) | (SEQ ID NO: 164) |
| IgGA03 | (SEQ ID NO: 189) | (SEQ ID NO: 164) |
| IgGA04 | (SEQ ID NO: 191) | (SEQ ID NO: 164) |
| IgGA05 | (SEQ ID NO: 193) | (SEQ ID NO: 164) |
| IgGA06 | (SEQ ID NO: 195) | (SEQ ID NO: 164) |
| IgGA08 | (SEQ ID NO: 169) | (SEQ ID NO: 198) |
| IgGA09 | (SEQ ID NO: 169) | (SEQ ID NO: 200) |
| IgGA10 | (SEQ ID NO: 169) | (SEQ ID NO: 202) |
| IgGA11 | (SEQ ID NO: 203) | (SEQ ID NO: 204) |
| IgGB01 | (SEQ ID NO: 169) | (SEQ ID NO: 206) |
| IgGB03 | (SEQ ID NO: 207) | (SEQ ID NO: 208) |
| IgGB04 | (SEQ ID NO: 203) | (SEQ ID NO: 210) |
| IgGB07 | (SEQ ID NO: 211) | (SEQ ID NO: 212) |
| IgGB08 | (SEQ ID NO: 203) | (SEQ ID NO: 214) |
| IgGC01 | (SEQ ID NO: 203) | (SEQ ID NO: 216) |
| IgGC03 | (SEQ ID NO: 169) | (SEQ ID NO: 218) |
| IgGC04 | (SEQ ID NO: 203) | (SEQ ID NO: 220) |
| IgGC05 | (SEQ ID NO: 169) | (SEQ ID NO: 222) |
| IgGC06 | (SEQ ID NO: 169) | (SEQ ID NO: 224) |
| IgGC09 | (SEQ ID NO: 169) | (SEQ ID NO: 226) |
| IgGD02 | (SEQ ID NO: 227) | (SEQ ID NO: 228) |
| IgGD03 | (SEQ ID NO: 211) | (SEQ ID NO: 230) |
| IgGD04 | (SEQ ID NO: 203) | (SEQ ID NO: 232) |
| IgGD07 | (SEQ ID NO: 203) | (SEQ ID NO: 234) |
| IgGD08 | (SEQ ID NO: 169) | (SEQ ID NO: 236) |
| IgG87 | (SEQ ID NO: 88) | (SEQ ID NO: 90) |
| IgG89 | (SEQ ID NO: 92) | (SEQ ID NO: 90) |
| IgG94 | (SEQ ID NO: 93) | (SEQ ID NO: 90) |
| IgG97 | (SEQ ID NO: 95) | (SEQ ID NO: 90) |
| IgG102 | (SEQ ID NO: 97) | (SEQ ID NO: 90) |

In some aspects, the nucleic acid molecule encodes the VH of SEQ ID NO: 239, 243, 247, 249, 251, 253, 256, 258, 260, 262, 264, or 266, as shown in Table 8 below.

In some aspects, the nucleic acid molecule encodes the VL of SEQ ID NO: 240, 244, or 250, as shown in Table 8 below.

TABLE 8
Variable Heavy Chain (VH) and Variable Light Chain (VL) Nucleic
Acid Sequences of Anti-Mouse Cleaved CDCP1 Antibodies

| | VH Nucleic Acid Sequence | VL Nucleic Acid Sequence |
|---|---|---|
| Antibody | (SEQ ID NO) | (SEQ ID NO) |
| Fab 0114 | (SEQ ID NO: 239) | (SEQ ID NO: 240) |
| IgG 0029 | (SEQ ID NO: 239) | (SEQ ID NO: 240) |
| (Fab 0114) | | |
| Fab 0123 | (SEQ ID NO: 243) | (SEQ ID NO: 244) |
| IgG 0030 | (SEQ ID NO: 243) | (SEQ ID NO: 244) |
| (Fab 0123) | | |
| IgG 0046 | (SEQ ID NO: 247) | (SEQ ID NO: 244) |
| IgG 0050 | (SEQ ID NO: 249) | (SEQ ID NO: 250) |
| IgG 0051 | (SEQ ID NO: 251) | (SEQ ID NO: 244) |
| IgG 0055 | (SEQ ID NO: 253) | (SEQ ID NO: 244) |
| IgG 0058 | (SEQ ID NO: 256) | (SEQ ID NO: 244) |
| IgG 0059 | (SEQ ID NO: 258) | (SEQ ID NO: 244) |
| IgG 0060 | (SEQ ID NO: 260) | (SEQ ID NO: 244) |
| IgG 0062 | (SEQ ID NO: 262) | (SEQ ID NO: 244) |
| IgG 0063 | (SEQ ID NO: 264) | (SEQ ID NO: 244) |
| IgG 0065 | (SEQ ID NO: 266) | (SEQ ID NO: 244) |

Also provided herein are nucleic acid molecules encoding VH and VL sequences that are homologous to those of the anti-cleaved CDCP1 antibodies and antigen-binding fragments thereof of the disclosure. Exemplary nucleic acid molecules encode VH and VL sequences that are at least 70% identical, for example, at least 75%, at least 80%, at least 85%, at least 90%, at least 95%, or at least 99% identical, to nucleic acid molecules encoding the VH and VL sequences of the anti-cleaved CDCP1 antibodies or antigen-binding fragments thereof of the disclosure (e.g., as shown in Tables 7 and 8 above). Also provided herein are nucleic acid molecules with conservative substitutions (i.e., substitutions that do not alter the resulting amino acid sequence upon translation of nucleic acid molecule), e.g., for codon optimization.

Also provided are nucleic acids encoding the VH and/or VL regions of anti-cleaved CDCP1 antibodies or antigen-binding fragments thereof, such as the anti-cleaved CDCP1 antibodies or antigen-binding fragments thereof described herein, which nucleic acids comprise a nucleotide sequence that is at least about 75%, 80%, 85%, 90%, 95%, 96%, 97%, 98% or 99% identical to any of the nucleotide sequences encoding the VH and/or VL regions of anti-cleaved CDCP1 antibodies or antigen-binding fragments described herein (e.g., as shown in Tables 7 and 8 above).

Also provided are nucleic acids encoding the heavy chain and/or the light chain of anti-cleaved CDCP1 antibodies or antigen-binding fragments thereof, such as the anti-cleaved CDCP1 antibodies described herein, which nucleic acids comprise a nucleotide sequence that is at least about 75%, 80%, 85%, 90%, 95%, 96%, 97%, 98% or 99% identical to any of the nucleotide sequences encoding the heavy and/or light chains of anti-cleaved CDCP1 antibodies described herein.

A method for making an anti-cleaved CDCP1 antibody or antigen-binding fragment thereof as disclosed herein can comprise expressing the heavy chain and the light chains in a cell line comprising the nucleotide sequences encoding the heavy and light chains with a signal peptide. Host cells comprising these nucleotide sequences are encompassed herein.

Once DNA fragments encoding VH and VL segments are obtained, these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (hinge, CH1, CH2, and/or CH3). The sequences of human heavy chain constant region genes are known in the art (see, e.g., Kabat, E. A., et al., (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM, or IgD constant region, for example, an IgG2 and/or IgG4 constant region. For a Fab fragment heavy chain gene, the VH-encoding DNA can be operatively linked to another DNA molecule encoding only the heavy chain CH1 constant region.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see, e.g., Kabat, E. A., et al., (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region.

To create a scFv gene, the VH- and VL-encoding DNA fragments are operatively linked to another fragment encoding a flexible linker, e.g., encoding the amino acid sequence (Gly4-Ser)3, such that the VH and VL sequences can be expressed as a contiguous single-chain protein, with the VL and VH regions joined by the flexible linker (see, e.g., Bird et al., (1988) Science 242:423-426; Huston et al., (1988) Proc. Natl. Acad. Sci. USA 85:5879-5883; McCafferty et al., (1990) Nature 348:552-554).

In some aspects, the present disclosure provides a vector comprising an isolated nucleic acid molecule comprising a nucleotide sequence encoding an antibody or antigen-binding fragment thereof. In other aspects, the vectors can be used for gene therapy.

Suitable vectors for the disclosure include expression vectors, viral vectors, and plasmid vectors. In one aspect, the vector is a viral vector.

As used herein, an expression vector refers to any nucleic acid construct which contains the necessary elements for the transcription and translation of an inserted coding sequence, or in the case of an RNA viral vector, the necessary elements for replication and translation, when introduced into an appropriate host cell. Expression vectors can include plasmids, phagemids, viruses, and derivatives thereof.

Expression vectors of the disclosure can include polynucleotides encoding the antibody or antigen-binding fragment thereof described herein. In one aspect, the coding sequences for the antibody or antigen-binding fragment thereof are operably linked to an expression control sequence. As used herein, two nucleic acid sequences are operably linked when they are covalently linked in such a way as to permit each component nucleic acid sequence to retain its functionality. A coding sequence and a gene expression control sequence are said to be operably linked when they are covalently linked in such a way as to place the expression or transcription and/or translation of the coding sequence under the influence or control of the gene expression control sequence. Two DNA sequences are said to be operably linked if induction of a promoter in the 5′ gene expression sequence results in the transcription of the coding sequence and if the nature of the linkage between the two DNA sequences does not (1) result in the introduction of a frame-shift mutation, (2) interfere with the ability of the promoter region to direct the transcription of the coding sequence, or (3) interfere with the ability of the corresponding RNA transcript to be translated into a protein. Thus, a gene expression sequence would be operably linked to a coding nucleic acid sequence if the gene expression sequence were capable of effecting transcription of that coding nucleic acid sequence such that the resulting transcript is translated into the desired antibody or antigen-binding fragment thereof.

Viral vectors include, but are not limited to, nucleic acid sequences from the following viruses: retrovirus, such as Moloney murine leukemia virus, Harvey murine sarcoma virus, murine mammary tumor virus, and Rous sarcoma virus; lentivirus; adenovirus; adeno-associated virus; SV40-type viruses; polyomaviruses; Epstein-Barr viruses; papilloma viruses; herpes virus; vaccinia virus; polio virus; and RNA virus such as a retrovirus. One can readily employ other vectors well-known in the art. Certain viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high efficiency transduction of genes in vivo. Standard protocols for producing replication-deficient retroviruses (including the steps of incorporation of exogenous genetic material into a plasmid, transfection of a packaging cell line with plasmid, production of recombinant retroviruses by the packaging cell line, collection of viral particles from tissue culture media, and infection of the target cells with viral particles) are provided in Kriegler, M., Gene Transfer and Expression, A Laboratory Manual, W.H. Freeman Co., New York (1990) and Murry, E. J., Methods in Molecular Biology, Vol. 7, Humana Press, Inc., Cliffton, N.J. (1991).

Other vectors include plasmid vectors. Plasmid vectors have been extensively described in the art and are well-known to those of skill in the art. See, e.g., Sambrook et al., Molecular Cloning: A Laboratory Manual, Second Edition, Cold Spring Harbor Laboratory Press, 1989. In the last few years, plasmid vectors have been found to be particularly advantageous for delivering genes to cells in vivo because of their inability to replicate within and integrate into a host genome. These plasmids, however, having a promoter compatible with the host cell, can express a peptide from a gene operably encoded within the plasmid. Some commonly used plasmids available from commercial suppliers include pBR322, pUC18, pUC19, various pcDNA plasmids, pRC/CMV, various pCMV plasmids, pSV40, and pBlueScript. Additional examples of specific plasmids include pcDNA3.1, catalog number V79020; pcDNA3.1/hygro, catalog number V87020; pcDNA4/myc-His, catalog number V86320; and pBudCE4.1, catalog number V53220, all from Invitrogen (Carlsbad, CA.). Other plasmids are well-known to those of ordinary skill in the art. Additionally, plasmids can be custom designed using standard molecular biology techniques to remove and/or add specific fragments of DNA.

### VI. Antibody Production

Antibodies or fragments thereof that specifically bind to cleaved CDCP1 (e.g., human or mouse cleaved CDCP1) can be produced by any method known in the art for the synthesis of antibodies, for example, by chemical synthesis or by recombinant expression techniques. The methods described herein employs, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described, for example, in the references cited herein and are fully explained in the literature. See, e.g., Maniatis T et al., (1982) Molecular Cloning: A Laboratory Manual, Cold Spring Harbor Laboratory Press; Sambrook J et al., (1989), Molecular Cloning: A Laboratory Manual, Second Edition, Cold Spring Harbor Laboratory Press; Sambrook J et al., (2001) Molecular Cloning: A Laboratory Manual, Cold Spring Harbor Laboratory Press, Cold Spring Harbor, NY; Ausubel F M et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren B et al., (eds.) (1999) Genome Analysis: A Laboratory Manual, Cold Spring Harbor Laboratory Press.

In a specific aspect, an antibody described herein is an antibody (e.g., monoloclonal antibody) prepared, expressed, created or isolated by any means that involves creation, e.g., via synthesis, genetic engineering of DNA sequences. In certain aspects, such antibody comprises sequences (e.g., DNA sequences or amino acid sequences) that do not naturally exist within the antibody germline repertoire of an animal or mammal (e.g., human) in vivo.

In a certain aspect, provided herein is a method of making an antibody or an antigen-binding fragment thereof which specifically binds to cleaved CDCP1 (e.g., human or mouse cleaved CDCP1) comprising culturing a cell or host cell described herein. In a certain aspect, provided herein is a method of making an antibody or an antigen-binding fragment thereof which specifically binds to cleaved CDCP1 (e.g., human or mouse cleaved CDCP1) comprising expressing (e.g., recombinantly expressing) the antibody or antigen-binding fragment thereof using a cell or host cell described herein (e.g., a cell or a host cell comprising polynucleotides encoding an antibody described herein). In a particular aspect, the cell is an isolated cell. In a particular aspect, the exogenous polynucleotides have been introduced into the cell. In a particular aspect, the method further comprises the step of purifying the antibody or antigen-binding fragment thereof obtained from the cell or host cell.

Methods for producing polyclonal antibodies are known in the art (see, for example, Chapter 11 in: Short Protocols in Molecular Biology, (2002) 5th Ed., Ausubel F M et al., eds., John Wiley and Sons, New York).

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow E & Lane D, Antibodies: A Laboratory Manual, (Cold Spring Harbor Laboratory Press, 2nd ed. 1988); Hammerling G J et al., in: Monoclonal Antibodies and T-Cell Hybridomas 563 681 (Elsevier, N. Y., 1981). The term “monoclonal antibody” as used herein is not limited to antibodies produced through hybridoma technology. For example, monoclonal antibodies can be produced recombinantly from host cells exogenously expressing an antibody described herein or a fragment thereof, for example, light chain and/or heavy chain of such antibody.

In specific aspects, a “monoclonal antibody,” as used herein, is an antibody produced by a single cell (e.g., hybridoma or host cell producing a recombinant antibody), wherein the antibody specifically binds to cleaved CDCP1 (e.g., human or mouse cleaved CDCP1) as determined, e.g., by ELISA or other antigen-binding or competitive binding assay known in the art or in the Examples provided herein. In particular aspects, a monoclonal antibody can be a chimeric antibody or a humanized antibody. In certain aspects, a monoclonal antibody is a monovalent antibody or multivalent (e.g., bivalent) antibody. In particular aspects, a monoclonal antibody is a monospecific or multispecific antibody (e.g., bispecific antibody). Monoclonal antibodies described herein can, for example, be made by the hybridoma method as described in Kohler G & Milstein C (1975) Nature 256:495 or can, e.g., be isolated from phage libraries using the techniques as described herein, for example. Other methods for the preparation of clonal cell lines and of monoclonal antibodies expressed thereby are well known in the art (see, for example, Chapter 11 in: Short Protocols in Molecular Biology, (2002) 5th Ed., Ausubel F M et al., supra).

Methods for producing and screening for specific antibodies using hybridoma technology are routine and well known in the art. For example, in the hybridoma method, a mouse or other appropriate host animal, such as a sheep, goat, rabbit, rat, hamster or macaque monkey, is immunized to elicit lymphocytes that produce or are capable of producing antibodies that will specifically bind to the protein (e.g., human or mouse cleaved CDCP1) used for immunization. Alternatively, lymphocytes can be immunized in vitro. Lymphocytes then are fused with myeloma cells using a suitable fusing agent, such as polyethylene glycol, to form a hybridoma cell (Goding J W (Ed), Monoclonal Antibodies: Principles and Practice, pp. 59-10³ (Academic Press, 1986)). Additionally, a RIMMS (repetitive immunization multiple sites) technique can be used to immunize an animal (Kilpatrick K E et al., (1997) Hybridoma 16:381-9, incorporated by reference in its entirety).

In some aspects, mice (or other animals, such as chickens, rats, monkeys, donkeys, pigs, sheep, hamster, or dogs) can be immunized with an antigen (e.g., cleaved CDCP1 such as human cleaved CDCP1) and once an immune response is detected, e.g., antibodies specific for the antigen are detected in the mouse serum, the mouse spleen is harvested and splenocytes isolated. The splenocytes are then fused by well-known techniques to any suitable myeloma cells, for example cells from cell line SP20 available from the American Type Culture Collection (ATCC) (Manassas, VA), to form hybridomas. Hybridomas are selected and cloned by limited dilution. In certain aspects, lymph nodes of the immunized mice are harvested and fused with NSO myeloma cells.

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Specific aspects employ myeloma cells that fuse efficiently, support stable high-level production of antibody by the selected antibody-producing cells, and are sensitive to a medium such as HAT medium. Among these myeloma cell lines are murine myeloma lines, such as NSO cell line or those derived from MOPC-21 and MPC-11 mouse tumors available from the Salk Institute Cell Distribution Center, San Diego, CA, USA, and SP-2 or X63-Ag8.653 cells available from the American Type Culture Collection, Rockville, MD, USA. Human myeloma and mouse-human heteromyeloma cell lines also have been described for the production of human monoclonal antibodies (Kozbor D (1984) J Immunol 133:3001-5; Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against cleaved CDCP1 (e.g., human or mouse cleaved CDCP1). The binding specificity of monoclonal antibodies produced by hybridoma cells is determined by methods known in the art, for example, immunoprecipitation or by an in vitro binding assay, such as radioimmunoassay (RIA) or enzyme-linked immunoabsorbent assay (ELISA).

After hybridoma cells are identified that produce antibodies of the desired specificity, affinity, and/or activity, the clones can be subcloned by limiting dilution procedures and grown by standard methods (Goding J W (Ed), Monoclonal Antibodies: Principles and Practice, supra). Suitable culture media for this purpose include, for example, D-MEM or RPMI 1640 medium. In addition, the hybridoma cells can be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Antibodies described herein include antibody fragments which recognize specific cleaved CDCP1 (e.g., human or mouse cleaved CDCP1) and can be generated by any technique known to those of skill in the art. For example, Fab and F(ab′)2 fragments described herein can be produced by proteolytic cleavage of immunoglobulin molecules, using enzymes such as papain (to produce Fab fragments) or pepsin (to produce F(ab′)2 fragments). A Fab fragment corresponds to one of the two identical arms of an antibody molecule and contains the complete light chain paired with the VH and CH1 domains of the heavy chain. A F(ab′)2 fragment contains the two antigen-binding arms of an antibody molecule linked by disulfide bonds in the hinge region.

In one aspect, to generate whole antibodies, PCR primers including VH or VL nucleotide sequences, a restriction site, and a flanking sequence to protect the restriction site can be used to amplify the VH or VL sequences from a template, e.g., scFv clones. Utilizing cloning techniques known to those of skill in the art, the PCR amplified VH domains can be cloned into vectors expressing a VH constant region, and the PCR amplified VL domains can be cloned into vectors expressing a VL constant region, e.g., human kappa or lambda constant regions. The VH and VL domains can also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

A chimeric antibody is a molecule in which different fragments of the antibody are derived from different immunoglobulin molecules. For example, a chimeric antibody can contain a variable region of a non-human animal (e.g., mouse, rat or chicken) monoclonal antibody fused to a constant region of a human antibody. Methods for producing chimeric antibodies are known in the art. See, e.g., Morrison S L (1985) Science 229:1202-7; Oi V T & Morrison S L (1986) BioTechniques 4:214-221; Gillies S D et al., (1989) J Immunol Methods 125:191-202; and U.S. Pat. Nos. 5,807,715, 4,816,567, 4,816,397, and 6,331,415.

A humanized antibody is capable of binding to a predetermined antigen and which comprises a framework region having substantially the amino acid sequence of a human immunoglobulin and CDRs having substantially the amino acid sequence of a non-human immunoglobulin (e.g., a murine or a chicken immunoglobulin). In particular aspects, a humanized antibody also comprises at least a fragment of an immunoglobulin constant region (Fc), typically that of a human immunoglobulin. The antibody also can include the CH1, hinge, CH2, CH3, and CH4 regions of the heavy chain. A humanized antibody can be selected from any class of immunoglobulins, including IgM, IgG, IgD, IgA, and IgE, and any isotype, including IgG1, IgG2, IgG3, and IgG4. Humanized antibodies can be produced using a variety of techniques known in the art, including but not limited to, CDR-grafting (European Patent No. EP 239400; International Publication No. WO 91/09967; and U.S. Pat. Nos. 5,225,539, 5,530,101, and 5,585,089), veneering or resurfacing (European Patent Nos. EP 592106 and EP 519596; Padlan E A (1991) Mol Immunol 28 (4/5): 489-498; Studnicka G M et al., (1994) Prot Engineering 7 (6): 805-814; and Roguska M A et al., (1994) PNAS 91:969-973), chain shuffling (U.S. Pat. No. 5,565,332), and techniques disclosed in, e.g., U.S. Pat. Nos. 6,407,213, 5,766,886, International Publication No. WO 93/17105; Tan P et al., (2002) J Immunol 169:1119-25; Caldas C et al., (2000) Protein Eng. 13 (5): 353-60; Morea V et al., (2000) Methods 20 (3): 267-79; Baca M et al., (1997) J Biol Chem 272 (16): 10678-84; Roguska M A et al., (1996) Protein Eng 9 (10): 895 904; Couto J R et al., (1995) Cancer Res. 55 (23 Supp): 5973s-5977s; Couto J R et al., (1995) Cancer Res 55 (8): 1717-22; Sandhu J S (1994) Gene 150 (2): 409-10 and Pedersen J T et al., (1994) J Mol Biol 235 (3): 959-73. See also U.S. Application Publication No. US 2005/0042664 A1 (Feb. 24, 2005), which is incorporated by reference herein in its entirety.

Methods for making multispecific (e.g., bispecific antibodies) have been described. See, for example, U.S. Pat. Nos. 7,951,917; 7,183,076; 8,227,577; 5,837,242; 5,989,830; 5,869,620; 6,132,992 and 8,586,713.

Single domain antibodies, for example, antibodies lacking the light chains, can be produced by methods well known in the art. See Riechmann L & Muyldermans S (1999). J Immunol 231:25-38; Nuttall S D et al., (2000) Curr Pharm Biotechnol 1 (3): 253-263; Muyldermans S, (2001) J Biotechnol 74 (4): 277-302; U.S. Pat. No. 6,005,079; and International Publication Nos. WO 94/04678, WO 94/25591 and WO 01/44301.

Further, antibodies that specifically bind to a cleaved CDCP1 antigen can, in turn, be utilized to generate anti-idiotype antibodies that “mimic” an antigen using techniques well known to those skilled in the art. (See, e.g., Greenspan N S & Bona C A (1989) FASEB J 7 (5): 437-444; and Nissinoff A (1991) J Immunol 147 (8): 2429-2438).

In particular aspects, an antibody described herein, which binds to the same epitope of cleaved CDCP1 (e.g., human or mouse cleaved CDCP1) as an anti-CDCP1 antibody described herein, is a human antibody or an antigen-binding fragment thereof. In particular aspects, an antibody described herein, which competitively blocks (e.g., in a dose-dependent manner) antibodies described herein, (e.g., CL03 and CL07) from binding to cleaved CDCP1 (e.g., human cleaved CDCP1), is a human antibody or an antigen-binding fragment thereof.

Human antibodies can be produced using any method known in the art. For example, transgenic mice which are incapable of expressing functional endogenous immunoglobulins, but which can express human immunoglobulin genes, can be used. In particular, the human heavy and light chain immunoglobulin gene complexes can be introduced randomly or by homologous recombination into mouse embryonic stem cells. Alternatively, the human variable region, constant region, and diversity region can be introduced into mouse embryonic stem cells in addition to the human heavy and light chain genes. The mouse heavy and light chain immunoglobulin genes can be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a fragment of an antigen (e.g., cleaved CDCP1). Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg N & Huszar D (1995) Int Rev Immunol 13:65-93. For a detailed discussion of this technology for producing human antibodies and human monoclonal antibodies and protocols for producing such antibodies, see, e.g., International Publication Nos. WO 98/24893, WO 96/34096 and WO 96/33735; and U.S. Pat. Nos. 5,413,923, 5,625,126, 5,633,425, 5,569,825, 5,661,016, 5,545,806, 5,814,318 and 5,939,598. Examples of mice capable of producing human antibodies include the XENOMOUSE™ (Abgenix, Inc.; U.S. Pat. Nos. 6,075,181 and 6,150,184), the HUAB-MOUSE™ (Mederex, Inc./Gen Pharm; U.S. Pat. Nos. 5,545,806 and 5,569,825), the TRANS CHROMO MOUSE™ (Kirin) and the KM MOUSE™ (Medarex/Kirin).

Human antibodies which specifically bind to cleaved CDCP1 (e.g., human cleaved CDCP1) can be made by a variety of methods known in the art including phage display methods described above using antibody libraries derived from human immunoglobulin sequences. See also U.S. Pat. Nos. 4,444,887, 4,716,111, and 5,885,793; and International Publication Nos. WO 98/46645, WO 98/50433, WO 98/24893, WO 98/16654, WO 96/34096, WO 96/33735, and WO 91/10741.

In some aspects, human antibodies can be produced using mouse-human hybridomas. For example, human peripheral blood lymphocytes transformed with Epstein-Barr virus (EBV) can be fused with mouse myeloma cells to produce mouse-human hybridomas secreting human monoclonal antibodies, and these mouse-human hybridomas can be screened to determine ones which secrete human monoclonal antibodies that specifically bind to a target antigen (e.g., cleaved CDCP1 such as human cleaved CDCP1). Such methods are known and are described in the art, see, e.g., Shinmoto H et al., (2004) Cytotechnology 46:19-23; Naganawa Y et al., (2005) Human Antibodies 14:27-31.

### VII. Cells and Vectors

In certain aspects, provided herein are cells (e.g., host cells) expressing (e.g., recombinantly) antibodies described herein (or an antigen-binding fragment thereof) which specifically bind to cleaved CDCP1 (e.g., human cleaved CDCP1) and related polynucleotides and expression vectors. Provided herein are vectors (e.g., expression vectors) comprising polynucleotides comprising nucleotide sequences encoding anti-cleaved CDCP1 antibodies or a fragment for recombinant expression in host cells, e.g., in mammalian cells. Also provided herein are host cells comprising such vectors for recombinantly expressing anti-cleaved CDCP1 antibodies described herein (e.g., human or humanized antibody). In a particular aspect, provided herein are methods for producing an antibody described herein, comprising expressing such antibody from a host cell.

Recombinant expression of an antibody described herein (e.g., a full-length antibody, heavy and/or light chain of an antibody, or a single chain antibody described herein) that specifically binds to cleaved CDCP1 (e.g., human cleaved CDCP1) involves construction of an expression vector containing a polynucleotide that encodes the antibody. Once a polynucleotide encoding an antibody molecule, heavy and/or light chain of an antibody, or a fragment thereof (e.g., heavy and/or light chain variable domains) described herein has been obtained, the vector for the production of the antibody molecule can be produced by recombinant DNA technology using techniques well known in the art. Thus, methods for preparing a protein by expressing a polynucleotide containing an antibody or antibody fragment (e.g., light chain or heavy chain) encoding nucleotide sequence are described herein. Methods which are well known to those skilled in the art can be used to construct expression vectors containing antibody or antibody fragment (e.g., light chain or heavy chain) coding sequences and appropriate transcriptional and translational control signals. These methods include, for example, in vitro recombinant DNA techniques, synthetic techniques, and in vivo genetic recombination. Also provided are replicable vectors comprising a nucleotide sequence encoding an antibody molecule described herein, a heavy or light chain of an antibody, a heavy or light chain variable domain of an antibody or a fragment thereof, or a heavy or light chain CDR, operably linked to a promoter. Such vectors can, for example, include the nucleotide sequence encoding the constant region of the antibody molecule (see, e.g., International Publication Nos. WO 86/05807 and WO 89/01036; and U.S. Pat. No. 5,122,464) and variable domains of the antibody can be cloned into such a vector for expression of the entire heavy, the entire light chain, or both the entire heavy and light chains.

An expression vector can be transferred to a cell (e.g., host cell) by conventional techniques and the resulting cells can then be cultured by conventional techniques to produce an antibody described herein (e.g., an antibody comprising the VH and/or VL, or one or more of the VH and/or VL CDRs, of the anti-human or mouse cleaved CDCP1 antibodies or antigen-binding fragments thereof) or a fragment thereof. Thus, provided herein are host cells containing a polynucleotide encoding an antibody described herein or fragments thereof, or a heavy or light chain thereof, or fragment thereof, or a single chain antibody described herein, operably linked to a promoter for expression of such sequences in the host cell. In certain aspects, for the expression of double-chained antibodies, vectors encoding both the heavy and light chains, individually, can be co-expressed in the host cell for expression of the entire immunoglobulin molecule, as detailed below. In certain aspects, a host cell contains a vector comprising a polynucleotide encoding both the heavy chain and light chain of an antibody described herein, or a fragment thereof. In specific aspects, a host cell contains two different vectors, a first vector comprising a polynucleotide encoding a heavy chain or a heavy chain variable region of an antibody described herein, or a fragment thereof, and a second vector comprising a polynucleotide encoding a light chain or a light chain variable region of an antibody described herein, or a fragment thereof. In other aspects, a first host cell comprises a first vector comprising a polynucleotide encoding a heavy chain or a heavy chain variable region of an antibody described herein, or a fragment thereof, and a second host cell comprises a second vector comprising a polynucleotide encoding a light chain or a light chain variable region of an antibody described herein. In specific aspects, a heavy chain/heavy chain variable region expressed by a first cell associated with a light chain/light chain variable region of a second cell to form an anti-cleaved CDCP1 antibody (e.g., anti-human or mouse cleaved CDCP1 antibody) described herein or an antigen-binding fragment thereof. In certain aspects, provided herein is a population of host cells comprising such first host cell and such second host cell.

In some aspects, provided herein is a population of vectors comprising a first vector comprising a polynucleotide encoding a light chain/light chain variable region of an anti-cleaved CDCP1 antibody described herein, and a second vector comprising a polynucleotide encoding a heavy chain/heavy chain variable region of an anti-cleaved CDCP1 antibody described herein.

In some aspects, provided herein is a population of vectors comprising a first vector comprising a polynucleotide encoding a cleaved CUB1 ectodomain and a second polynucleotide encoding a CUB2/CUB3 ectodomain.

A variety of host-expression vector systems can be utilized to express antibody molecules described herein. Such host-expression systems represent vehicles by which the coding sequences of interest can be produced and subsequently purified, but also represent cells which can, when transformed or transfected with the appropriate nucleotide coding sequences, express an antibody molecule described herein in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems (e.g., green algae such as Chlamydomonas reinhardtii) infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS (e.g., COS1 or COS), CHO, BHK, MDCK, HEK 293, NSO, PER.C6, VERO, CRL7030, HsS78Bst, HeLa, NIH 3T3, HEK-293T, HepG2, SP210, R1.1, B-W, L-M, BSCl, BSC40, YB/20, SP2/0, Sf9, human lymphoblastoid, NSO, bow melanoma, HT-1080, PERC.6, and BMT10 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In a specific aspect, cells for expressing antibodies described herein or an antigen-binding fragment thereof are CHO cells, for example CHO cells from the CHO GS SYSTEM™ (Lonza). In a particular aspect, cells for expressing antibodies described herein are human cells, e.g., human cell lines. In a specific aspect, a mammalian expression vector is POPTIVEC™ or pcDNA3.3. In a particular aspect, bacterial cells such as Escherichia coli, or eukaryotic cells (e.g., mammalian cells), especially for the expression of whole recombinant antibody molecule, are used for the expression of a recombinant antibody molecule. For example, mammalian cells such as Chinese hamster ovary (CHO) cells, in conjunction with a vector such as the major intermediate early gene promoter element from human cytomegalovirus is an effective expression system for antibodies (Foecking M K & Hofstetter H (1986) Gene 45:101-5; and Cockett M I et al., (1990) Biotechnology 8 (7): 662-7). In certain aspects, antibodies described herein are produced by HEK-293T cells. In a specific aspect, the expression of nucleotide sequences encoding antibodies described herein which specifically bind cleaved CDCP1 (e.g., human cleaved CDCP1) is regulated by a constitutive promoter, inducible promoter or tissue specific promoter.

In bacterial systems, a number of expression vectors can be advantageously selected depending upon the use intended for the antibody molecule being expressed. For example, when a large quantity of such an antibody is to be produced, for the generation of pharmaceutical compositions of an antibody molecule, vectors which direct the expression of high levels of fusion protein products that are readily purified can be desirable. Such vectors include, but are not limited to, the E. coli expression vector pUR278 (Ruether U & Mueller-Hill B (1983) EMBO J 2:1791-1794), in which the antibody coding sequence can be ligated individually into the vector in frame with the lac Z coding region so that a fusion protein is produced; pIN vectors (Inouye S & Inouye M (1985) Nuc Acids Res 13:3101-3109; Van Heeke G & Schuster S M (1989) J Biol Chem 24:5503-5509); and the like. For example, pGEX vectors can also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV), for example, can be used as a vector to express foreign genes. The virus grows in Spodoptera frugiperda cells. The antibody coding sequence can be cloned individually into non-essential regions (for example the polyhedrin gene) of the virus and placed under control of an AcNPV promoter (for example the polyhedrin promoter).

In mammalian host cells, a number of viral-based expression systems can be utilized. In cases where an adenovirus is used as an expression vector, the antibody coding sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan J & Shenk T (1984) PNAS 81 (12): 3655-9). Specific initiation signals can also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see, e.g., Bitter G et al., (1987) Methods Enzymol. 153:516-544).

In addition, a host cell strain can be chosen which modulates the expression of the inserted sequences, or modifies and processes the gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used. Such mammalian host cells include but are not limited to CHO, SKOV-3, B16-F1, NCI-H522, VERO, BHK, Hela, MDCK, HEK-293, NIH 3T3, W138, BT483, Hs578T, HTB2, BT20 and T47D, NSO (a murine myeloma cell line that does not endogenously produce any immunoglobulin chains), CRL7030, COS (e.g., COS 1 or COS), PER.C6, VERO, HsS78Bst, HEK-293T, HepG2, SP210, RI. 1, B-W, L-M, BSC1, BSC40, YB/20, BMT10 and HsS78Bst cells. In certain aspects, anti-cleaved CDCP1 antibodies described herein are produced in mammalian cells, such as HEK-293T cells.

In a specific aspect, the antibodies described herein or antigen-binding fragments thereof have reduced fucose content or no fucose content. Such antibodies can be produced using techniques known one skilled in the art. For example, the antibodies can be expressed in cells deficient or lacking the ability of to fucosylate. In a specific example, cell lines with a knockout of both alleles of 1,6-fucosyltransferase can be used to produce antibodies or antigen-binding fragments thereof with reduced fucose content. The POTELLIGENT® system (Lonza) is an example of such a system that can be used to produce antibodies or antigen-binding fragments thereof with reduced fucose content.

For long-term, high-yield production of recombinant proteins, stable expression cells can be generated. For example, cell lines which stably express an anti-cleaved CDCP1 antibody described herein or an antigen-binding fragment thereof can be engineered. In specific aspects, a cell provided herein stably expresses a light chain/light chain variable domain and a heavy chain/heavy chain variable domain which associate to form an antibody described herein or an antigen-binding fragment thereof.

In certain aspects, rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA/polynucleotide, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express an anti-cleaved CDCP1 antibody described herein or an antibody binding fragment thereof. Such engineered cell lines can be particularly useful in screening and evaluation of compositions that interact directly or indirectly with the antibody molecule.

A number of selection systems can be used, including but not limited to, the herpes simplex virus thymidine kinase (Wigler M et al., (1977) Cell 11 (1): 223-32), hypoxanthineguanine phosphoribosyltransferase (Szybalska E H & Szybalski W (1962) PNAS 48 (12): 2026-2034) and adenine phosphoribosyltransferase (Lowy I et al., (1980) Cell 22 (3): 817-23) genes can be employed in tk-, hgprt- or aprt-cells, respectively. Also, antimetabolite resistance can be used as the basis of selection for the following genes: dhfr, which confers resistance to methotrexate (Wigler M et al., (1980) PNAS 77 (6): 3567-70; O'Hare K et al., (1981) PNAS 78:1527-31); gpt, which confers resistance to mycophenolic acid (Mulligan R C & Berg P (1981) PNAS 78 (4): 2072-6); neo, which confers resistance to the aminoglycoside G-418 (Wu G Y & Wu C H (1991) Biotherapy 3:87-95; Tolstoshev P (1993) Ann Rev Pharmacol Toxicol 32:573-596; Mulligan R C (1993) Science 260:926-932; and Morgan R A & Anderson W F (1993) Ann Rev Biochem 62:191-217; Nabel G J & Feigner P L (1993) Trends Biotechnol 11 (5): 211-5); and hygro, which confers resistance to hygromycin (Santerre R F et al., (1984) Gene 30 (1-3): 147-56). Methods commonly known in the art of recombinant DNA technology can be routinely applied to select the desired recombinant clone and such methods are described, for example, in Ausubel F M et al., (eds.), Current Protocols in Molecular Biology, John Wiley & Sons, NY (1993); Kriegler M, Gene Transfer and Expression, A Laboratory Manual, Stockton Press, NY (1990); and in Chapters 12 and 13, Dracopoli N C et al., (eds.), Current Protocols in Human Genetics, John Wiley & Sons, NY (1994); Colbere-Garapin F et al., (1981) J Mol Biol 150:1-14, which are incorporated by reference herein in their entireties.

In some aspects, the antibody or antigen-binding fragment thereof of the present disclosure can be expressed on an immune cell, e.g., a T cell and/or an NK cell. In some aspects, the antibody or antigen-binding fragment thereof can be expressed as a chimeric antigen receptor (CAR). A CAR-T cell is a T cell that expresses a chimeric antigen receptor. The phrase “chimeric antigen receptor (CAR),” as used herein, refers to a recombinant fusion protein that has an antigen-specific extracellular (or ectodomain) domain coupled to an intracellular domain that directs the cell to perform a specialized function upon binding of an antigen to the extracellular domain. Chimeric antigen receptors are distinguished from other antigen binding agents by their ability to both bind MHC-independent antigen and transduce activation signals via their intracellular domain.

In some aspects, the antigen-specific extracellular domain of a chimeric antigen receptor recognizes and specifically binds an antigen, i.e., cleaved CDCP1. A cleaved CDCP1-specific extracellular domain suitable for use in a CAR of the present disclosure can be any antigen-binding polypeptide, a wide variety of which are known in the art. In some instances, the antigen-binding domain is a single chain Fv (scFv) or Fab. In other aspects, the antigen binding fragment useful for a CAR of the present disclosure includes an antigen-binding fragment disclosed anywhere herein.

In some aspects, the transmembrane domain useful for a CAR is connected to the extracellular domain and can include a naturally occurring transmembrane domain. In other aspects, the transmembrane domain useful for a CAR can be derived from alpha chain, beta chain, or zeta chain of T cell receptor, CD28, CD3 E, CD45, CD4, CD5, CDS, CD9, CD16, CD22, CD33, CD37, CD64, CD80, CD86, CD134, CD154, CD8, or any other known in the art.

The term “intracellular domain” refers to the portion of a CAR that transduces the effector function signal upon binding of an antigen to the extracellular domain and directs the T cell to perform a specialized function. In one aspect, an intracellular domain for a CAR comprises an immune receptor tyrosine-based activation motif activation motif (ITAM). In some aspects, the ITAM is derived from CD3 zeta ((, zeta), FcR gamma, FcR beta, CD3 gamma, CD3 delta, CD3 epsilon, CDS, CD22, CD79a, CD79b, CD66d, 4-1 BB, DAP-1 0, OX40, or Fc [epsilon] RI [gamma]

In some aspects, the CAR of the present disclosure further comprises a costimultatory domain that can be linked to the intracellular domain. The co-stimulatory domain in a CAR construct can transmit signals and activate the cells as a part of an intracellular portion of the CAR. In some aspects, the costimulatory domain is derived from CD27, CD28, 4-1BB, OX40, CD30, CD40, PD-1, ICOS, lymphocyte function-associated antigen-1 (LFA-1), CD7, LIGHT, NKG2C, or B7-H3.

In other aspects, a CAR of the present disclosure further comprises a linker. A short oligopeptide or polypeptide linker can be present between the transmembrane domain and the intracellular domain. In some aspects, the linker is not limited to a particular length as long as the intracellular domain of the CAR when the extracellular domain is bound to the antigen, i.e., cleaved CDCP1, is capable of inducing T cell activation. In some aspects, the linker comprises (Gly4Ser)3 linker.

In other aspects, the present disclosure includes a polynucleotide encoding the CAR of the present disclosure or a vector comprising the polynucleotide.

As used herein, the term “T cell” is a lymphocyte derived from the thymus and contrite to cell's immune response. The T cells include CD4+ T cells (helper T cells, TH cells), CD8+ T cells (cytotoxic T cells, CTL), memory T cells, regulatory T cells (Treg), or natural killer T cells. In some aspects, the T cell into which a CAR is introduced is a CD8+ T cell.

### VIII. Immunoconjugates, Antibody Derivatives and Diagnostics

Anti-cleaved CDCP1 antibodies described herein can be used for diagnostic purposes, including sample testing and in vivo imaging, and for this purpose the antibody (or binding fragment thereof) can be conjugated to an appropriate detectable agent, to form an immunoconjugate. For diagnostic purposes, appropriate agents are detectable labels that include radioisotopes, for whole body imaging, and radioisotopes, enzymes, fluorescent labels and other suitable antibody tags for sample testing.

The detectable labels that can be linked to any anti-cleaved CDCP1 antibody described herein can be any of the various types used currently in the field of in vitro diagnostics, including particulate labels including metal sols such as colloidal gold, isotopes such as I¹²⁵ or Tc⁹⁹ presented for instance with a peptidic chelating agent of the N₂S₂, N₃S or N₄ type, chromophores including fluorescent markers, luminescent markers, phosphorescent markers and the like, as well as enzyme labels that convert a given substrate to a detectable marker, and polynucleotide tags that are revealed following amplification such as by polymerase chain reaction. Suitable enzyme labels include horseradish peroxidase, alkaline phosphatase and the like. For instance, the label can be the enzyme alkaline phosphatase, detected by measuring the presence or formation of chemiluminescence following conversion of 1,2 dioxetane substrates such as adamantyl methoxy phosphoryloxy phenyl dioxetane (AMPPD), disodium 3-(4-(methoxyspiro {1,2-dioxetane-3,2′-(5′-chloro)tricyclo {3.3.1.1 3,7}decan}-4-yl) phenyl phosphate (CSPD), as well as CDP and CDP-STARR or other luminescent substrates well-known to those in the art, for example the chelates of suitable lanthanides such as Terbium (III) and Europium (III). The detection means is determined by the chosen label. Appearance of the label or its reaction products can be achieved using the naked eye, in the case where the label is particulate and accumulates at appropriate levels, or using instruments such as a spectrophotometer, a luminometer, a fluorimeter, and the like, all in accordance with standard practice.

In some aspects, conjugation methods result in linkages which are substantially (or nearly) non-immunogenic, e.g., peptide- (i.e., amide-), sulfide-, (sterically hindered), disulfide-, hydrazone-, and ether linkages. These linkages are nearly non-immunogenic and show reasonable stability within serum (see e.g., Senter, P. D., Curr. Opin. Chem. Biol. 13 (2009) 235-244; WO 2009/059278; WO 95/17886).

Depending on the biochemical nature of the moiety and the antibody, different conjugation strategies can be employed. In case the moiety is naturally-occurring or recombinant of between 50 to 500 amino acids, there are standard procedures in text books describing the chemistry for synthesis of protein conjugates, which can be easily followed by the skilled artisan (see, e.g., Hackenberger, C. P. R., and Schwarzer, D., Angew. Chem. Int. Ed. Engl. 47 (2008) 10030-10074). In some aspects the reaction of a maleinimido moiety with a cysteine residue within the antibody or the moiety is used. This is an especially suited coupling chemistry in case e.g., a

Fab or Fab′-fragment of an antibody is used. Alternatively, in some aspects, coupling to the C-terminal end of the antibody or moiety is performed. C-terminal modification of a protein, e.g., of a Fab-fragment, can be performed as described (Sunbul, M. and Yin, J., Org. Biomol. Chem. 7 (2009) 3361-3371).

In general, site specific reaction and covalent coupling is based on transforming a natural amino acid into an amino acid with a reactivity which is orthogonal to the reactivity of the other functional groups present. For example, a specific cysteine within a rare sequence context can be enzymatically converted in an aldehyde (see Frese, M. A., and Dierks, T., ChemBioChem. 10 (2009) 425-427). It is also possible to obtain a desired amino acid modification by utilizing the specific enzymatic reactivity of certain enzymes with a natural amino acid in a given sequence context (see, e.g., Taki, M. et al., Prot. Eng. Des. Sel. 17 (2004) 119-126; Gautier, A. et al. Chem. Biol. 15 (2008) 128-136; and Protease-catalyzed formation of C—N bonds is used by Bordusa, F., Highlights in Bioorganic Chemistry (2004) 389-403). Site specific reaction and covalent coupling can also be achieved by the selective reaction of terminal amino acids with appropriate modifying reagents.

The reactivity of an N-terminal cysteine with benzonitrils (see Ren, H. et al., Angew. Chem. Int. Ed. Engl. 48 (2009) 9658-9662) can be used to achieve a site-specific covalent coupling.

Native chemical ligation can also rely on C-terminal cysteine residues (Taylor, E. Vogel; Imperiali, B, Nucleic Acids and Molecular Biology (2009), 22 (Protein Engineering), 65-96).

U.S. Pat. No. 6,437,095 B1 describes a conjugation method which is based on the faster reaction of a cysteine within a stretch of negatively charged amino acids with a cysteine located in a stretch of positively charged amino acids.

The moiety can also be a synthetic peptide or peptide mimic. In case a polypeptide is chemically synthesized, amino acids with orthogonal chemical reactivity can be incorporated during such synthesis (see e.g., de Graaf, A. J. et al., Bioconjug. Chem. 20 (2009) 1281-1295). Since a great variety of orthogonal functional groups is at stake and can be introduced into a synthetic peptide, conjugation of such peptide to a linker is standard chemistry.

In order to obtain a mono-labeled polypeptide, the conjugate with 1:1 stoichiometry can be separated by chromatography from other conjugation side-products. This procedure can be facilitated by using a dye labeled binding pair member and a charged linker. By using this kind of labeled and highly negatively charged binding pair member, mono conjugated polypeptides are easily separated from non-labeled polypeptides and polypeptides which carry more than one linker, since the difference in charge and molecular weight can be used for separation. The fluorescent dye can be useful for purifying the complex from un-bound components, like a labeled monovalent binder.

In some aspects the moiety attached to an anti-cleaved CDCP1 antibody is selected from the group consisting of a binding moiety, a labeling moiety, and a biologically active moiety.

Anti-cleaved CDCP1 antibodies described herein can also be conjugated to a therapeutic agent to form an immunoconjugate such as an antibody-drug conjugate (ADC). Suitable therapeutic agents include antimetabolites, alkylating agents, DNA minor groove binders, DNA intercalators, DNA crosslinkers, histone deacetylase inhibitors, nuclear export inhibitors, proteasome inhibitors, topoisomerase I or II inhibitors, heat shock protein inhibitors, tyrosine kinase inhibitors, antibiotics, and anti-mitotic agents. In the ADC, the antibody and therapeutic agent preferably are conjugated via a linker cleavable such as a peptidyl, disulfide, or hydrazone linker. In some aspects, the linker is a peptidyl linker such as Val-Cit, Ala-Val, Val-Ala-Val, Lys-Lys, Pro-Val-Gly-Val-Val (SEQ ID NO: 108), Ala-Asn-Val, Val-Leu-Lys, Ala-Ala-Asn, Cit-Cit, Val-Lys, Lys, Cit, Ser, or Glu. The ADCs can be prepared as described in U.S. Pat. Nos. 7,087,600; 6,989,452; and 7,129,261; PCT Publications WO 02/096910; WO 07/038658; WO 07/051081; WO 07/059404; WO 08/083312; and WO 08/103693; U.S. Patent Publications 20060024317; 20060004081; and 20060247295.

In some aspects, the therapeutic agent is selected from the group consisting of a cytotoxin, a non-cytotoxic drug, a radioactive agent, a second antibody, an enzyme, an anti-neoplastic agent, and any combination thereof.

In some aspects, the immunoconjugate comprises an anti-cleaved CDCP1 antibody and a cytotoxin. The cytotoxin can be selected from any cytotoxin known in the art. In some aspects, the cytotoxin is selected from the group consisting of dolastatin, monomethyl auristatin E (MMAE), cantansine, duocarmycin, calicheamicin, pyrrolobenzodiazepine, duocarmycin, centanamycin, SN38, doxorubicin, a derivative thereof, a synthetic analog thereof, and any combination thereof. In certain aspects, the immunoconjugate comprises an anti-CDCP1 antibody and Cytotoxin A. In other aspects, the immunoconjugate comprises an anti-CDCP1 antibody and a non-cytotoxic drug.

In some aspects, the immunoconjugate comprises an anti-cleaved CDCP1 antibody and a radioactive agent. In some aspects, the radioactive agent is a radionucleotide. In certain aspects, the radioactive agent comprises radioactive iodine. In particular aspects, the radioactive agent comprises 131-iodine. In other aspects, the radioactive agent comprises the radioactive isotope Yttrium-90.

In some aspects, the immunoconjugate comprises an anti-cleaved CDCP1 antibody and a second antibody. The second antibody can be any antibody described in the present disclosure, including, but not limited to, an antibody that specifically binds a protein selected from the group consisting of PD-1, PD-L1, CTLA-4, LAG3, TIGIT, TIM3, NKG2a, OX40, ICOS, CD137, KIR, TGFβ, IL-10, IL-2, IL-8, B7-H4, Fas ligand, CXCR4, mesothelin, VISTA, CD96, CD27, GITR, and any combination thereof. In some aspects, the immunoconjugate comprises an anti-cleaved CDCP1 antibody and an anti-PD-1 antibody. In another embodiment, the immunoconjugate comprises an anti-cleaved CDCP1 antibody and nivolumab.

In some aspects, the immunoconjugate comprises an anti-cleaved CDCP1 antibody and a pegylated IL-2 or pegylated IL-10.

In certain aspects, the immunoconjugate comprises an anti-cleaved CDCP1 antibody and an enzyme. In some aspects, the enzyme comprises glucose oxidase. In some aspects, the enzyme comprises a peroxidase. In some aspects, the enzyme comprises myeloperoxidase. In some aspects, the enzyme comprises glucose oxidase. In some aspects, the enzyme comprises horseradish peroxidase.

In certain aspects, the immunoconjugate comprises an anti-cleaved CDCP1 antibody and an anti-neoplastic agent. The anti-neoplastic agent can be any such agent known in the art. In some aspects, the anti-neoplastic agent is epirubicin. In some aspects, the anti-neoplastic agent is a super antigen. In certain aspects, the super antigen is staphylococcal enterotoxin A (SEA/E-120; estafenatox).

Anti-cleaved CDCP1 antibodies, e.g., those described herein, can also be used for detecting cleaved CDCP1, such as human cleaved CDCP1, e.g., human cleaved CDCP1 on the surface of a cell. The antibodies can be used, e.g., in an ELISA assay or in flow cytometry. In some aspects, an anti-cleaved CDCP1 antibody is contacted with cells or serum for a time appropriate for specific binding to occur, and then a reagent, e.g., an antibody that detects the anti-cleaved CDCP1 antibody, is added. Exemplary assays are provided in the Examples. Exemplary methods for detecting cleaved CDCP1, e.g., surface expressed cleaved CDCP1 comprise (i) contacting a sample with an anti-cleaved CDCP1 antibody, for a time sufficient for allowing specific binding of the anti-cleaved CDCP1 antibody to cleaved CDCP1 in the sample, and (2) contacting the sample with a detection reagent, e.g., an antibody, that specifically binds to the anti-cleaved CDCP1 antibody, such as to the Fc region of the anti-cleaved CDCP1 antibody, to thereby detect cleaved CDCP1 bound by the anti-cleaved CDCP1 antibody. Wash steps can be included after the incubation with the antibody and/or detection reagent. Anti-cleaved CDCP1 antibodies for use in these methods do not have to be linked to a label or detection agents, as a separate detection agent can be used.

Other uses for anti-cleaved CDCP1 antibodies, e.g., as monotherapy or combination therapy, are provided elsewhere herein, e.g., in the section pertaining to combination treatments.

### IX. Bispecific Molecules

Anti-cleaved CDCP1 antibodies described herein can be used for forming bispecific molecules. An anti-cleaved CDCP1 antibody, or antigen-binding fragments thereof, can be derivatized or linked to another functional molecule, e.g., another peptide or protein (e.g., another antibody or ligand for a receptor) to generate a bispecific molecule that binds to at least two different binding sites or target molecules. For example, an anti-cleaved CDCP1 antibody can be linked to an antibody or scFv that binds specifically to any protein that can be used as potential targets for combination treatments, such as the proteins described herein (e.g., antibodies to PD-1, PD-L1, CTLA-4, LAG3, TIGIT, TIM3, NKG2a, OX40, ICOS, CD137, KIR, TGFβ, IL-10, IL-2, IL-8, B7-H4, Fas ligand, CXCR4, mesothelin, CD27, CD96, VISTA, or GITR, or pegylated IL-2 or pegylated IL-10). The antibody described herein can in fact be derived or linked to more than one other functional molecule to generate multispecific molecules that bind to more than two different binding sites and/or target molecules; such multispecific molecules are also intended to be encompassed by the term “bispecific molecule” as used herein. To create a bispecific molecule described herein, an antibody described herein can be functionally linked (e.g., by chemical coupling, genetic fusion, noncovalent association or otherwise) to one or more other binding molecules, such as another antibody, antibody fragment, peptide or binding mimetic, such that a bispecific molecule results.

Accordingly, provided herein are bispecific molecules comprising at least one first binding specificity for cleaved CDCP1 (e.g., human cleaved CDCP1) and a second binding specificity for a second target epitope. In some aspects described herein in which the bispecific molecule is multispecific, the molecule can further include a third binding specificity.

In some aspects, the bispecific molecules described herein comprise as a binding specificity at least one antibody, or an antibody fragment thereof, including, e.g., an Fab, Fab′, F(ab′)2, Fv, or a single chain Fv (scFv). The antibody can also be a light chain or heavy chain dimer, or any minimal fragment thereof such as a Fv or a single chain construct as described in Ladner et al. U.S. Pat. No. 4,946,778.

While human monoclonal antibodies are preferred, other antibodies which can be employed in the bispecific molecules described herein are murine, chimeric and humanized monoclonal antibodies.

The bispecific molecules described herein can be prepared by conjugating the constituent binding specificities using methods known in the art. For example, each binding specificity of the bispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitrobenzoic acid) (DTNB), o-phenylenedimaleimide (oPDM), N-succinimidyl-3-(2-pyridyldithio) propionate (SPDP), and sulfosuccinimidyl 4-(N-maleimidomethyl) cyclohaxane-1-carboxylate (sulfo-SMCC) (see, e.g., Karpovsky et al. (1984) J. Exp. Med. 160:1686; Liu, M A et al. (1985) Proc. Natl. Acad. Sci. USA 82:8648). Other methods include those described in Paulus (1985) Behring Ins. Mitt. No. 78, 118-132; Brennan et al. (1985) Science 229:81-83), and Glennie et al. (1987) J. Immunol. 139:2367-2375). Some conjugating agents are SATA and sulfo-SMCC, both available from Pierce Chemical Co. (Rockford, IL).

When the binding specificities are antibodies, they can be conjugated via sulfhydryl bonding of the C-terminus hinge regions of the two heavy chains. In some aspects, the hinge region is modified to contain an odd number of sulfhydryl residues, preferably one, prior to conjugation.

Alternatively, both binding specificities can be encoded in the same vector and expressed and assembled in the same host cell. This method is particularly useful where the bispecific molecule is a mAb×mAb, mAb×Fab, mAb×(scFv)₂, Fab×F(ab′)₂ or ligand×Fab fusion protein. A bispecific antibody can comprise an antibody comprising an scFv at the C-terminus of each heavy chain. A bispecific molecule described herein can be a single chain molecule comprising one single chain antibody and a binding determinant, or a single chain bispecific molecule comprising two binding determinants. Bispecific molecules can comprise at least two single chain molecules. Methods for preparing bispecific molecules are described for example in U.S. Pat. Nos. 5,260,203; 5,455,030; 4,881,175; 5,132,405; 5,091,513; 5,476,786; 5,013,653; 5,258,498; and 5,482,858.

Binding of the bispecific molecules to their specific targets can be confirmed using art-recognized methods, such as enzyme-linked immunosorbent assay (ELISA), radioimmunoassay (RIA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest.

### X. Compositions

Further provided are compositions, e.g., a pharmaceutical compositions, containing one or a combination of anti-cleaved CDCP1 antibodies or combination with antibodies to other targets, or antigen-binding fragment(s) thereof, described herein, formulated together with a pharmaceutically acceptable carrier. Such compositions can include one or a combination of (e.g., two or more different) antibodies, or immunoconjugates or bispecific molecules described herein. For example, a pharmaceutical composition described herein can comprise a combination of antibodies (or immunoconjugates or bispecifics) that bind to different epitopes on the target antigen or that have complementary activities.

In some aspects, a composition comprises an anti-cleaved CDCP1 antibody at a concentration of at least 1 mg/ml, 5 mg/ml, 10 mg/ml, 50 mg/ml, 100 mg/ml, 150 mg/ml, 200 mg/ml, 1-300 mg/ml, or 100-300 mg/ml.

Pharmaceutical compositions described herein also can be administered in combination therapy, i.e., combined with other agents. For example, the combination therapy can include an anti-cleaved CDCP1 antibody described herein combined with at least one other anti-cancer and/or immunomodulating, e.g., T-cell stimulating (e.g., activating) agent. Examples of therapeutic agents that can be used in combination therapy are described in greater detail below in the section on uses of the anti-cleaved CDCP1 antibodies described herein or antigen-binding fragments thereof.

In some aspects, the anti-cleaved CDCP1 antibody or antigen-binding fragments thereof can be combined with at least one other agent selected from chemotherapy drugs, small molecule drugs and antibodies that stimulate the immune response to a given cancer. In some instances, the anti-cleaved CDCP1 antibody can be combined with, for example, one or more of an anti-CTLA-4 antibody, an anti-PD-1 antibody, an anti-PD-L1 antibody, an anti-OX40 (also known as CD134, TNFRSF4, ACT35 and/or TXGP1L) antibody (e.g., BMS986178, or MDX-1803), an anti-CD137 antibody, an anti-LAG-3 antibody, an anti-GITR antibody, an anti-KIR antibody, an anti-TGFβ antibody, an anti-IL-10 antibody, a long-acting IL-10 molecule (e.g. IL-10-Fc fusion, or Pegylated IL-10, such as AM0010 of ARMO BioSciences), a long-acting IL-2 (e.g., Pegylated IL-2 molecules, such as NKTR-214 of Nektar; see U.S. Pat. No. 8,252,275, WO12/065086 and WO15/125159), an anti-VISTA antibody, an anti-CD96 antibody, an anti-IL-8 antibody, an anti-B7-H4, an anti-Fas ligand antibody, an anti-CXCR4 antibody, an anti-mesothelin antibody, an anti-CD27 antibody, or any combination thereof.

In other aspects, the anti-cleaved CDCP1 antibody described herein or antigen-binding fragment thereof can be formulated with a second antibody. In some aspects, the second antibody specifically binds a protein selected from the group consisting of PD-1, PD-L1, CTLA-4, LAG3, TIGIT, TIM3, NKG2a, OX40, ICOS, CD137, KIR, TGFβ, IL-10, IL-2, VISTA, CD96, IL-8, B7-H4, Fas ligand, CXCR4, mesothelin, CD27, GITR, and any combination thereof.

In some aspects, the second antibody can be an anti-PD-1 antibody. The anti-PD-1 antibody can be any antibody that binds PD-1 and inhibits the interaction of PD-1 and PD-L1. In some aspects, the anti-PD-1 antibody is any anti-PD-1 antibody disclosed herein. In some aspects, the second antibody can be nivolumab. In some aspects, the second antibody can be pembrolizumab.

In some aspects, the second antibody can be an anti-PD-L1 antibody. The anti-PD-L1 antibody can be any antibody that binds PD-L1 and inhibits the interaction of PD-1 and PD-L1. In some aspects, the anti-PD-L1 antibody is any anti-PD-L1 antibody disclosed herein. In some aspects, the second antibody can be atezolizumab. In some aspects, the second antibody can be durvalumab. In some aspects the second antibody can be avelumab.

In some aspects, the second antibody can be an anti-CTLA-4 antibody. The anti-CTLA-4 antibody can be any antibody that binds CTLA-4 and inhibits its activity. In some aspects, the anti-CTLA-4 antibody is any anti-CTLA-4 antibody disclosed herein. In some aspects, the second antibody can be tremelimumab. In some aspects, the second antibody can be ipilimumab.

In some aspects, the second antibody can be an anti-LAG3 antibody. The anti-LAG3 antibody can be any antibody that binds LAG-3 and inhibits its activity. In some aspects, the anti-LAG3 antibody is any anti-LAG3 antibody disclosed herein. In some aspects, the second antibody can be 25F7.

In some aspects, the second antibody can be an anti-CD137 antibody. The anti-CD137 antibody can be any antibody that binds CD137 and inhibits its activity. In some aspects, the anti-CD137 antibody is any anti-CD137 antibody disclosed herein. In some aspects, the second antibody can be urelumab.

In some aspects, the second antibody can be an anti-KIR antibody. The anti-KIR antibody can be any antibody that binds KIR and inhibits its activity. In some aspects, the anti-KIR antibody is any anti-KIR antibody disclosed herein. In some aspects, the second antibody can be lirilumab.

In some aspects, the second antibody can be an anti-GITR antibody. The anti-GITR antibody can be any antibody that binds GITR and inhibits its activity. In some aspects, the anti-GITR antibody is any anti-GITR antibody disclosed herein. In some aspects, the second antibody can be MK4166. In some aspects, the second antibody can be TRX518.

In some aspects, the second antibody can be an anti-CD96 antibody.

In some aspects, the second antibody can be an anti-TIM3 antibody.

In some aspects, the second antibody can be an anti-VISTA antibody.

In some aspects, the second antibody can be an anti-NKG2a antibody.

In some aspects, the second antibody can be an anti-ICOS antibody.

In some aspects, the second antibody can be an anti-OX40 antibody.

In some aspects, the second antibody can be an anti-IL8 antibody, such as HuMax®-IL8 (BMS-986253).

In some aspects, the anti-cleaved CDCP1 antibody can be formulated with a long-acting IL-10 molecule. In some aspects, the anti-cleaved CDCP1 antibody can be formulated with IL-10-Fc fusion molecule. In some aspects, the anti-cleaved CDCP1 antibody can be formulated with Pegylated IL-10, such as AM0010 of ARMO BioSciences.

In some aspects, the anti-cleaved CDCP1 antibody, e.g., described herein, or antigen-binding fragment thereof can be formulated with a long-acting IL-2. In some aspects, the anti-cleaved CDCP1 antibody can be formulated with Pegylated IL-2 molecules, such as NKTR-214 of Nektar; see U.S. Pat. No. 8,252,275, WO12/065086 and WO15/125159.

In some aspects, the composition of the invention further comprises a bulking agent. A bulking agent can be selected from the group consisting of NaCl, mannitol, glycine, alanine, and any combination thereof. In other aspects, the composition of the invention comprises a stabilizing agent. The stabilizing agent can be selected from the group consisting of sucrose, trehalose, raffinose, arginine; or any combination thereof. In other aspects, the composition of the invention comprises a surfactant. The surfactant can be selected from the group consisting of polysorbate 80 (PS80), polysorbate 20 (PS20), and any combination thereof. In certain aspects, the composition further comprises a chelating agent. The chelating agent can be selected from the group consisting of diethylenetriaminepentaacetic acid (DTPA), ethylenediaminetetraacetic acid, nitrilotriacetic acid, and any combination thereof.

In other aspects, the composition comprises a third antibody. In some aspects, the third antibody is any antibody disclosed herein.

In some aspects, the composition further comprises NaCl, mannitol, pentetic acid (DTPA), sucrose, PS80, and any combination thereof.

As used herein, “pharmaceutically acceptable carrier” includes any and all solvents, dispersion media, coatings, antibacterial and antifungal agents, isotonic and absorption delaying agents, and the like that are physiologically compatible. In some aspects, the carrier is suitable for intravenous, intramuscular, subcutaneous, parenteral, spinal or epidermal administration (e.g., by injection or infusion). An option for subcutaneous injection is based on Halozyme Therapeutics' ENHANZE® drug-delivery technology, involving a co-formulation of an Ab with recombinant human hyaluronidase enzyme (rHuPH20) that removes traditional limitations on the volume of biologics and drugs that can be delivered subcutaneously due to the extracellular matrix (U.S. Pat. No. 7,767,429). Depending on the route of administration, the active compound, i.e., antibody, immunoconjugate, or bispecific molecule, can be coated in a material to protect the compound from the action of acids and other natural conditions that can inactivate the compound.

The pharmaceutical compounds described herein can include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition described herein can also include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers that can be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions can also contain adjuvants such as preservatives, wetting agents, emulsifying agents and dispersing agents. Prevention of presence of microorganisms can be ensured both by sterilization procedures, supra, and by the inclusion of various antibacterial and antifungal agents, for example, paraben, chlorobutanol, phenol sorbic acid, and the like. It can also be desirable to include isotonic agents, such as sugars, sodium chloride, and the like into the compositions. In addition, prolonged absorption of the injectable pharmaceutical form can be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions described herein is contemplated. A pharmaceutical composition can comprise a preservative or can be devoid of a preservative. Supplementary active compounds can be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, the compositions can include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated herein. In the case of sterile powders for the preparation of sterile injectable solutions, some methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, from about 0.1 percent to about 70 percent, or from about 1 percent to about 30 percent of active ingredient in combination with a pharmaceutically acceptable carrier.

Dosage regimens are adjusted to provide the optimum desired response (e.g., a therapeutic response). For example, a single bolus can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms described herein are dictated by and directly dependent on (a) the unique characteristics of the active compound and the particular therapeutic effect to be achieved, and (b) the limitations inherent in the art of compounding such an active compound for the treatment of sensitivity in individuals.

For administration of an anti-cleaved CDCP1 antibody, e.g., described herein, the dosage ranges from about 0.0001 to 100 mg/kg. An anti-CDCP1 antibody can be administered at a flat dose (flat dose regimen). In some aspects, an anti-cleaved CDCP1 antibody can be administered at a fixed dose with another antibody. In some aspects, an anti-CDCP1 antibody is administered at a dose based on body weight.

In some methods, two or more monoclonal antibodies with different binding specificities are administered simultaneously, in which case the dosage of each antibody administered falls within the ranges indicated. Antibody is usually administered on multiple occasions. Intervals between single dosages can be, for example, weekly, monthly, every three months or yearly. Intervals can also be irregular as indicated by measuring blood levels of antibody to the target antigen in the patient. In some methods, dosage is adjusted to achieve a plasma antibody concentration of about 1-1000 μg/ml and in some methods about 25-300 μg/ml.

An anti-cleaved CDCP1 antibody described herein or antigen-binding fragment thereof can be administered with another antibody at the dosage regimen of the other antibody. For example, an anti-cleaved CDCP1 antibody can be administered with an anti-PD-1 antibody, such as nivolumab (OPDIVO®), every two weeks as an i.v. infusion over 60 minutes until disease progression or unacceptable toxicity occurs. An anti-cleaved CDCP1 antibody can be administered with pembrolizumab (KEYTRUDA®) every 3 weeks as an i.v. infusion over 30 minutes until disease progression or unacceptable toxicity occurs. An anti-cleaved CDCP1 antibody can be administered with atezolizumab (TECENTRIQ™) every 3 weeks as an i.v. infusion over 60 or 30 minutes until disease progression or unacceptable toxicity occurs.

An antibody can be administered as a sustained release formulation, in which case less frequent administration is required. Dosage and frequency vary depending on the half-life of the antibody in the patient. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and until the patient shows partial or complete amelioration of symptoms of disease. Thereafter, the patient can be administered a prophylactic regime.

A composition described herein can be administered via one or more routes of administration using one or more of a variety of methods known in the art. As will be appreciated by the skilled artisan, the route and/or mode of administration will vary depending upon the desired results. Routes of administration for the anti-cleaved CDCP1 antibodies described herein or antigen-binding fragments thereof can include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Alternatively, an antibody described herein could potentially be administered via a non-parenteral route, such as a topical, epidermal or mucosal route of administration, for example, intranasally, orally, vaginally, rectally, sublingually or topically.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

### XI. Uses and Methods

Certain aspects of the present disclosure are directed to method of treating a subject, comprising administering to the subject an anti-cleaved CDCP1 antibody disclosed herein, a bispecific antibody comprising an anti-cleaved CDCP1 antibody, a multispecific antibody comprising an anti-cleaved CDCP1 antibody, a polynucleotide encoding the anti-cleaved CDCP1 antibody, a vector comprising the polynucleotide, a host cell comprising the polynucleotide, an immunoconjugate comprising an anti-cleaved CDCP1 antibody, or any combination thereof.

Certain aspects of the present disclosure are directed to a method of treating a cancer in a subject in need thereof, comprising administering to the subject an effective dose of a composition disclosed herein (e.g., an antibody, polynucleotide, vector, host cell, immunoconjugate, or pharmaceutical composition). In other aspects, the present disclosure is directed to a method of inhibiting shedding of cleaved CDCP1 by a tumor cell in a subject in need thereof, comprising administering to the subject an effective dose of a composition disclosed herein. In other aspects, the present disclosure is directed to a method of reducing shed cleaved CDCP1 in the serum and/or retaining cleaved CDCP1 on the cell surface in a subject in need thereof, comprising administering to the subject an effective dose of a composition disclosed herein. In other aspects, the present disclosure is directed to a method of killing a tumor cell in a subject in need thereof, comprising administering to the subject an effective dose of a composition disclosed herein. In other aspects, the present disclosure is directed to a method of reducing the size of a tumor in a subject in need thereof, comprising administering to the subject an effective dose of a composition disclosed herein. In other aspects, the present disclosure is directed to reducing or inhibiting metastasis of a tumor in a subject in need thereof, comprising administering to the subject an effective dose of a composition disclosed herein. In some aspects, the subject is a human.

The compositions of the present disclosure can be administered using any pharmaceutically acceptable route. In some aspects, the composition (e.g., antibody, polynucleotide, vector, host cell, immunoconjugate, or pharmaceutical composition) is administered intravenously, intraperitoneally, intramuscularly, intraarterially, intrathecally, intralymphaticly, intralesionally, intracapsularly, intraorbitally, intracardiacly, intradermally, transtracheally, subcutaneously, subcuticularly, intraarticularly, subcapsularly, subarachnoidly, intraspinally, epidurally, intrasternally, topically, epidermally, mucosally, or any combination thereof. In some aspects, the composition is administered intravenously. In some aspects, the composition is administered subcutaneously.

In certain aspects, the method reduces the size of a cancer, e.g., the size of a tumor, in the subject. In some aspects, the size of the caner is reduced by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, or at least about 90%.

In some aspects, the method comprises administering an anti-cleaved CDCP1 antibody (or a polynucleotide, vector, host cell, or immunoconjugate) disclosed herein and a second therapy. In some aspects, the second therapy is administered prior to the anti-cleaved CDCP1 antibody. In some aspects, the second therapy is administered after the anti-cleaved CDCP1 antibody. In some aspects, the second therapy is administered concurrently with the anti-cleaved CDCP1 antibody. In certain aspects, the anti-cleaved CDCP1 antibody and the second therapy are administered separately. In other aspects, the anti-cleaved CDCP1 antibody and the second therapy are administered in a single formulation.

The second therapy can be any other therapy known in the art. In some aspects, the second therapy comprises an immunotherapy. In some aspects, the second therapy comprises a chemotherapy. In some aspects, the second therapy comprises a radiotherapy. In some aspects, the second therapy comprises a surgery. In some aspects, the second therapy comprises administering a second therapeutic agent.

In certain aspects, the second therapeutic agent comprises a second antibody. In some aspects, the second therapeutic agent comprises an effective amount of an antibody that specifically binds a protein selected from Inducible T cell Co-Stimulator (ICOS), CD137 (4-1BB), CD134 (OX40), NKG2A, CD27, Glucocorticoid-Induced TNFR-Related protein (GITR), and Herpes Virus Entry Mediator (HVEM), Programmed Death-1 (PD-1), Programmed Death Ligand-1 (PD-L1), CTLA-4, B and T Lymphocyte Attenuator (BTLA), T cell Immunoglobulin and Mucin domain-3 (TIM-3), Lymphocyte Activation Gene-3 (LAG-3), adenosine A2a receptor (A2aR), Killer cell Lectin-like Receptor G1 (KLRG-1), Natural Killer Cell Receptor 2B4 (CD244), CD160, T cell Immunoreceptor with Ig and ITIM domains (TIGIT), and the receptor for V-domain Ig Suppressor of T cell Activation (VISTA), NKG2a, KIR, TGFβ, IL-10, IL-8, B7-H4, Fas ligand, CXCR4, mesothelin, CEACAM-1, CD96, CD52, HER2, and any combination thereof.

### XI.A. Anti-PD-1 Antibodies

In some aspects, the second antibody can be an anti-PD-1 antibody. Anti-PD-1 antibodies that are known in the art can be used in the presently described compositions and methods. Various human monoclonal antibodies that bind specifically to PD-1 with high affinity have been disclosed in U.S. Pat. No. 8,008,449. Anti-PD-1 human antibodies disclosed in U.S. Pat. No. 8,008,449 have been demonstrated to exhibit one or more of the following characteristics: (a) bind to human PD-1 with a K_{D} of 1×10⁻⁷ M or less, as determined by surface plasmon resonance using a Biacore biosensor system; (b) do not substantially bind to human CD28, CTLA-4 or ICOS; (c) increase T-cell proliferation in a Mixed Lymphocyte Reaction (MLR) assay; (d) increase interferon-γ production in an MLR assay; (e) increase IL-2 secretion in an MLR assay; (f) bind to human PD-1 and cynomolgus monkey PD-1; (g) inhibit the binding of PD-L1 and/or PD-L2 to PD-1; (h) stimulate antigen-specific memory responses; (i) stimulate antibody responses; and (j) inhibit tumor cell growth in vivo. Anti-PD-1 antibodies usable in the present invention include monoclonal antibodies that bind specifically to human PD-1 and exhibit at least one, in some aspects, at least five, of the preceding characteristics.

Other anti-PD-1 monoclonal antibodies have been described in, for example, U.S. Pat. Nos. 6,808,710, 7,488,802, 8,168,757 and 8,354,509, US Publication No. 2016/0272708, and PCT Publication Nos. WO 2012/145493, WO 2008/156712, WO 2015/112900, WO 2012/145493, WO 2015/112800, WO 2014/206107, WO 2015/35606, WO 2015/085847, WO 2014/179664, WO 2017/020291, WO 2017/020858, WO 2016/197367, WO 2017/024515, WO 2017/025051, WO 2017/123557, WO 2016/106159, WO 2014/194302, WO 2017/040790, WO 2017/133540, WO 2017/132827, WO 2017/024465, WO 2017/025016, WO 2017/106061, WO 2017/19846, WO 2017/024465, WO 2017/025016, WO 2017/132825, and WO 2017/133540 each of which is incorporated by reference in its entirety.

In some aspects, the anti-PD-1 antibody is selected from the group consisting of nivolumab (also known as OPDIVO®, 5C4, BMS-936558, MDX-1106, and ONO-4538), pembrolizumab (Merck; also known as KEYTRUDA®, lambrolizumab, and MK-3475; see WO2008/156712), PDR001 (Novartis; see WO 2015/112900), MEDI-0680 (AstraZeneca; also known as AMP-514; see WO 2012/145493), cemiplimab (Regeneron; also known as REGN-2810; see WO 2015/112800), JS001 (TAIZHOU JUNSHI PHARMA; see Si-Yang Liu et al., J. Hematol. Oncol. 10:136 (2017)), BGB-A317 (Beigene; see WO 2015/35606 and US 2015/0079109), INCSHR1210 (Jiangsu Hengrui Medicine; also known as SHR-1210; see WO 2015/085847; Si-Yang Liu et al., J. Hematol. Oncol. 10:136 (2017)), TSR-042 (Tesaro Biopharmaceutical; also known as ANB011; see WO2014/179664), GLS-010 (Wuxi/Harbin Gloria Pharmaceuticals; also known as WBP3055; see Si-Yang Liu et al., J. Hematol. Oncol. 10:136 (2017)), AM-0001 (Armo), STI-1110 (Sorrento Therapeutics; see WO 2014/194302), AGEN2034 (Agenus; see WO 2017/040790), MGA012 (Macrogenics, see WO 2017/19846), and IBI308 (Innovent; see WO 2017/024465, WO 2017/025016, WO 2017/132825, and WO 2017/133540).

In some aspects, the anti-PD-1 antibody is nivolumab. Nivolumab is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., 2014 Cancer Immunol Res. 2 (9): 846-56).

In another embodiment, the anti-PD-1 antibody is pembrolizumab. Pembrolizumab is a humanized monoclonal IgG4 (S228P) antibody directed against human cell surface receptor PD-1 (programmed death-1 or programmed cell death-1). Pembrolizumab is described, for example, in U.S. Pat. Nos. 8,354,509 and 8,900,587.

Anti-PD-1 antibodies usable in the disclosed compositions and methods also include isolated antibodies that bind specifically to human PD-1 and cross-compete for binding to human PD-1 with any anti-PD-1 antibody disclosed herein, e.g., nivolumab (see, e.g., U.S. Pat. Nos. 8,008,449 and 8,779,105; WO 2013/173223). In some aspects, the anti-PD-1 antibody binds the same epitope as any of the anti-PD-1 antibodies described herein, e.g., nivolumab. The ability of antibodies to cross-compete for binding to an antigen indicates that these monoclonal antibodies bind to the same epitope region of the antigen and sterically hinder the binding of other cross-competing antibodies to that particular epitope region. These cross-competing antibodies are expected to have functional properties very similar those of the reference antibody, e.g., nivolumab, by virtue of their binding to the same epitope region of PD-1. Cross-competing antibodies can be readily identified based on their ability to cross-compete with nivolumab in standard PD-1 binding assays such as Biacore analysis, ELISA assays or flow cytometry (see, e.g., WO 2013/173223).

In certain aspects, the antibodies that cross-compete for binding to human PD-1 with, or bind to the same epitope region of human PD-1 antibody, nivolumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-PD-1 antibodies usable in the compositions and methods of the disclosed invention also include antigen-binding fragments of the above antibodies. It has been amply demonstrated that the antigen-binding function of an antibody can be performed by fragments of a full-length antibody.

Anti-PD-1 antibodies suitable for use in the disclosed compositions and methods are antibodies that bind to PD-1 with high specificity and affinity, block the binding of PD-L1 and or PD-L2, and inhibit the immunosuppressive effect of the PD-1 signaling pathway. In any of the compositions or methods disclosed herein, an anti-PD-1 “antibody” includes an antigen-binding fragment or fragment that binds to the PD-1 receptor and exhibits the functional properties similar to those of whole antibodies in inhibiting ligand binding and up-regulating the immune system. In certain aspects, the anti-PD-1 antibody or antigen-binding fragment thereof cross-competes with nivolumab for binding to human PD-1.

### XI.B. Anti-PD-L1 Antibodies

In some aspects, the second antibody can be an anti-PD-L1 antibody. Anti-PD-L1 antibodies that are known in the art can be used in the compositions and methods of the present disclosure. Examples of anti-PD-L1 antibodies useful in the compositions and methods of the present disclosure include the antibodies disclosed in U.S. Pat. No. 9,580,507. Anti-PD-L1 human monoclonal antibodies disclosed in U.S. Pat. No. 9,580,507 have been demonstrated to exhibit one or more of the following characteristics: (a) bind to human PD-L1 with a K_{D} of 1×10⁻⁷ M or less, as determined by surface plasmon resonance using a Biacore biosensor system; (b) increase T-cell proliferation in a Mixed Lymphocyte Reaction (MLR) assay; (c) increase interferon-γ production in an MLR assay; (d) increase IL-2 secretion in an MLR assay; (e) stimulate antibody responses; and (f) reverse the effect of T regulatory cells on T cell effector cells and/or dendritic cells. Anti-PD-L1 antibodies usable in the present invention include monoclonal antibodies that bind specifically to human PD-L1 and exhibit at least one, in some aspects, at least five, of the preceding characteristics.

In certain aspects, the anti-PD-L1 antibody is selected from the group consisting of BMS-936559 (also known as 12A4, MDX-1105; see, e.g., U.S. Pat. No. 7,943,743 and WO 2013/173223), atezolizumab (Roche; also known as TECENTRIQ®; MPDL3280A, RG7446; see U.S. Pat. No. 8,217,149; see, also, Herbst et al. (2013) J Clin Oncol 31 (suppl): 3000), durvalumab (AstraZeneca; also known as IMFINZI™, MEDI-4736; see WO 2011/066389), avelumab (Pfizer; also known as BAVENCIO®, MSB-0010718C; see WO 2013/079174), STI-1014 (Sorrento; see WO2013/181634), CX-072 (Cytomx; see WO2016/149201), KN035 (3D Med/Alphamab; see Zhang et al., Cell Discov. 7:3 (March 2017), LY3300054 (Eli Lilly Co.; see, e.g., WO 2017/034916), and CK-301 (Checkpoint Therapeutics; see Gorelik et al., AACR: Abstract 4606 (April 2016)).

In certain aspects, the PD-L1 antibody is atezolizumab (TECENTRIQ®). Atezolizumab is a fully humanized IgG1 monoclonal anti-PD-L1 antibody.

In certain aspects, the PD-L1 antibody is durvalumab (IMFINZI™). Durvalumab is a human IgG1 kappa monoclonal anti-PD-L1 antibody.

In certain aspects, the PD-L1 antibody is avelumab (BAVENCIO®). Avelumab is a human IgG1 lambda monoclonal anti-PD-L1 antibody.

In other aspects, the anti-PD-L1 monoclonal antibody is selected from the group consisting of 28-8, 28-1, 28-12, 29-8, 5H1, and any combination thereof.

In certain aspects, the antibodies that cross-compete for binding to human PD-L1 with, or bind to the same epitope region of human PD-L1 antibody as, atezolizumab, durvalumab, and/or avelumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-PD-L1 antibodies usable in the compositions and methods of the disclosed invention also include antigen-binding fragments of the above antibodies. It has been amply demonstrated that the antigen-binding function of an antibody can be performed by fragments of a full-length antibody.

Anti-PD-L1 antibodies suitable for use in the disclosed compositions and methods are antibodies that bind to PD-L1 with high specificity and affinity, block the binding of PD-1, and inhibit the immunosuppressive effect of the PD-1 signaling pathway. In any of the compositions or methods disclosed herein, an anti-PD-L1 “antibody” includes an antigen-binding fragment or fragment that binds to PD-L1 and exhibits the functional properties similar to those of whole antibodies in inhibiting receptor binding and up-regulating the immune system. In certain aspects, the anti-PD-L1 antibody or antigen-binding fragment thereof cross-competes with atezolizumab, durvalumab, and/or avelumab for binding to human PD-L1.

### XI.C. Anti-CTLA-4 Antibodies

In some aspects, the second antibody can be an anti-CTLA-4 antibody. Anti-CTLA-4 antibodies that are known in the art can be used in the compositions and methods of the present disclosure. Anti-CTLA-4 antibodies of the instant invention bind to human CTLA-4 so as to disrupt the interaction of CTLA-4 with a human B7 receptor. Because the interaction of CTLA-4 with B7 transduces a signal leading to inactivation of T-cells bearing the CTLA-4 receptor, disruption of the interaction effectively induces, enhances or prolongs the activation of such T cells, thereby inducing, enhancing or prolonging an immune response.

Human monoclonal antibodies that bind specifically to CTLA-4 with high affinity have been disclosed in U.S. Pat. Nos. 6,984,720. Other anti-CTLA-4 monoclonal antibodies have been described in, for example, U.S. Pat. Nos. 5,977,318, 6,051,227, 6,682,736, and 7,034,121 and International Publication Nos. WO 2012/122444, WO 2007/113648, WO 2016/196237, and WO 2000/037504, each of which is incorporated by reference herein in its entirety. The anti-CTLA-4 human monoclonal antibodies disclosed in U.S. Pat. No. 6,984,720 have been demonstrated to exhibit one or more of the following characteristics: (a) binds specifically to human CTLA-4 with a binding affinity reflected by an equilibrium association constant (Kₐ) of at least about 10⁷ M⁻¹, or about 10⁹ M⁻¹, or about 10¹⁰ M⁻¹ to 10¹¹ M⁻¹ or higher, as determined by Biacore analysis; (b) a kinetic association constant (kₐ) of at least about 10³, about 10⁴, or about 10⁵ m⁻¹ s⁻¹; (c) a kinetic disassociation constant (k_{d}) of at least about 10³, about 10⁴, or about 10⁵ m⁻¹ s⁻¹; and (d) inhibits the binding of CTLA-4 to B7-1 (CD80) and B7-2 (CD86). Anti-CTLA-4 antibodies useful for the present invention include monoclonal antibodies that bind specifically to human CTLA-4 and exhibit at least one, at least two ted from the group consisting of 28-8, 28-1, 28-12, 29-8, 5H1, and any combination thereof.

Anti-PD-L1 antibodies usable in the disclosed compositions and methods also include isolated antibodies that bind specifically to human PD-L1 and cross-compete for binding to human PD-L1 with any anti-PD-L1 antibody disclosed herein, e.g., atezolizumab, durvalumab, and/or avelumab. In some aspects, the anti-PD-L1 antibody binds the same epitope as any of the anti-PD-L1 antibodies described herein, e.g., atezolizumab, durvalumab, and/or avelumab. The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar those of the reference antibody, e.g., atezolizumab and/or avelumab, by virtue of their binding to the same epitope region of PD-L1. Cross-competing antibodies can be readily identified based on their ability to cross-compete with atezolizumab and/or avelumab in standard PD-L1 binding assays such as Biacore analysis, ELISA assays or flow cytometry (see, e.g., WO 2013/173223).

In certain aspects, the CTLA-4 antibody is selected from the group consisting of ipilimumab (also known as YERVOY®, MDX-010, 10D1; see U.S. Pat. No. 6,984,720), MK-1308 (Merck), AGEN-1884 (Agenus Inc.; see WO 2016/196237), and tremelimumab (AstraZeneca; also known as ticilimumab, CP-675,206; see WO 2000/037504 and Ribas, Update Cancer Ther. 2 (3): 133-39 (2007)). In particular aspects, the anti-CTLA-4 antibody is ipilimumab.

In particular aspects, the CTLA-4 antibody is ipilimumab for use in the compositions and methods disclosed herein. Ipilimumab is a fully human, IgG1 monoclonal antibody that blocks the binding of CTLA-4 to its B7 ligands, thereby stimulating T cell activation and improving overall survival (OS) in patients with advanced melanoma.

In particular aspects, the CTLA-4 antibody is tremelimumab.

In particular aspects, the CTLA-4 antibody is MK-1308.

In particular aspects, the CTLA-4 antibody is AGEN-1884.

In some aspects, the CTLA-4 antibody is nonfucosylated or hypofucosylated. In some aspects, the CTLA-4 antibody exhibits enhanced ADCC and/or ADCP activity. In some aspects, the CTLA-4 antibody is BMS-986218, as described in PCT/US18/19868.

Anti-CTLA-4 antibodies usable in the disclosed compositions and methods also include isolated antibodies that bind specifically to human CTLA-4 and cross-compete for binding to human CTLA-4 with any anti-CTLA-4 antibody disclosed herein, e.g., ipilimumab and/or tremelimumab. In some aspects, the anti-CTLA-4 antibody binds the same epitope as any of the anti-CTLA-4 antibodies described herein, e.g., ipilimumab and/or tremelimumab. The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar those of the reference antibody, e.g., ipilimumab and/or tremelimumab, by virtue of their binding to the same epitope region of CTLA-4. Cross-competing antibodies can be readily identified based on their ability to cross-compete with ipilimumab and/or tremelimumab in standard CTLA-4 binding assays such as Biacore analysis, ELISA assays or flow cytometry (see, e.g., WO 2013/173223).

In certain aspects, the antibodies that cross-compete for binding to human CTLA-4 with, or bind to the same epitope region of human CTLA-4 antibody as, ipilimumab and/or tremelimumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-CTLA-4 antibodies usable in the compositions and methods of the disclosed invention also include antigen-binding fragments of the above antibodies. It has been amply demonstrated that the antigen-binding function of an antibody can be performed by fragments of a full-length antibody.

Anti-CTLA-4 antibodies suitable for use in the disclosed methods or compositions are antibodies that bind to CTLA-4 with high specificity and affinity, block the activity of CTLA-4, and disrupt the interaction of CTLA-4 with a human B7 receptor. In any of the compositions or methods disclosed herein, an anti-CTLA-4 “antibody” includes an antigen-binding fragment or fragment that binds to CTLA-4 and exhibits the functional properties similar to those of whole antibodies in inhibiting the interaction of CTLA-4 with a human B7 receptor and up-regulating the immune system. In certain aspects, the anti-CTLA-4 antibody or antigen-binding fragment thereof cross-competes with ipilimumab and/or tremelimumab for binding to human CTLA-4.

### XI.D. Anti-LAG-3 Antibodies

In some aspects, the second antibody can be an anti-LAG-3 antibody. Anti-LAG-3 antibodies of the instant disclosure bind to human LAG-3. Antibodies that bind to LAG-3 have been disclosed in Int'l Publ. No. WO/2015/042246 and U.S. Publ. Nos. 2014/0093511 and 2011/0150892.

An exemplary LAG-3 antibody useful in the present disclosure is 25F7 (described in U.S. Publ. No. 2011/0150892). An additional exemplary LAG-3 antibody useful in the present disclosure is BMS-986016. In some aspects, an anti-LAG-3 antibody useful for the composition cross-competes with 25F7 or BMS-986016. In another embodiment, an anti-LAG-3 antibody useful for the composition binds to the same epitope as 25F7 or BMS-986016. In other aspects, an anti-LAG-3 antibody comprises six CDRs of 25F7 or BMS-986016.

### XI.E. Anti-CD137 Antibodies

In some aspects, the second antibody can be an anti-CD137 antibody. Anti-CD137 antibodies specifically bind to and activate CD137-expressing immune cells, stimulating an immune response, in particular a cytotoxic T cell response, against tumor cells. Antibodies that bind to CD137 have been disclosed in U.S. Publ. No. 2005/0095244 and U.S. Pat. Nos. 7,288,638, 6,887,673, 7,214,493, 6,303,121, 6,569,997, 6,905,685, 6,355,476, 6,362,325, 6,974,863, and 6,210,669.

In some aspects, the anti-CD137 antibody is urelumab (BMS-663513), described in U.S. Pat. No. 7,288,638 (20H4.9-IgG4 [10C7 or BMS-663513]). In some aspects, the anti-CD137 antibody is BMS-663031 (20H4.9-IgG1), described in U.S. Pat. No. 7,288,638. In some aspects, the anti-CD137 antibody is 4E9 or BMS-554271, described in U.S. Pat. No. 6,887,673. In some aspects, the anti-CD137 antibody is an antibody disclosed in U.S. Pat. Nos. 7,214,493; 6,303,121; 6,569,997; 6,905,685; or 6,355,476. In some aspects, the anti-CD137 antibody is 1D8 or BMS-469492; 3H3 or BMS-469497; or 3E1, described in U.S. Pat. No. 6,362,325. In some aspects, the anti-CD137 antibody is an antibody disclosed in issued U.S. Pat. No. 6,974,863 (such as 53A2). In some aspects, the anti-CD137 antibody is an antibody disclosed in issued U.S. Pat. No. 6,210,669 (such as 1D8, 3B8, or 3E1). In some aspects, the antibody is Pfizer's PF-05082566 (PF-2566). In other aspects, an anti-CD137 antibody useful for the invention cross-competes with the anti-CD137 antibodies disclosed herein. In some aspects, an anti-CD137 antibody binds to the same epitope as the anti-CD137 antibody disclosed herein. In other aspects, an anti-CD137 antibody useful in the disclosure comprises six CDRs of the anti-CD137 antibodies disclosed herein.

### XI.F. Anti-KIR Antibodies

In some aspects, the second antibody can be an anti-KIR3 antibody. Antibodies that bind specifically to KIR block the interaction between Killer-cell immunoglobulin-like receptors (KIR) on NK cells with their ligands. Blocking these receptors facilitates activation of NK cells and, potentially, destruction of tumor cells by the latter. Examples of anti-KIR antibodies have been disclosed in Int'l Publ. Nos. WO/2014/055648, WO 2005/003168, WO 2005/009465, WO 2006/072625, WO 2006/072626, WO 2007/042573, WO 2008/084106, WO 2010/065939, WO 2012/071411 and WO/2012/160448.

One anti-KIR antibody useful in the present disclosure is lirilumab (also referred to as BMS-986015, IPH2102, or the S241P variant of 1-7F9), first described in Int'l Publ. No. WO 2008/084106. An additional anti-KIR antibody useful in the present disclosure is 1-7F9 (also referred to as IPH2101), described in Int'l Publ. No. WO 2006/003179. In some aspects, an anti-KIR antibody for the present composition cross competes for binding to KIR with lirilumab or I-7F9. In another embodiment, an anti-KIR antibody binds to the same epitope as lirilumab or I-7F9. In other aspects, an anti-KIR antibody comprises six CDRs of lirilumab or I-7F9.

### XI.G. Anti-GITR Antibodies

In some aspects, the second antibody can be an anti-GITR antibody. Anti-GITR antibodies can be any anti-GITR antibody that binds specifically to human GITR target and activates the glucocorticoid-induced tumor necrosis factor receptor (GITR). GITR is a member of the TNF receptor superfamily that is expressed on the surface of multiple types of immune cells, including regulatory T cells, effector T cells, B cells, natural killer (NK) cells, and activated dendritic cells (“anti-GITR agonist antibodies”). Specifically, GITR activation increases the proliferation and function of effector T cells, as well as abrogating the suppression induced by activated T regulatory cells. In addition, GITR stimulation promotes anti-tumor immunity by increasing the activity of other immune cells such as NK cells, antigen presenting cells, and B cells. Examples of anti-GITR antibodies have been disclosed in Int'l Publ. Nos. WO/2015/031667, WO2015/184,099, WO2015/026,684, WO11/028683 and WO/2006/105021, U.S. Pat. Nos. 7,812,135 and 8,388,967 and U.S. Publ. Nos. 2009/0136494, 2014/0220002, 2013/0183321 and 2014/0348841.

In some aspects, an anti-GITR antibody useful in the present disclosure is TRX518 (described in, for example, Schaer et al. Curr Opin Immunol. (2012) April; 24 (2): 217-224, and WO/2006/105021). In another embodiment, the anti-GITR antibody is selected from MK4166, MK1248, and antibodies described in WO11/028683 and U.S. Pat. No. 8,709,424, and comprising, e.g., a VH chain comprising SEQ ID NO: 104 and a VL chain comprising SEQ ID NO: 105 (wherein the SEQ ID NOs are from WO11/028683 or U.S. Pat. No. 8,709,424). In certain aspects, an anti-GITR antibody is an anti-GITR antibody that is disclosed in WO2015/031667, e.g., an antibody comprising VH CDRs 1-3 comprising SEQ ID NOs: 31, 71 and 63 of WO2015/031667, respectively, and VL CDRs 1-3 comprising SEQ ID NOs: 5, 14 and 30 of WO2015/031667. In certain aspects, an anti-GITR antibody is an anti-GITR antibody that is disclosed in WO2015/184099, e.g., antibody Hum231 #1 or Hum23 1 #2, or the CDRs thereof, or a derivative thereof (e.g., pab 1967, pab 1975 or pab 1979). In certain aspects, an anti-GITR antibody is an anti-GITR antibody that is disclosed in JP2008278814, WO09/009116, WO2013/039954, US20140072566, US20140072565, US20140065152, or WO2015/026684, or is INBRX-110 (INHIBRx), LKZ-145 (Novartis), or MEDI-1873 (MedImmune). In certain aspects, an anti-GITR antibody is an anti-GITR antibody that is described in PCT/US2015/033991 (e.g., an antibody comprising the variable regions of 28F3, 18E10 or 19D3).

In certain aspects, the anti-GITR antibody cross-competes with an anti-GITR antibody described herein, e.g., TRX518, MK4166 or an antibody comprising a VH domain and a VL domain amino acid sequence described herein. In some aspects, the anti-GITR antibody binds the same epitope as that of an anti-GITR antibody described herein, e.g., TRX518, MK4166 or an antibody comprising a VH domain and a VL domain amino acid sequence described herein. In certain aspects, the anti-GITR antibody comprises the six CDRs of TRX518, MK4166 or those of an antibody comprising a VH domain and a VL domain amino acid sequence described herein.

### XI.H. Anti-TIM3 Antibodies

In some aspects, the second antibody can be an anti-TIM3 antibody. In some aspects, the anti-TIM3 antibody can be selected from the anti-TIM3 antibodies disclosed in Int'l Publ. Nos. WO2018013818, WO/2015/117002 (e.g., MGB453, Novartis), WO/2016/161270 (e.g., TSR-022, Tesaro/AnaptysBio), WO2011155607, WO2016/144803 (e.g., STI-600, Sorrento Therapeutics), WO2016/071448, WO17055399; WO17055404, WO17178493, WO18036561, WO18039020 (e.g., Ly-3221367, Eli Lilly), WO2017205721, WO17079112; WO17079115; WO17079116, WO11159877, WO13006490, WO2016068802 WO2016068803, WO2016/111947, WO/2017/031242.

### XI.I. Anti-OX40 Antibodies

In some aspects, the second antibody can be an anti-OX40 (also known as CD134, TNFRSF4, ACT35 and/or TXGP1L) antibody. In some aspects, the anti-OX40 antibody can be BMS-986178 (Bristol-Myers Squibb Company), described in Int'l Publ. No. WO20160196228. In some aspects, the anti-OX40 antibody can be selected from the anti-OX40 antibodies described in Int'l Publ. Nos. WO95012673, WO199942585, WO14148895, WO15153513, WO15153514, WO13038191, WO16057667, WO03106498, WO12027328, WO13028231, WO16200836, WO 17063162, WO17134292, WO 17096179, WO 17096281, and WO 17096182.

### XI.J. Anti-NKG2A Antibodies

In some aspects, the second antibody can be an anti-NKG2A antibody. NKG2A is a member of the C-type lectin receptor family that is expressed on natural killer (NK) cells and a subset of T lymphocytes. Specifically, NKG2A primarily expressed on tumor infiltrating innate immune effector NK cells, as well as on some CD8+ T cells. Its natural ligand human leukocyte antigen E (HLA-E) is expressed on solid and hematologic tumors. NKG2A is an inhibitory receptor that blinds HLA-E.

In some aspects, the anti-NKG2A antibody can be BMS-986315, a human monoclonal antibody that blocks the interaction of NKG2A to its ligand HLA-E, thus allowing activation of an anti-tumor immune response. In some aspects, the anti-NKG2A antibody can be a checkpoint inhibitor that activates T cells, NK cells, and/or tumor-infiltrating immune cells. In some aspects, the anti-NKG2A antibody can be selected from the anti-NKG2A antibodies described in, for example, WO 2006/070286 (Innate Pharma S.A.; University of Genova); U.S. Pat. No. 8,993,319 (Innate Pharma S.A.; University of Genova); WO 2007/042573 (Innate Pharma S/A; Novo Nordisk A/S; University of Genova); U.S. Pat. No. 9,447,185 (Innate Pharma S/A; Novo Nordisk A/S; University of Genova); WO 2008/009545 (Novo Nordisk A/S); U.S. Pat. Nos. 8,206,709; 8,901,283; 9,683,041 (Novo Nordisk A/S); WO 2009/092805 (Novo Nordisk A/S); U.S. Pat. Nos. 8,796,427 and 9,422,368 (Novo Nordisk A/S); WO 2016/134371 (Ohio State Innovation Foundation); WO 2016/032334 (Janssen); WO 2016/041947 (Innate); WO 2016/041945 (Academisch Ziekenhuis Leiden H.O.D.N. LUMC); WO 2016/041947 (Innate Pharma); and WO 2016/041945 (Innate Pharma).

### XI.K. Anti-ICOS Antibodies

In some aspects, the second antibody can be an anti-ICOS antibody. ICOS is an immune checkpoint protein that is a member of the CD28-superfamily. ICOS is a 55-60 kDa type I transmembrane protein that is expressed on T cells after T cell activation and co-stimulates T-cell activation after binding its ligand, ICOS-L (B7H2). ICOS is also known as inducible T-cell co-stimulator, CVID1, AILIM, inducible costimulator, CD278, activation-inducible lymphocyte immunomediatory molecule, and CD278 antigen.

In some aspects, the anti-ICOS antibody can be BMS-986226, a humanized IgG monoclonal antibody that binds to and stimulates human ICOS. In some aspects, the anti-ICOS antibody can be selected from anti-ICOS antibodies described in, for example, WO 2016/154177 (Jounce Therapeutics, Inc.), WO 2008/137915 (MedImmune), WO 2012/131004 (INSERM, French National Institute of Health and Medical Research), EP3147297 (INSERM, French National Institute of Health and Medical Research), WO 2011/041613 (Memorial Sloan Kettering Cancer Center), EP 2482849 (Memorial Sloan Kettering Cancer Center), WO 1999/15553 (Robert Koch Institute), U.S. Pat. Nos. 7,259,247 and 7,722,872 (Robert Kotch Institute); WO 1998/038216 (Japan Tobacco Inc.), U.S. Pat. Nos. 7,045,615; 7,112,655, and 8,389,690 (Japan Tobacco Inc.), U.S. Pat. Nos. 9,738,718 and 9,771,424 (GlaxoSmithKline), and WO 2017/220988 (Kymab Limited).

### XI.L. Anti-TIGIT Antibodies

In some aspects, the second antibody can be an anti-TIGIT antibody. In some aspects, the anti-TIGIT antibody can be BMS-986207. In some aspects, the anti-TIGIT antibody can be clone 22G2, as described in WO 2016/106302. In some aspects, the anti-TIGIT antibody can be MTIG7192A/RG6058/RO7092284, or clone 4.1D3, as described in WO 2017/053748. In some aspects, the anti-TIGIT antibody can be selected from the anti-TIGIT antibodies described in, for example, WO 2016/106302 (Bristol-Myers Squibb Company) and WO 2017/053748 (Genentech).

### XI.M. Additional Anti-Cancer Therapy

In some aspects, the anti-cleaved CDCP1 antibody described herein or antigen-binding fragment thereof can be used in combination with an additional anti-cancer therapy.

In some aspects, the additional anti-cancer therapy comprises a standard of care therapy.

In some aspects, the additional anti-cancer therapy comprises a checkpoint inhibitor.

In some aspects, the additional anti-cancer therapy comprises an antibody or an antigen binding portion thereof that specifically binds a protein selected from Inducible T cell Co-Stimulator (ICOS), CD137 (4-1BB), CD134 (OX40), NKG2A, CD27, CD96, Glucocorticoid-Induced TNFR-Related protein (GITR), and Herpes Virus Entry Mediator (HVEM), Programmed Death-1 (PD-1), Programmed Death Ligand-1 (PD-L1), CTLA-4, B and T Lymphocyte Attenuator (BTLA), T cell Immunoglobulin and Mucin domain-3 (TIM-3), Lymphocyte Activation Gene-3 (LAG-3), adenosine A2a receptor (A2aR), Killer cell Lectin-like Receptor G1 (KLRG-1), Natural Killer Cell Receptor 2B4 (CD244), CD160, T cell Immunoreceptor with Ig and ITIM domains (TIGIT), and the receptor for V-domain Ig Suppressor of T cell Activation (VISTA), KIR, TGFβ, IL-10, IL-8, B7-H4, Fas ligand, CXCR4, mesothelin, CEACAM-1, CD52, HER2, and any combination thereof.

In some aspects, the additional anti-cancer therapy comprises CAR-T cell therapy.

In some aspects, the anti-cleaved CDCP1 antibody described herein or antigen-binding fragment thereof can be used in combination with an anti-IL-10 antibody. In some aspects, the anti-cleaved CDCP1 antibody described herein or antigen-binding fragment thereof can be used in combination with a long-acting IL-10 molecule. In some aspects, the long-acting IL-10 molecule can be an IL-10-Fc fusion molecule. In some aspects, the long-acting IL-10 molecule can be a Pegylated IL-10, such as AM0010 (ARMO BioSciences).

In some aspects, the anti-cleaved CDCP1 antibody described herein or antigen-binding fragment thereof can be used in combination with an anti-IL-2 antibody. In some aspects, the anti-cleaved CDCP1 antibody can be used in combination with a long-acting IL-2 molecule. In some aspects, the long-acting IL-2 can be a Pegylated IL-2, such as NKTR-214 (Nektar; see U.S. Pat. No. 8,252,275, WO12/065086 and WO15/125159).

In some aspects, the anti-cleaved CDCP1 antibody described herein or antigen-binding fragment thereof can be used in combination with an anti-VISTA antibody.

In some aspects, the anti-cleaved CDCP1 antibody described herein or antigen-binding fragment thereof can be used in combination with an anti-CD96 antibody.

In some aspects, the anti-cleaved CDCP1 antibody can be used in combination with an anti-IL-8 antibody, e.g., with HuMax®-IL8.

In some aspects, the anti-cleaved CDCP1 antibody described herein or antigen-binding fragment thereof can be used in combination with an anti-TGFβ antibody.

In some other aspects, the anti-cleaved CDCP1 antibody described herein or antigen-binding fragment thereof can be used in combination with an anti-B7-H4 antibody. In certain aspects, the anti-B7-H4 antibody is an anti-B7-H4 disclosed in Int'l Publ. No. WO/2009/073533.

In certain aspects, the anti-cleaved CDCP1 antibody described herein or antigen-binding fragment thereof can be used in combination with an anti-Fas ligand antibody. In certain aspects, the anti-Fas ligand antibody is an anti-Fas ligand disclosed in Int'l Publ. No. WO/2009/073533.

In some aspects, the anti-cleaved CDCP1 antibody described herein or antigen-binding fragment thereof can be used in combination with an anti-CXCR4 antibody. In certain aspects, the anti-CXCR4 antibody is an anti-CXCR4 disclosed in U.S. Publ. No. 2014/0322208 (e.g., Ulocuplumab (BMS-936564)).

In some aspects, the anti-cleaved CDCP1 antibody described herein or antigen-binding fragment thereof can be used in combination with an anti-mesothelin antibody. In certain aspects, the anti-mesothelin antibody is an anti-mesothelin disclosed in U.S. Pat. No. 8,399,623.

In some aspects, the anti-cleaved CDCP1 antibody described herein or antigen-binding fragment thereof can be used in combination with an anti-HER2 antibody. In certain aspects, the anti-HER2 antibody is Herceptin (U.S. Pat. No. 5,821,337), trastuzumab, or ado-trastuzumab emtansine (Kadcyla, e.g., WO/2001/000244).

In aspects, the anti-cleaved CDCP1 antibody described herein or antigen-binding fragment thereof can be used in combination with an anti-CD27 antibody. In aspects, the anti-CD-27 antibody is Varlilumab (also known as “CDX-1127” and “1F5”), which is a human IgG1 antibody that is an agonist for human CD27, as disclosed in, for example, U.S. Pat. No. 9,169,325.

In some aspects, the anti-cleaved CDCP1 antibody described herein or antigen-binding fragment thereof can be used in combination with an anti-CD73 antibody. In certain aspects, the anti-CD73 antibody is CD73.4.IgG2C219S.IgG1.1f.

In certain aspects, the second therapy comprises administering a chemotherapeutic agent. In some aspects, the chemotherapeutic agent induces cleaved CDCP1 expression on tumor cells. In some aspects, the chemotherapeutic agent is selected from a proteasome inhibitor, an immunomodulatory drug (IMiD), a Bet inhibitor, and any combination thereof. In some aspects, the proteasome inhibitor is selected from bortezomib, ixazomib, carfilzomib, oprozomib and marizomib. In certain aspects, the proteasome inhibitor comprises bortezomib.

In some aspects, the second therapy comprises a radiotherapy. Any radiotherapy known in the art can be used as the second therapy.

In some aspects, the second therapy comprises administering an agent that activates innate immune cells. In some aspects, the agent that activates innate immune cells comprises an NLRP3 agonist. In some aspects, the NLRP3 agonist comprises monosodium urate monohydrate (MSU) and/or the vaccine adjuvant alum. In some aspects, the agent that activates innate immune cells is a toll like receptor 7 (TLR7) agonist. In some aspects, the TLR7 agonist comprises imiquimod (R837), GS-9620 (see Tsai et al., J. Virology doi: 10.1128/JVI.02166-16 (Feb. 8, 2017)), ORN R-2336 (Miltenyl Biotec), or any combination thereof.

In some aspects, the second therapy comprises administering an agent that enhances the survival of natural killer (NK) cells, CD8″ T cells, or both. In some aspects, the agent comprises IL-2. In certain aspects, the agent comprises pegylated IL-2.

In certain aspects, the second therapy comprises administering an agent selected from the group consisting of doxorubicin (ADRIAMYCIN®), cisplatin, carboplatin, bleomycin sulfate, carmustine, chlorambucil (LEUKERAN®), cyclophosphamide (CYTOXAN®; NEOSAR®), lenalidomide (REVLIMID®), bortezomib (VELCADE®), dexamethasone, mitoxantrone, etoposide, cytarabine, bendamustine (TREANDA®), rituximab (RITUXAN®), ifosfamide, vincristine (ONCOVIN®), fludarabine (FLUDARA®), thalidomide (THALOMID®), alemtuzumab (CAMPATH®), ofatumumab (ARZERRA®), everolimus (AFINITOR®, ZORTRESS®), carfilzomib (KYPROLIS™), and any combination thereof.

### XI.N. Cancer

Anti-CDCP1 antibodies described herein or antigen-binding fragments thereof can enhance the immune response to cancerous cells in a patient having cancer. Provided herein are methods for treating a subject having cancer, comprising administering to the subject an anti-cleaved CDCP1 antibody described herein or antigen-binding fragment thereof, such that the subject is treated, e.g., such that growth of cancerous tumors is inhibited or reduced and/or that the tumors regress and/or that prolonged survival is achieved. An anti-cleaved CDCP1 antibody described herein or antigen-binding fragment thereof can be used alone to inhibit the growth of cancerous tumors. Alternatively, an anti-cleaved CDCP1 antibody described herein or antigen-binding fragment thereof can be used in conjunction with another agent, e.g., another immunogenic agent, a standard cancer treatment, or another antibody, as described below.

Accordingly, provided herein are methods of treating cancer, e.g., by inhibiting growth of tumor cells, in a subject, comprising administering to the subject a therapeutically effective amount of an anti-cleaved CDCP1 antibody described herein, e.g., anti-human cleaved CDCP1 antibody (e.g., CL03 or CL07), or antigen-binding fragment thereof. The antibody can be a human anti-cleaved CDCP1 antibody (such as any of the human anti-human cleaved CDCP1 antibodies described herein or antigen-binding fragments thereof). Cancers whose growth can be inhibited using the antibodies of the disclosure include cancers typically responsive to immunotherapy and those that are not typically responsive to immunotherapy. Cancers that can be treated also include cleaved CDCP1 positive cancers. Cancers can be cancers with solid tumors or hematolotical malignancies (liquid tumors). Non-limiting examples of cancers for treatment include small-cell lung cancer (SCLC), non-small cell lung cancer (NSCLC), squamous NSCLC, nonsquamous NSCLC, glioma, gastrointestinal cancer, renal cancer, clear cell carcinoma, ovarian cancer, liver cancer, colorectal cancer, endometrial cancer, kidney cancer, renal cell carcinoma (RCC), prostate cancer, hormone refractory prostate adenocarcinoma, thyroid cancer, neuroblastoma, pancreatic cancer, glioblastoma (glioblastoma multiforme), cervical cancer, stomach cancer, bladder cancer, hepatoma (hepatocellular carcinoma), breast cancer, colon carcinoma, head and neck cancer (or carcinoma), head and neck squamous cell carcinoma (HNSCC), gastric cancer, germ cell tumor, pediatric sarcoma, sinonasal natural killer, melanoma, metastatic malignant melanoma, cutaneous or intraocular malignant melanoma, mesothelioma, bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, neoplasm of the central nervous system (CNS), primary CNS lymphoma, tumor angiogenesis, spinal axis tumor, brain cancer, brain stem glioma, pituitary adenoma, Kaposi's sarcoma, epidermoid cancer, squamous cell cancer, environmentally-induced cancers including those induced by asbestos, virus-related cancers or cancers of viral origin, human papilloma virus (HPV)-related or -originating tumors, acute leukemia (ALL), acute myelogenous leukemia (AML), chronic lymphocytic leukemia (CLL), and chronic myelogenous leukemia (CML), undifferentiated AML, myeloblastic leukemia, myeloblastic leukemia, promyelocytic leukemia, myelomonocytic leukemia, monocytic leukemia, erythroleukemia, megakaryoblastic leukemia, isolated granulocytic sarcoma, chloroma, Hodgkin's lymphoma (HL), non-Hodgkin's lymphoma (NHL), B-cell lymphoma, T-cell lymphoma, lymphoplasmacytoid lymphoma, monocytoid B-cell lymphoma, mucosa-associated lymphoid tissue (MALT) lymphoma, anaplastic large-cell lymphoma, adult T-cell lymphoma/leukemia, mantle cell lymphoma, angio immunoblastic T-cell lymphoma, angiocentric lymphoma, intestinal T-cell lymphoma, primary mediastinal B-cell lymphoma, precursor T-lymphoblastic lymphoma, T-lymphoblastic; peripheral T-cell lymphoma, lymphoblastic lymphoma, post-transplantation lymphoproliferative disorder, true histiocytic lymphoma, primary central nervous system lymphoma, primary effusion lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T-cell lymphoma (CTLC), lymphoplasmacytoid lymphoma (LPL) with Waldenstrom's macroglobulinemia; myeloma, IgG myeloma, light chain myeloma, nonsecretory myeloma, smoldering myeloma (indolent myeloma), solitary plasmocytoma, multiple myeloma, chronic lymphocytic leukemia (CLL), hairy cell lymphoma; and any combinations of said cancers.

The methods described herein can also be used for treatment of metastatic cancers, unresectable, refractory cancers (e.g., cancers refractory to previous immunotherapy, e.g., with a blocking CTLA-4 or PD-1 antibody), and/or recurrent cancers.

In some aspects, the subject has a cancer selected from non-small cell lung cancer (NSCLC), head and neck squamous cell carcinoma (HNSCC), melanoma, bladder cancer, pancreatic cancer, gastric cancer, colon cancer, renal cell carcinoma (RCC), small-cell lung cancer (SCLC), mesothelioma, hepatocellular carcinoma, prostate cancer, multiple myeloma, and combinations of said cancers.

In some aspects, the subject has a cancer selected from non-small cell lung cancer (NSCLC), head and neck squamous cell carcinoma (HNSCC), melanoma, bladder cancer, pancreatic cancer, gastric cancer, colon cancer, and combinations of said cancers.

In some aspects, an anti-cleaved CDCP1 antibody described herein or antigen-binding fragment thereof is administered to patients having a cancer that exhibited an inadequate response to, or progressed on, a prior treatment, e.g., a prior treatment with an immuno-oncology or immunotherapy drug, or patients having a cancer that is refractory or resistant, either intrinsically refractory or resistant (e.g., refractory to a PD-1 pathway antagonist), or a wherein the resistance or refractory state is acquired. For example, subjects who are not responsive or not sufficiently responsive to a first therapy or who see disease progression following treatment, e.g., anti-PD-1 treatment, can be treated by administration of an anti-cleaved CDCP1 antibody described herein or antigen-binding fragment thereof alone or in combination with another therapy (e.g., with an anti-PD-1 therapy).

In some aspects, an anti-cleaved CDCP1 antibody described herein or antigen-binding fragment thereof is administered to patients who have not previously received (i.e., been treated with) an immuno-oncology agent, e.g., a PD-1 pathway antagonist.

In some aspects, a method of treating cancer in a subject comprises first determining whether the subject is CDCP1 positive, e.g., has tumor cells that express cleaved CDCP1, and if the subject has a cleaved CDCP1 positive cancer, then administering to the subject an anti-cleaved CDCP1 antibody, e.g., described herein, or antigen-binding fragment thereof. A method of treating a subject having cancer with an anti-cleaved CDCP1 antibody described herein or antigen-binding fragment thereof can comprise administering to a subject who has cancer cells that express CDCP1, a therapeutically effective amount of a CDCP1 antibody. Also provided herein are methods for predicting whether a subject will respond to treatment with an anti-cleaved CDCP1 antibody, e.g., described herein, or antigen-binding fragment thereof wherein the methods comprise determining the level of cleaved CDCP1 in cancer cells of the patient, and if cancer cells of the subject are cleaved CDCP1 positive, then the subject is likely to respond to a treatment with an anti-cleaved CDCP1 antibody described herein or antigen-binding fragment thereof.

In some aspects, a method of treating cancer in a subject comprises first determining whether the subject is PD-L1 or PD-1 positive, e.g., has tumor cells or TILs that express PD-L1 or PD-1, and if the subject has PD-L1 or PD-1 positive cancer or TIL cells, then administering to the subject an anti-cleaved CDCP1 antibody (and optionally a PD-1 or PD-L1 antagonist), e.g., described herein, or antigen-binding fragment thereof. A method of treating a subject having cancer with an anti-cleaved CDCP1 antibody (and optionally a PD-1 or PD-L1 antagonist) can comprise administering to a subject who has cancer cells or TIL cells that express PD-L1 or PD-1, a therapeutically effective amount of an anti-cleaved CDCP1 antibody (and optionally a PD-1 or PD-L1 antagonist). Also provided herein are methods for predicting whether a subject will respond to treatment with an anti-cleaved CDCP1 antibody (and optionally a PD-1 or PD-L1 antagonist), wherein the methods comprise determining the level of PD-L1 or PD-1 in cancer or TIL cells of the patient, and if cancer or TIL cells of the subject are PD-L1 or PD-1 positive, then the subject is likely to respond to a treatment with an anti-cleaved CDCP1 antibody, e.g., described herein, or antigen binding fragment thereof (and optionally a PD-1 or PD-L1 antagonist).

An anti-cleaved CDCP1 antibody, e.g., described herein, or antigen-binding fragment thereof can be administered with a standard of care treatment. An anti-cleaved CDCP1 antibody, e.g., described herein, or antigen-binding fragment thereof can be administered as a maintenance therapy, e.g., a therapy that is intended to prevent the occurrence or recurrence of tumors.

An anti-cleaved CDCP1 antibody, e.g., described herein, or antigen-binding fragment thereof can be administered with another treatment, e.g., radiation, surgery, or chemotherapy. For example, anti-cleaved CDCP1 antibody adjunctive therapy can be administered when there is a risk that micrometastases can be present and/or in order to reduce the risk of a relapse.

An anti-cleaved CDCP1 antibody, e.g., described herein, or antigen-binding fragment thereof can be administered as a monotherapy, or as the only immuno stimulating therapy. Antibodies to CDCP1, e.g., the anti-cleaved CDCP1, e.g., described herein, or antigen-binding fragment thereof can also be combined with an immunogenic agent, such as cancerous cells, purified tumor antigens (including recombinant proteins, peptides, and carbohydrate molecules), cells, and cells transfected with genes encoding immune stimulating cytokines (He et al., (2004) J. Immunol. 173:4919-28). Non-limiting examples of tumor vaccines that can be used include peptides of melanoma antigens, such as peptides of gp100, MAGE antigens, Trp-2, MARTI and/or tyrosinase, or tumor cells transfected to express the cytokine GM-CSF (discussed further below).

An anti-cleaved CDCP1 antibody, e.g., an anti-human cleaved CDCP1 antibody described herein, or antigen-binding fragment thereof, can be combined with a vaccination protocol. Many experimental strategies for vaccination against tumors have been devised (see Rosenberg, S., 2000, Development of Cancer Vaccines, ASCO Educational Book Spring: 60-62; Logothetis, C, 2000, ASCO Educational Book Spring: 300-302; Khayat, D. 2000, ASCO Educational Book Spring: 414-428; Foon, K. 2000, ASCO Educational Book Spring: 730-738; see also Restifo, N. and Sznol, M., Cancer Vaccines, Ch. 61, pp. 3023-3043 in DeVita et al. (eds.), 1997, Cancer: Principles and Practice of Oncology, Fifth Edition). In one of these strategies, a vaccine is prepared using autologous or allogeneic tumor cells. These cellular vaccines have been shown to be most effective when the tumor cells are transduced to express GM-CSF. GM-CSF has been shown to be a potent activator of antigen presentation for tumor vaccination (Dranoff et al. (1993) Proc. Natl. Acad. Sci U.S.A. 90:3539-43).

The study of gene expression and large scale gene expression patterns in various tumors has led to the definition of so called tumor specific antigens (Rosenberg, S A (1999) Immunity 10:281-7). In many cases, these tumor specific antigens are differentiation antigens expressed in the tumors and in the cell from which the tumor arose, for example melanocyte antigens gp100, MAGE antigens, and Trp-2. More importantly, many of these antigens can be shown to be the targets of tumor specific T cells found in the host. Anti-cleaved CDCP1 antibody treatment can be used in conjunction with a collection of recombinant proteins and/or peptides expressed in a tumor in order to generate an immune response to these proteins. These proteins are normally viewed by the immune system as self-antigens and are therefore tolerant to them. The tumor antigen can include the protein telomerase, which is required for the synthesis of telomeres of chromosomes and which is expressed in more than 85% of human cancers and in only a limited number of somatic tissues (Kim et al. (1994) Science 266:2011-2013). Tumor antigen can also be “neo-antigens” expressed in cancer cells because of somatic mutations that alter protein sequence or create fusion proteins between two unrelated sequences (i.e., bcr-abl in the Philadelphia chromosome), or idiotype from B cell tumors.

Other tumor vaccines can include the proteins from viruses implicated in human cancers such a Human Papilloma Viruses (HPV), Hepatitis Viruses (HBV and HCV) and Kaposi's Herpes Sarcoma Virus (KHSV). Another form of tumor specific antigen, which can be used in conjunction with administration of an anti-cleaved CDCP1 antibody, e.g., described herein, or antigen-binding fragment thereof, is purified heat shock proteins (HSP) isolated from the tumor tissue itself. These heat shock proteins contain fragments of proteins from the tumor cells and these HSPs are highly efficient at delivery to antigen presenting cells for eliciting tumor immunity (Suot & Srivastava (1995) Science 269:1585-1588; Tamura et al. (1997) Science 278:117-120).

Dendritic cells (DC) are potent antigen presenting cells that can be used to prime antigen-specific responses. DCs can be produced ex vivo and loaded with various protein and peptide antigens as well as tumor cell extracts (Nestle et al. (1998) Nature Medicine 4:328-332). DCs can also be transduced by genetic means to express these tumor antigens as well. DCs have also been fused directly to tumor cells for the purposes of immunization (Kugler et al. (2000) Nature Medicine 6:332-336). As a method of vaccination, DC immunization can be effectively combined with administration of an anti-cleaved CDCP1 antibody, e.g., described herein, or antigen-binding fragment thereof to activate more potent anti-tumor responses.

Administration of an anti-cleaved CDCP1 antibody, e.g., described herein, or antigen-binding fragment thereof can also be combined with standard cancer treatments (e.g., surgery, radiation, and chemotherapy). Administration of an anti-cleaved CDCP1 antibody, e.g., described herein, or antigen-binding fragment thereof can be effectively combined with chemotherapeutic regimes. In these instances, it can be possible to reduce the dose of chemotherapeutic reagent administered (Mokyr et al. (1998) Cancer Research 58:5301-5304). An example of such a combination is an anti-cleaved CDCP1 antibody, e.g., described herein, or antigen-binding fragment thereof in combination with decarbazine for the treatment of melanoma. Another example of such a combination is an anti-cleaved CDCP1 antibody, e.g., described herein, or antigen-binding fragment thereof in combination with interleukin-2 (IL-2), e.g. pegylated IL-2, for the treatment of melanoma. The scientific rationale behind the combined use of an anti-cleaved CDCP1 antibody and chemotherapy is that cell death, that is a consequence of the cytotoxic action of most chemotherapeutic compounds, should result in increased levels of tumor antigen in the antigen presentation pathway. Other combination therapies that can result in synergy with administration of an anti-cleaved CDCP1 antibody through cell death are radiation, surgery, and hormone deprivation. Each of these protocols creates a source of tumor antigen in the host. Angiogenesis inhibitors can also be combined with administration of an anti-cleaved CDCP1 antibody. Inhibition of angiogenesis leads to tumor cell death which can feed tumor antigen into host antigen presentation pathways.

The anti-cleaved CDCP1 antibodies described herein, or antigen-binding fragments thereof can also be used in combination with bispecific antibodies that target Fcα or Fcγ receptor-expressing effectors cells to tumor cells (see, e.g, U.S. Pat. Nos. 5,922,845 and 5,837,243). Bispecific antibodies can be used to target two separate antigens. For example, anti-Fc receptor/anti-tumor antigen (e.g., Her-2/neu) bispecific antibodies have been used to target macrophages to sites of tumor. This targeting can more effectively activate tumor specific responses. The T cell arm of these responses would be augmented by the action of the anti-cleaved CDCP1 antibody, e.g., described herein, or antigen-binding fragment thereof. Alternatively, antigen can be delivered directly to DCs by the use of bispecific antibodies which bind to tumor antigen and a dendritic cell specific cell surface marker.

Tumors evade host immune surveillance by a large variety of mechanisms. Many of these mechanisms can be overcome by the inactivation of proteins which are expressed by the tumors and which are immunosuppressive. These include among others TGF-β (Kehrl et al. (1986) J. Exp. Med. 163:1037-1050), IL-10 (Howard & O'Garra (1992) Immunology Today 13:198-200), and Fas ligand (Hahne et al. (1996) Science 274:1363-1365). Antibodies to each of these entities can be used in combination with anti-cleaved CDCP1 antibodies to counteract the effects of the immunosuppressive agent and favor tumor immune responses by the host.

Other antibodies which activate host immune responsiveness can be used in combination with anti-cleaved CDCP1 antibodies. These include molecules on the surface of dendritic cells which activate DC function and antigen presentation. Anti-CD40 antibodies are able to substitute effectively for T cell helper activity (Ridge et al. (1998) Nature 393:474-478) and can be used in conjunction with anti-cleaved CDCP1 antibodies. Activating antibodies to T cell costimulatory molecules such as CTLA-4 (e.g., U.S. Pat. No. 5,811,097), OX-40 (Weinberg et al. (2000) Immunol 164:2160-2169), 4-1BB (Melero et al. (1997) Nature Medicine 3:682-685 (1997), and ICOS (Hutloff et al. (1999) Nature 397:262-266) can also provide for increased levels of T cell activation. Inhibitors of PD1 or PD-L1 can also be used in conjunction with an anti-cleaved CDCP1 antibody. Other combinations are provided elsewhere herein.

Bone marrow transplantation is currently being used to treat a variety of tumors of hematopoietic origin. While graft versus host disease is a consequence of this treatment, therapeutic benefit can be obtained from graft vs. tumor responses. A cleaved CDCP1 inhibition can be used to increase the effectiveness of the donor engrafted tumor specific T cells.

There are also several experimental treatment protocols that involve ex vivo activation and expansion of antigen specific T cells and adoptive transfer of these cells into recipients in order to stimulate antigen-specific T cells against tumor (Greenberg & Riddell (1999) Science 285:546-51). These methods can also be used to activate T cell responses to infectious agents such as CMV. Ex vivo activation in the presence of anti-cleaved CDCP1 antibodies can increase the frequency and activity of the adoptively transferred T cells.

### XI.O Combination Therapies

In addition to the combinations therapies provided above, anti-cleaved CDCP1 antibodies, e.g., those described herein, or antigen-binding fragments thereof can also be used in combination therapy, e.g., for treating cancer, as described below.

Provided herein are methods of combination therapy in which an anti-cleaved CDCP1 antibody, e.g., described herein, or antigen-binding fragment thereof is coadministered with one or more additional agents, e.g., small molecule drugs, antibodies or antigen binding fragments thereof, that are effective in stimulating immune responses to thereby further enhance, stimulate or upregulate immune responses in a subject.

Generally, an anti-cleaved CDCP1 antibody, e.g., described herein, or antigen-binding fragment thereof can be combined with (i) an agonist of a stimulatory (e.g., co-stimulatory) molecule (e.g., receptor or ligand) and/or (ii) an antagonist of an inhibitory signal or molecule (e.g., receptor or ligand) on immune cells, such as T cells, both of which result in amplifying immune responses, such as antigen-specific T cell responses. In some aspects, an immuno-oncology agent is (i) an agonist of a stimulatory (including a co-stimulatory) molecule (e.g., receptor or ligand) or (ii) an antagonist of an inhibitory (including a co-inhibitory) molecule (e.g., receptor or ligand) on cells, e.g., those inhibiting T cell activation or those involved in innate immunity, e.g., NK cells, and wherein the immuno-oncology agent enhances innate immunity. Such immuno-oncology agents are often referred to as immune checkpoint regulators, e.g., immune checkpoint inhibitor or immune checkpoint stimulator.

In some aspects, an anti-cleaved CDCP1 antibody, e.g., described herein, or antigen-binding fragment thereof is administered with an agent that targets a stimulatory or inhibitory molecule that is a member of the immunoglobulin super family (IgSF). For example, anti-cleaved CDCP1 antibodies, e.g., described herein, or antigen-binding fragments thereof can be administered to a subject with an agent that targets a member of the IgSF family to increase an immune response. For example, an anti-cleaved CDCP1 antibody, e.g., described herein, or antigen-binding fragment thereof can be administered with an agent that targets (or binds specifically to) a member of the B7 family of membrane-bound ligands that includes B7-1, B7-2, B7-H1 (PD-L1), B7-DC (PD-L2), B7-H2 (ICOS-L), B7-H3, B7-H4, B7-H5 (VISTA), and B7-H6 or a co-stimulatory or co-inhibitory receptor or ligand binding specifically to a B7 family member.

An anti-cleaved CDCP1 antibody, e.g., described herein, or antigen-binding fragment thereof can also be administered with an agent that targets a member of the TNF and TNFR family of molecules (ligands or receptors), such as CD40 and CD40L, OX-40, OX-40L, CD70, CD27L, CD30, CD30L, 4-1BBL, CD137, TRAIL/Apo2-L, TRAILR1/DR4, TRAILR2/DR5, TRAILR3, TRAILR4, OPG, RANK, RANKL, TWEAKR/Fn 14, TWEAK, BAFFR, EDAR, XEDAR, TACI, APRIL, BCMA, LTpR, LIGHT, DcR3, HVEM, VEGI/TL1A, TRAMP/DR3, EDA1, EDA2, TNFR1, Lymphotoxin a/TNFp, TNFR2, TNFa, LTpR, Lymphotoxin a 182, FAS, FASL, RELT, DR6, TROY, and NGFR (see, e.g., Tansey (2009) Drug Discovery Today 00:1).

T cell responses can be stimulated by a combination of anti-cleaved CDCP1 antibodies, e.g., described herein, or antigen-binding fragments thereof and one or more of the following agents:
(1) An antagonist (inhibitor or blocking agent) of a protein that inhibits T cell activation (e.g., immune checkpoint inhibitors), such as CTLA-4, PD-1, PD-L1, PD-L2, GITR, and LAG-3, Galectin 9, CEACAM-1, BTLA, CD69, Galectin-1, TIGIT, CD113, GPR56, VISTA, B7-H3, B7-H4, 2B4, CD48, GARP, PD1H, LAIR1, TIM-1, TIM-3, and TIM-4; and/or
(2) An agonist of a protein that stimulates T cell activation, such as B7-1, B7-2, CD28, 4-1BB (CD137), 4-1BBL, GITR, ICOS, ICOS-L, OX40, OX40L, CD70, CD27, CD40, DR3 and CD28H.

Exemplary agents that modulate one of the above proteins and can be combined with anti-cleaved CDCP1 antibodies, e.g., those described herein, or antigen-binding fragments thereof for treating cancer, include: YERVOY® (ipilimumab) or Tremelimumab (to CTLA-4), galiximab (to B7.1), BMS-936558 (to PD-1), MK-3475 (to PD-1), atezolizumab (TECENTRIQ®), AMP224 (to B7DC), BMS-936559 (to B7-H1), MPDL3280A (to B7-H1), MEDI-570 (to ICOS), AMG557 (to B7H2), MGA271 (to B7H3), IMP321 (to LAG-3), BMS-663513 (to CD137), PF-05082566 (to CD137), CDX-1127 (to CD27), anti-OX40 (Providence Health Services), huMAbOX40L (to OX40L), Atacicept (to TACI), CP-870893 (to CD40), Lucatumumab (to CD40), Dacetuzumab (to CD40), Muromonab-CD3 (to CD3); anti-GITR antibodies MK4166, TRX518, Medi1873, INBRX-110, LK2-145, GWN-323, GITRL-Fc, or any combination thereof.

Other molecules that can be combined with anti-cleaved CDCP1 antibodies, e.g., described herein, or antigen-binding fragments thereof for the treatment of cancer include antagonists of inhibitory receptors on NK cells or agonists of activating receptors on NK cells. For example, anti-CDCP1 antibodies can be combined with antagonists of KIR (e.g., lirilumab).

T cell activation is also regulated by soluble cytokines, and anti-cleaved CDCP1 antibodies, e.g., described herein, or antigen-binding fragments thereof can be administered to a subject, e.g., having cancer, with antagonists of cytokines that inhibit T cell activation or agonists of cytokines that stimulate T cell activation.

In some aspects, anti-cleaved CDCP1 antibodies, e.g., described herein, or antigen-binding fragments thereof can be used in combination with (i) antagonists (or inhibitors or blocking agents) of proteins of the IgSF family or B7 family or the TNF family that inhibit T cell activation or antagonists of cytokines that inhibit T cell activation (e.g., IL-6, IL-10, TGF-β, VEGF; “immunosuppressive cytokines”) and/or (ii) agonists of stimulatory receptors of the IgSF family, B7 family or the TNF family or of cytokines that stimulate T cell activation, for stimulating an immune response, e.g., for treating proliferative diseases, such as cancer.

Yet other agents for combination therapies include agents that inhibit or deplete macrophages or monocytes, including but not limited to CSF-1R antagonists such as CSF-1R antagonist antibodies including RG7155 (WO11/70024, WO11/107553, WO11/131407, WO13/87699, WO13/119716, WO13/132044) or FPA-008 (WO11/140249; WO13169264; WO14/036357).

Anti-CDCP1 antibodies, e.g., described herein, or antigen-binding fragments thereof can also be administered with agents that inhibit TGF-β signaling.

Additional agents that can be combined with an anti-cleaved CDCP1 antibody, e.g., described herein, or antigen-binding fragment thereof include agents that enhance tumor antigen presentation, e.g., dendritic cell vaccines, GM-CSF secreting cellular vaccines, CpG oligonucleotides, and imiquimod, or therapies that enhance the immunogenicity of tumor cells (e.g., anthracyclines).

Yet other therapies that can be combined with an anti-cleaved CDCP1 antibody, e.g., described herein, or antigen-binding fragment thereof include therapies that deplete or block Treg cells, e.g., an agent that specifically binds to CD25.

Another therapy that can be combined with an anti-cleaved CDCP1 antibody, e.g., described herein, or antigen-binding fragment thereof is a therapy that inhibits a metabolic enzyme such as indoleamine dioxigenase (IDO), dioxigenase, arginase, or nitric oxide synthetase. Suitable IDO antagonists include, for example, INCB-024360 (WO2006/122150, WO07/75598, WO08/36653, WO08/36642), indoximod, NLG-919 (WO09/73620, WO09/1156652, WO11/56652, WO12/142237) or F001287.

Another class of agents that can be used with an anti-cleaved CDCP1 antibody, e.g., described herein, or antigen-binding fragment thereof includes agents that inhibit the formation of adenosine, e.g., CD73 inhibitors, or inhibit the adenosine A2A receptor.

Other therapies that can be combined with an anti-cleaved CDCP1 antibody, e.g., described herein, or antigen-binding fragment thereof for treating cancer include therapies that reverse/prevent T cell anergy or exhaustion and therapies that trigger an innate immune activation and/or inflammation at a tumor site.

An anti-cleaved CDCP1 antibody, e.g., described herein, or antigen-binding fragment thereof can be combined with more than one immuno-oncology agent, and can be, e.g., combined with a combinatorial approach that targets multiple elements of the immune pathway, such as one or more of the following: a therapy that enhances tumor antigen presentation (e.g., dendritic cell vaccine, GM-CSF secreting cellular vaccines, CpG oligonucleotides, imiquimod); a therapy that inhibits negative immune regulation e.g., by inhibiting CTLA-4 and/or PD1/PD-L1/PD-L2 pathway and/or depleting or blocking Tregs or other immune suppressing cells; a therapy that stimulates positive immune regulation, e.g., with agonists that stimulate the CD-137, OX-40, and/or CD40 or GITR pathway and/or stimulate T cell effector function; a therapy that increases systemically the frequency of anti-tumor T cells; a therapy that depletes or inhibits Tregs, such as Tregs in the tumor, e.g., using an antagonist of CD25 (e.g., daclizumab) or by ex vivo anti-CD25 bead depletion; a therapy that impacts the function of suppressor myeloid cells in the tumor; a therapy that enhances immunogenicity of tumor cells (e.g., anthracyclines); adoptive T cell or NK cell transfer including genetically modified cells, e.g., cells modified by chimeric antigen receptors (CAR-T therapy); a therapy that inhibits a metabolic enzyme such as indoleamine dioxigenase (IDO), dioxigenase, arginase, or nitric oxide synthetase; a therapy that reverses/prevents T cell anergy or exhaustion; a therapy that triggers an innate immune activation and/or inflammation at a tumor site; administration of immune stimulatory cytokines; or blocking of immuno repressive cytokines.

Anti-cleaved CDCP1 antibodies described herein, e.g., described herein, or antigen-binding fragments thereof can be used together with one or more of agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell anergy or exhaustion) and agents that trigger innate immune activation and/or inflammation at tumor sites.

In some aspects, an anti-cleaved CDCP1 antibody, e.g., described herein, or antigen-binding fragment thereof is administered to a subject together with a BRAF inhibitor if the subject is BRAF V600 mutation positive.

In some aspects, the anti-cleaved CDCP1 antibody, e.g., described herein, or antigen-binding fragment thereof is administered to a subject together with an antibody that specifically binds PD-1, PD-L1, CTLA-4, LAG3, TIGIT, TIM3, NKG2a, OX40, ICOS, CD137, KIR, TGFβ, IL-10, IL-8, IL-2, B7-H4, Fas ligand, CXCR4, mesothelin, CD27, VISTA, CD96, GITR or any combination thereof.

The anti-cleaved CDCP1 antibodies, e.g., described herein, or antigen-binding fragments thereof and combination therapies described herein can also be used in conjunction with other well-known therapies that are selected for their particular usefulness against the indication being treated (e.g., cancer). Combinations of the anti-cleaved CDCP1 antibodies described herein, or antigen-binding fragments thereof can be used sequentially with known pharmaceutically acceptable agent(s).

For example, the anti-cleaved CDCP1 antibodies, e.g., described herein, or antigen-binding fragments thereof and combination therapies described herein can be used in combination (e.g., simultaneously or separately) with an additional treatment, such as irradiation and/or chemotherapy, e.g., using camptothecin (CPT-11), 5-fluorouracil (5-FU), cisplatin, doxorubicin, irinotecan, paclitaxel, gemcitabine, cisplatin, paclitaxel, carboplatin-paclitaxel (Taxol), doxorubicin, or camptothecin+apo21/TRAIL (a 6× combo)), one or more proteasome inhibitors (e.g., bortezomib or MG132), one or more Bcl-2 inhibitors (e.g., BH31-2′ (bcl-xl inhibitor), indoleamine dioxygenase-1 inhibitor (e.g., INCB24360, indoximod, NLG-919, or F001287), AT-101 (R-(−)-gossypol derivative), ABT-263 (small molecule), GX-15-070 (obatoclax), or MCL-1 (myeloid leukemia cell differentiation protein-1) antagonists), iAP (inhibitor of apoptosis protein) antagonists (e.g., smac7, smac4, small molecule smac mimetic, synthetic smac peptides (see Fulda et al., Nat Med 2002; 8:808-15), ISIS23722 (LY2181308), or AEG-35156 (GEM-640)), HDAC (histone deacetylase) inhibitors, anti-CD20 antibodies (e.g., rituximab), angiogenesis inhibitors (e.g., bevacizumab), anti-angiogenic agents targeting VEGF and VEGFR (e.g., Avastin), synthetic triterpenoids (see Hyer et al, Cancer Research 2005; 65:4799-808), c-FLIP (cellular FLICE-inhibitory protein) modulators (e.g., natural and synthetic ligands of PPARy (peroxisome proliferator-activated receptor Y), 5809354 or 5569100), kinase inhibitors (e.g., Sorafenib), Trastuzumab, Cetuximab, Temsirolimus, mTOR inhibitors such as rapamycin and temsirolimus, Bortezomib, JAK2 inhibitors, HSP90 inhibitors, PI3K-AKT inhibitors, Lenalildomide, GSK3P inhibitors, IAP inhibitors and/or genotoxic drugs.

The anti-cleaved CDCP1 antibodies, e.g., described herein, or antigen-binding fragments thereof and combination therapies described herein can further be used in combination with one or more anti-proliferative cytotoxic agents. Classes of compounds that can be used as anti-proliferative cytotoxic agents include, but are not limited to, the following:

Alkylating agents (including, without limitation, nitrogen mustards, ethylenimine derivatives, alkyl sulfonates, nitrosoureas and triazenes): Uracil mustard, Chlormethine, Cyclophosphamide (CYTOXAN®) fosfamide, Melphalan, Chlorambucil, Pipobroman, Triethylenemelamine, Triethylenethiophosphoramine, Busulfan, Carmustine, Lomustine, Streptozocin, Dacarbazine, and Temozolomide.

Antimetabolites (including, without limitation, folic acid antagonists, pyrimidine analogs, purine analogs and adenosine deaminase inhibitors): Methotrexate, 5-Fluorouracil, Floxuridine, Cytarabine, 6-Mercaptopurine, 6-Thioguanine, Fludarabine phosphate, Pentostatine, and Gemcitabine.

Suitable anti-proliferative agents for combining with anti-cleaved CDCP1 antibodies, e.g., described herein, or antigen-binding fragments thereof without limitation, taxanes, paclitaxel (paclitaxel is commercially available as TAXOL™), docetaxel, discodermolide (DDM), dictyostatin (DCT), Peloruside A, epothilones, epothilone A, epothilone B, epothilone C, epothilone D, epothilone E, epothilone F, furanoepothilone D, desoxyepothilone BI, [17]-dehydrodesoxyepothilone B, [18]dehydrodesoxyepothilones B, C12, 13-cyclopropyl-epothilone A, C6-C8 bridged epothilone A, trans-9,10-dehydroepothilone D, cis-9, 10-dehydroepothilone D, 16-desmethylepothilone B, epothilone BIO, discoderomolide, patupilone (EPO-906), KOS-862, KOS-1584, ZK-EPO, ABJ-789, XAA296A (Discodermolide), TZT-1027 (soblidotin), ILX-651 (tasidotin hydrochloride), Halichondrin B, Eribulin mesylate (E-7389), Hemiasterlin (HTI-286), E-7974, Cyrptophycins, LY-355703, Cantansinoid immunoconjugates (DM-1), MKC-1, ABT-751, Tl-38067, T-900607, SB-715992 (ispinesib), SB-743921, MK-0731, STA-5312, eleutherobin, 17beta-acetoxy-2-ethoxy-6-oxo-B-homo-estra-1,3,5 (10)-trien-3-ol, cyclostreptin, isolaulimalide, laulimalide, 4-epi-7-dehydroxy-14, 16-didemethyl-(+)-discodermolides, and cryptothilone 1, in addition to other microtubuline stabilizing agents known in the art.

In cases where it is desirable to render aberrantly proliferative cells quiescent in conjunction with or prior to treatment with anti-cleaved CDCP1 antibodies described herein, or antigen-binding fragments thereof hormones and steroids (including synthetic analogs), such as 17a-Ethinylestradiol, Diethylstilbestrol, Testosterone, Prednisone, Fluoxymesterone, Dromostanolone propionate, Testolactone, Megestrolacetate, Methylprednisolone, Methyl-testosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, ZOLADEXR, can also be administered to the patient. When employing the methods or compositions described herein, other agents used in the modulation of tumor growth or metastasis in a clinical setting, such as antimimetics, can also be administered as desired.

In some aspects, the combination of the anti-cleaved CDCP1 antibody, e.g., described herein, or antigen-binding fragment thereof and a second agent discussed herein can be administered concurrently as a single composition in a pharmaceutically acceptable carrier, or concurrently as separate compositions with the anti-cleaved CDCP1 antibody, e.g., described herein, or antigen-binding fragment thereof and the second agent in a pharmaceutically acceptable carrier. In some aspects, the combination of the anti-cleaved CDCP1 antibody, e.g., described herein, or antigen-binding fragment thereof and the second agent can be administered sequentially. The administration of the two agents can start at times that are, e.g., 30 minutes, 60 minutes, 90 minutes, 120 minutes, 3 hours, 6 hours, 12 hours, 24 hours, 36 hours, 48 hours, 3 days, 5 days, 7 days, or one or more weeks apart, or administration of the second agent can start, e.g., 30 minutes, 60 minutes, 90 minutes, 120 minutes, 3 hours, 6 hours, 12 hours, 24 hours, 36 hours, 48 hours, 3 days, 5 days, 7 days, or one or more weeks after the first agent has been administered.

In some aspects, an anti-neoplastic antibody that can be combined with an anti-cleaved CDCP1 antibody, e.g., described herein, or antigen-binding fragment thereof and/or a second agent includes RITUXAN® (rituximab), HERCEPTIN® (trastuzumab), BEXXAR® (tositumomab), ZEVALIN® (ibritumomab), CAMPATH® (alemtuzumab), LYMPHOCIDE® (eprtuzumab), AVASTIN® (bevacizumab), and TARCEVA® (erlotinib), or any combination thereof. In some aspects, the second antibody useful for the combination therapy with an anti-cleaved CDCP1 antibody, e.g., described herein, or antigen-binding fragment thereof can be an antibody drug conjugate.

In some aspects, an anti-cleaved CDCP1 antibody, e.g., described herein, or antigen-binding fragment thereof alone or in combination with another agent is used concurrently or sequentially with bone marrow transplantation to treat a variety of tumors of hematopoietic origin.

Provided herein are methods for altering an adverse event associated with treatment of a hyperproliferative disease (e.g., cancer) with an immuno stimulatory agent, comprising administering an anti-cleaved CDCP1 antibody, e.g., described herein, or antigen-binding fragment thereof with or without a second agent, to a subject. For example, the methods described herein provide for a method of reducing the incidence of immuno stimulatory therapeutic antibody-induced colitis or diarrhea by administering a non-absorbable steroid to the patient. As used herein, a “non-absorbable steroid” is a glucocorticoid that exhibits extensive first pass metabolism such that, following metabolism in the liver, the bioavailability of the steroid is low, i.e., less than about 20%. In some aspects described herein, the non-absorbable steroid is budesonide. Budesonide is a locally-acting glucocorticosteroid, which is extensively metabolized, primarily by the liver, following oral administration. ENTOCORT EC® (Astra-Zeneca) is a pH- and time-dependent oral formulation of budesonide developed to optimize drug delivery to the ileum and throughout the colon. ENTOCORT EC® is approved in the U.S. for the treatment of mild to moderate Crohn's disease involving the ileum and/or ascending colon. In some aspects, an anti-cleaved CDCP1 antibody, e.g., described herein, or antigen-binding fragment thereof in conjunction with a non-absorbable steroid can be further combined with a salicylate. Salicylates include 5-ASA agents such as, for example: sulfasalazine (AZULFIDINE®, Pharmacia & Up John); olsalazine (DJPENTUM®, Pharmacia & Up John); balsalazide (COLAZAL®, Salix Pharmaceuticals, Inc.); and mesalamine (ASACOL®, Procter & Gamble Pharmaceuticals; PENTASA®, Shire US; CANASA®, Axcan Scandipharm, Inc.; ROWASA®, Solvay).

### XII. Kits

Provided herein are kits comprising one or more antibodies described herein, or antigen-binding fragments thereof, bispecific antibodies, multispecific antibodies, or immunoconjugates thereof. In a specific aspect, provided herein is a pharmaceutical pack or kit comprising one or more containers filled with one or more of the ingredients of the pharmaceutical compositions described herein, such as one or more antibodies provided herein or an antigen-binding fragment thereof, optional an instructing for use. In some aspects, the kits contain a pharmaceutical composition described herein and any prophylactic or therapeutic agent, such as those described herein.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatisasdade, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Caner and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986);); Crooks, Antisense drug Technology: Principles, strategies and applications, 2^{nd} Ed. CRC Press (2007) and in Ausubel et al. (1989) Current Protocols in Molecular Biology (John Wiley and Sons, Baltimore, Md.).

The following examples are offered by way of illustration and not by way of limitation.

### EXAMPLES

### Example 1: Materials and Methods

Unless provided otherwise, the Examples described below use one or more of the following materials and methods:

### 1.1 Cloning, Protein Expression, and Purification

Plasmids encoding CDCP1 Fc or non-Fc fusions, the heavy chain and light chains of IgGs, and the heavy chain and light chain fused to scFv of BiTE were generated by Gibson cloning into pFUSE (InvivoGen). Fabs were PCR-ed from the Fab phagemid and subcloned into pBL347. Plasmids for stable cell line construction were generated by Gibson cloning into pCDH-EF1-CymR-T2A-Neo (System Bioscience).

A previously described vector for expression of Fabs was used. See Hornsby, M., et al., Mol. Cell. Proteomics 14:2833-2847 (2015). Briefly, C43 (DE3) Pro+ E. coli transformed with the Fab expression plasmid were grown in TB autoinduction media at 37° C. for 6 hrs, then switched to 30° C. for 16-18 hrs. Cells were harvested by centrifugation (6000×g for 20 min) and lysed with B-PER Bacterial Protein Extraction Reagent (Thermo Fischer). Lysate was incubated at 60° C. for 20 min and centrifuged at 14,000×g for 30 min. Clarified supernatant was passed through a 0.45 μm syringe filter. Fabs were purified by Protein A affinity chromatography on an AKTA Pure system. Fab purity and integrity were assessed by SDS-PAGE and intact mass spectrometry.

Constructs encoding CDCP1 were generated by transfection of BirA-Expi293 cells (Life Technologies) with one or two plasmids encoding cleaved or uncleaved CDCP1 antigen as Fc or non-Fc fusions. IgGs and BiTE were generated by transfection of BirA-Expi293 cells with two plasmids encoding the heavy chain and light chain at 1:1 ratio. The EXPIFECTAMINE™ 293 transfection kit (Life Technologies) was used for transfections as per manufacturer's instructions. Cells were incubated for 5 days at 37° C. in 5% CO2 at 125 rpm before the supernatants were harvested by centrifugation. Proteins were purified by Protein A affinity chromatography (CDCP1 Fc-fusions, IgGs, and BiTE) or Ni-NTA affinity chromatography (non-Fc fusion CDCP1) and assessed for quality and integrity by SDS-PAGE.

### 1.2 Lentiviral Cell Line Construction

The HEK293T stable cell lines used throughout the examples were generated by lentiviral transduction. To produce virus, HEK293T Lenti-X cells were transfected with a mixture of second-generation lentiviral packaging plasmids at ˜80% confluence. FuGene HD (Promega) was used for transfection of the plasmids using 3 μg DNA (1.35 μg pCMV delta8.91, 0.15 μg pMD2-G, 1.5 μg pCDH vectors encoding gene of interest) and 7.5 μL of FuGene HD per well of a six-well plate. Media was changed to complete DMEM after 6 hrs of incubation with transfection mixture. The supernatant was harvested and cleared by passing through a 0.45 μm filter 72 hrs post transfection. Cleared supernatant was added to target HEK293T wild-type cells (˜1 million cells per mL) with 8 μg/mL polybrene and cells were centrifuged at 1000×g at 33° C. for 2 hrs. Cells were then incubated with viral supernatant mixture overnight before the media was changed to fresh complete DMEM. Cells were expanded for a minimum of 48 hrs before they were grown in drug selection media. Drug selection for stable cell lines was started by the addition of 2 μg/mL puromycin. Following at least 72 hrs of incubation in puromycin containing media, cells were analyzed by flow cytometry for expression of CDCP1.

Mouse pancreatic cancer cell line Fc1245 overexpressing c-CDCP1 were generated using the same protocol except using a Hygromycin B selection marker.

### 1.3 Mammalian Cell Culture

HPAC, PL5, PL45, and HPNE cells were maintained in IMDM+10% FBS+1× Pen/Strep. The HEK293T cell lines were cultured in DMEM+10% FBS+1× Pen/Strep. Jurkat NFAT-GFP reporter cells lines were cultured in RPMI+10% FBS+2 mg/mL G418+1× Pen/Strep. Fc1245 was cultured in DMEM+10% FBS+1× Pen/Strep. Cell line identities were authenticated by morphological inspection.

### 1.4 Immunoprecipitation (IP)

Cells confluent in a 15-cm dish were washed by PBS buffer three times before being lysed on plate with 1 mL ice-cold NP-40 lysis buffer supplemented with protease inhibitor cocktail (Roche) and PhosSTOP (Roche). The cell lysate was incubated at 4° C. with gentle shaking for 30 min and centrifuged at 14,000×g for 30 min at 4° C. The supernatant was then transferred into a new tube. Protein concentration was determined by BCA assay (Bio-Rad). For IP-WB, 5 μL CDCP1 antibody (D1W9N, Cell Signaling) was added into 200 μL cell lysate precleared by 20 μL Protein A magnetic beads (EMD Millipore) and incubated overnight at 4° C. On the second day, 20 μL Protein A magnetic beads was added to the lysate and incubated for 20 min at room temperature. The Protein A magnetic beads were washed 5× with 0.5 mL lysis buffer. 20 μL 4×SDS loading buffer was added and the beads were heated to elute protein at 95° C. for 5 min. The sample was then analyzed by Western blot analysis. For IP-MS, 20 μL CDCP1 antibody (D1W9N, Cell Signaling) was added into 1 mL cell lysate and incubated overnight at 4° C. On the second day, 100 μL of Protein A magnetic beads was added to the lysate and incubated for 20 min. Protein A magnetic beads were washed 5× with 1 mL lysis buffer and PBS buffer. CDCP1 was then eluted with 200 μL 0.1 M acetic acid and neutralized by 20 μL pH 11 Tris Buffer. Samples were then used for proteomics analysis.

### 1.5 Mass Spectrometry (MS)

Samples were reduced with 5 mM TCEP at 55° C. for 30 min and alkylated with 10 mM iodoacetamide at room temperature for 30 min. Proteins were subjected to digestion using 20 μg sequencing grade Glu-C (Promega) in 1 M urea at 37° C. overnight. The eluted fraction was collected and then desalted using Sola column (Thermo Fisher) following standard protocol. Desalted peptides were dried and dissolved in mass spectrometry buffer (0.1% formic acid+2% acetonitrile) prior to LC-MS/MS analysis.

1 μg of peptide was injected into a pre-packed 0.075 mm×150 mm Acclaim Pepmap C18 LC column (2 μm pore size, Thermo Fisher) attached to a Q Exactive Plus (Thermo Fisher) mass spectrometer. Peptides were separated using a linear gradient of 3-35% solvent B (Solvent A: 0.1% formic acid, Solvent B: 80% acetonitrile, 0.1% formic acid) over 170 min at 300 μL/min. Data were collected in data-dependent acquisition mode using a top 20 method with a dynamic exclusion of 35 sec and a charge exclusion restricted to charges of 2, 3, or 4. Full (MS1) scan spectrums were collected as profile data with a resolution of 140,000 (at 200 m/z), AGC target of 3E6, maximum injection time of 120 msec, and scan range of 400-1800 m/z. Fragment ion (MS2) scans were collected as centroid data with a resolution of 17,500 (at 200 m/z), AGC target of 5E4, maximum injection time of 60 msec with a normalized collision energy at 27, and an isolation window of 1.5 m/z with an isolation offset of 0.5 m/z. Peptide search and MS1 peak area quantification were performed using ProteinProspector (v.5.13.2) against user-defined CDCP1 sequence (Uniprot Accession ID: Q9H5V8).

### 1.6 Western Blot

The confluent cells were washed with PBS and lysed with ice-cold NP-40 Lysis Buffer supplemented with protease inhibitor cocktail (Roche) and PhosSTOP (Roche). Lysate was incubated with gentle shaking at 4° C. for 30 min and centrifuged at 14,000×g for 30 min at 4° C. The supernatant was then transferred into a new tube and protein concentration was determined by BCA assay (Bio-Rad). Immunoblotting was performed using CDCP1 (D1W9N) (Cell Signaling, 13794S), Phospho-CDCP1 (Tyr707) (Cell Signaling, 13111S), Phospho-CDCP1 (Tyr806) (Cell Signaling, 13024S), Phospho-CDCP1 (Try734) (Cell Signaling, 9050S), Phospho-CDCP1 (Try743) (D2G2J) (Cell Signaling, 14965S), Src (36D10) (Cell Signaling, 2109S), Phospho Src Family (Tyr416) (Cell Signaling, 2101S), PKC8 (Cell Signaling, 2058S), Phospho-PKCdelta (Tyr311) (Cell Signaling, 2055S), alpha-Tubulin (DM1A) (Cell Signaling, 3873S), IRDye 680RD Goat anti-Mouse (LiCOR, 925-68070), and IRDye 800CW Goat anti-Rabbit (LiCOR, 926-32211) antibodies.

### 1.7 Flow Cytometry

Cells were lifted with Versene (0.04% EDTA, PBS pH 7.4 Mg/Ca free), washed once with PBS pH 7.4, and subsequently blocked with flow cytometry buffer (PBS, pH 7.4, 3% BSA). Primary Abs were added to cells for 30 minutes at 4° C. Bound Abs were detected with addition of AlexaFluor-488 or AlexaFluor-647 conjugated Goat anti-human IgG, F(ab′)₂ fragment specific (Jackson ImmunoResearch: 1:1000). Cells were washed 3× with PBS+3% BSA and fluorescence was quantified using a CytoFLEX (Beckman Coulter) flow cytometer. All flow cytometry data analysis was performed using FlowJo software and Prism software (GraphPad).

### 1.8 Bio-Layer Interferometry (BLI) Experiments

BLI experiments were performed using an Octet RED384 instrument (ForteBio). Biotinylated proteins were immobilized on a streptavidin (SA) biosensor and His-tagged proteins were immobilized on a Ni-NTA biosensor. Different concentrations of analyte in PBS pH 7.4+0.05% Tween-20+0.2% BSA (PBSTB) were used as analyte. Affinities (K_{D}S) were calculated from a global fit (1:1) of the data using the Octet RED384 software.

### 1.9 Size Exclusion Chromatography (SEC)

SEC analysis was performed using an Agilent HPLC 1260 Infinity II LC System using an AdvanceBio SEC column (300 Å, 2.7 μm, Agilent). Each analyte was injected at 1-10 μM and run with a constant mobile phase of 0.15 M sodium phosphate for 15 min. Absorbance at 280 nm was measured.

### 1.10 Circular Dichroism (CD) Spectroscopy

CD spectra were measured using an Aviv 410 CD spectrophotometer. The CD signal from 200 nm to 300 nm was collected in a 0.1-cm path length cuvette at 25° C. Samples contained 0.4 mg/mL of protein in PBS. All CD spectra were blanked with PBS in the absence of protein.

### 1.11 SEC-SAXS

Small angle X-ray scattering in-line with size exclusion chromatography (SEC-SAXS) data were collected at the SIBLYS beamline 12.3.1 of the Advanced Light Source at the Lawrence Berkeley National Laboratory (Classen, S., et al., J. Appl. Crystallogr. 46:1-13 (2013)) at 1.127 Å wavelength and sample-to-detector distance of 2,105 mm. The resulting scattering vectors, defined as q=4π sin θ/λ, where 20 is the scattering angle, ranged from 0.01 to 0.4 Å-1. Data were collected using a Dectris PILATUS3 2M detector at 20° C. and processed as previously described. See Dyer, K. et al. High-Throughput SAXS for the Characterization of Biomolecules in Solution: A Practical Approach. Methods Mol. Biol. 1091, 245-258 (2014); and Hura, G. L., et al., Nat. Methods 6:606-612 (2009).

The SEC-SAXS flow cell was directly coupled with an Agilent 1260 Infinity HPLC system using a Shodex KW-803 column. The column was equilibrated with running buffer (1×PBS-10 mM phosphate, 137 mM NaCl, 2.7 mM KCl; pH 7.4) at a flow rate of 0.45 mL/min. fl-CDCP1 and c-CDCP1 (Cut 3) proteins were injected at ˜ 5 mg/ml and 3 sec X-ray exposures were collected continuously over the 30 min SEC elution in the running buffer for each sample. The SAXS frames recorded prior to the protein elution peak were used to subtract the signal for SAXS frames across the elution peak. Radius of gyration (R_{g}) were determined based on the Guinier approximation (Guinier, A., Acta Metall. 3:510-512 (1955)), I(q)=I(0) exp (−q²R_{g}²/3), with the limits qR_{g}<1.3. Further, scattering intensity at q=0 Å-1 (I (0)) and R_{g} values were compared for each collected SAXS curve across the entire elution peak. The elution peak was mapped by plotting the scattering intensity at q-0 A1 (I (0)) relative to the recorded frame. Interference-free SAXS curves with least R_{g} variation were averaged and merged in SCATTER to produce the highest signal-to-noise SAXS curves. These merged SAXS curves were used to generate the Guinier plots, volumes-of-correlation (V_{c}), pair distribution functions, P(r), and normalized Kratky plots. Pair distribution function P(r) was computed using program GNOM. See König, S., et al., Biochemistry 31:8726-8731 (1992). P(r) functions were normalized based on the molecular weight (MW) of the macromolecules, as determined based on their calculated V_{c}. See Rambo, R. P. & Tainer, J. A., Nature 496:477-481 (2013).

### 1.12 SEC-MALS Data Collection and Analysis

The SEC eluent was split (4 to 1) between the SAXS line and a multiple wavelength detector (UV-vis) at 280 nm, multi-angle light scattering (MALS), and refractometer. MALS experiments were collected using an 18-angle DAWN HELEOS II light scattering detector connected in tandem to an Optilab refractive index concentration detector (Wyatt Technology). Bovine serum albumin was used for system normalization and calibration. The MALS data complimented the MW determined by SAXS analyses. The MALS data were also used to align the SAXS and UV-vis peaks along the X-axis (elution volume in mL) to compensate for fluctuations in timing and band broadening. MALS and differential refractive index data were analyzed using Wyatt Astra seven software to monitor the homogeneity of the sample molecular weights across the elution peak.

### 1.13 Differential Scanning Fluorimetry (DSF)

Protein in PBS were mixed with Sypro Orange dye (20× stock) to make a final protein concentration of 2 μM and dye concentration 4×. 10 μL of each mixture was transferred to a Biorad 384-well PCR white plate and the plate was covered by qPCR Sealing Tape. The assay was performed over temperature range of 25° C. to 95° C. with a temperature ramping rate of 0.5° C./30 seconds on a Roche LC480 Light Cycler.

### 1.14 Negative-Stain Electron Microscopy

The samples were negatively stained and observed on a Tecnai T12 microscope using discharged continuous carbon grids (Ted Pella, Inc.). Images were acquired at room temperature with a pixel size of 2.21 Å/pixel (T12, operated at 120 kV) on the level of specimen using a 4K×4K CCD camera (UltraScan 4000, Gatan Inc., USA). After all micrographs were visually screened, contrast transfer function (CTF) was estimated for each micrograph by Gctf40. Particle was selected using the Gautomatch (https://www.mrc-lmb.cam.ac.uk/kzhang/Gautomatch) without template. Individual particles were extracted from the raw images with 100×100-pixel window for T12 images and were subjected to 25 cycles of 2D classification with mask diameter of 200 Å in Relion 3.041.

### 1.15 Cell Proliferation Assay

Cell proliferation assays were performed using an MTT modified assay to measure cell viability. In brief, 5,000 HEK293T cells expressing different CDCP1 constructs were plated in each well of a 96-well plate on day 0. Cells were incubated at 37° C. under 5% CO₂. On day 1, 2, 3 and 4, 10 μL of 5 mg/mL of Thiazolyl Blue Tetrazolium Bromide (Sigma Aldrich) was added to each well and incubated at 37° C. under 5% CO₂ for 2 hrs. Following, 100 μL of 10% SDS+0.01 M HCl was added to lyse the cells to dissolve the MTT product. After 4 hrs, absorbance at 595 nm was quantified using an Infinite M200 PRO-plate reader (Tecan). Data points were plotted using Prism software (GraphPad).

### 1.16 Cell Adhesion Assay

The day prior to the assay, a 96-well tissue culture plate was coated with MaxGel™ ECM (Millipore Sigma) 1:10 diluted in serum-free DMEM. At the same time, the cell culture medium for HEK293T cells with expressing different CDCP1 constructs was changed to serum-free medium. The next day, media was removed and the culture plates were blocked with 100 μL serum-free DMEM with 0.1% BSA for 2 hrs and washed with PBS. 100,000 cells in 100 μL of serum-free (0.1% BSA) medium were added to each well and incubated at 37° C. in a 5% CO₂ for 2 hrs. The non-adherent cells were removed by washing with media three times, and the remaining cells were quantified by MTT assay. Data points were plotted using Prism software (GraphPad).

### 1.17 Phage Selection

All phage selections were done according to previously established protocols. See Hornsby, M., et al., Proteomics 14:2833-2847 (2015). Briefly, selections were performed using biotinylated c-CDCP1-Fc captured on SA-coated magnetic beads (Promega). Prior to each selection, the phage pool was incubated with 1 μM of biotinylated fl-CDCP1-Fc captured on streptavidin beads in order to deplete the library of any binders to fl-CDCP1. Four rounds of selection were performed with decreasing amounts of c-CDCP1-Fc (100 nM, 50 nM, 10 nM and 10 nM). A “catch and release” strategy was employed, where bound Fab-phage were eluted from the magnetic beads by the addition of 2 μg/mL of TEV protease. Individual phage clones from the third and fourth round of selection were analyzed for binding by phage ELISA.

### 1.18 Phage ELISA

Phage ELISAs were performed according to standard protocols. Briefly, 384-well Maxisorp plates were coated with NeutrAvidin (10 μg/mL) overnight at 4° C. and subsequently blocked with PBS+2% BSA for 1 hr at 20° C. 20 nM of biotinylated c-CDCP1-Fc or fl-CDCP1 ECD-Fc was captured on the NeutrAvidin-coated wells for 30 min followed by the addition phage supernatants diluted 1:5 in PBSTB for 30 min. Bound phage were detected using a horseradish peroxidase (HRP)-conjugated anti-M13 phage antibody (GE Lifesciences 27-9421-01).

### 1.19 Immunofluorescence

HPAC, PL5, and HPNE cells were plated on glass-bottom imaging plates (MatTek) and incubated for 24 hrs at 37° C. under 5% CO₂. Cells were treated with IgG (1 μg/mL) for 30 min and washed with media to remove unbound IgG. Bound IgG was detected by the addition of a Alexa Fluor® 488-conjugated AffiniPure F(ab′)2 Fragment Goat Anti-Human IgG, F(ab′)2 Fragment Specific (Jackson ImmunoResearch, 143225) in Invitrogen Molecular Probes Live Cell Imaging Solution (Thermo Fisher) containing Hoescht blue (2 μg/mL). Cells were imaged on a Nikon Ti Microscope Yokogawa CSU-22 with Spinning Disk Confocal.

### 1.20 In Vitro Antibody Drug Conjugate (ADC) Assays

In vitro antibody drug conjugate cell killing assays were performed either using a secondary antibody conjugated to MMAF, or by direct conjugation of MMAF to the primary antibody. Secondary ADC assays used a Fab Anti-Human IgG Fc-MMAF Antibody with Cleavable Linker (Moradec) as a secondary antibody following manufacture's protocol. For ADC assays using direct conjugation of MMAF to the primary antibody, the antibody was labeled with DBCO-PEG4-ValCit-MMAF (Levena Biosciences) site-specifically at residue T74M of the light chain using oxazirdine chemistry using a previously described protocol. See Elledge, S. K., et al., Proc. Natl. Acad. Sci. U.S.A 117:5733-5740 (2020). A day prior to the assay, 5000 cells were seeded on a 96-well poly-lysine-coated white plate (Corning). The next day, media was removed and 100 μL of primary IgG and secondary ADC at a 1:4 ratio or 100 μL of MMAF-labeled primary IgG in media was added. Cells were incubated for 72 hrs at 37° C. under 5% CO₂. After the incubation period, 100 μL of CellTiter-Glo Reagent (Promega) was added to each well followed by incubation at room temperature for 10 min with gentle shaking. Luminescence was measured using an Infinite M200 PRO plate reader (Tecan).

### 1.21 Bi-Specific T-Cell Engager (BiTE) Assay

A day prior to the assay, 25,000 target cells were seeded on a 96-well plate (Corning). The next day, media was removed and 50,000 Jurkat NFAT-GFP reporter cells and BiTE (10-fold dilution) were added to a final media volume of 200 μL. After incubation for 20 hrs at 37° C., Jurkat cells were recovered by gentle pipetting, washed in PBS+3% BSA, and GFP expression was quantified by flow cytometry using a CytoFLEX (Beckman Coulter) flow cytometer.

### 1.22 Mouse Positron Emission Tomography (PET) Imaging Study

110 μL of IgG at a concentration of 4.2 mg/mL was dispersed in 100 μL of 0.1 M sodium bicarbonate buffer (pH 9.0). The pH was adjusted to 9.0 and the final reaction mixture was adjusted to a total volume of 0.5 mL by adding a sufficient amount of 0.1 M sodium bicarbonate buffer. Df-Bz-NCS (p-isothiocyanatobenzyl-desferrioxamine) was dissolved in DMSO at a concentration of 10 mM. Df-Bz-NCS solution was added to the antibody solution to give a three molar excess of the chelator over the molar amount of Fab. The Df-Bz-NCS was added in steps of 2 μL and mix rigorously during the addition. The concentration of DMSO was kept below 2% of the total reaction mixture in order to avoid any precipitation. After 30 min at 37° C., the reaction mixture was purified via a PD-10 column pre equilibrated by 20 mL of gentisic acid solution (5 mg/mL of gentisic acid in 0.25 M sodium acetate (pH 5.4-5.6)). The Fab-DFO solution was eluated in gentisic acid solution, and the pH was adjusted to seven by addition of NaOH (1 M). Then, the solution was aliquoted and stored at −20° C. until the day of radiolabeling.

[89Zr]Zr-oxalic acid solution (5 mCi; 10 μL) was neutralized with 2 M Na2CO3 (5 μL). After 3 min, 0.30 mL of 0.5 M HEPES (pH 7.1-7.3) and 0.5 mg of DFO-Fab (pH 7) were added into the reaction vial. After incubation (120 min) at 37° C., the radiolabeling efficiency was determined by ITLC using chromatography strips and 20 mM citric acid (pH 4.9-5.1). The radiolabeling efficiency was consistently >98.5%.

### Example 2. Analysis of CDCP1 Proteolysis

In the present example, proteolysis of CDCP1 was evaluated as follows. Recombinant protein and cell lines expressing CDCP1 with an engineered cut site were generated using standard methods. Briefly, plasmids encoding CDCP1 constructs were generated by Gibson cloning into pFUSE (InvivoGen), which were used to transfect BirA-Expi293 cells (Life Technologies). The EXPIFECTAMINE™ 293 transfection kit (Life Technologies) was used for transfections as per manufacturer's instructions. Proteins were purified by standard methods and assessed for quality and integrity by SDS-PAGE. HEK293T stable cell lines expressing constructs of interest were generated using standard lentiviral transduction methods.

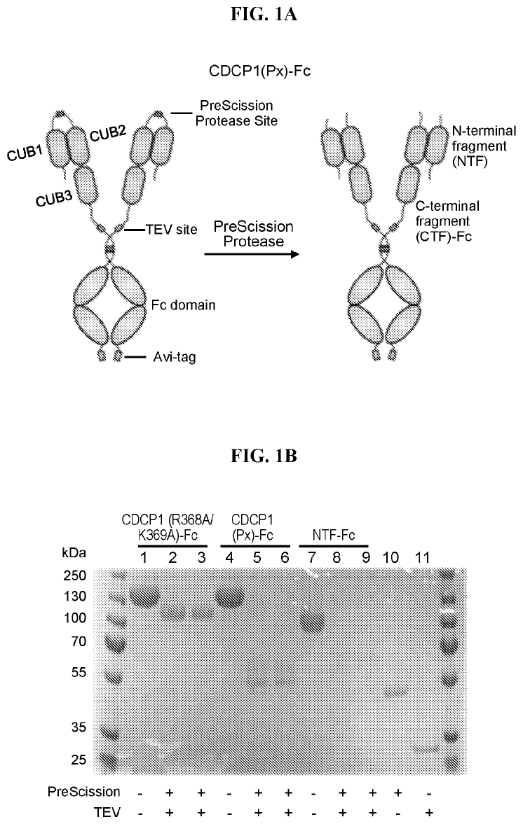

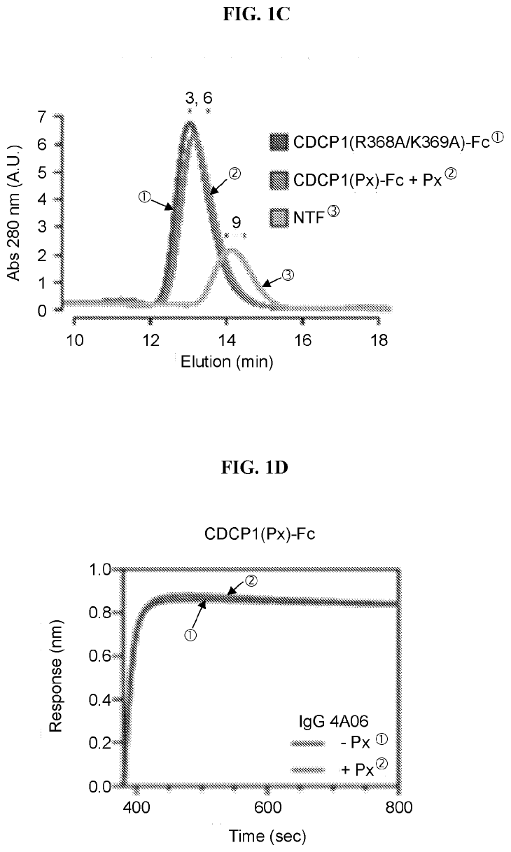

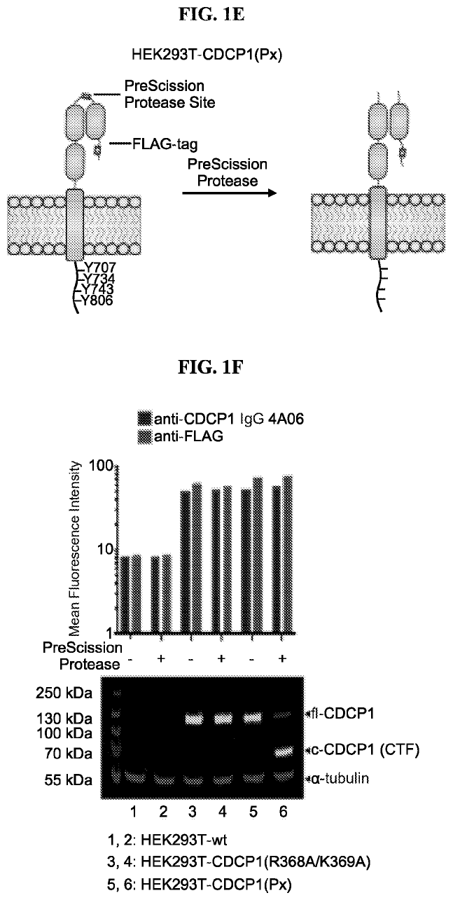

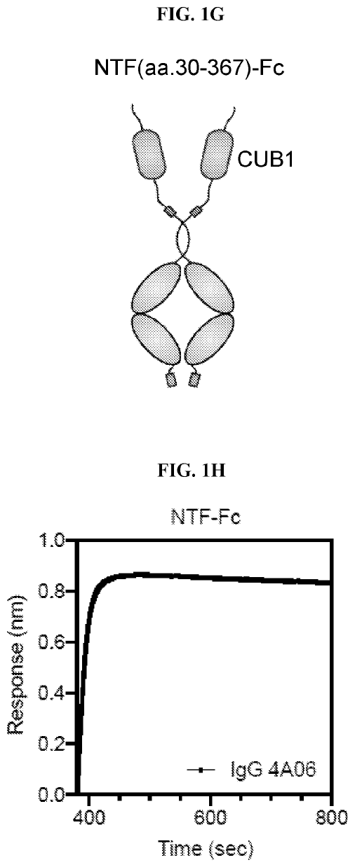

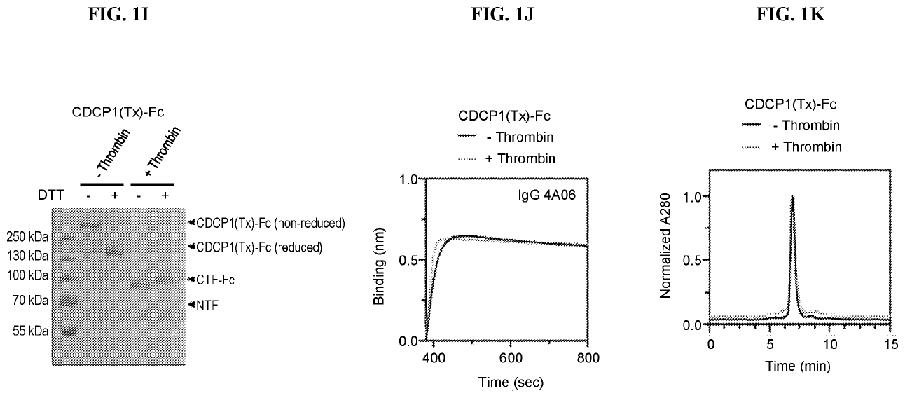

CDCP1 has been reported to be cleaved at the dibasic arginine-lysine residues between the first and second CUB domains (R368/K369). See, for example, Uekita, T. & Sakai, R., Cancer Sci. 102:1943-1948 (2011), and He, Y., et al., J. Biol. Chem. 285:26162-26173 (2010). This reported cut site was replaced with a PreScission Protease (Px) recognition sequence (GS₅-LEVLFQGP-GS₅) to readily control the proteolysis of CDCP1. The ectodomain of CDCP1 with this engineered cut site was recombinantly expressed as an TEV-releasable Fc-fusion protein with a C-terminal biotinylated Avi-tag (CDCP1 (Px)-Fc) (FIG. 1A). An additional variant was generated where R368 and K369 were replaced with alanine so that the construct to prevent cleavage of the construct by basic residue-specific proteases (CDCP1 (R368A/K369A)-Fc). A further additional variant was generated where only the N-terminal fragment (NTF, aa 30-367) was fused to an Fc domain (NTF-Fc) (FIG. 1G).

Px cleavage of CDCP1 (Px)-Fc was performed, which resulted in two fragments of expected molecular weight for NTF and CTF (FIG. 1B). Surprisingly, it was found by size exclusion chromatography (SEC) using an Agilent HPLC 1260 Infinity II LC System and standard methods that Px-treated CDCP1 (Px)-Fc had the same elution profile as uncleaved CDCP1-Fc, with no evidence of an unbound NTF (FIG. 1C).

Additionally, bio-layer interferometry (BLI) assays performed using an Octet RED384 instrument (ForteBio) and standard methods demonstrated that when CDCP1 (Px)-Fc was immobilized via its C-termini, there was robust binding of an antibody (IgG 4A06) that recognizes the NTF regardless of Px treatment (FIG. 1D and FIG. 1H). As a control to demonstrate that this observation was not an artifact of engineering or treatment with PreScission protease, a Thrombin protease-cleavable CDCP1-Fc, CDCP1(Tx)-Fc was generated (FIG. 1I-FIG. 1K). Thrombin treatment using standard methods generated the expected sized fragments and it was found that the NTF did not dissociate from the CTF upon proteolysis. BLI using an Octet RED384 instrument (ForteBio) and standard methods demonstrated of IgG 4A06 binding to C-terminally immobilized CDCP1(Tx)-Fc treated or untreated with thrombin demonstrated that IgG 4A06, that recognized the NTF, could still bind cleaved CDCP1 (FIG. 1J). SEC was performed, and an SEC trace of CDCP1(Tx)-Fc treated or untreated with thrombin protease that cleaved and uncleaved CDCP1 had similar elution profiles, suggesting the NTF and CTF-Fc remained associated (FIG. 1K). Based on these results, it was concluded that recombinant cleaved CDCP1 with engineered proteolysis sites remained a complex upon protease treatment.

To determine whether cleaved CDCP1 was a complex on the cell membrane, a HEK293T cell line expressing the full CDCP1 protein sequence with an N-terminal FLAG-tag was engineered, and the R368/K369 proteolysis site was replaced with the Px recognition sequence (FIG. 1E). As a control a HEK293T cell line expressing the R368A/R369A uncleavable variant was generated. Using a commercial antibody (D1W9N) that recognizes the intracellular C-terminal region of CDCP1, it was observed that addition of Px to cells cleaved CDCP1 (Px) at the expected molecular weight (see, for example, Western blot of FIG. 1F). Probing by flow cytometry with an anti-FLAG antibody or by IgG 4A06, it was demonstrated that the NTF of CDCP1 remained associated on cells after proteolysis. Thus, the results indicated that the specific proteolysis of the CDCP1 ectodomain did not lead to large conformational changes nor dissociation of the NTF and CTF in solution or on cells.

### Example 3: Characterization of Pancreatic Ductal Adenocarcinoma (PDAC) Cell Lines Expressing CDCP1

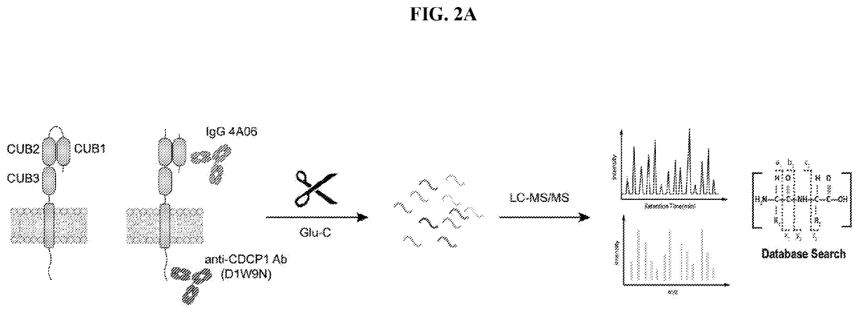

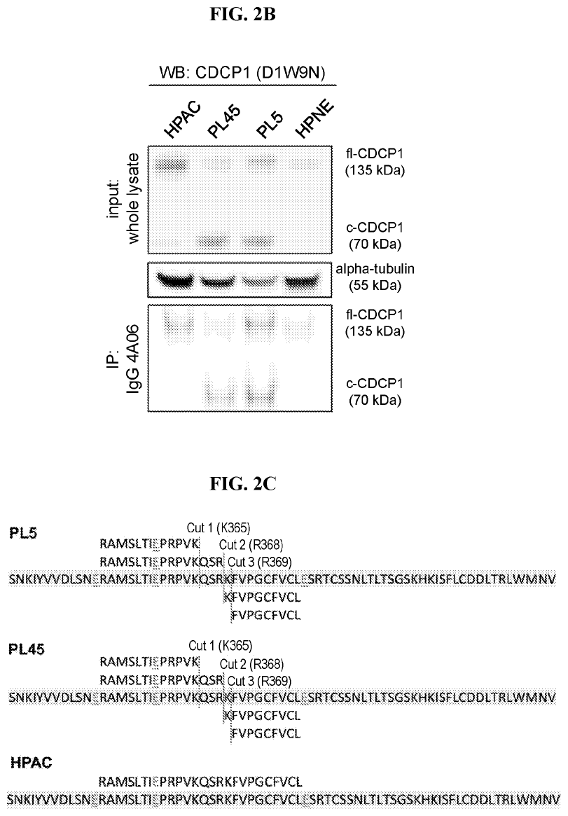

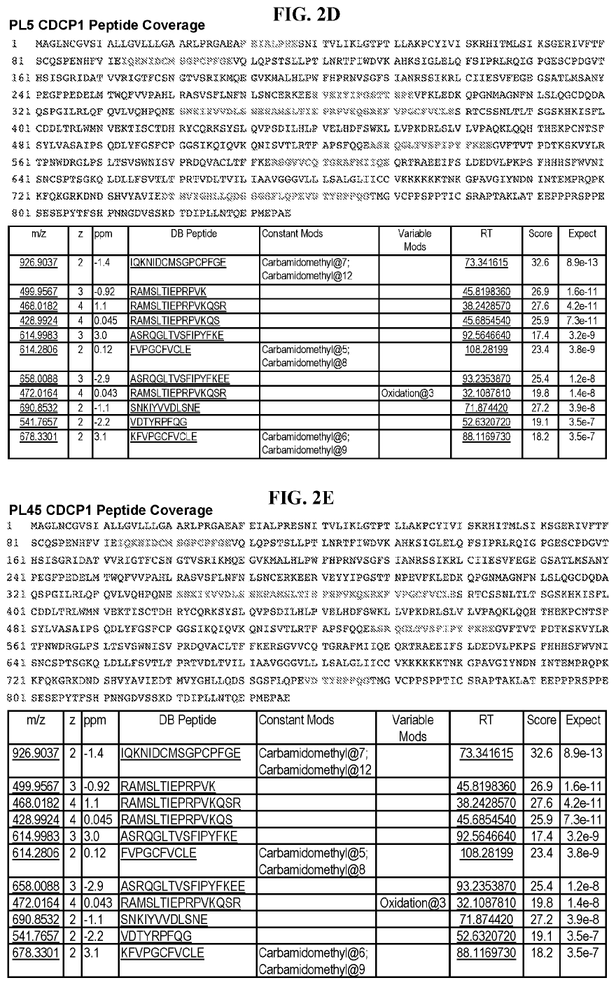

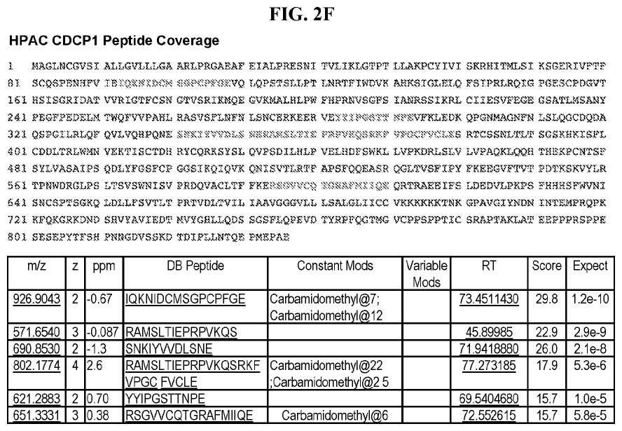

In the present example, characterization of a panel of pancreatic ductal adenocarcinoma (PDAC) cell lines expressing differential amounts of full-length and cleaved CDCP1 was performed. Characterization included immunoprecipitation mass spectrometry (IP-MS) to map peptides near the reported cut site of CDCP1 (FIG. 2A).

Cell lines used in the present example include HPAC, which expressed mostly uncleaved CDCP1; PL5 and PL45, which expressed mostly cleaved CDCP1; and HPNE, which was a non-malignant pancreatic cell line with little CDCP1 expression (FIG. 2B).

IP was performed with either a commercial anti-CDCP1 Ab (D1W9N) that recognized the C-terminal intracellular region, or with an in-house antibody IgG 4A06 that recognized the NTF. It was found that the CTF of CDCP1 co-IPs with the NTF, thereby indicating that cleaved CDCP1 was a complex on PDAC cells (FIG. 2B). For analysis by MS, the IP-ed sample was treated with Glu-C to preserve the expected basic cut site of CDCP1. Peptides corresponding to both proteolytic fragments of CDCP1 in PL5 and PL45 cells were observed, regardless of which Ab was used for the IP, further indicating that endogenous cleaved CDCP1 was a complex on PDAC cells (FIG. 2C and FIG. 2D-FIG. 2F). Peptides corresponding to the uncleaved sequence were exclusively observed for HPAC, and no peptides mapping to CDCP1 were observed for HPNE reflecting the expected absence of CDCP1 expression. For PL5 and PL45 cells, three unique cut sites were identified, all of which lie between the CUB1 and CUB2 domain boundaries; these included proteolysis after K365 (Cut1), R368 (Cut2), K369 (Cut3). Cut2 and Cut3 were previously reported, while Cut1 was found to be a novel site. These findings confirmed that endogenous cleaved CDCP1 remained a complex on PDAC cells and showed that CDCP1 could be proteolytically processed between CUB1 and CUB2 and produced a heterogenous set of cleaved forms.

### Example 4: Generation of Recombinant CDCP1 Antigen with Endogenous Cut Sites

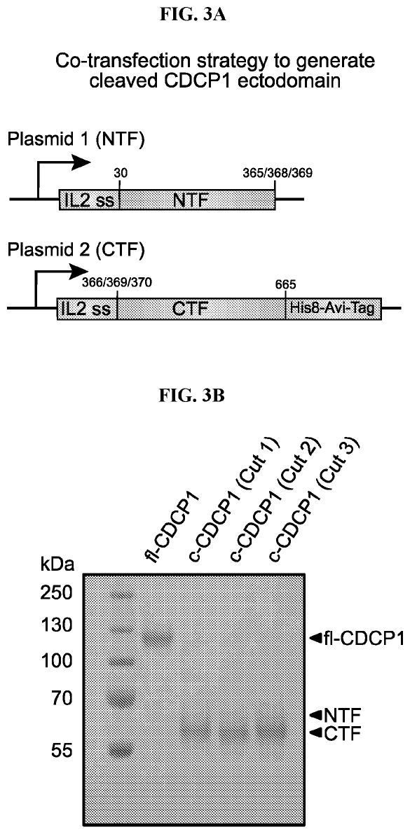

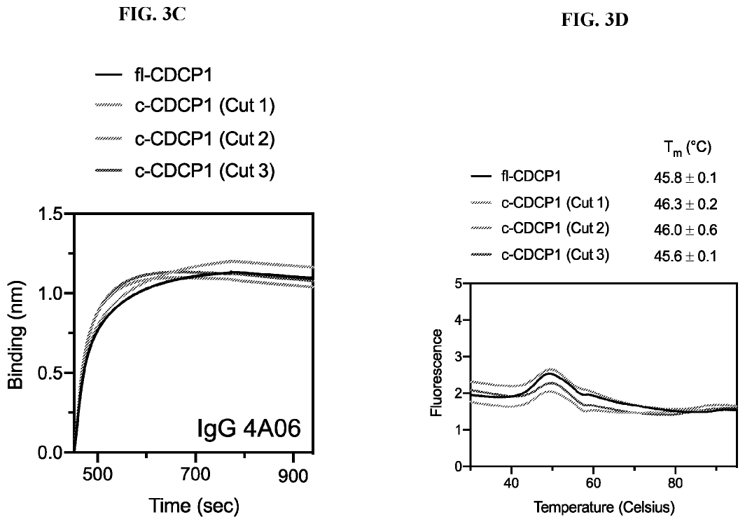

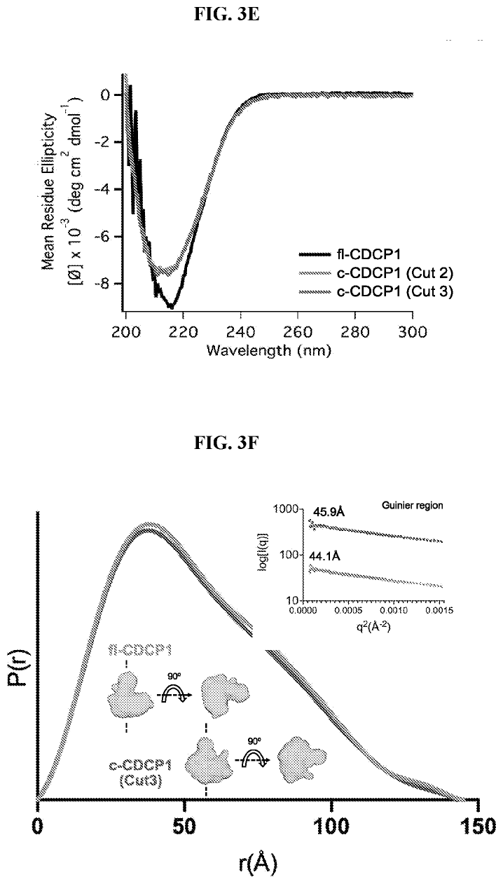

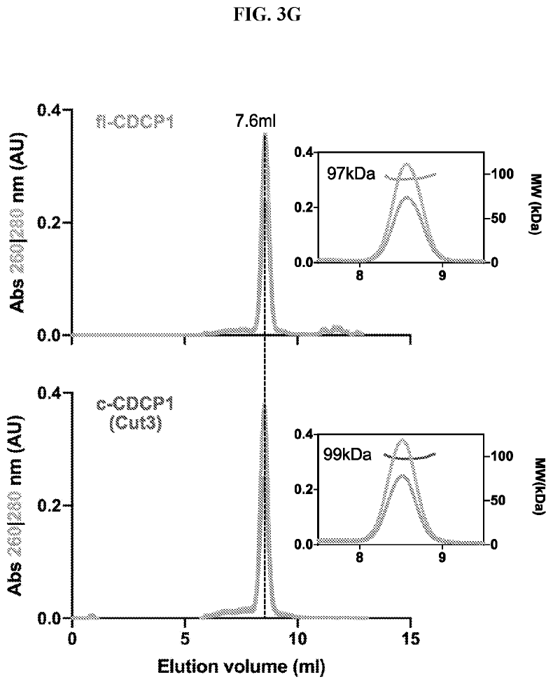

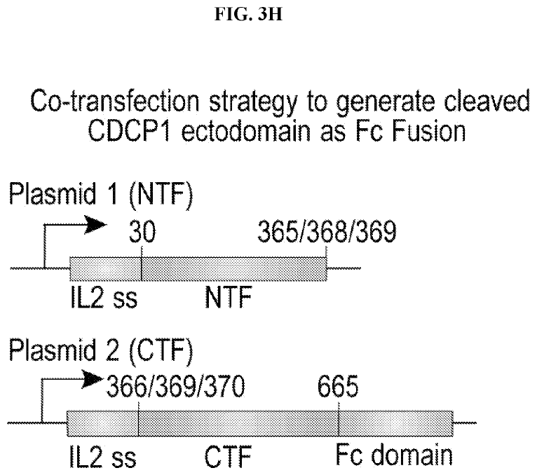

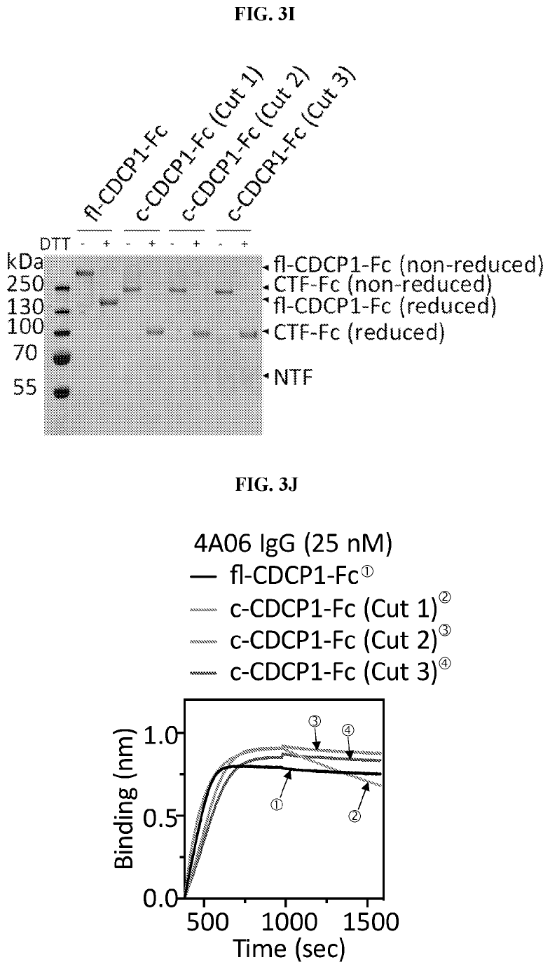

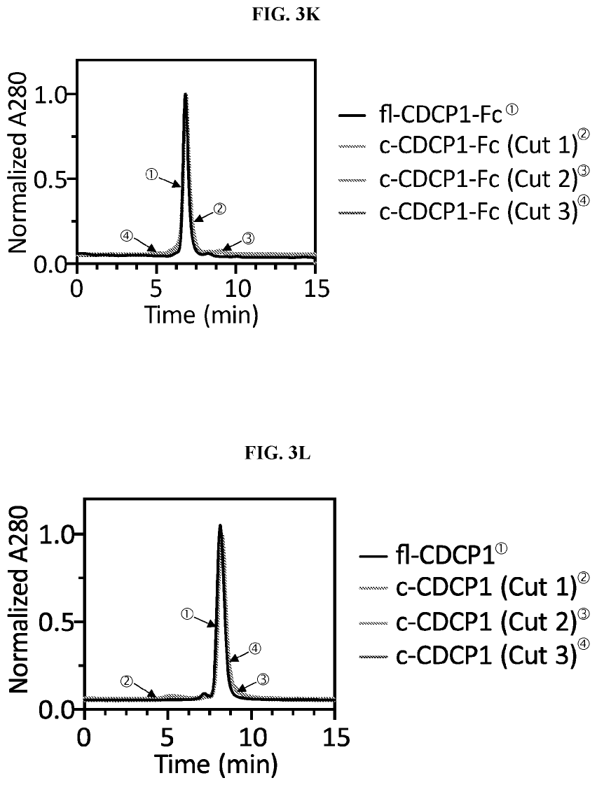

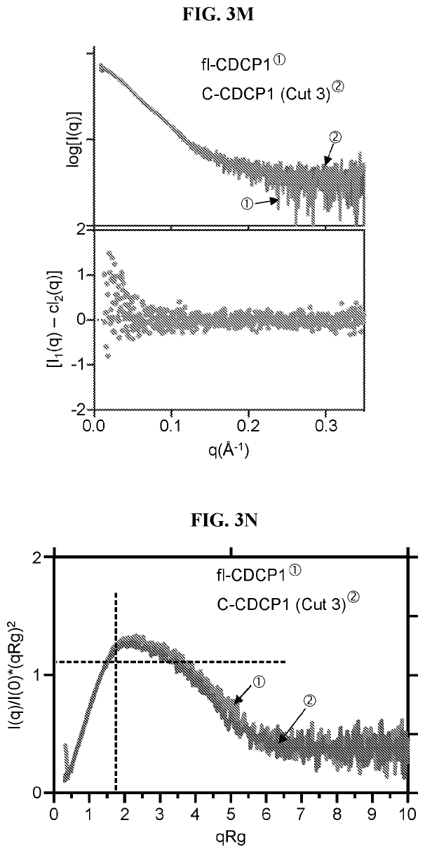

In the present example, CDCP1 antigen with endogenous cut sites was generated. Generation of the CDCP1 antigen proceeded by co-transfection of the two fragments on separate plasmids. One set of plasmids encoding the NTF of CDCP1, ending after the PI residue of the 3 different cut sites, was designed; and a second set of plasmids encoding the C-terminal ectodomain of CDCP1 starting at the P1′ residue of the 3 different cut sites (c-CDCP1 Cut 1, Cut 2, Cut 3), was designed (FIG. 3A). Uncleaved CDCP1 ectodomain was generated with a single plasmid encoding the entire ectodomain (fl-CDCP1). Following co-transfection of pairs of NTF and CTF plasmids, an intact cleaved CDCP1 complex was purified by Ni-NTA affinity chromatography for all of the NTF+CTF cut variants (FIG. 3B). In parallel, fl-CDCP1 and c-CDCP1 ectodomains fused to an Fc domain were generated for use in phage selection, using the same co-transfection strategy (FIG. 3H-FIG. 3L). Three pairs of NTF and CTF-Fc plasmids were made that ends or starts at the 3 different cut sites of cleaved CDCP1. An SDS-PAGE gel of fl-CDCP1-Fc and c-CDCP1-Fc (Cut 1, Cut 2, Cut 3) was run (FIG. 3I). BLI assays demonstrated robust binding (nm) of IgG 4A06 to fl-CDCP1-Fc and c-CDCP1-Fc (FIG. 3J).

Both fl-CDCP1 and c-CDCP1 demonstrated robust binding to IgG 4A06 (FIG. 3C) and had similar elution profiles by SEC (FIG. 3L). These data demonstrated that cleaved CDCP1 with the endogenous cut sites was indeed a complex. Remarkably, fl-CDCP1 and c-CDCP1 (Cut 1, Cut 2, Cut 3) all had similar melting temperatures (FIG. 3D), which indicated that the cleaved complex was stable and the two fragments did not dissociate until the full unfolding of the entire ectodomain. Tm was reported as an average and standard deviation of two replicates. Additionally, the conformation of fl-CDCP1 and c-CDCP1 ectodomains was evaluated by circular dichroism (CD) spectroscopy (FIG. 3E and FIG. 3M), SEC-small-angle X-ray scattering (SEC-SAXS) (FIG. 3F and FIG. 3N-FIG. 3P) and SEC-Multi-angle light scattering (SEC-MALS) (FIG. 3G). The CD spectra of fl-CDCP1 and c-CDCP1 showed a classic β-sheet signal, consistent with the CUB domain fold. Curiously, a noticeable difference in the spectral shape between fl-CDCP1 and c-CDCP1 and a shift in the minima of the curve was observed, which suggested that proteolysis caused a small change in the secondary structure of CDCP1. However, there was little difference in the SAXS profile and radius of gyration (R_{g}) between fl-CDCP1 and c-CDCP1, which indicated there was no large-scale conformational change as a result of proteolysis, and SEC-MALS showed that the two CDCP1 isoforms have the same elution profile and molecular weight (˜97-99 kDa) corresponding to the approximate size of a monomeric ectodomain (77 kDa plus glycosylation). Overall, these data demonstrated that, other than small differences in the β-sheet signature, the conformation of CDCP1 was remarkably similar between the uncleaved and cleaved form.

### Example 5: Generation of HEK293T Cell Lines Expressing Cleaved and Uncleaved CDCP1

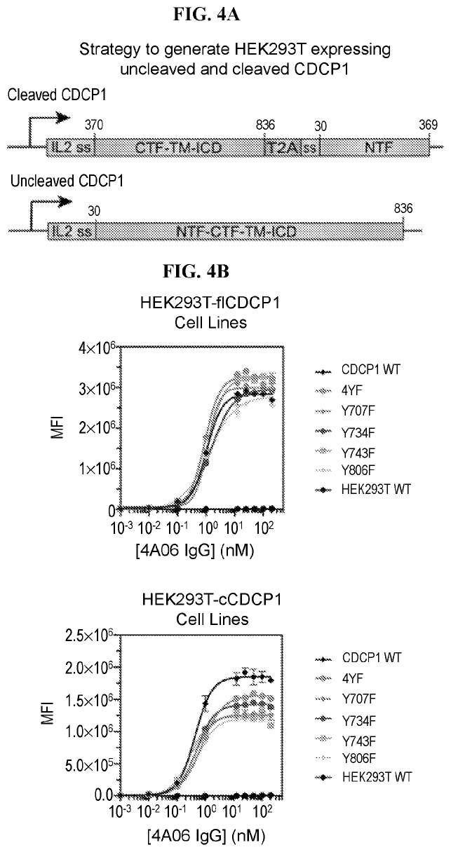

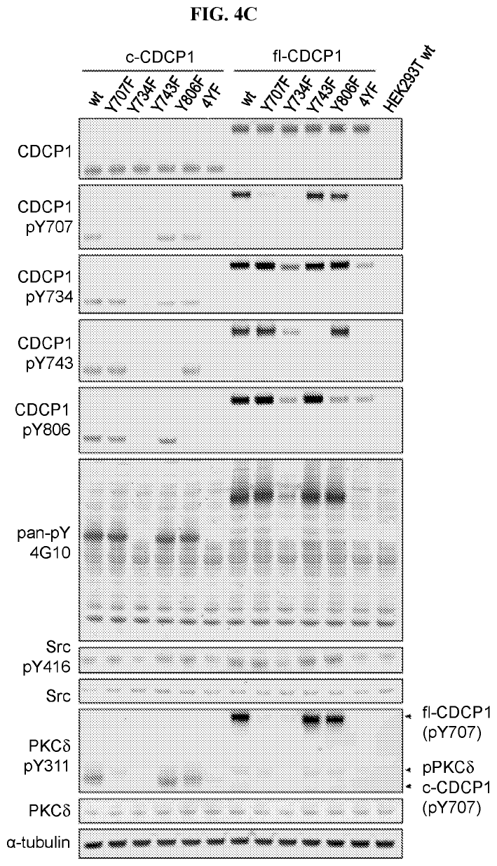

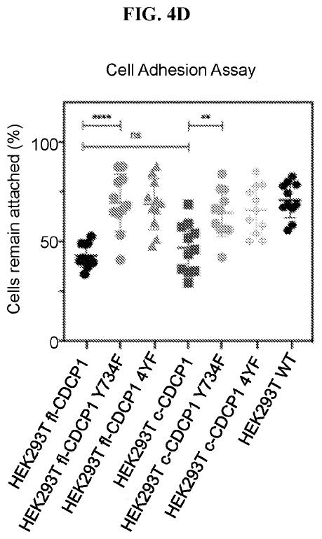

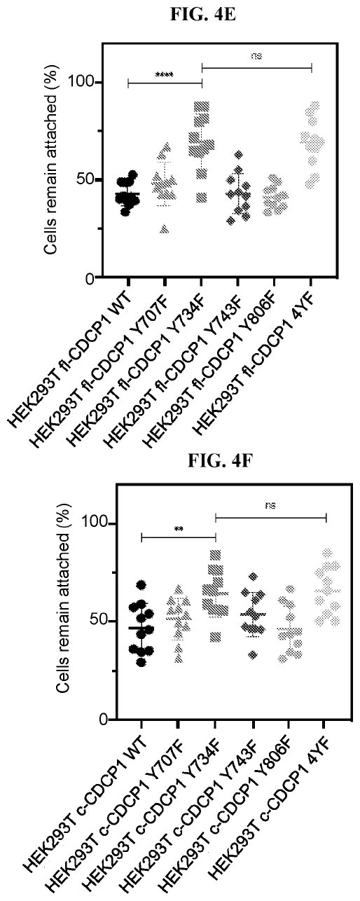

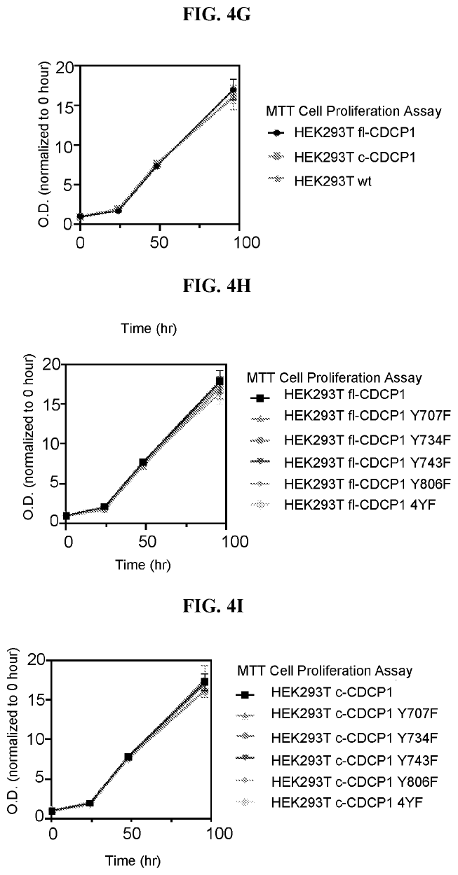

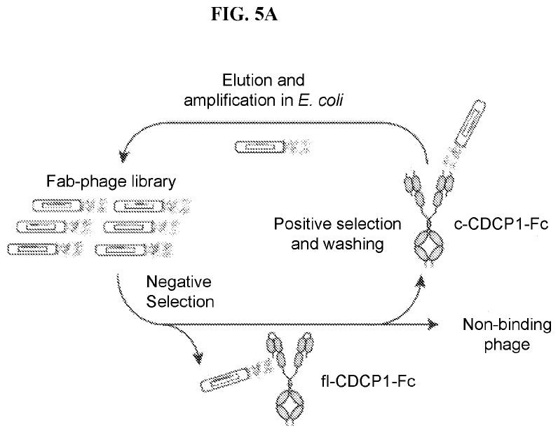

In the present example, a set of lentiviral vectors were designed and used to generate stable HEK293T cell lines expressing c-CDCP1 (Cut 3) or fl-CDCP1 for use in cell-based studies (FIG. 4A). For fl-CDCP1, a vector encoding the full CDCP1 protein sequence was used. For c-CDCP1, a vector in which a T2A self-cleaving sequence was inserted between the CTF and the NTF was designed, where the T2A self-cleaving sequence was intended to be cleaved during translation to generate two polypeptides. This approach was designed to allow for the expression of c-CDCP1 complex on the cell surface from a single lentiviral vector. In addition to wild-type c-CDCP1 and fl-CDCP1, cell lines expressing variants were generated using standard methods. The cell lines expressing variants included variants where the four intracellular tyrosine residues (Y707, Y734, Y743, Y806), which were known to be phosphorylated upon activation (see Leroy, C., et al., Oncogene 34:5593-5598 (2015)), were mutated to phenylalanine one by one (Y707F, Y734F, Y743F, Y806F) or altogether (4YF: Y707F/Y734F/Y743F/Y806F) to investigate the downstream signaling and functional consequences. It was observed by flow cytometry using IgG 4A06 (FIG. 4B) and by western blot using D1W9N Ab (FIG. 4C) that this approach successfully generated HEK293T cells expressing endogenous cleaved CDCP1 complex and uncleaved CDCP1 with the different intercellular tyrosine variants.

### Example 6: Functional Characterization of Cleaved CDCP1 Complex on HEK293T Cells

In the present example, the HEK293T cell lines expressing fl-CDCP1 and c-CDCP1 of Example 5 were used to examine the signaling pathways and functional outputs associated with proteolytic isoform.

Overexpression of CDCP1 has been known to be associated with phosphorylation of its intracellular tyrosine residues and the initiation of signaling pathways involving Src and PKC8 to promote pro-tumorigenic processes such as loss of adhesion and anoikis. See, for example, He, Y., et al., Oncogene 35:468-478 (2016); and Leroy, C., et al., Oncogene 34:5593-5598 (2015). Furthermore, both cleaved and uncleaved CDCP1 had been shown to contribute to outside-in signaling. See, for example, He, Y., et al., Oncogene 35:468-478 (2016).

Western blot analysis of phosphorylation of CDCP1, Src, and PKC8 in the different HEK293T cell lines indicated that intracellular tyrosine residues on both full-length and cleaved CDCP1 could be phosphorylated (FIG. 4C). Phosphorylation of Src and PKC8 was also observed. Additionally, it was found that Y734 was important for the phosphorylation of the other intracellular tyrosine residues of CDCP1 and the phosphorylation of Src and PKCδ.

The effects of CDCP1 expression on cell growth and adhesion were then evaluated. Cell proliferation (growth) assays were performed using an MTT modified assay to measure cell viability. In brief, 5,000 HEK293T cells expressing different CDCP1 constructs were plated in each well of a 96-well plate on day 0. Cells were incubated at 37° C. under 5% CO₂. On day 1, 2, 3 and 4, 10 μL of 5 mg/mL of Thiazolyl Blue Tetrazolium Bromide (Sigma Aldrich) was added to each well and incubated at 37° C. under 5% CO2 for 2 hrs. Following, 100 μL of 10% SDS+0.01 M HCl was added to lyse the cells to dissolve the MTT product. After 4 hrs, absorbance at 595 nm was quantified using an Infinite M200 PRO-plate reader (Tecan). Data points were plotted using Prism software (GraphPad).

Cell adhesion assays were performed as follows. The day prior to the assay, a 96-well tissue culture plate was coated with MaxGel™ ECM (Millipore Sigma) 1:10 diluted in serum-free DMEM. At the same time, the cell culture medium for HEK293T cells with expressing different CDCP1 constructs was changed to serum-free medium. The next day, media was removed and the culture plates were blocked with 100 μL serum-free DMEM with 0.1% BSA for 2 hrs and washed with PBS. 100,000 cells in 100 μL of serum-free (0.1% BSA) medium were added to each well and incubated at 37° C. in a 5% CO₂ for 2 hrs. The non-adherent cells were removed by washing with media three times, and the remaining cells were quantified by MTT assay. Data points were plotted using Prism software (GraphPad).

Both overexpression of fl-CDCP1 and c-CDCP1 significantly decreased cell adhesion and was dependent on intracellular tyrosine phosphorylation, specifically of residue Y734 (FIG. 4D-FIG. 4F). It was observed that CDCP1 overexpression did not have a significant effect on cell growth, as cell lines expressing both c-CDCP1 and fl-CDCP1 and the panel of tyrosine mutations all had similar growth rates relative to wild-type HEK293T (FIG. 4G-FIG. 4I). Taken together, the results demonstrated that cleaved CDCP1 complex was associated with known signaling pathways and cellular phenotypes of CDCP1 and validated the biological role and function of this new model of cleaved CDCP1.

### Example 7: Generation of IgG CL03.2, an Antibody Specific to Cleaved CDCP1

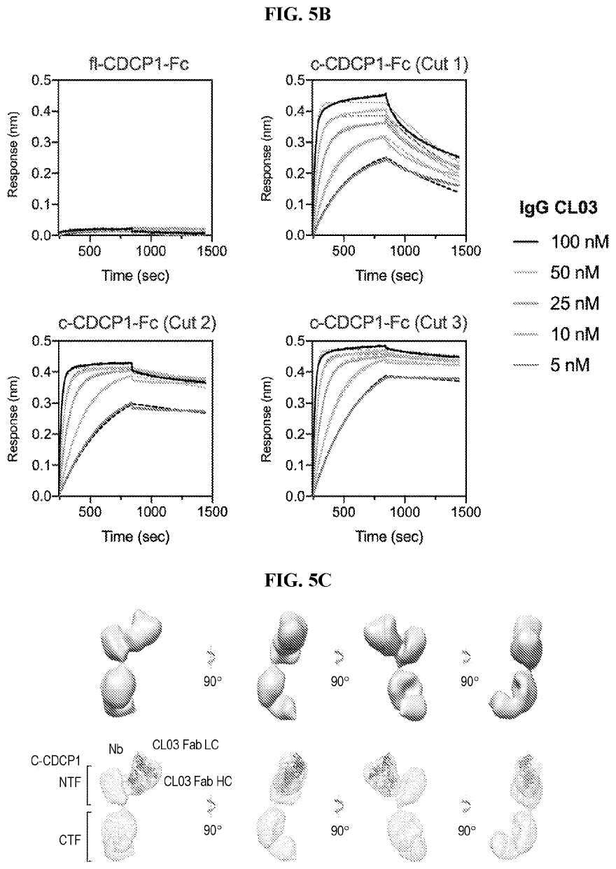

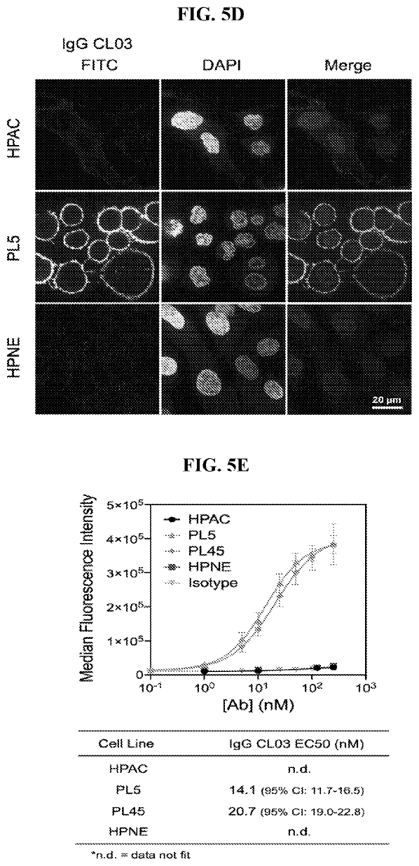

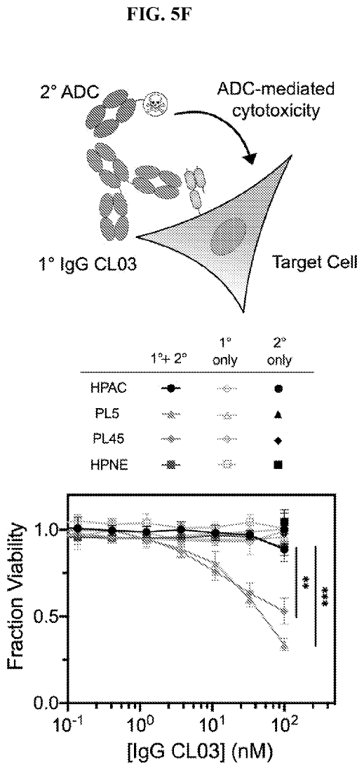

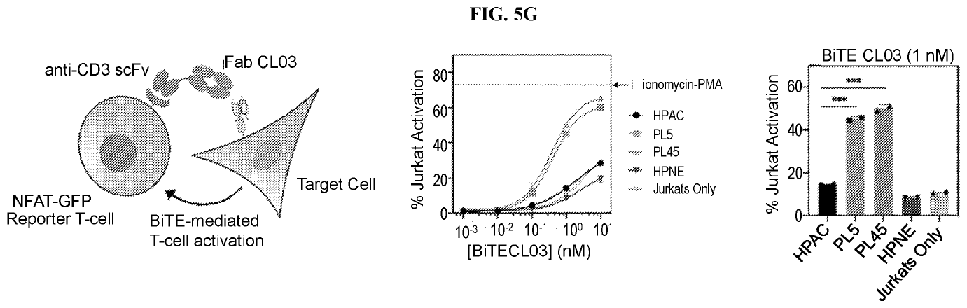

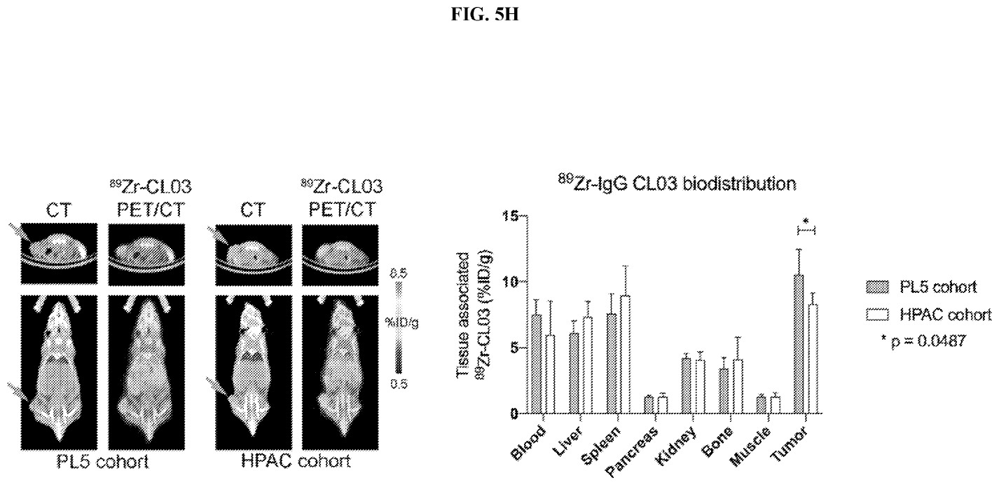

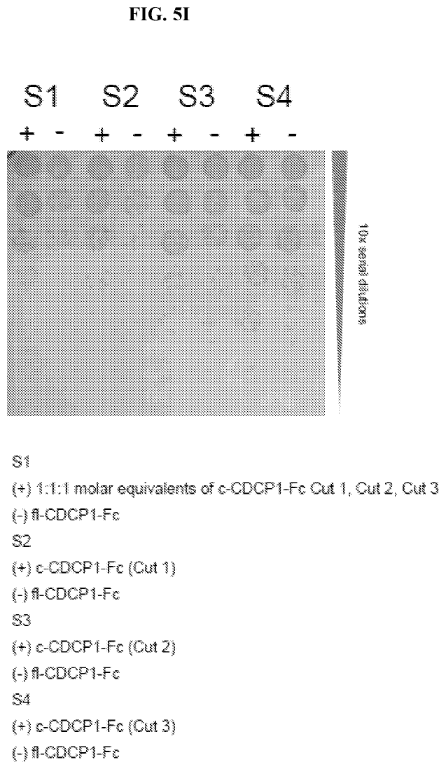

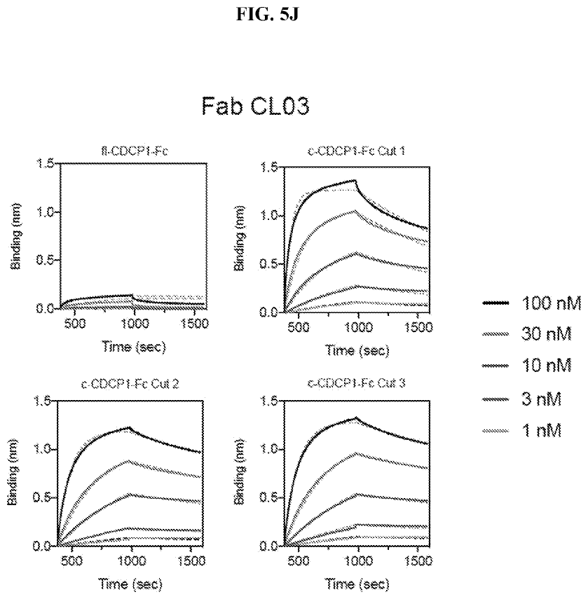

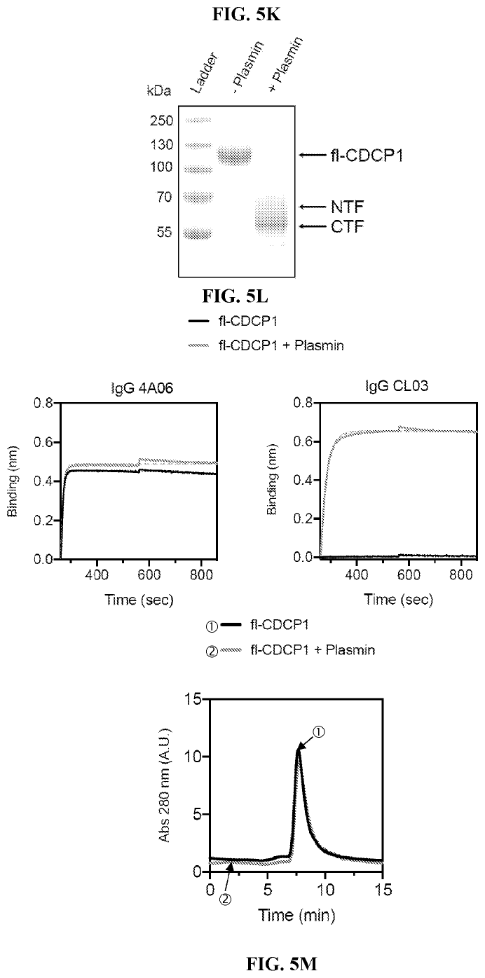

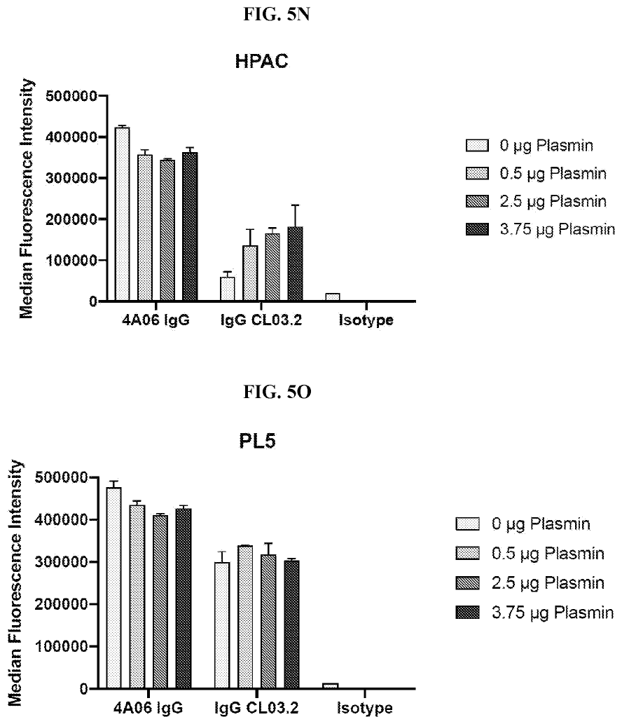

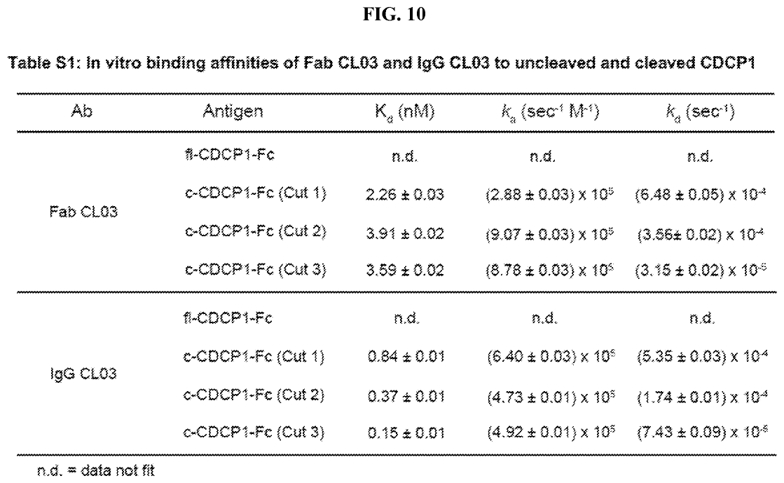

In the present example, an antibody (IgG CL03.2) specific to cleaved CDCP1 was generated. For identification of an antibody that can specifically recognize cleaved CDCP1, a differential phage selection strategy using a custom Fab-phage library was used (FIG. 5A). See, Hornsby, M., et al., Mol. Cell. Proteomics 14:2833-2847 (2015). Prior to each round of selection, the phage pool was cleared with fl-CDCP1-Fc before positive selection with c-CDCP1-Fc. The three different cut sites were selected for individually, or as a pooled c-CDCP1 antigen mix with all 3 cut site variants at 1:1:1 ratio. After 3-4 rounds of selection, there was enrichment for Fab-phage that recognized c-CDCP1-Fc over fl-CDCP1-Fc (FIG. 5I). Individual clones were analyzed by phage ELISA, and a unique clone, CL03, was identified, which clone bound all three c-CDCP1 antigens selectively over fl-CDCP1 as a Fab (FIG. 5J and FIG. 10). After conversion to an IgG scaffold and an affinity maturation campaign, IgG CL03 was identified as a lead antibody with high affinity for c-CDCP1 (K_{D}=150-840 pM), with no detectable binding to fl-CDCP1 (FIG. 5B and FIG. 10). Additionally, it was demonstrated that plasmin-treated CDCP1 could also be recognized by IgG CL03 (FIG. 5K-FIG. 5M). Since plasmin was previously reported to be one of the proteases that could cleave CDCP1 (see, for example, He, Y., et al., J. Biol. Chem. 285:26162-26173 (2010); and Deryugina, E. I., et al., Mol. Cancer Res. 7, 1197-1211 (2009)), this result indicated that IgG CL03 could recognize a physiologically relevant cleaved form of CDCP1.

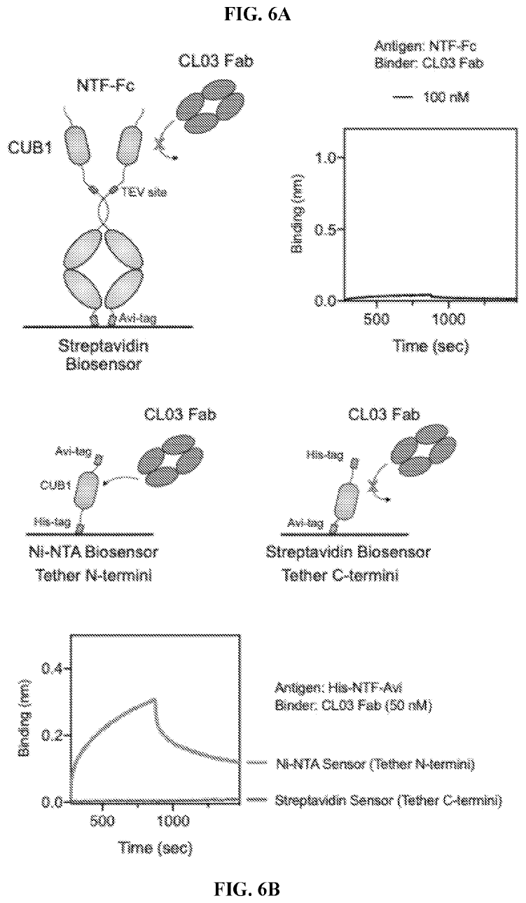

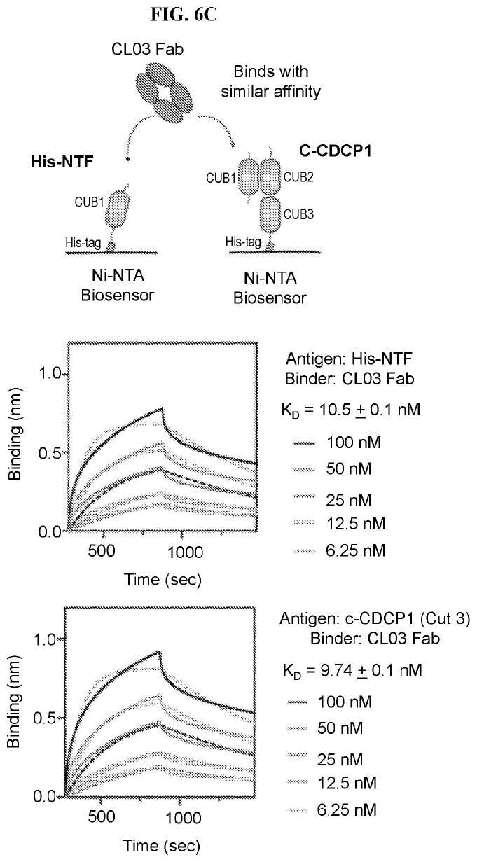

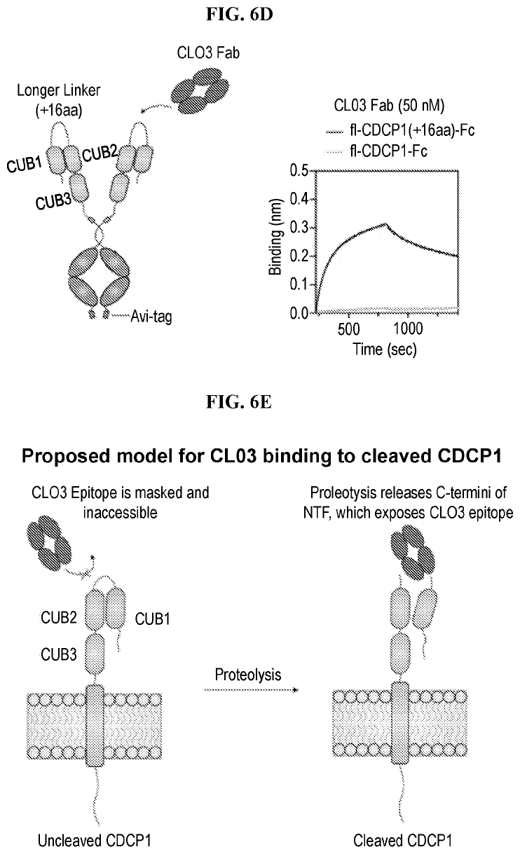

To investigate the binding mechanism of CL03, the binding of CL03 to different CDCP1 antigens was measured by BLI using standard methods. In instances when the C-termini of the NTF was immobilized either via an Fc domain or directly to the biosensor surface, there was no binding of CL03 (FIG. 6A-FIG. 6B). In instances when the NTF was immobilized via the N-termini, CL03 bound NTF at similar affinity to the full c-CDCP1 antigen (FIG. 6B-FIG. 6C). Additionally, it was observed that CL03 recognized an uncleaved CDCP1 variant where a 16-amino acid linker was inserted between CUB1 and CUB2 at the R368/K369 proteolysis site (FIG. 6D). This indicated that, akin to proteolysis, extending the linker between CUB1 and CUB2 could unmask the CL03 epitope.

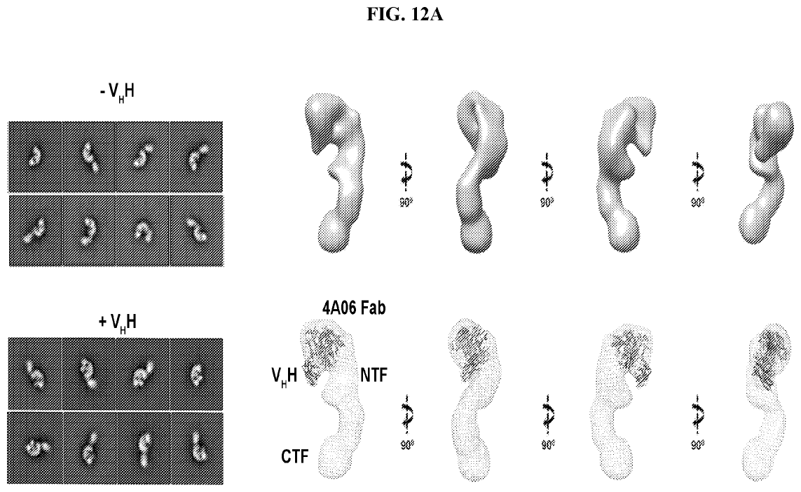

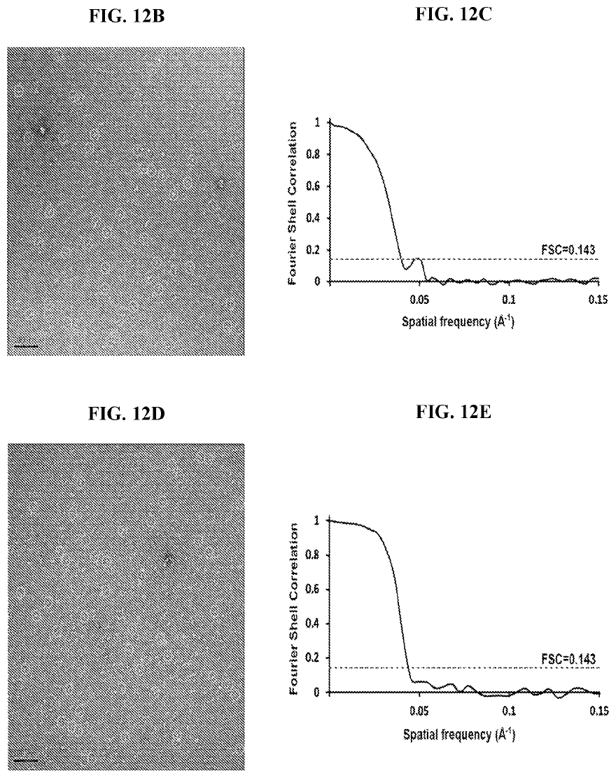

This was further investigated by negative-stain EM. A 3D reconstruction of c-CDCP1 (Cut 3) ectodomain bound to CL03 Fab at 25 Å resolution (FIG. 5C and FIG. 12B-FIG. 12C) and bound to 4A06 Fab at 23 Å resolution (FIG. 12A and FIG. 12D-FIG. 12E) was obtained. c-CDCP1 (Cut3) was incubated with CL03 Fab and a nanobody that binds at the “elbow” of the light chain, which allowed for determination of the orientation and “handedness” of the Fab. CL03 Fab appeared to bind the NTF of c-CDCP1 at its side. Taken together, the results indicated that epitope of CL03 was located on the NTF but was inaccessible in the uncleaved form. Proteolysis released the C-termini of NTF and revealed this epitope, such as, for example, via unmasking or a conformational change, and was necessary for CL03 recognition (FIG. 6E).

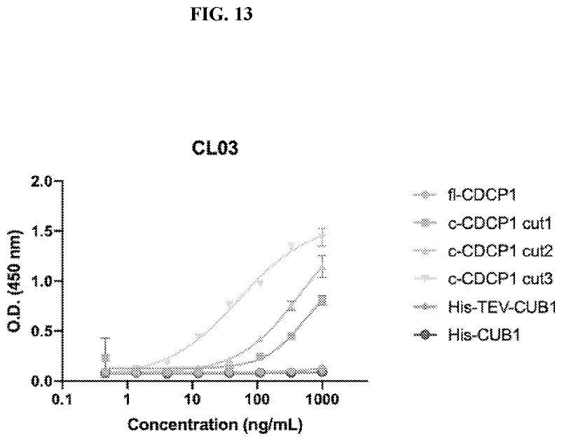

CUB1 N-terminal fragment (NTF) is present only in human serum. Since CL03 binds to an epitope on CUB1 domain that is exposed upon proteolysis, CUB1 (NTF) would serve as a sink to CL03 that decreases the viable concentration of CL03 on tumor site if it binds to CUB1 only in human serum. To further investigate the binding mechanism of CL03, an ELISA experiment was performed in which the binding of CL03 to CUB1 in human serum was evaluated. ELISA assays were performed as generally described in Example 1. Briefly, either IgG CL03 or 4A06 was used as the capture antibody; the target antigens evaluated were as presented in Table 9 below; and an anti-CDCP1 polyclonal Ab was used as the detection antibody (R&D: Cat. #AF2666). Referring now to FIG. 13, the ELISA results indicated that CL03 did not bind to CUB1 (NTF) in human serum.

The results of the ELISA assays are further summarized in Table 9 below.

TABLE 9

| Target Antigen | CL03 Binding |
|---|---|
| fl-CDCP1 ECD no Fc | − |
| cCDCP1 ECD cut 1 | + |
| cCDCP1 ECD cut 2 | + |
| cCDCP1 ECD cut 3 | + |
| CUB1 (His-TEV-CUB1, soluble NTF) | − |
| CUB1 (His-CUB1, soluble NTF) | − |

This result indicated that CUB1 only in human serum will not serve as a sink to CL03.

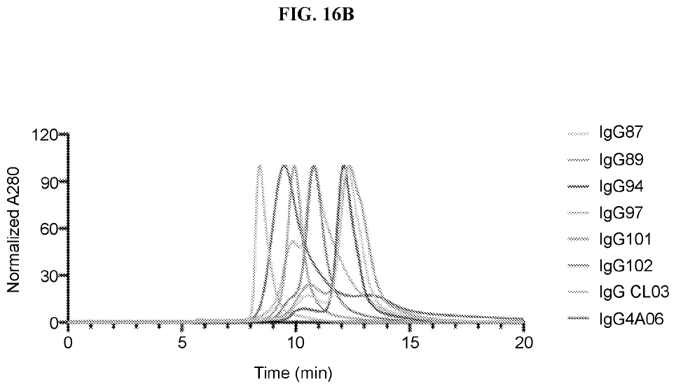

Furthermore, variants of CL03 were evaluated for binding to c-CDCP1-Fc (Cut2) by BLI assays using an Octet RED384 instrument (ForteBio) (FIG. 16A) using a procedure as generally described in Example 1. The variants also were evaluated by SEC-based analysis (FIG. 16B) as generally described in Example 1. The variants evaluated by BLI assays and SEC-based analysis included the variants referred to as IgG87, IgG89, IgG94, IgG97, IgG101 (CL03 IgG H1-NtoD H3-MtoA), and IgG102.

### Example 8: IgG CL03.2 Targets Cleaved CDCP1-Expressing PDAC Cells

In the present example, recognition of cleaved CDCP1 on cancer cells by IgG CL03 was evaluated. An immunofluorescence assay was performed using standard methods, and immunofluorescence with Alexa Fluor 488-labeled IgG CL03 showed specific staining of cleaved CDCP1-expressing PL5 cells, with no detectable binding to HPAC or HPNE cells (FIG. 5D). Dose-response flow cytometry experiments showed that IgG CL03 binds PL5 and PL45 cells with EC50 values of 14.1 and 20.7 nM, respectively with very little binding to HPAC or HPNE cells (FIG. 5E). Additionally, treatment with increasing amounts of plasmin increased IgG CL03 binding to HPAC cells, which demonstrated that treatment with a serine protease known to cleave CDCP1 could generate c-CDCP1 on the cell surface that could be recognized by IgG CL03 (FIG. 5N-FIG. 5O).

Use of CL03 in different modalities for delivery of cytotoxic or immunotherapy payloads to cleaved CDCP1-expressing PDAC cells was then evaluated. First, antibody-drug-conjugate (ADC) strategy was evaluated. HPAC, PL5, PL45, and HPNE cells were treated with IgG CL03 as the primary Ab along with a secondary antibody conjugated to cytotoxin monomethyl auorstatin F (MMAF) (FIG. 5F). Binding to cleaved CDCP1, followed by internalization of the primary and secondary antibody, delivered MMAF to cleaved CDCP1-expressing cells. Indeed, dose-dependent cell killing of only cleaved CDCP1-expressing PL5 and PL45 cells was observed, while HPAC and HPNE cells were spared. This effect was only observed in the presence of both the primary and secondary Ab, demonstrating that IgG CL03 could be used to selectively deliver a cytotoxic payload to cells expressing the cleaved isoform.

Next, re-engineering of CL03 into a bi-specific T-cell engager (BiTE) as an immunotherapy to selectively recruit and activate immune cells in the presence of cleaved CDCP1-expressing target cells was tested. Fab CL03 was genetically fused to an anti-CD3 OKT3 scFv and whether this BiTE molecule could mediate T-cell activation of an NFAT-GFP reporter Jurkat T-cell in co-culture with PDAC cells was evaluated (FIG. 5G). Significant dose-dependent T-cell activation of up to 60% when co-cultured with PL5 and PL45 cells that expressed c-CDCP1 was observed, while co-culture with HPAC and HPNE cells that did not have appreciable levels of c-CDCP1 resulted in only baseline activation.

Localization of IgG CL03 to cleaved CDCP1-expressing tumors in a mouse xenograft to be used as an in vivo imaging agent was evaluated. 89Zr-labeled IgG CL03 was injected into mice harboring HPAC or PL5 xenografts and examined 48 hours later by position-emission tomography (PET) imaging (FIG. 5H). High localization of 8°Zr-labeled IgG CL03 was observed to PL5 tumors, and the radiolabel signal was higher than to HPAC tumors (p=0.0483). Taken together, these in vitro and in vivo studies demonstrated that Ab CL03 could be used in a variety of modalities to target cleaved CDCP1 on the surface of pancreatic cancer cells.

To further evaluate the recognition of cleaved CDCP1 on cancer cells by CL03, a competition assay was performed to evaluate whether soluble CUB1 (NTF) could compete with the binding of CL03 on cleaved CDCP1 positive cells. Cells were lifted with Versene (0.04% EDTA, PBS pH 7.4 Mg/Ca free), washed once with PBS pH 7.4, and subsequently blocked with flow cytometry buffer (PBS, pH 7.4, 3% BSA). Primary Ab (CL03 IgG) and different concentrations of CUB1 (NTF) were added to cells for 30 minutes at 4° C. Bound Abs were detected with addition of AlexaFluor-488 conjugated Goat anti-human IgG, F(ab′)2 fragment specific (Jackson ImmunoResearch: 1:1000). Cells were washed 3× with PBS+3% BSA and fluorescence was quantified using a CytoFLEX (Beckman Coulter) flow cytometer. All flow cytometry data analysis was performed using FlowJo software and Prism software (GraphPad).

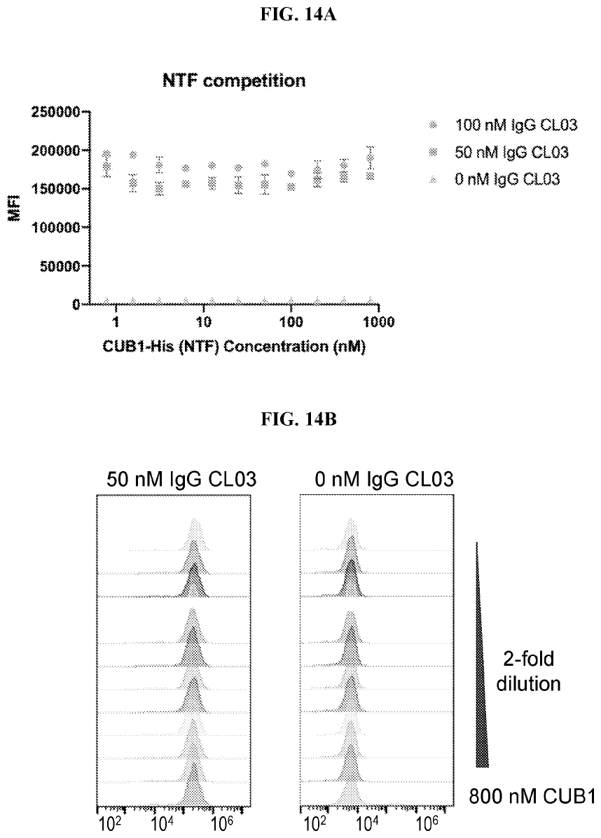

Referring now to FIG. 14A-FIG. 14B, the results of the competition assay demonstrated that soluble CUB1 (NTF) did not compete with the binding of CL03 on cleaved CDCP1 positive cells. This result further indicated that CUB1 (NTF) only in human serum will not serve as a sink to CL03.

### Example 9: Development of a Cleaved-Specific Antibody to the Mouse Homolog of CDCP1

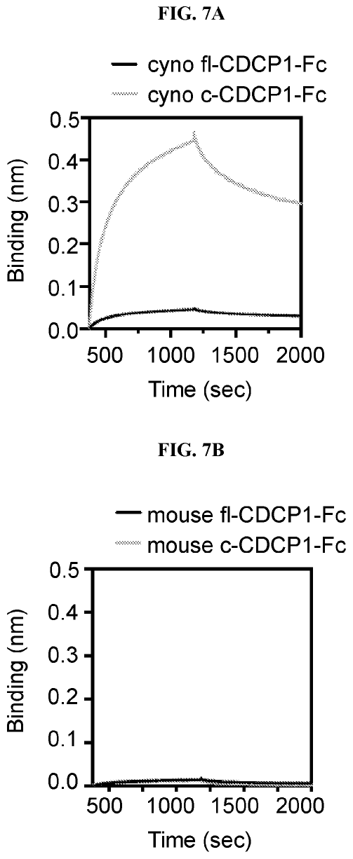

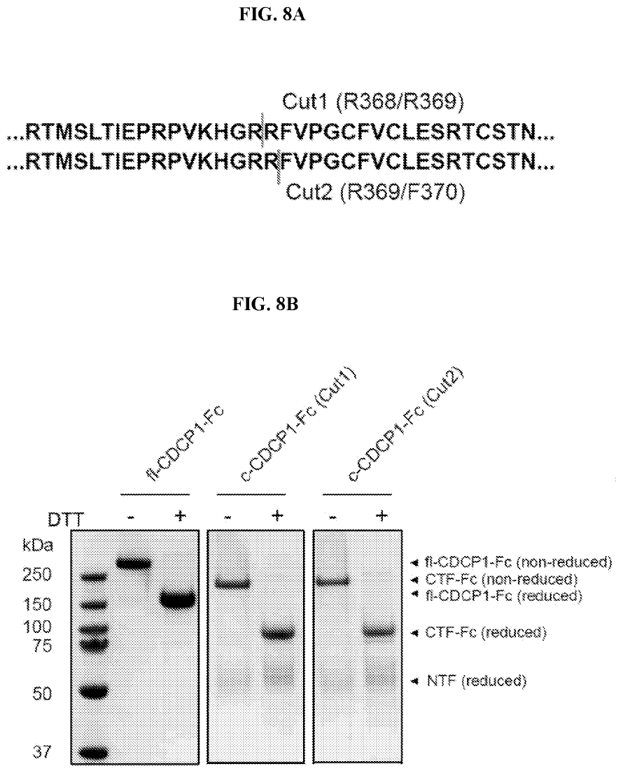

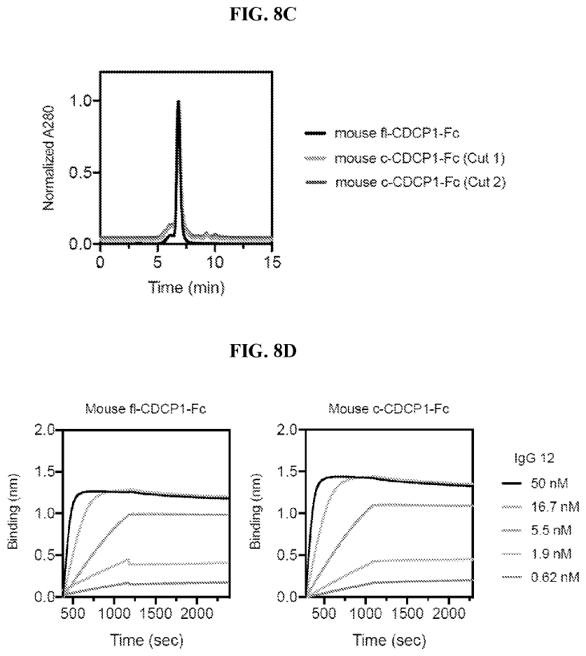

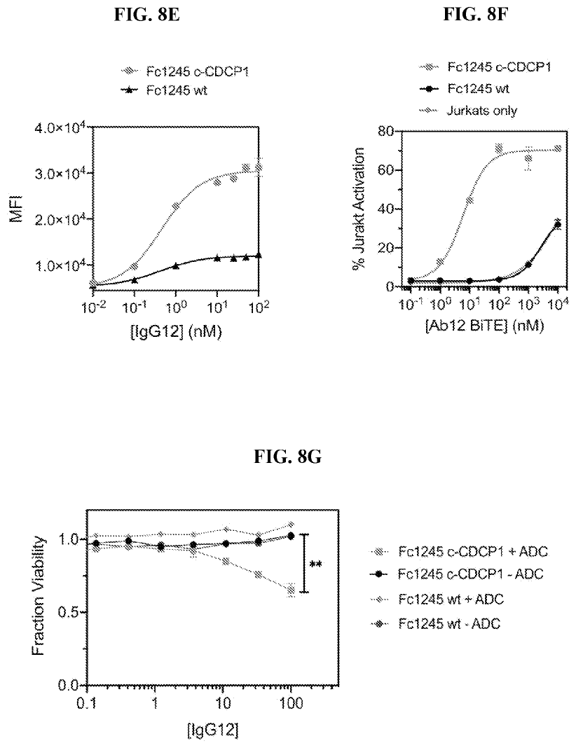

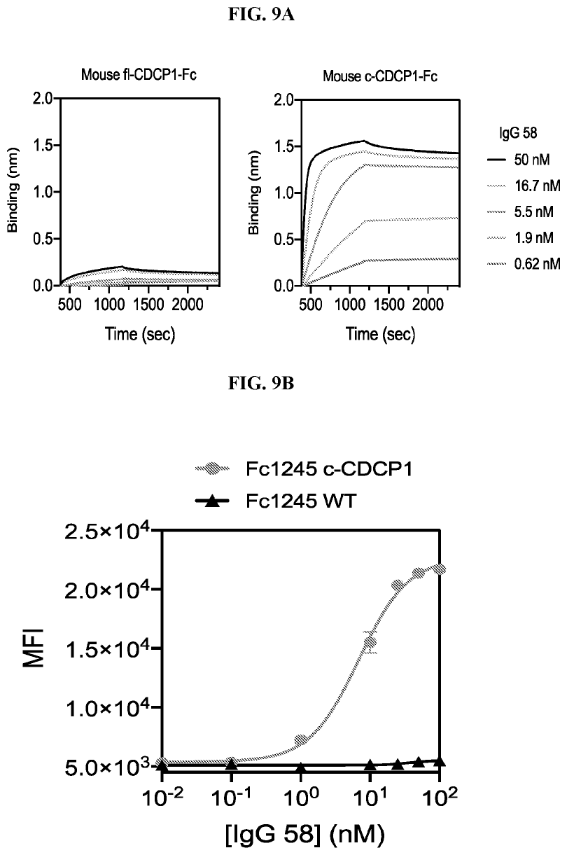

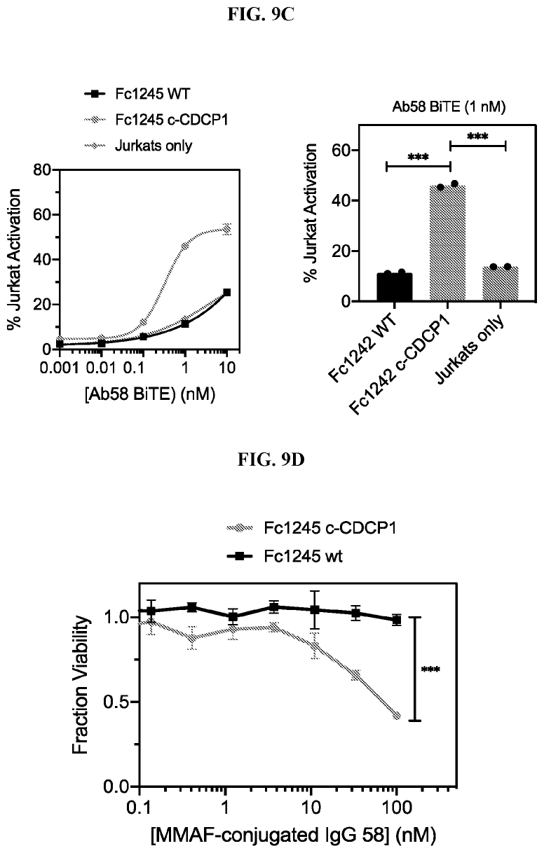

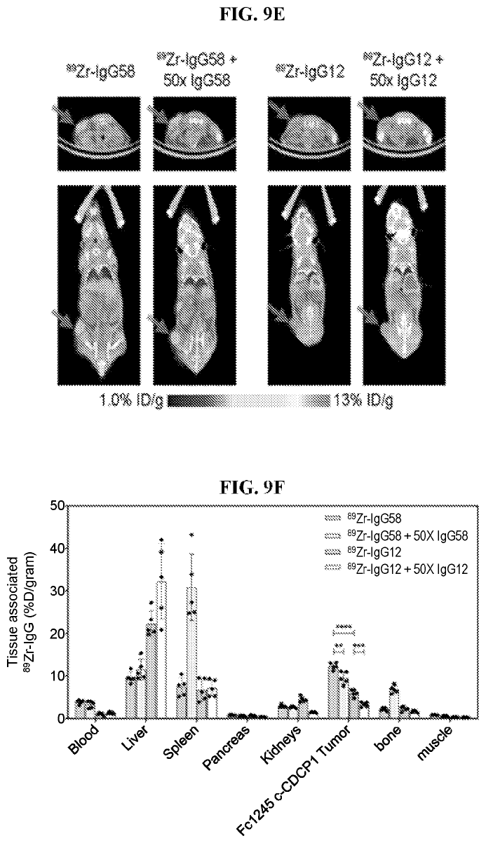

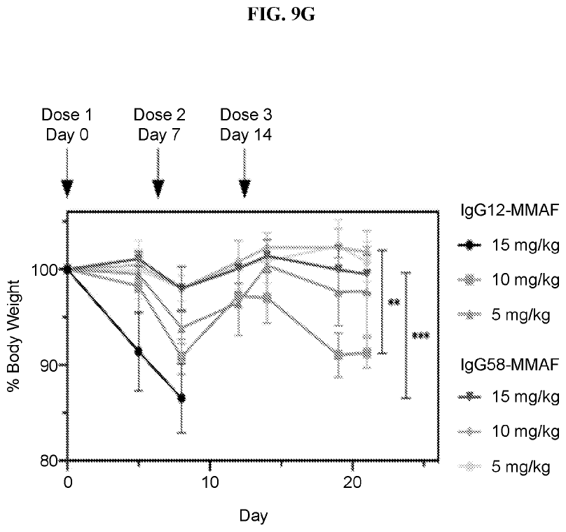

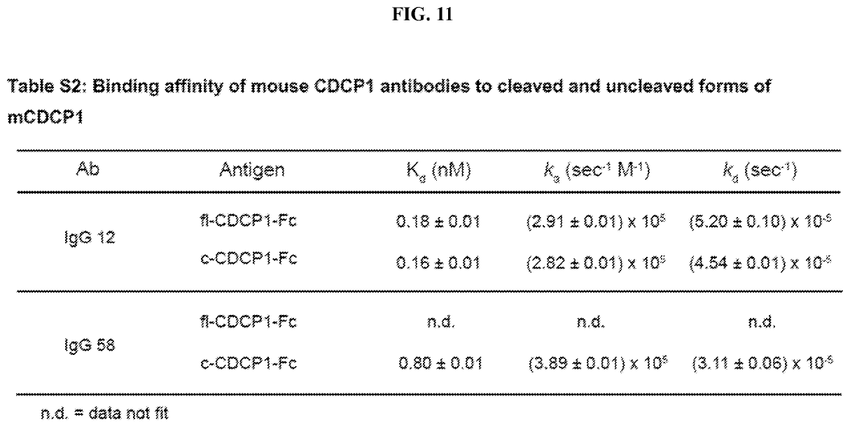

In the present example, a cleaved-specific antibody (IgG 58) to the mouse homolog of CDCP1 was developed. It was observed that CL03 was cross-reactive to the cleaved CDCP1 homolog from cynomolgous, but not to the mouse homolog (FIG. 7A-FIG. 7B). To examine the tumor-specificity of cleaved CDCP1 in a syngeneic model, the differential phage display selection strategy described supra was used against the mouse isoform of c-CDCP1 to identify binders that specifically recognize mouse c-CDCP1 but not mouse fl-CDCP1. Mouse CDCP1 antigens were characterized (FIG. 8A-FIG. 8C). Referring to FIG. 8B, an SDS-PAGE gel of mouse CDCP1 antigens: fl-CDCP1-Fc, c-CDCP1-Fc (Cut 1), c-CDCP1-Fc (Cut 2) showed proteins at the expected molecular weights. Referring to FIG. 8C, an SEC trace from SEC analysis of mouse CDCP1 antigens: fl-CDCP1-Fc, c-CDCP1-Fc (Cut 1), c-CDCP1-Fc (Cut 2) demonstrated similar elution profiles, indicating the two fragments of cleaved CDCP1 was intact as a complex. After characterization and affinity maturation, a lead mouse cleaved-specific CDCP1 antibody, IgG 58, was identified, which lead antibody binds mouse c-CDCP1 with high affinity and specificity (FIG. 9A and FIG. 11). Additionally, an antibody known as IgG 12 was identified, which, akin to the human CDCP1-specific IgG 4A06, could recognize both uncleaved and cleaved mouse CDCP1 with similar affinities (FIG. 8D and FIG. 11).

In parallel, a stable mouse cell line expressing mouse c-CDCP1 was generated using the same T2A self-cleavage sequence strategy described supra (FIG. 4A) in the background of Fc1245, a mouse KPC cancer cell line. See Roy, I., et al., Cancer Res 75:3529-3542 (2015). IgG 58 specifically recognized Fc1245 c-CDCP1 cells, binding with an EC50 of 6.9 nM and 0.46 nM, respectively (FIG. 9B and FIG. 8E). IgG 58, when re-formatted to a BiTE molecule, activated NFAT-GFP Jurkat cells in the presence of Fc1245 c-CDCP1 cells (FIG. 9C and FIG. 8F). Additionally, MMAF was directly conjugated to IgG 58, and it was shown that IgG 58 could be used as an ADC to specifically deliver cytotoxic payloads to Fc1245 c-CDCP1 cells (FIG. 9D and FIG. 8G) while sparing Fc1245 WT cells that did not express c-CDCP1.

Furthermore, it was tested whether IgG12 and IgG58 can localize to Fc1245 c-CDCP1 tumors in vivo. ⁸⁹Zr-labeled IgG12 or IgG58 was injected into mice harboring a subcutaneous Fc1245 c-CDCP1 tumor and examined 48 hours later by position-emission tomography (PET) imaging (FIG. 9E and FIG. 9F).

High tumor localization of ⁸⁹Zr-IgG58 was observed and decreased when 50× unlabeled IgG58 was co-administered, indicating tumor-specific localization driven by target engagement. Little localization of ⁸⁹Zr-IgG58 was observed outside of the tumor. In contrast, although specific localization of ⁸⁹Zr-IgG12 to the tumor was observed, the signal was lower than that of IgG58. Additionally, a broader normal tissue distribution of IgG12 was observed, which indicates that IgG12 may be localizing to normal tissue that express uncleaved CDCP1.

Next, the anti-tumor activity and toxicity profile of MMAF-labeled IgG12 or IgG58 was examined (FIG. 9G). Non-tumor-bearing mice were dosed weekly with 15, 10, or 5 mg/kg of MMAF-labeled IgG12 and IgG58, and their body weight (%) was monitored for 21 days. There was a significant difference between the treatment arms (F(5,32)=3.11, p=0.0002, ANOVA), with IgG58-MMAF treatment being better tolerated. None of the mice injected with IgG58-MMAF at the 3 different doses exhibited significant changes in body weight. Mice treated with IgG12-MMAF experienced significant body weight loss following the administration of each dose, indicative of treatment-induced toxicity.

Tukey's multiple comparisons test revealed significant differences in the outcomes between IgG12-MMAF and IgG58-MMAF treatment at the 15 mg/kg dose (***p=0.0068) and 10 mg/kg dose (**p=0.0067). All of the mice receiving the 15 mg/kg dose of IgG12-MMAF had to be euthanized due to body weight loss and did not survive past day 8, and 2 of the 5 mice receiving the 10 mg/kg dose of IgG12-MMAF had to be euthanized on day 19. These results suggest that a cleaved CDCP1-targeting therapy would have a superior safety profile compared to a pan-CDCP1-targeting approach.

### Example 10: In Vivo Syngeneic Mouse Studies of IgG 58

In the present example, in vivo syngeneic mouse studies were performed to evaluate the anti-tumor efficacy of the cleaved CDCP1 specific antibody IgG58 in a mouse model of pancreatic cancer. Briefly, Fc1245 c-CDCP1 tumor-bearing mice were injected with either PBS (control) or a 400 uCi single injection of 177Lu-labeled IgG58 (test). Five mice were used for each of the control and test groups. The injections were performed five days after tumor implantation. Tumor size and body weight were monitored until the tumor size became too large, such as can be indicated by the development of ulcerated tumors.

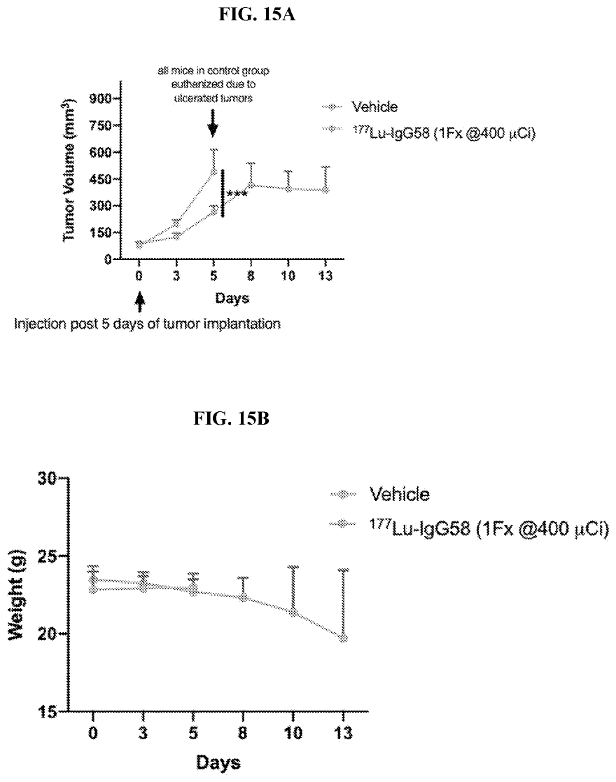

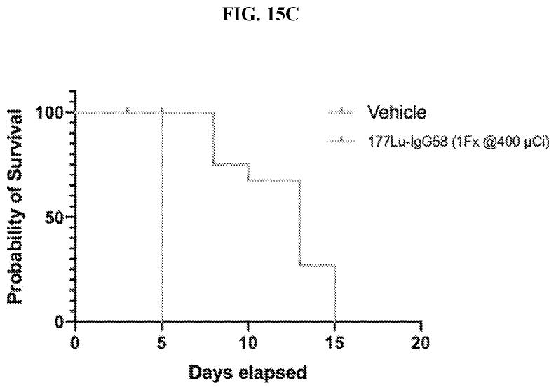

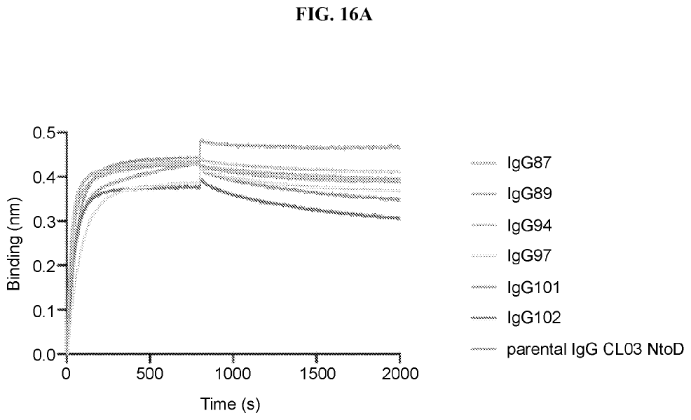

Referring now to FIG. 15A-FIG. 15C, 177Lu-labelled IgG58 efficiently inhibited tumor progression of an aggressive mouse pancreatic tumor as evaluated by tumor volume (FIG. 15A), mouse weight (FIG. 15B), and probability of survival (FIG. 15C). The mice of the control group were euthanized due to the development of ulcerated tumors as indicated by the arrow in FIG. 15A.

The foregoing description of the specific aspects will so fully reveal the general nature of the invention that others can, by applying knowledge within the skill of the art, readily modify and/or adapt for various applications such specific aspects, without undue experimentation, without departing from the general concept of the present invention. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

Other aspects of the invention will be apparent to those skilled in the art from consideration of the specification and practice of the invention disclosed herein. It is intended that the specification and examples be considered as exemplary only, with a true scope and spirit of the invention being indicated by the following claims.

All publications, patents, and patent applications disclosed herein are incorporated by reference to the same extent as if each individual publication, patent or patent application was specifically and individually indicated to be incorporated by reference.

What is claimed is:

1. An isolated antibody or antigen-binding fragment thereof that specifically binds to a cleaved human complement C1r/C1s, Uegf, Bmp1 (CUB)-domain containing protein 1 (CDCP1), wherein the antibody or antigen-binding fragment thereof preferentially binds to the cleaved CDCP1.

2. The antibody or antigen-binding fragment thereof of claim 1, which does not bind to a full-length human CDCP1 at a detectable level.

3. The antibody or antigen-binding fragment thereof of claim 1 or 2, wherein the binding between the antibody or antigen-binding fragment thereof and the cleaved CDCP1 or the full-length CDCP1 is measured by biolayer interferometry analysis using an Octet instrument (ForteBio).

4. The antibody or antigen-binding fragment thereof of any one of claims 1 to 3, wherein the cleaved CDCP1 comprises a first cleaved domain and second cleaved domain, wherein the first cleaved domain and the second cleaved domain are not linked.

5. The antibody or antigen-binding fragment thereof of any one of claims 1 to 3, wherein the cleaved CDCP1 comprises a membrane-bound complex.

6. The antibody or antigen-binding fragment thereof of claim 4 or 5, wherein the first cleaved domain consists of the amino acid sequence as set forth in SEQ ID NO: 63, 68, or 74.

7. The antibody or antigen-binding fragment thereof of any one of claims 4 to 6, wherein the second cleaved domain consists of the amino acid sequence as set forth in SEQ ID NO: 64, 70, or 77.

8. The antibody or antigen-binding fragment thereof of any one of claims 1 to 7, wherein the cleaved CDCP1 is generated by being cleaved at residue K365, R368, and/or K369 of SEQ ID NO: 273.

9. The antibody or antigen-binding fragment thereof of any one of claims 1 to 8, wherein the cleaved CDCP1 is post translationally modified, wherein the post translational modification comprises phosphorylation and N-linked glycosylation.

10. An isolated antibody or antigen-binding fragment thereof that specifically binds to a cleaved human CDCP1 and comprises a light chain variable region (VL) and a heavy chain variable region (VH); wherein the VL comprises a VL complementarity determining region (CDR) 1, a VL-CDR2, and a VL-CDR3, and the VH comprises a VH-CDR1, a VH-CDR2, and a VH-CDR3 sequences of SEQ ID NOs: 1 (SVSSAVA), 2 (SASSLY), 268 (SX₁X₂X₃X₄X₅), 269 (X₆FSSX₇SI), 270 (SIYPYSGSTX₈), and 271 (X₉X₁₀X₁₂SX₁₂YSHTWWVSYGX₁₃) or 272 (X₁₄YWVX₁₅FWYGHFSYYRPAL), respectively, wherein:
X₁=Glycine (G), Serine(S), Methionine (M), Leucine (L), Valine (V), or Arginine (R);
X₂=Glutamine (Q), Serine(S), Glutamic acid (E), Asparagine (N), Lysine (K), Proline (P), Arginine (R), Leucine (L), or Histidine (H);
X₃=Arginine (R), Serine(S), Valine (V), Tryptophan (W), Leucine (L), Lysine (K), Methionine (M), Glutamine (Q), or Proline (P);
X₄=Proline (P), Leucine (L), Threonine (T), or Serine(S);
X₅=Isoleucine (I), Alanine (A), Methionine (M), Lysine (K), Valine (V), Leucine (L), Phenylalanine (F);
X₆=No Amino Acid, Aspartic acid (D), or Asparagine (N);
X₇=Serine(S) or Tyrosine (Y);
X₈=Serine(S) or Tyrosine (Y);
X₉=Glutamine (Q), Arginine (R), or Lysine (K);
X₁₀=Serine(S), Asparagine (N), Threonine (T), Glycine (G), Alanine (A), or Aspartic acid (D);
X₁₁=Glutamine (Q) or Histidine (H);
X₁₂=Tyrosine (Y) or Phenylalanine (F);
X₁₃=Methionine (M), Alanine (A), Isoleucine (I), Leucine (L), or Valine (V);
X₁₄=Threonine (T) or Isoleucine (I); and
X₁₅=Glutamine (Q) or Aspartic acid (D).

11. An isolated antibody or antigen-binding fragment thereof that specifically binds to a cleaved human CDCP1 and comprises a light chain variable region (VL) and a heavy chain variable region (VH); wherein the VL comprises a VL complementarity determining region (CDR) 1 sequence of SEQ ID NO: 1 (SVSSAVA), a VL-CDR2 sequence of SEQ ID NO: 2 (SASSLY), and a VL-CDR3 sequence of SEQ ID NOs: 8 (TGQRPM), 23 (FMRPAF), 16 (TAQSPL), 11 (VELVPM), 12 (AGKRPL), or 14 (LGVRAA), and the VH comprises a VH-CDR1 sequence of SEQ ID NO: 269 (X₁FSSX₂SI), a VH-CDR2 sequence of SEQ ID NO: 270 (SIYPYSGSTX₃), and a VH-CDR3 sequence of SEQ ID NO: 271 (X₄X₅X₆SX₇YSHTWWVSYGX₈) or SEQ ID NO: 272 (X₉YWVX₁₀FWYGHFSYYRPAL), respectively, wherein:
X₁=No Amino Acid, Aspartic acid (D), or Asparagine (N);
X₂=Serine(S) or Tyrosine (Y);
X₃-Serine(S) or Tyrosine (Y);
X₄=Glutamine (Q), Arginine (R), or Lysine (K);
X₅=Serine(S), Asparagine (N), Threonine (T), Glycine (G), Alanine (A), or Aspartic acid (D);
X₆-Glutamine (Q) or Histidine (H);
X₇=Tyrosine (Y) or Phenylalanine (F);
X₈=Methionine (M), Alanine (A), Isoleucine (I), Leucine (L), or Valine (V);
X₉=Threonine (T) or Isoleucine (I); and
X₁₀=Glutamine (Q) or Aspartic acid (D).

12. An isolated antibody or antigen-binding fragment thereof that specifically binds to a cleaved human CDCP1 and comprises a light chain variable region (VL) and a heavy chain variable region (VH); wherein the VL comprises a VL complementarity determining region (CDR) 1, a VL-CDR2, and a VL-CDR3 and the VH comprises a VH-CDR1, a VH-CDR2, and a VH-CDR3; wherein the VL-CDR3 comprises an amino acid sequence selected from the group consisting of SEQ ID NOs: 3-25.

13. The antibody or antigen-binding fragment thereof of claim 12, wherein the VL-CDR2 comprises an amino acid of SEQ ID NO: 2.

14. The antibody or antigen-binding fragment thereof of claim 12 or 13, wherein the VL-CDR1 comprises an amino acid sequence of SEQ ID NO: 1.

15. The antibody or antigen-binding fragment thereof of any one of claims 12 to 14, wherein the VH-CDR1 comprises an amino acid sequence selected from the group consisting of SEQ ID NOs: 26-29, 109, and 111.

16. The antibody or antigen-binding fragment thereof of any one of claims 12 to 15, wherein the VH-CDR2 comprises an amino acid sequence of SEQ ID NO: 30 or 31.

17. The antibody or antigen-binding fragment thereof of any one of claims 12 to 16, wherein the VH-CDR3 comprises an amino acid sequence selected from the group consisting of SEQ ID NOs: 20, 32-47, and 105.

18. The antibody or antigen-binding fragment thereof of any one of claims 12 to 17, wherein
(a) the VL-CDR1 comprises the amino acid sequence set forth in SEQ ID NO: 1, the VL-CDR2 comprises the amino acid sequence set forth in SEQ ID NO: 2, the VL-CDR3 comprises the amino acid sequence set forth in SEQ ID NO: 3, the VH-CDR1 comprises the amino acid sequence set forth in SEQ ID NO: 26, the VH-CDR2 comprises the amino acid sequence set forth in SEQ ID NO: 30, and the VH-CDR3 comprises the amino acid sequence set forth in SEQ ID NO: 32;
(b) the VL-CDR1 comprises the amino acid sequence set forth in SEQ ID NO: 1, the VL-CDR2 comprises the amino acid sequence set forth in SEQ ID NO: 2, the VL-CDR3 comprises the amino acid sequence set forth in SEQ ID NO: 3, the VH-CDR1 comprises the amino acid sequence set forth in SEQ ID NO: 26, the VH-CDR2 comprises the amino acid sequence set forth in SEQ ID NO: 30, and the VH-CDR3 comprises the amino acid sequence set forth in SEQ ID NO: 33;
(c) the VL-CDR1 comprises the amino acid sequence set forth in SEQ ID NO: 1, the VL-CDR2 comprises the amino acid sequence set forth in SEQ ID NO: 2, the VL-CDR3 comprises the amino acid sequence set forth in SEQ ID NO: 3, the VH-CDR1 comprises the amino acid sequence set forth in SEQ ID NO: 26, the VH-CDR2 comprises the amino acid sequence set forth in SEQ ID NO: 30, and the VH-CDR3 comprises the amino acid sequence set forth in SEQ ID NO: 34;
(d) the VL-CDR1 comprises the amino acid sequence set forth in SEQ ID NO: 1, the VL-CDR2 comprises the amino acid sequence set forth in SEQ ID NO: 2, the VL-CDR3 comprises the amino acid sequence set forth in SEQ ID NO: 3, the VH-CDR1 comprises the amino acid sequence set forth in SEQ ID NO: 26, the VH-CDR2 comprises the amino acid sequence set forth in SEQ ID NO: 30, and the VH-CDR3 comprises the amino acid sequence set forth in SEQ ID NO: 35; or
(e) the VL-CDR1 comprises the amino acid sequence set forth in SEQ ID NO: 1, the VL-CDR2 comprises the amino acid sequence set forth in SEQ ID NO: 2, the VL-CDR3 comprises the amino acid sequence set forth in SEQ ID NO: 3, the VH-CDR1 comprises the amino acid sequence set forth in SEQ ID NO: 26, the VH-CDR2 comprises the amino acid sequence set forth in SEQ ID NO: 30, and the VH-CDR3 comprises the amino acid sequence set forth in SEQ ID NO: 36.

19. The antibody or antigen-binding fragment thereof of any one of claims 12 to 18, wherein the VH comprises an amino acid sequence at least about 70%, at least about 80%, at least about 90%, at least about 95%, at least about 96%, at least about 97%, at least about 98%, or at least about 99% sequence identity to an amino acid sequence selected from the group consisting of SEQ ID NOs: 61, 65, 67, 69, 71, 73, 75, 79, 80, 81, 82, 83, 84, 85, 86, 87, 89, 91, 99, 103, 107, 123, and 133.

20. The antibody or antigen-binding fragment thereof of any one of claims 10 to 17, the VH comprises an amino acid sequence selected from the group consisting of SEQ ID NOs: 61, 65, 67, 69, 71, 73, 75, 79, 80, 81, 82, 83, 84, 85, 86, 87, 89, 91, 99, 103, 107, 123, and 133.

21. The antibody or antigen-binding fragment thereof of any one of claims 12 to 20, wherein the VL comprises an amino acid sequence at least about 70%, at least about 80%, at least about 90%, at least about 95%, at least about 96%, at least about 97%, at least about 98%, or at least about 99% sequence identity to an amino acid sequence selected from the group consisting of SEQ ID NOs: 62, 76, 94, 96, 98, 100, 102, 104, 106, 108, 110, 112, 114, 116, 118, 120, 122, 124, 126, 128, 130, and 132.

22. The antibody or antigen-binding fragment thereof of any one of claims 10 to 19, wherein the VL comprises an amino acid sequence selected from the group consisting of SEQ ID NOs: 62, 76, 94, 96, 98, 100, 102, 104, 106, 108, 110, 112, 114, 116, 118, 120, 122, 124, 126, 128, 130, and 132.

23. The antibody or antigen-binding fragment thereof of any one of claims 12 to 22, wherein
(a) the VH of the reference antibody comprises the amino acid sequence set forth in SEQ ID NO: 61 and the VL of the reference antibody comprises the amino acid sequence set forth in SEQ ID NO: 62;
(b) the VH of the reference antibody comprises the amino acid sequence set forth in SEQ ID NO: 65 and the VL of the reference antibody comprises the amino acid sequence set forth in SEQ ID NO: 62;
(c) the VH of the reference antibody comprises the amino acid sequence set forth in SEQ ID NO 67 and the VL of the reference antibody comprises the amino acid sequence set forth in SEQ ID NO: 62;
(d) the VH of the reference antibody comprises the amino acid sequence set forth in SEQ ID NO: 69 and the VL of the reference antibody comprises the amino acid sequence set forth in SEQ ID NO: 62;
(e) the VH of the reference antibody comprises the amino acid sequence set forth in SEQ ID NO 71; and the VL of the reference antibody comprises the amino acid sequence set forth in SEQ ID NO: 62;
(f) the VH of the reference antibody comprises the amino acid sequence set forth in SEQ ID NO: 73 and the VL of the reference antibody comprises the amino acid sequence set forth in SEQ ID NO: 62;
(g) the VH of the reference antibody comprises the amino acid sequence set forth in SEQ ID NO: 75 and the VL of the reference antibody comprises the amino acid sequence set forth in SEQ ID NO: 76;
(h) the VH of the reference antibody comprises the amino acid sequence set forth in SEQ ID NO: 79 and the VL of the reference antibody comprises the amino acid sequence set forth in SEQ ID NO: 76;
(i) the VH of the reference antibody comprises the amino acid sequence set forth in SEQ ID NO: 65 and the VL of the reference antibody comprises the amino acid sequence set forth in SEQ ID NO: 76;
(j) the VH of the reference antibody comprises the amino acid sequence set forth in SEQ ID NO: 83 and the VL of the reference antibody comprises the amino acid sequence set forth in SEQ ID NO: 62;
(k) the VH of the reference antibody comprises the amino acid sequence set forth in SEQ ID NO: 85 and the VL of the reference antibody comprises the amino acid sequence set forth in SEQ ID NO: 62;
(l) the VH of the reference antibody comprises the amino acid sequence set forth in SEQ ID NO: 87 and the VL of the reference antibody comprises the amino acid sequence set forth in SEQ ID NO: 62;
(m) the VH of the reference antibody comprises the amino acid sequence set forth in SEQ ID NO: 89 and the VL of the reference antibody comprises the amino acid sequence set forth in SEQ ID NO: 62;
(n) the VH of the reference antibody comprises the amino acid sequence set forth in SEQ ID NO: 91 and the VL of the reference antibody comprises the amino acid sequence set forth in SEQ ID NO: 62;
(o) the VH of the reference antibody comprises the amino acid sequence set forth in SEQ ID NO: 65 and the VL of the reference antibody comprises the amino acid sequence set forth in SEQ ID NO: 94;
(p) the VH of the reference antibody comprises the amino acid sequence set forth in SEQ ID NO: 65 and the VL of the reference antibody comprises the amino acid sequence set forth in SEQ ID NO: 96;
(q) the VH of the reference antibody comprises the amino acid sequence set forth in SEQ ID NO: 65 and the VL of the reference antibody comprises the amino acid sequence set forth in SEQ ID NO: 98;
(r) the VH of the reference antibody comprises the amino acid sequence set forth in SEQ ID NO: 99 and the VL of the reference antibody comprises the amino acid sequence set forth in SEQ ID NO: 100;
(s) the VH of the reference antibody comprises the amino acid sequence set forth in SEQ ID NO: 65 and the VL of the reference antibody comprises the amino acid sequence set forth in SEQ ID NO: 102;
(t) the VH of the reference antibody comprises the amino acid sequence set forth in SEQ ID NO:103 and the VL of the reference antibody comprises the amino acid sequence set forth in SEQ ID NO: 104;
(u) the VH of the reference antibody comprises the amino acid sequence set forth in SEQ ID NO: 99 and the VL of the reference antibody comprises the amino acid sequence set forth in SEQ ID NO: 106;
(v) the VH of the reference antibody comprises the amino acid sequence set forth in SEQ ID NO: 107 and the VL of the reference antibody comprises the amino acid sequence set forth in SEQ ID NO: 108;
(w) the VH of the reference antibody comprises the amino acid sequence set forth in SEQ ID NO: 99 and the VL of the reference antibody comprises the amino acid sequence set forth in SEQ ID NO: 110;
(x) the VH of the reference antibody comprises the amino acid sequence set forth in SEQ ID NO: 99 and the VL of the reference antibody comprises the amino acid sequence set forth in SEQ ID NO: 112;
(y) the VH of the reference antibody comprises the amino acid sequence set forth in SEQ ID NO: 65 and the VL of the reference antibody comprises the amino acid sequence set forth in SEQ ID NO: 114;
(aa) the VH of the reference antibody comprises the amino acid sequence set forth in SEQ ID NO: 99 and the VL of the reference antibody comprises the amino acid sequence set forth in SEQ ID NO: 116;
(bb) the VH of the reference antibody comprises the amino acid sequence set forth in SEQ ID NO: 65 and the VL of the reference antibody comprises the amino acid sequence set forth in SEQ ID NO: 118;
(cc) the VH comprises the amino acid sequence set forth in SEQ ID NO: 65 and the VL comprises the amino acid sequence set forth in SEQ ID NO: 120;
(dd) the VH of the reference antibody comprises the amino acid sequence set forth in SEQ ID NO: 65 and the VL of the reference antibody comprises the amino acid sequence set forth in SEQ ID NO: 122;
(ee) the VH of the reference antibody comprises the amino acid sequence set forth in SEQ ID NO: 123 and the VL of the reference antibody comprises the amino acid sequence set forth in SEQ ID NO: 124;
(ff) the VH of the reference antibody comprises the amino acid sequence set forth in SEQ ID NO: 107 and the VL of the reference antibody comprises the amino acid sequence set forth in SEQ ID NO: 126;
(gg) the VH of the reference antibody comprises the amino acid sequence set forth in SEQ ID NO: 99 and the VL of the reference antibody comprises the amino acid sequence set forth in SEQ ID NO: 128;
(hh) the VH of the reference antibody comprises the amino acid sequence set forth in SEQ ID NO: 99 and the VL of the reference antibody comprises the amino acid sequence set forth in SEQ ID NO: 130;
(ii) the VH of the reference antibody comprises the amino acid sequence set forth in SEQ ID NO: 65 and the VL of the reference antibody comprises the amino acid sequence set forth in SEQ ID NO: 132; or
(jj) the VH of the reference antibody comprises the amino acid sequence set forth in SEQ ID NO: 133 and the VL of the reference antibody comprises the amino acid sequence set forth in SEQ ID NO: 76;
(kk) the VH of the reference antibody comprises the amino acid sequence set forth in SEQ ID NO: 80 and the VL of the reference antibody comprises the amino acid sequence set forth in SEQ ID NO: 62;
(ll) the VH of the reference antibody comprises the amino acid sequence set forth in SEQ ID NO: 81 and the VL of the reference antibody comprises the amino acid sequence set forth in SEQ ID NO: 62;
(mm) the VH of the reference antibody comprises the amino acid sequence set forth in SEQ ID NO: 82 and the VL of the reference antibody comprises the amino acid sequence set forth in SEQ ID NO: 62;
(nn) the VH of the reference antibody comprises the amino acid sequence set forth in SEQ ID NO: 84 and the VL of the reference antibody comprises the amino acid sequence set forth in SEQ ID NO: 62; or
(oo) the VH of the reference antibody comprises the amino acid sequence set forth in SEQ ID NO: 86 and the VL of the reference antibody comprises the amino acid sequence set forth in SEQ ID NO: 62.

24. An isolated antibody or antigen-binding fragment thereof that specifically binds to the same cleaved human CDCP1 epitope as a reference antibody comprising a heavy chain variable region (VH) and a light chain variable region (VL), wherein:
(a) the VH of the reference antibody comprises the amino acid sequence set forth in SEQ ID NO: 61 and the VL of the reference antibody comprises the amino acid sequence set forth in SEQ ID NO: 62;
(b) the VH of the reference antibody comprises the amino acid sequence set forth in SEQ ID NO: 65 and the VL of the reference antibody comprises the amino acid sequence set forth in SEQ ID NO: 62;
(c) the VH of the reference antibody comprises the amino acid sequence set forth in SEQ ID NO 67 and the VL of the reference antibody comprises the amino acid sequence set forth in SEQ ID NO: 62;
(d) the VH of the reference antibody comprises the amino acid sequence set forth in SEQ ID NO: 69 and the VL of the reference antibody comprises the amino acid sequence set forth in SEQ ID NO: 62;
(e) the VH of the reference antibody comprises the amino acid sequence set forth in SEQ ID NO 71; and the VL of the reference antibody comprises the amino acid sequence set forth in SEQ ID NO: 62;
(f) the VH of the reference antibody comprises the amino acid sequence set forth in SEQ ID NO: 73 and the VL of the reference antibody comprises the amino acid sequence set forth in SEQ ID NO: 62;
(g) the VH of the reference antibody comprises the amino acid sequence set forth in SEQ ID NO: 75 and the VL of the reference antibody comprises the amino acid sequence set forth in SEQ ID NO: 76;
(h) the VH of the reference antibody comprises the amino acid sequence set forth in SEQ ID NO: 79 and the VL of the reference antibody comprises the amino acid sequence set forth in SEQ ID NO: 76;
(i) the VH of the reference antibody comprises the amino acid sequence set forth in SEQ ID NO: 65 and the VL of the reference antibody comprises the amino acid sequence set forth in SEQ ID NO: 76;
(j) the VH of the reference antibody comprises the amino acid sequence set forth in SEQ ID NO: 83 and the VL of the reference antibody comprises the amino acid sequence set forth in SEQ ID NO: 62;
(k) the VH of the reference antibody comprises the amino acid sequence set forth in SEQ ID NO: 85 and the VL of the reference antibody comprises the amino acid sequence set forth in SEQ ID NO: 62;
(l) the VH of the reference antibody comprises the amino acid sequence set forth in SEQ ID NO: 87 and the VL of the reference antibody comprises the amino acid sequence set forth in SEQ ID NO: 62;
(m) the VH of the reference antibody comprises the amino acid sequence set forth in SEQ ID NO: 89 and the VL of the reference antibody comprises the amino acid sequence set forth in SEQ ID NO: 62;
(n) the VH of the reference antibody comprises the amino acid sequence set forth in SEQ ID NO: 91 and the VL of the reference antibody comprises the amino acid sequence set forth in SEQ ID NO: 62;
(o) the VH of the reference antibody comprises the amino acid sequence set forth in SEQ ID NO: 65 and the VL of the reference antibody comprises the amino acid sequence set forth in SEQ ID NO: 94;
(p) the VH of the reference antibody comprises the amino acid sequence set forth in SEQ ID NO: 65 and the VL of the reference antibody comprises the amino acid sequence set forth in SEQ ID NO: 96;
(q) the VH of the reference antibody comprises the amino acid sequence set forth in SEQ ID NO: 65 and the VL of the reference antibody comprises the amino acid sequence set forth in SEQ ID NO: 98;
(r) the VH of the reference antibody comprises the amino acid sequence set forth in SEQ ID NO: 99 and the VL of the reference antibody comprises the amino acid sequence set forth in SEQ ID NO: 100;
(s) the VH of the reference antibody comprises the amino acid sequence set forth in SEQ ID NO: 65 and the VL of the reference antibody comprises the amino acid sequence set forth in SEQ ID NO: 102;
(t) the VH of the reference antibody comprises the amino acid sequence set forth in SEQ ID NO:103 and the VL of the reference antibody comprises the amino acid sequence set forth in SEQ ID NO: 104;
(u) the VH of the reference antibody comprises the amino acid sequence set forth in SEQ ID NO: 99 and the VL of the reference antibody comprises the amino acid sequence set forth in SEQ ID NO: 106;
(v) the VH of the reference antibody comprises the amino acid sequence set forth in SEQ ID NO: 107 and the VL of the reference antibody comprises the amino acid sequence set forth in SEQ ID NO: 108;
(w) the VH of the reference antibody comprises the amino acid sequence set forth in SEQ ID NO: 99 and the VL of the reference antibody comprises the amino acid sequence set forth in SEQ ID NO: 110;
(x) the VH of the reference antibody comprises the amino acid sequence set forth in SEQ ID NO: 99 and the VL of the reference antibody comprises the amino acid sequence set forth in SEQ ID NO: 112;
(y) the VH of the reference antibody comprises the amino acid sequence set forth in SEQ ID NO: 65 and the VL of the reference antibody comprises the amino acid sequence set forth in SEQ ID NO: 114;
(aa) the VH of the reference antibody comprises the amino acid sequence set forth in SEQ ID NO: 99 and the VL of the reference antibody comprises the amino acid sequence set forth in SEQ ID NO: 116;
(bb) the VH of the reference antibody comprises the amino acid sequence set forth in SEQ ID NO: 65 and the VL of the reference antibody comprises the amino acid sequence set forth in SEQ ID NO: 118;
(cc) the VH comprises the amino acid sequence set forth in SEQ ID NO: 65 and the VL comprises the amino acid sequence set forth in SEQ ID NO: 120;
(dd) the VH of the reference antibody comprises the amino acid sequence set forth in SEQ ID NO: 65 and the VL of the reference antibody comprises the amino acid sequence set forth in SEQ ID NO: 122;
(ee) the VH of the reference antibody comprises the amino acid sequence set forth in SEQ ID NO: 123 and the VL of the reference antibody comprises the amino acid sequence set forth in SEQ ID NO: 124;
(ff) the VH of the reference antibody comprises the amino acid sequence set forth in SEQ ID NO: 107 and the VL of the reference antibody comprises the amino acid sequence set forth in SEQ ID NO: 126;
(gg) the VH of the reference antibody comprises the amino acid sequence set forth in SEQ ID NO: 99 and the VL of the reference antibody comprises the amino acid sequence set forth in SEQ ID NO: 128;
(hh) the VH of the reference antibody comprises the amino acid sequence set forth in SEQ ID NO: 99 and the VL of the reference antibody comprises the amino acid sequence set forth in SEQ ID NO: 130;
(ii) the VH of the reference antibody comprises the amino acid sequence set forth in SEQ ID NO: 65 and the VL of the reference antibody comprises the amino acid sequence set forth in SEQ ID NO: 132; or
(jj) the VH of the reference antibody comprises the amino acid sequence set forth in SEQ ID NO: 133 and the VL of the reference antibody comprises the amino acid sequence set forth in SEQ ID NO: 76;
(kk) the VH of the reference antibody comprises the amino acid sequence set forth in SEQ ID NO: 80 and the VL of the reference antibody comprises the amino acid sequence set forth in SEQ ID NO: 62;
(ll) the VH of the reference antibody comprises the amino acid sequence set forth in SEQ ID NO: 81 and the VL of the reference antibody comprises the amino acid sequence set forth in SEQ ID NO: 62;
(mm) the VH of the reference antibody comprises the amino acid sequence set forth in SEQ ID NO: 82 and the VL of the reference antibody comprises the amino acid sequence set forth in SEQ ID NO: 62;
(nn) the VH of the reference antibody comprises the amino acid sequence set forth in SEQ ID NO: 84 and the VL of the reference antibody comprises the amino acid sequence set forth in SEQ ID NO: 62; or
(oo) the VH of the reference antibody comprises the amino acid sequence set forth in SEQ ID NO: 86 and the VL of the reference antibody comprises the amino acid sequence set forth in SEQ ID NO: 62.
(kk) the VH of the reference antibody comprises the amino acid sequence set forth in SEQ ID NO: 80 and the VL of the reference antibody comprises the amino acid sequence set forth in SEQ ID NO: 62;
(ll) the VH of the reference antibody comprises the amino acid sequence set forth in SEQ ID NO: 81 and the VL of the reference antibody comprises the amino acid sequence set forth in SEQ ID NO: 62;
(mm) the VH of the reference antibody comprises the amino acid sequence set forth in SEQ ID NO: 82 and the VL of the reference antibody comprises the amino acid sequence set forth in SEQ ID NO: 62;
(nn) the VH of the reference antibody comprises the amino acid sequence set forth in SEQ ID NO: 84 and the VL of the reference antibody comprises the amino acid sequence set forth in SEQ ID NO: 62; or
(oo) the VH of the reference antibody comprises the amino acid sequence set forth in SEQ ID NO: 86 and the VL of the reference antibody comprises the amino acid sequence set forth in SEQ ID NO: 62.

25. An isolated antibody or antigen-binding fragment thereof that cross-competes for binding to cleaved human CDCP1 epitope with a reference antibody comprising a heavy chain variable region (VH) and a light chain variable region (VL), wherein:
(a) the VH of the reference antibody comprises the amino acid sequence set forth in SEQ ID NO: 61 and the VL of the reference antibody comprises the amino acid sequence set forth in SEQ ID NO: 62;
(b) the VH of the reference antibody comprises the amino acid sequence set forth in SEQ ID NO: 65 and the VL of the reference antibody comprises the amino acid sequence set forth in SEQ ID NO: 62;
(c) the VH of the reference antibody comprises the amino acid sequence set forth in SEQ ID NO 67 and the VL of the reference antibody comprises the amino acid sequence set forth in SEQ ID NO: 62;
(d) the VH of the reference antibody comprises the amino acid sequence set forth in SEQ ID NO: 69 and the VL of the reference antibody comprises the amino acid sequence set forth in SEQ ID NO: 62;
(e) the VH of the reference antibody comprises the amino acid sequence set forth in SEQ ID NO 71; and the VL of the reference antibody comprises the amino acid sequence set forth in SEQ ID NO: 62;
(f) the VH of the reference antibody comprises the amino acid sequence set forth in SEQ ID NO: 73 and the VL of the reference antibody comprises the amino acid sequence set forth in SEQ ID NO: 62;
(g) the VH of the reference antibody comprises the amino acid sequence set forth in SEQ ID NO: 75 and the VL of the reference antibody comprises the amino acid sequence set forth in SEQ ID NO: 76;
(h) the VH of the reference antibody comprises the amino acid sequence set forth in SEQ ID NO: 79 and the VL of the reference antibody comprises the amino acid sequence set forth in SEQ ID NO: 76;
(i) the VH of the reference antibody comprises the amino acid sequence set forth in SEQ ID NO: 65 and the VL of the reference antibody comprises the amino acid sequence set forth in SEQ ID NO: 76;
(j) the VH of the reference antibody comprises the amino acid sequence set forth in SEQ ID NO: 83 and the VL of the reference antibody comprises the amino acid sequence set forth in SEQ ID NO: 62;
(k) the VH of the reference antibody comprises the amino acid sequence set forth in SEQ ID NO: 85 and the VL of the reference antibody comprises the amino acid sequence set forth in SEQ ID NO: 62;
(l) the VH of the reference antibody comprises the amino acid sequence set forth in SEQ ID NO: 87 and the VL of the reference antibody comprises the amino acid sequence set forth in SEQ ID NO: 62;
(m) the VH of the reference antibody comprises the amino acid sequence set forth in SEQ ID NO: 89 and the VL of the reference antibody comprises the amino acid sequence set forth in SEQ ID NO: 62;
(n) the VH of the reference antibody comprises the amino acid sequence set forth in SEQ ID NO: 91 and the VL of the reference antibody comprises the amino acid sequence set forth in SEQ ID NO: 62;
(o) the VH of the reference antibody comprises the amino acid sequence set forth in SEQ ID NO: 65 and the VL of the reference antibody comprises the amino acid sequence set forth in SEQ ID NO: 94;
(p) the VH of the reference antibody comprises the amino acid sequence set forth in SEQ ID NO: 65 and the VL of the reference antibody comprises the amino acid sequence set forth in SEQ ID NO: 96;
(q) the VH of the reference antibody comprises the amino acid sequence set forth in SEQ ID NO: 65 and the VL of the reference antibody comprises the amino acid sequence set forth in SEQ ID NO: 98;
(r) the VH of the reference antibody comprises the amino acid sequence set forth in SEQ ID NO: 99 and the VL of the reference antibody comprises the amino acid sequence set forth in SEQ ID NO: 100;
(s) the VH of the reference antibody comprises the amino acid sequence set forth in SEQ ID NO: 65 and the VL of the reference antibody comprises the amino acid sequence set forth in SEQ ID NO: 102;
(t) the VH of the reference antibody comprises the amino acid sequence set forth in SEQ ID NO: 103 and the VL of the reference antibody comprises the amino acid sequence set forth in SEQ ID NO: 104;
(u) the VH of the reference antibody comprises the amino acid sequence set forth in SEQ ID NO: 99 and the VL of the reference antibody comprises the amino acid sequence set forth in SEQ ID NO: 106;
(v) the VH of the reference antibody comprises the amino acid sequence set forth in SEQ ID NO: 107 and the VL of the reference antibody comprises the amino acid sequence set forth in SEQ ID NO: 108;
(w) the VH of the reference antibody comprises the amino acid sequence set forth in SEQ ID NO: 99 and the VL of the reference antibody comprises the amino acid sequence set forth in SEQ ID NO: 110;
(x) the VH of the reference antibody comprises the amino acid sequence set forth in SEQ ID NO: 99 and the VL of the reference antibody comprises the amino acid sequence set forth in SEQ ID NO: 112;
(y) the VH of the reference antibody comprises the amino acid sequence set forth in SEQ ID NO: 65 and the VL of the reference antibody comprises the amino acid sequence set forth in SEQ ID NO: 114;
(aa) the VH of the reference antibody comprises the amino acid sequence set forth in SEQ ID NO: 99 and the VL of the reference antibody comprises the amino acid sequence set forth in SEQ ID NO: 116;
(bb) the VH of the reference antibody comprises the amino acid sequence set forth in SEQ ID NO: 65 and the VL of the reference antibody comprises the amino acid sequence set forth in SEQ ID NO: 118;
(cc) the VH comprises the amino acid sequence set forth in SEQ ID NO: 65 and the VL comprises the amino acid sequence set forth in SEQ ID NO: 120;
(dd) the VH of the reference antibody comprises the amino acid sequence set forth in SEQ ID NO: 65 and the VL of the reference antibody comprises the amino acid sequence set forth in SEQ ID NO: 122;
(ee) the VH of the reference antibody comprises the amino acid sequence set forth in SEQ ID NO: 123 and the VL of the reference antibody comprises the amino acid sequence set forth in SEQ ID NO: 124;
(ff) the VH of the reference antibody comprises the amino acid sequence set forth in SEQ ID NO: 107 and the VL of the reference antibody comprises the amino acid sequence set forth in SEQ ID NO: 126;
(gg) the VH of the reference antibody comprises the amino acid sequence set forth in SEQ ID NO: 99 and the VL of the reference antibody comprises the amino acid sequence set forth in SEQ ID NO: 128;
(hh) the VH of the reference antibody comprises the amino acid sequence set forth in SEQ ID NO: 99 and the VL of the reference antibody comprises the amino acid sequence set forth in SEQ ID NO: 130;
(ii) the VH of the reference antibody comprises the amino acid sequence set forth in SEQ ID NO: 65 and the VL of the reference antibody comprises the amino acid sequence set forth in SEQ ID NO: 132; or
(jj) the VH of the reference antibody comprises the amino acid sequence set forth in SEQ ID NO: 133 and the VL of the reference antibody comprises the amino acid sequence set forth in SEQ ID NO: 76;
(kk) the VH of the reference antibody comprises the amino acid sequence set forth in SEQ ID NO: 80 and the VL of the reference antibody comprises the amino acid sequence set forth in SEQ ID NO: 62;
(ll) the VH of the reference antibody comprises the amino acid sequence set forth in SEQ ID NO: 81 and the VL of the reference antibody comprises the amino acid sequence set forth in SEQ ID NO: 62;
(mm) the VH of the reference antibody comprises the amino acid sequence set forth in SEQ ID NO: 82 and the VL of the reference antibody comprises the amino acid sequence set forth in SEQ ID NO: 62;
(nn) the VH of the reference antibody comprises the amino acid sequence set forth in SEQ ID NO: 84 and the VL of the reference antibody comprises the amino acid sequence set forth in SEQ ID NO: 62; or
(oo) the VH of the reference antibody comprises the amino acid sequence set forth in SEQ ID NO: 86 and the VL of the reference antibody comprises the amino acid sequence set forth in SEQ ID NO: 62.

26. The antibody or antigen-binding fragment thereof of any one of claims 1 to 25, wherein the antibody or antigen-binding fragment thereof has one or more properties selected from the group consisting of:
(a) the antibody inhibits tumor growth and/or metastasis;
(b) the antibody reduces tumor volume;
(c) the antibody increases progression-free survival;
(d) the antibody increases overall survival;
(e) the antibody promotes CDCP1 internalization and/or degradation; and
(f) any combination thereof.

27. The antibody or antigen-binding fragment thereof of any one of claims 1 to 26, wherein the antibody or antigen-binding fragment thereof specifically binds cleaved human CDCP1 with a K_{D} of about 1×10⁻⁴ M or less, wherein K_{D} is measured by biolayer interferometry analysis using an Octet instrument (ForteBio).

28. The antibody or antigen-binding fragment thereof of any one of claims 1 to 27, wherein the antibody or antigen-binding fragment thereof specifically binds cleaved human CDCP1 with an on rate (kₒₙ) of about 1×10⁻⁴ 1/Ms or more, wherein the kₒₙ rate is measured by biolayer interferometry analysis using an Octet instrument (ForteBio).

29. The antibody or antigen-binding fragment thereof of any one of claims 1 to 28, wherein the antibody or antigen-binding fragment thereof specifically binds cleaved human CDCP1 with an off rate (k_{off}) of about 1×10⁻⁴ M 1/s or less, wherein the k_{off} is measured by biolayer interferometry analysis using an Octet instrument (ForteBio).

30. The antibody or antigen-binding fragment thereof of any one of claims 1 to 29, wherein the antibody or antigen-binding fragment thereof binds to cleaved cynomolgus monkey CDCP1.

31. The antibody or antigen-binding fragment thereof of any one of claims 1 to 30, wherein the antibody or antigen-binding fragment is selected from the group consisting of an IgG1, an IgG2, an IgG3, an IgG4 or a variant thereof.

32. The antibody or antigen-binding fragment thereof of any one of claims 1 to 31, wherein the antibody or antigen-binding fragment is an IgG1 antibody.

33. The antibody or antigen-binding fragment thereof of any one of claims 1 to 32, wherein the antibody or antigen-binding fragment is modified to remove a glycosylation site.

34. The antibody or antigen-binding fragment thereof of claim 33, wherein the glycosylation site removal is accomplished via substitution of the asparagine (N) to Aspartic acid (D) at a position that corresponds to residue 31 in SEQ ID NO: 61.

35. The antibody or antigen-binding fragment thereof of any one of claims 1 to 34, wherein the antibody or antigen-binding fragment comprises substitution of methionine (M) to alanine (A), isoleucine (I), leucine (L), or valine (V) at a position that corresponds to residue 114 in SEQ ID NO: 61 or 65.

36. The antibody or antigen-binding fragment thereof of any one of claims 1 to 35, wherein the antibody or antigen-binding fragment thereof is a human, a humanized antibody, a chimeric antibody, or antigen-binding fragment thereof.

37. The antibody or antigen-binding fragment thereof of any one of claims 1 to 36, wherein the antibody or antigen-binding fragment thereof is suitable for administration to a human subject.

38. The antibody or antigen binding fragment thereof of any one of claims 1 to 37, which is a full length antibody.

39. The antibody or antigen binding fragment thereof of any one of claims 1 to 37, which is an antigen binding fragment.

40. The antigen binding fragment of claim 39, wherein the antigen binding fragment is a Fab, Fab′, F(ab′)₂, single chain Fv (scFv), disulfide linked Fv, IgNar, intrabody, IgGΔCH2, minibody, F(ab′)₃, tetrabody, triabody, diabody, single-domain antibody, DVD-Ig, Fcab, mAb², (scFv)₂, or scFv-Fc.

41. A bispecific antibody comprising the antibody or antigen-binding fragment thereof of any one of claims 1-40.

42. A multispecific antibody comprising the bispecific antibody of claim 41 or the antibody or antigen-binding fragment thereof of any one of claims 1 to 40.

43. The antibody or antigen-binding fragment thereof of any one of claims 1 to 40, the bispecific antibody of claim 41, or the multispecific antibody of claim 42 further comprising a detectable label.

44. A polynucleotide or a set of polynucleotides encoding the antibody or antigen-binding fragment thereof of any one of claims 1 to 40, the bispecific antibody of claim 41, or the multispecific antibody of claim 42.

45. A polynucleotide comprising a nucleic acid molecule encoding the heavy chain variable region or heavy chain of the antibody or antigen-binding fragment thereof of any one of claims 10 to 40.

46. The polynucleotide of claim 45, wherein the nucleic acid molecule encodes the VH of SEQ ID NO: 88, 92, 93, 95, 97, 163, 165, 169, 171, 173, 175, 177, 181, 183, 187, 189, 191, 193, 195, 203, 207, 211, or 227.

47. A polynucleotide comprising a nucleic acid molecule encoding the light chain variable region or light chain of the antibody or antigen-binding fragment thereof of any one of claims 10-40.

48. The polynucleotide of claim 47, wherein the nucleic acid molecule encodes the VL of SEQ ID NO: 90,164,166, 180, 198, 200, 202, 204, 206, 208, 210, 212, 214, 216, 218, 220, 222, 224, 226, 228, 230, 232, 234, or 236.

49. A polynucleotide comprising a first nucleic acid molecule encoding the heavy chain variable region of SEQ ID NO: 88, 92, 93, 95, 97, 163, 165, 169, 171, 173, 175, 177, 181, 183, 187, 189, 191, 193, 195, 203, 207, 211, or 227, and a second nucleic acid molecule encoding the light chain variable region of SEQ ID NO: 90,164,166, 180, 198, 200, 202, 204, 206, 208, 210, 212, 214, 216, 218, 220, 222, 224, 226, 228, 230, 232, 234, or 236.

50. A mixture of polynucleotides comprising a first polynucleotide which comprises a nucleic acid molecule encoding the heavy chain variable region of SEQ ID NO:, and a second polynucleotide which comprises a nucleic acid molecule encoding the light chain variable region of SEQ ID NO: 90,164,166, 180, 198, 200, 202, 204, 206, 208, 210, 212, 214, 216, 218, 220, 222, 224, 226, 228, 230, 232, 234, or 236.

51. A polynucleotide comprising a nucleic acid molecule encoding the heavy chain variable region or heavy chain of the antibody or antigen-binding fragment thereof of any one of claims 1 to 40 and the light chain variable region or light chain of the antibody or antigen-binding fragment thereof of any one of claims 1 to 40.

52. A vector comprising the polynucleotide of any one of claims 44 to 51.

53. A host cell comprising (a) the antibody or antigen-binding fragment thereof of any one of claims 1 to 40, (b) the bispecific antibody of claim 41, (c) the multispecific antibody of claim 42, (d) the polynucleotide of any one of claims 44 to 51, (e) the vector of claim 52, or (f) a first vector comprising the polynucleotide of claim 45 or 47 and a second vector comprising the polynucleotide of claim 46 or 48.

54. The host cell of claim 53, which is selected from the group consisting of E. coli, Pseudomonas, Bacillus, Streptomyces, yeast, HPAC, PL5, PL45, HPNE, Expi293F human cell, C6 (rat glioma cell line), U2OS, Chem-1, CHO, YB/20, NSO, PER-C6, HEK-293T, HEK293T-cCDCP1, NIH-3T3, HeLa, BHK, Hep G2, SP2/0, R1.1, B-W, L-M, COS 1, COS 7, BSC1, BSC40, BMT10 cell, PANC-1, Panc 03.27, Hs766T, CFPAC-1, CAPAN-1, Mia PaCa-2, CAPAN-2, BXPC3, mouse Fc1245, mouse Fc1242, mouse Fc1245-cCDCP1mouse PyMT, mouse P53, mouse 4T1, mouse EMT6, mouse TRAMP, mouse C2, mouse MC38, mouse CT26, plant cell, insect cell, and human cell in tissue culture.

55. An immunoconjugate comprising the antibody or antigen-binding fragment thereof of any one of claims 1 to 40, the bispecific antibody of claim 41, or the multispecific antibody of claim 42 and a therapeutic agent.

56. The immunoconjugate of claim 55, wherein the therapeutic agent is selected from the group consisting of a cytotoxin, a non-cytotoxic drug, a radioactive agent, a second antibody, an enzyme, an anti-neoplastic agent, and any combination thereof.

57. A method of producing an antibody or antigen-binding fragment thereof that binds to cleaved human CDCP1 comprising culturing the host cell of claim 53 or 54 so that the nucleic acid molecule is expressed and the antibody or antigen-binding fragment thereof is produced.

58. The method of claim 57, wherein the method further comprises isolating the antibody or antigen-binding fragment thereof from the culture.

59. An isolated antibody or antigen-binding fragment thereof that specifically binds to cleaved human CDCP1 and is encoded by the polynucleotide of any one of claims 44 to 51 or produced by the method of claim 57 or 58.

60. A pharmaceutical composition comprising the antibody or antigen-binding fragment thereof of any one of claims 1 to 40 and 59, the bispecific antibody of claim 41, the multispecific antibody of claim 42, the polynucleotide of any one of claims 44 to 51, the vector of claim 52, or the immunoconjugate of claim 55 or 56, and a pharmaceutically acceptable excipient.

61. The pharmaceutical composition of claim 60, which is formulated for intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural, intrasternal, topical, epidermal, or mucosal administration.

62. A method of treating a cancer in a subject in need thereof, comprising administering to the subject the antibody or antigen binding fragment thereof of any one of claims 1 to 40 and 59, the bispecific antibody of claim 41, the multispecific antibody of claim 42, the polynucleotide of any one of claims 44 to 51, the vector of claim 52, the immunoconjugate of claim 55 or 56, or the pharmaceutical composition of claim 60 or 61.

63. The method of claim 62, wherein administration of the antibody or antigen binding fragment thereof reduces or inhibits metastasis of the cancer in the subject.

64. A method of reducing or inhibiting cancer metastasis in a subject in need thereof, comprising administering to the subject the antibody or antigen binding fragment thereof of any one of claims 1 to 41 and 59, the bispecific antibody of claim 41, the multispecific antibody of claim 42, the polynucleotide of any one of claims 44 to 51, the vector of claim 52, the immunoconjugate of claim 55 or 56, or the pharmaceutical composition of claim 60 or 61.

65. The method of claim 64, wherein the subject is afflicted with a cancer.

66. The method of any one of claims 62 to 65, wherein the cancer has the cleaved CDCP1 present on the cancer cell surface.

67. The method of any one of claims 62 to 66, wherein the cancer comprises a tumor.

68. The method of any one of claims 62 to 67, wherein the cancer is wherein the cancer is selected from the group consisting of small-cell lung cancer (SCLC), non-small cell lung cancer (NSCLC), squamous NSCLC, nonsquamous NSCLC, glioma, gastrointestinal cancer, renal cancer, clear cell carcinoma, ovarian cancer, liver cancer, colorectal cancer, endometrial cancer, kidney cancer, renal cell carcinoma (RCC), prostate cancer, hormone refractory prostate adenocarcinoma, thyroid cancer, neuroblastoma, pancreatic cancer, glioblastoma (glioblastoma multiforme), cervical cancer, stomach cancer, bladder cancer, hepatoma (hepatocellular carcinoma), breast cancer, colon carcinoma, head and neck cancer (or carcinoma), head and neck squamous cell carcinoma (HNSCC), gastric cancer, germ cell tumor, pediatric sarcoma, sinonasal natural killer, melanoma, metastatic malignant melanoma, cutaneous or intraocular malignant melanoma, mesothelioma, bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, neoplasm of the central nervous system (CNS), primary CNS lymphoma, tumor angiogenesis, spinal axis tumor, brain cancer, brain stem glioma, pituitary adenoma, Kaposi's sarcoma, epidermoid cancer, squamous cell cancer, environmentally-induced cancers including those induced by asbestos, virus-related cancers or cancers of viral origin, human papilloma virus (HPV)-related or -originating tumors, acute leukemia (ALL), acute myelogenous leukemia (AML), chronic lymphocytic leukemia (CLL), and chronic myelogenous leukemia (CML), undifferentiated AML, myeloblastic leukemia, myeloblastic leukemia, promyelocytic leukemia, myelomonocytic leukemia, monocytic leukemia, erythroleukemia, megakaryoblastic leukemia, isolated granulocytic sarcoma, chloroma, Hodgkin's lymphoma (HL), non-Hodgkin's lymphoma (NHL), B-cell lymphoma, T-cell lymphoma, lymphoplasmacytoid lymphoma, monocytoid B-cell lymphoma, mucosa-associated lymphoid tissue (MALT) lymphoma, anaplastic large-cell lymphoma, adult T-cell lymphoma/leukemia, mantle cell lymphoma, angio immunoblastic T-cell lymphoma, angiocentric lymphoma, intestinal T-cell lymphoma, primary mediastinal B-cell lymphoma, precursor T-lymphoblastic lymphoma, T-lymphoblastic; peripheral T-cell lymphoma, lymphoblastic lymphoma, post-transplantation lymphoproliferative disorder, true histiocytic lymphoma, primary central nervous system lymphoma, primary effusion lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T-cell lymphoma (CTLC), lymphoplasmacytoid lymphoma (LPL) with Waldenstrom's macroglobulinemia; myeloma, IgG myeloma, light chain myeloma, nonsecretory myeloma, smoldering myeloma (indolent myeloma), solitary plasmocytoma, multiple myeloma, chronic lymphocytic leukemia (CLL), hairy cell lymphoma; and any combinations of said cancers.

69. A method of killing a tumor cell in a subject in need thereof, comprising administering the antibody or antigen binding fragment thereof of any one of claims 1 to 40 and 59, the bispecific antibody of claim 41, the multispecific antibody of claim 42, the polynucleotide of any one of claims 44 to 51, the vector of claim 52, the immunoconjugate of claim 55 or 56, or the pharmaceutical composition of claim 60 or 61.

70. The method of claim 69, wherein tumor cell is metastatic.

71. The method of any one of claims 62 to 70, further comprising administering to the subject an additional anti-cancer therapy.

72. The method of claim 71, wherein the additional anti-cancer therapy comprises a chemotherapy, an immunotherapy, a surgery, a radiotherapy, or any combination thereof.

73. The method of claim 71 or 72, wherein the additional anti-cancer therapy comprises a standard of care therapy.

74. The method of any one of claims 71 to 73, wherein the additional anti-cancer therapy comprises a checkpoint inhibitor.

75. The method of any one of claims 71 to 74, wherein the additional anti-cancer therapy comprises an antibody or an antigen binding fragment thereof that specifically binds a protein selected from Inducible T cell Co-Stimulator (ICOS), CD137 (4-1BB), CD134 (OX40), NKG2A, CD27, CD96, Glucocorticoid-Induced TNFR-Related protein (GITR), and Herpes Virus Entry Mediator (HVEM), Programmed Death-1 (PD-1), Programmed Death Ligand-1 (PD-L1), CTLA-4, B and T Lymphocyte Attenuator (BTLA), T cell Immunoglobulin and Mucin domain-3 (TIM-3), Lymphocyte Activation Gene-3 (LAG-3), adenosine A2a receptor (A2aR), Killer cell Lectin-like Receptor G1 (KLRG-1), Natural Killer Cell Receptor 2B4 (CD244), CD160, T cell Immunoreceptor with Ig and ITIM domains (TIGIT), and the receptor for V-domain Ig Suppressor of T cell Activation (VISTA), KIR, TGFβ, IL-10, IL-8, B7-H4, Fas ligand, CXCR4, mesothelin, CEACAM-1, CD52, HER2, and any combination thereof.

76. The method of claim 75, wherein the anti-PD-1 antibody comprises nivolumab or pembrolizumab.

77. The method of claim 71 or 72, wherein the additional anti-cancer therapy comprises CAR-T cell therapy.

78. The method of any one of claims 62 to 77, wherein the antibody or antigen binding fragment thereof, the bispecific antibody, the multispecific antibody, the polynucleotide, the vector, the host cell, the immunoconjugate, or the pharmaceutical composition is administered intravenously, intraperitoneally, intramuscularly, intraarterially, intrathecally, intralymphaticly, intralesionally, intracapsularly, intraorbitally, intracardiacly, intradermally, transtracheally, subcutaneously, subcuticularly, intraarticularly, subcapsularly, subarachnoidly, intraspinally, epidurally, intrasternally, topically, epidermally, or mucosally.

79. The method of any one of claims 62 to 78, wherein the subject is a human.

80. A method for detecting cleaved human CDCP1 in a sample comprising contacting the sample with the antibody or antigen-binding fragment thereof of any one of claims 1 to 40 and 59, the bispecific antibody of claim 41, the multispecific antibody of claim 42, the immunoconjugate of claim 55 or 56, or the pharmaceutical composition of claim 60 or 61.

81. The method of claim 80, wherein the sample is obtained from a human subject.

82. The method of claim 80 or 81, wherein the sample is a cancer sample.

83. The method of any one of claims 80 to 82, wherein the sample is an in vitro sample.

84. A method of identifying a cancer drug candidate comprising generating an antibody or an antigen-binding fragment thereof that specifically binds to a cleaved human complement C1r/C1s, Uegf, Bmp1 (CUB)-domain containing protein 1 (CDCP1), wherein the antibody or antigen-binding fragment thereof preferentially binds to the cleaved CDCP1.

85. The method of claim 84, wherein the antibody or antigen-binding fragment thereof does not bind to a full-length human CDCP1 at a detectable level.

86. The method of claim 84 or 85, wherein the binding between the antibody or antigen-binding fragment thereof and the cleaved CDCP1 or the full-length CDCP1 is measured by biolayer interferometry analysis using an Octet instrument (ForteBio).

87. The method of any one of claims 84 to 86, wherein the cleaved CDCP1 is generated by being proteolytically cleaved after residue K365, R368, and/or K369 corresponding to SEQ ID NO: 273 from a full-length human CDCP1.

88. The method of any one of claims 84 to 87, wherein the cleaved CDCP1 comprises a membrane-bound complex.

89. The method of claim 88, wherein the membrane-bound complex comprises a first cleaved domain associated with a second cleaved domain

90. The method of claim 89, wherein (i) the first cleaved domain consists of the amino acid sequence as set forth in SEQ ID NO: 63 and the second cleaved domain consists of the amino acid sequence as set forth in SEQ ID NO: 64; (ii) the first cleaved domain consists of the amino acid sequence as set forth in SEQ ID NO: 68 and the second cleaved domain consists of the amino acid sequence as set forth in SEQ ID NO: 70; or (iii) the first cleaved domain consists of the amino acid sequence as set forth in SEQ ID NO: 74 and the second cleaved domain consists of the amino acid sequence as set forth in SEQ ID NO: 77.

91. The method of any one of claims 84 to 90, wherein the cleaved CDCP1 is generated by (a) culturing a host cell comprising a first polynucleotide encoding the first cleaved domain and a second polynucleotide encoding the second cleaved domain and (b) isolating the cleaved CDCP1.

92. An isolated antigen consisting of or consisting essentially of a cleaved CDCP1 protein.

93. The antigen of claim 92, wherein the cleaved CDCP1 is a complex of an N-terminal fragment of CDCP1 and a C-terminal fragment of CDCP1 which have the amino acid sequences as set forth in
(i) SEQ ID NOs: 63 and 64, respectively;
(ii) SEQ ID NOs: 68 and 70, respectively; or
(iii) SEQ ID NOs: 74 and 77, respectively.
